(19) United States
(12) Patent Application Publication
(10) Pub. No.: US 2008/0300147 A1
(43) Pub. Date: Dec. 4, 2008

(54) Detection and Treatment of Fibrotic Disorders

(76) Inventors: Nasser Chegini, Gainesville, FL (US); Xiaoping Luo, Gainesville, FL (US); Li Ding, Gainesville, FL (US); R. Stan Williams, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/590,675

(22) PCT Filed: Mar. 28, 2005
(86) PCT No.: PCT/US05/10257

§ 371 Date: Oct. 18, 2007

Related U.S. Application Data
(60) Provisional application No. 60/556,546, filed on Mar. 26, 2004.
(60) Provisional application No. 60/620,444, filed on Oct. 19, 2004.
(60) Provisional application No. 60/636,240, filed on Dec. 15, 2004.

(51) Int. Cl. C40B 40/06; C12Q 1/68

(52) U.S. Cl. 506/16; 435/6

(57) ABSTRACT

The present invention provides a method for detecting a fibrotic disorder in a subject by: (a) providing a biological sample obtained from the subject (such as endometrium, peritoneal fluid, and/or smooth muscle cells); (b) analyzing the expression of at least one gene that is differentially expressed in the fibrotic disorder of interest; and (c) correlating the expression of the gene(s) with the presence or absence of the fibrotic disorder in the subject. The present invention also provides a method and compositions for modulating the expression of genes that are differentially expressed in fibrotic tissues, compared to normal tissues. Restoration of gene expression to levels associated with normal tissue is expected to ameliorate at least some of the symptoms of the fibrotic disorder. This method includes the step of contacting the tissue with an agent that modulates expression of one or more differentially expressed genes in the tissue. The present invention also includes arrays, such as microfluidic cards, for detecting differential gene expression in samples of fibrotic tissue.

### CROSS-REFERENCE TO RELATED APPLICATIONS

The present application claims the benefit of U.S. Application Ser. Nos. 60/556,546, filed Mar. 26, 2004, 60/620,444, filed Oct. 19, 2004, and 60/636,240, filed Dec. 15, 2004, each of which is hereby incorporated by reference herein in its entirety, including any figures, tables, nucleic acid sequences, amino acid sequences, and drawings.

### GOVERNMENT SUPPORT

The subject invention was made with government support under a research project supported by the National Institutes of Health Grant No. HD37432.

The Sequence Listing for this application is four compact discs labeled “Copy 1”, “Copy 2”, “Copy 3”, and “CRF”. Each copy contains only one file named “03-28-05.ST25.txt” which was created on Mar. 28, 2005. The file is 9,994 KB. The entire contents of each of the computer discs are incorporated herein by reference in their entireties.

### BACKGROUND OF INVENTION

Leiomyomas are benign uterine smooth muscle tumors, accounting for more than 30% of hysterectomies performed in the United States annually. Leiomyomas consist mainly of smooth muscle cells of myometrial origin and a network of connective tissue (Anderson, Semin. Reprod. Endocrinol., 1996, 14:269-282; Chegini, Cytokines and Reproduction, 1999, 133-162).

Abnormal vaginal bleeding, pelvic pain and pelvic masses are among the major symptoms associated with leiomyomas. Leiomyomas are considered to originate from cellular transformation of myometrial smooth muscle cells and/or connective tissue fibroblasts during the reproductive years. The identity of factors that initiate such cellular transformation is not known; however, ovarian steroids are essential for leiomyoma growth, and GnRH anolog (GnRHa) therapy, creating a hypoestrogenic condition, is often used for their medical management (Chegini, N “Implication of growth factor and cytokine networks in leiomyomas” In Cytokines in human reproduction. J. Hill ed. New York, Wiley & Sons Publisher, 2000, 133-162; Maruo, T et al. Hum Reprod Update, 2004, 10:207-20; Takeuchi, H et al. J Obstet Gynaecol Res, 2000, 26:325-331; Steinauer, J et al. Obstet Gynecol, 2004, 103:1331-6; Palomba, S et al. Hum Reprod, 2002, 17:3213-3219; DeManno, D et al. Steroids, 2003, 68:1019-32; Carr, B R et al. J Clin Endocrinol Metab, 1993, 76:1217-1223).

Hypoestrogenic conditions created by GnRHa therapy affect both leiomyoma and myometrium; however, clinical observations indicate a difference in their response to changes in the hormonal environment (Carr, B R et al. J Clin Endocrinol Metab, 1993, 76:1217-1223). In addition to GnRHa therapy, clinical and preclinical assessments of selective estrogen and progesterone receptor modulators, either alone or in combination with GnRHa therapy, have shown efficacy in leiomyoma regression (Steinauer, J et al. Obstet Gynecol, 2004, 103:1331-6; Palomba, S et al. Hum Reprod, 2002, 17:3213-3219; DeManno, D et al. Steroids, 2003, 68:1019-32).

GnRHa-induced leiomyoma regression is accompanied by alterations in uterine arteriole size, blood flow, and cellular content as well as changes in the expression of several growth factors, cytokines, extracellular matrix, proteases, and protease inhibitors (reviewed in Chegini, Cytokines in Human Reproduction, 2000, 133-162; Nowak, Bailliere Best Pract Res. Clin Obstet. Gynaecol., 1999, 13:223-238). Differential expression and autocrine/paracrine action of many of these molecules are considered to play a central role in leiomyoma growth and GnRHa-induced regression (Chegini, Cytokines in Human Reproduction, 2000, 133-162; Nowak, Bailliere Best Pract Res. Clin Obstet. Gynaecol., 1999, 13:223-238).

At the cellular level, a combination of mitotic activity, cellular hypertrophy, and accumulation of extracellular matrix (ECM) are considered to participate in leiomyoma growth (Anderson, Semin. Reprod. Endocrinol., 1996, 14:269-282; Chegini, Cytokines and Reproduction, 1999, 133-162; Stewart et al., J. Clin. Endocrinal Metab., 1994, 79:900-906; Wolanska et al., Mol Cell Biochem., 1998, 189:145-152). Compared to myometrium, leiomyomas are reported to overexpress estrogen and progesterone receptors, and GnRHa therapy lowers their content in both tissues (Stewart et al., Semin, Reprod. Endocrinol., 1995, 10:344-357; Englund et al., J. Clin. Endocrinol Metab., 1998, 83:4092-4092). Clinical and basic science research shows that GnRHa acting through suppression of the pituitary-gonadal axis cause leiomyoma to regress by affecting uterine arteriole size, blood flow at the tumor level. But its effect at cellular and molecular levels in leiomyoma has not been investigated.

With respect to the leiomyoma molecular environment, several genome-wide allel-typing studies have evaluated the association between genomic instability and the pathogenesis of leiomyoma (for review; Ligon, A H and Morton, CC Hum Reprod Update, 2001, 7:8-14). These studies have led to the identification of several candidate genes, however in the majority of cases evidence of genomic instability is either lacking or inconsistent (Ligon, A H and Morton, C C Hum Reprod Update, 2001, 7:8-14), implying the existence of unrecognized pathways that can lead to the development of leiomyoma. Further studies have provided support for various autocrine/paracrine regulators in the pathogenesis of leiomyoma including local estrogen production, growth factors, cytokines, chemokines and their receptors, whose expression are regulated by ovarian steroids (Chegini, N “Implication of growth factor and cytokine networks in leiomyomas” In Cytokines in human reproduction. J. Hill ed. New York, Wiley & Sons Publisher, 2000, 133-162; Maruo, T et al. Hum Reprod Update, 2004, 10:207-20). These studies in many instances demonstrated altered expression of these factors and/or their receptors in leiomyoma compared to normal myometrium. In recent years cDNA microarray has been utilized as a high throughput method to identify a large number of differentially expressed and regulated genes in various tissues and cells. Using this approach, several recent studies have further assisted in fingerprinting the gene expression profile of leiomyoma and myometrium during the menstrual cycle (Tsibris, J C M et al. Fertil Steril, 2002, 78:114-121; Chegini, N et al. J Soc Gynecol Investig, 2003, 10:161-71; Wang, H et al. Fertil Steril, 2003, 80:266-76; Weston, G et al. Mol Hum Reprod, 2003, 9:541-9; Ahn, W S et al. Int J Exp Pathol, 2003, 84:267-79; Quade, B J et al. Genes Chromosomes Cancer, 2004, 40:97-108). However, only the expression of a few of these newly identified genes has been validated, and their regulation and correlation with pathogenesis of leiomyoma remains to be investigated.

With respect to GnRHa therapeutic action, it is traditionally believed to act primarily at the level of the pituitary-gonadal axis, and by suppressing ovarian steroid production causes leiomyoma regression. However, the identification of GnRH and GnRH receptor expression in several peripheral tissues, including the uterus, has implicated an autocrine/paracrine role for GnRH and additional sites of action for GnRHa therapy (Chegini, N et al. J Clin Endocrinol Metab, 1996, 81:3215-3221; Ding, L et al. J Clin Endocrinol Metab, 2004, 89:5549-5557; Chegini, N et al. Mol Cell Endocrinol, 2003, 209:9-16; Xu, J et al. J Clin Endocrinol Metab, 2003, 88:1350-61; Chegini, N and Kornberg, L J Soc Gynecol Investig, 2003, 10:21-6; Chegini, N et al. Mol Hum Reprod, 2002, 8:1071-8). Demonstration of the expression of GnRH, as well as GnRH I and II receptors mRNA in leiomyoma and myometrium and their isolated smooth muscle cells has provided support for this concept (Chegini, N et al. J Clin Endocrinol Metab, 1996, 81:3215-3221; Ding, L et al. J Clin Endocrinol Metab, 2004, 89:5549-5557). Several in vitro studies have also demonstrated GnRHa direct action on various cell types derived from peripheral tissues resulting in alteration of cell growth, apoptosis, the expression of cell cycle proteins, growth factors, pro- and anti-inflammatory cytokines, proteases, and protease inhibitors (Chegini, N “Implication of growth factor and cytokine networks in leiomyomas” In Cytokines in human reproduction. J. Hill ed. New York, Wiley & Sons Publisher, 2000, 133-162; Ding, L et al. J Clin Endocrinol Metab, 2004, 89:5549-5557; Chegini, N et al. Mol Cell Endocrinol, 2003, 209:9-16; Xu, J et al. J Clin Endocrinol Metab, 2003, 88:1350-61; Chegini, N and Kornberg, L J Soc Gynecol Investig, 2003, 10:21-6; Chegini, N et al. Mol Hum Reprod, 2002, 8:1071-8; Klausen, C et al. Prog Brain Res, 2002, 141:111-128; Mizutani, T et al. J Clin Endocrinol Metab, 1998, 83:1253-1255; Wu, X et al. Acta Obstet Gynecol Scand, 2001, 80:497-504). Local expression and differential regulation of these genes influences cell proliferation, differentiation, migration, inflammatory response, angiogenesis, expression of adhesion molecules, ECM turnover and apoptosis, etc., processes that are central to leiomyoma growth and regression (Chegini, N “Implication of growth factor and cytokine networks in leiomyomas” In Cytokines in human reproduction. J. Hill ed. New York, Wiley & Sons Publisher, 2000, 133-162; Maruo, T et al. Hum Reprod Update, 2004, 10:207-20; Chegini, N et al. J Clin Endocrinol Metab, 1996, 81:3215-3221; Ding, L et al. J Clin Endocrinol Metab, 2004, 89:5549-5557; Chegini, N et al. Mol Cell Endocrinol, 2003, 209:9-16; Xu, J et al. J Clin Endocrinol Metab, 2003, 88:1350-61; Chegini, N and Kornberg, L J Soc Gynecol Investig, 2003, 10:21-6; Chegini, N et al. Mol Hum Reprod, 2002, 8:1071-8; Klausen, C et al. Prog Brain Res, 2002, 141:111-128; Mizutani, T et al. J Clin Endocrinol Metab, 1998, 83:1253-1255; Wu, X et al. Acta Obstet Gynecol Scand, 2001, 80:497-504; Dou, Q et al. Mol Hum Reprod, 1997, 3:1005-1014; Chegini, N et al. J Clin Endocrinol Metab, 1999, 84:4138-4143; Senturk, L M et al. Am J Obstet Gynecol, 2001, 184:559-566; Sozen, I et al Fertil Steril, 1998, 69:1095-1102; Gustavsson, I et al. Mol Hum Reprod, 2000, 6:55-59; Orii, A et al. J Clin Endocrinol Metab, 2002, 87:3754-9; Fukuhara, K et al. J Clin Endocrinol Metab, 2002, 87:1729-36; Zhai, Y L et al. Int J Cancer, 1999, 84:244-50; Ma, C and Chegini, N Mol Hum Repord, 1999, 5:950-954). Microarray studies, including a small-scaled array, have also identified the expression profile of additional genes targeted by GnRHa in murine gonadotrope tumor cell line LβT2, human breast tumor cell line MCF-7 and leiomyoma and myometrium (Chegini, N et al. J Soc Gynecol Investig, 2003, 10:161-71; Ma, C and Chegini, N Mol Hum Repord, 1999, 5:950-954; Kakar, S S et al. Gene, 2003, 308:67-77).

Transforming growth factors beta (TGF-β) is a multifunctional cytokine and key regulator of cell growth and differentiation, inflammation, apoptosis and tissue remodeling (Blobe, G C et al. N Engl J Med, 2000, 342:1350-1358; Flanders, K C Int J Exp Pathol, 2004, 85:47-64; Schnaper, H W et al. Am J Physiol Renal Physiol, 2003, 284:F243-252; Clancy, R M and Buyon, J P J Leukoc Biol, 2003, 74:959-960; Olman, M A and Matthay, M A Am J Physiol Lung Cell Mol Physiol, 2003, 285:L522-6). While under normal physiological conditions the expression and autocrine/paracrine actions of TGF-β are highly regulated, alteration in TGF-β and TGF-β receptor expression and their signaling mechanisms often result in various pathological disorders, including fibrosis (Blobe, G C et al. N Engl J Med, 2000, 342:1350-1358; Flanders, K C Int J Exp Pathol, 2004, 85:47-64; Schnaper, H W et al. Am J Physiol Renal Physiol, 2003, 284:F243-252; Clancy, R M and Buyon, J P J Leukoc Biol, 2003, 74:959-960; Olman, M A and Matthay, M A Am J Physiol Lung Cell Mol Physiol, 2003, 285:L522-6). Altered expression of TGF-β isoforms (TGF-β1, β2 and β3) and TGF-β receptors (type I, II and III) in leiomyoma and their isolated smooth muscle cells (LSMC) compared to normal myometrium has been observed (Dou, Q et al. J Clin Endocrinol Metab, 1996, 81:3222-3230; Chegini, N et al. J Clin Endocrinol Metab, 1999, 84:4138-43; Chegini, N et al. Mol Hum Reprod, 2002, 8:1071-1078; Chegini, N et al. Mol Cell Endocrinol, 2003, 209:9-16). Recently, it has also been demonstrated that leiomyoma and LSMC express elevated levels of Smads, components of the TGF-β receptor signaling pathway, compared to myometrium and MSMC (Chegini, N et al. Mol Cell Endocrinol, 2003, 209:9-16; Xu, J et al. J Clin Endocrinol Metab, 2003, 88:1350-1361). TGF-β regulates its own expression and the expression of Smad in LSMC and MSMC, and through downstream signaling from this and MAPK pathways regulates the expression of c-fos, c-jun, fibronectin, type I collagen and plasminogen activator inhibitor 1 in these cells (Chegini, N et al. J Clin Endocrinol Metab, 1999, 84:4138-43; Chegini, N et al. Mol Hum Reprod, 2002, 8:1071-1078; Ding, L et al. J Clin Endocrinol Metab, 2004, 89:5549-5557). Additionally, data have demonstrated the ability of TGF-β to regulate LSMC and MSMC cell growth (Tang, X M et al. Mol Hum Reprod, 1997, 3:233-40; Arici, A and Sozen, I Am J Obstet Gynecol, 2003, 188:76-83; Lee, B S and Nowak, R A J Clin Endocrinol Metab, 2001, 86:913-920; Arici, A and Sozen, I Fertil Steril, 2000, 73:1006-1011).

Because leiomyoma growth is dependent on ovarian steroids, GnRHa therapy and most recently selective estrogen and progesterone receptors modulators are used for their medical management (Steinauer, J et al. Obstet Gynecol, 2004, 103:1331-6; Palomba, S et al. Hum Reprod, 2002, 17:3213-3219; DeManno, D et al. Steroids, 2003, 68:1019-32). It has been demonstrated that GnRHa therapy results in a marked down-regulation of TGF-β isoforms and TGF-β receptors expression and alters the expression and activation of Smads in leiomyoma as well as LSMC (Dou, Q et al. J Clin Endocrinol Metab, 1996, 81:3222-3230; Chegini, N et al Mol Hum Reprod, 2002, 8:1071-1078; Chegini, N et al. Mol Cell Endocrinol, 2003, 209:9-16). It has also been shown that TGF-β expression in LSMC and MSMC is inversely regulated by ovarian steroid compared to their antagonists, ICI-182780, ZK98299, and RU486 (Chegini, N et al. Mol Hum Reprod, 2002, 8:1071-1078). In addition, it has been shown that other cytokines such as GM-CSF, IL-13 and IL-15, which promotes myofibroblast transition, granulation tissue formation and inflammatory response, respectively, may mediate their action either directly or through induction of TGF-β expression in LSMC and MSMC (Chegini, N et al. J Clin Endocrinol Metab, 1999, 84:4138-43; Chegini, N et al. Mol Cell Endocrinol, 2003, 209:9-16; Ding, L et al. J Soc Gyncol Invest, 2004, 00, 00). From these observations, it was proposed that the TGF-β system serves as a major autocrine/paracrine regulator of fibrosis in leiomyoma (Dou, Q et al. J Clin Endocrinol Metab, 1996, Chegini, N et al. J Clin Endocrinol Metab, 1999; 81:3222-3230; Chegini, N et al. Mol Hum Reprod, 2002, 8:1071-1078; Chegini, N et al. Mol Cell Endocrinol, 2003, 209:9-16; Xu, J et al. J Clin Endocrinol Metab, 2003, 88:1350-1361; Ding, L et al. J Clin Endocrinol Metab, 2004, 89:5549-5557; Tang, X M et al. Mol Hum Reprod, 1997, 3:233-40). Evidence has been developed reflecting the molecular environments directed by GnRHa therapy in leiomyoma and myometrium, as well as by GnRHa direct action in LSMC and MSMC (Chegini, N et al. J Soc Gynecol Investig, 2003, 10:161-71).

### BRIEF SUMMARY OF INVENTION

The present invention provides a method for detecting a fibrotic disorder in a subject by: (a) providing a biological sample obtained from the subject (such as endometrium, peritoneal fluid, and/or smooth muscle cells); (b) analyzing the expression of at least one gene that is differentially expressed in the fibrotic disorder of interest as compared to normal tissue (such as myometrium); and (c) correlating the expression of the gene(s) with the presence or absence of the fibrotic disorder in the subject. Preferably, the fibrotic disorder is a fibrotic disorder of the female reproductive tract. Examples of reproductive tract disorders include, but are not limited to, leiomyoma, endometriosis, ovarian hyperstimulation syndrome, adhesions, endometrial cancer, and other tissue fibroses. Fibrosis involves the deposition of large amounts of extracellular matrix molecules, notably collagen. Fibrosis is involved in normal physiological responses (e.g., wound healing) as well as pathophysiological conditions such as renal failure, liver cirrhosis and heart disease. The compositions and methods of the present invention are useful for detecting or treating abnormal fibrotic changes in the tissue of a subject.

Differentially expressed genes include those that are differentially expressed in a given fibrotic disorder (such as leiomyoma), including but not limited to, docking protein 1, 62 kD (downstream of tyrosine kinase 1); centromere protein A (17 kD); catenin (cadherin-associated protein), beta 1 (88 kD); nuclear receptor subfamily 1, group I, member 2; v-rel avian reticuloendotheliosis viral oncogene homolog A; LGN Protein; CDC28 protein kinase 1; hypothetical protein; solute carrier family 17 (sodium phosphate), member 1; FOS-like antigen-1; nuclear matrix protein p84; LERK-6 (EPLG6); visinin-like 1; phosphodiesterase 10A; KH-type splicing regulatory protein (FUSE binding protein 2); Polyposis locus (DP1 gene) mRNA; microtubule-associated protein 2; CDC5 (cell division cycle 5, S pombe, homolog)-like; Centromere autoantigen C (CENPC) mRNA; RNA guanylyltransferase and 5′-phosphatase; Nijmegen breakage syndrome 1 (nibrin); ribonuclease, RNase A family, 4; keratin 10 (epidermolytic hyperkeratosis; keratosis palmaris et plantaris); basic helix-loop-helix domain containing, class B, 2; dual specificity phosphatase 1; annexin A11; putative receptor protein; Human endogenous retrovirus HERV-K(HML6); mitogen-activated protein kinase kinase kinase 12; TXK tyrosine kinase; kynureninase (L-kynurenine hydrolase); ubiquitin specific protease 4 (proto-oncogene); peroxisome biogenesis factor 13; olfactory receptor, family 2, subfamily F, member 1; membrane protein, palmitoylated 3 (MAGUK p55 subfamily member 3); origin recognition complex, subunit 1 (yeast homolog)-like; dTDP-D-glucose 4,6-dehydratase; cytochrome c oxidase subunit VIa polypeptide 2; gamma-tubulin complex protein 2; Monocyte chemotactic protein-3; myelin transcription factor 1; inhibitor of growth family, member 1-like; thyroid hormone receptor, alpha myosin-binding protein C, slow-type; fragile X mental retardation 2; sonic hedgehog (Drosophila) homolog; 6-phosphofructo-2-kinase/fructose-2,6-biphosphatase 2; SFRS protein kinase 2; excision repair cross-complementing rodent repair deficiency; cyclin-dependent kinase 5, regulatory subunit 1 (p35); poly(A)-specific ribonuclease (deadenylation nuclease); solute carrier family 12 (potassium/chloride transporters), member 4; Pseudogene for metallothionein; natriuretic peptide precursor A; intercellular adhesion molecule 2; apoptosis antagonizing transcription factor; similar to rat HREV107; major histocompatibility complex, class II, DP beta 1; MpV17 transgene, murine homolog, glomerulosclerosis; uroporphyrinogen decarboxylase; proteasome (prosome, macropain) 26S subunit, ATPase, 1; fms-related tyrosine kinase 3 ligand; actin, gamma 1; Protein Kinase Pitslre, Alpha, Alt. Splice 1-Feb; nuclear factor of kappa light polypeptide gene enhancer in B-cells inhibitor, alpha; pyruvate kinase, muscle; telomeric repeat binding factor 2; cell division cycle 2, G1 to S and G2 to M; ADP-ribosylation factor 3; NRF1 Protein; H factor (complement)-like 3; serine (or cysteine) proteinase inhibitor, clade B (ovalbumin), member 6; mRNA of muscle specific gene M9; solute carrier family 25 (mitochondrial carrier; phosphate carrier), member 3; ribosomal protein L36a; suppressor of Ty (S. cerevisiae) 4 homolog 1; amino-terminal enhancer of split; ubiquitin A-52 residue ribosomal protein fusion product 1; hydroxyacyl-Coenzyme A dehydrogenase/3-ketoacyl-Coenzyme A thiolase; chaperonin containing TCP1, subunit 2 (beta); tyrosine kinase with immunoglobulin and epidermal growth factor homology; domains; Fc fragment of IgG, receptor, transporter, alpha; NRD1 convertase; ADP-ribosylation factor 5; transcription elongation factor A (SII), 1; like mouse brain protein E46; titin; fibromodulin; Abi-interactor 2 (Abi-2); and other differentially expressed genes disclosed herein.

In one embodiment, the differentially expressed gene is at least one of CDKN1B, CDKN1C, CTGF, fibromodulin, and Abi-2.

In another embodiment, the differentially expressed gene is at least one of IL-11, IL-13, EGR1, EGR2, EGR3, CITED2, P300, E2F1, E2F2, E2F3, E2F4, E2F5, MCP3, CXCL5, CCL7, SMAD3, TYMS, GT198, SMAD7, NCOR2, TIMP-1, and ADAM17, wherein elevated expression of IL-11, IL-13, EGR1, EGR2, EGR3, CITED2, P300, E2F1, E2F2, E2F3, E2F4, E2F5, MCP3, CXCL5, CCL7, SMAD3, TYMS, and/or GT198 is indicative of a fibrotic disorder; and wherein reduced expression of SMAD7, NCOR2, TIMP-1, and/or ADAM17 is indicative of a fibrotic disorder.

In another embodiment, the differentially expressed gene is at least one listed in Table 9 herein.

The step of analyzing expression of the differentially expressed gene can be performed by quantifying the amount of differentially expressed gene product present in the sample, e.g., by contacting the sample with an antibody that specifically binds the gene product. This step can also be performed by quantifying the amount of a nucleic acid (e.g., DNA or RNA) that encodes the gene product present in the sample, e.g., by contacting the sample with a polynucleotide that hybridizes under stringent conditions to the nucleic acid that encodes the gene product. The latter can also be performed using a polymerase chain reaction (PCR), for example.

Preferably, expression of a plurality of differentially expressed genes is analyzed. In this case, step (c) of correlating the expression of the differentially expressed gene with the presence or absence of the fibrotic disorder in the subject can include determining the ratio of two or more differentially expressed gene products in the sample.

In another aspect, the invention features a method for modulating gene expression in fibrotic tissue. This method includes contacting the fibrotic tissue in vitro or in vivo with an agent that modulates expression of a differentially expressed gene in the tissue. Preferably, the fibrotic tissue is tissue from a subject with leiomyoma, endometriosis, ovarian hyperstimulation syndrome, adhesions, or other tissue fibroses of the female reproductive tract, for example. The agent can be one that specifically binds the product that is expressed by a differentially expressed gene. The agent can also be a nucleic acid that modulates (i.e., increases or decreases) expression of one or more differentially expressed genes in a cell. The agent can also be one that modulates transcription or translation of a nucleic acid encoding the product of one or more differentially expressed genes, such as antisense oligonucleotide, ribozyme, or small interfering RNA (siRNA). Nucleic acid molecules that are modulators of differentially expressed genes in fibrotic tissue can be administered, for example, in a viral vector (such as lentivirus) or non-viral vector (such as a liposome). In other variations of this method, the agent can be an ovarian steroid, such as estradiol and medroxyprogesterone actetate. However, the agent is preferably not a hormone, but is nonetheless capable of modulating the expression of one or more genes that are differentially expressed in a fibrotic disorder, such as those genes that are differentially expressed upon GnRHa therapy.

In a preferred embodiment, the agent that modulates expression of a differentially expressed gene in fibrotic tissue is one that decreases or down-regulates the action or expression of one or more genes selected from the group consisting of IL-11, IL-13, EGR1, EGR2, EGR3, CITED2, P300, E2F1, E2F2, E2F3, E2F4, E2F5, MCP3, CXCL5, CCL7, SMAD3, TYMS, and/or GT198. In a another preferred embodiment, the agent that modulates expression of a differentially expressed gene in fibrotic tissue is one that increases or up-regulates the action or expression of one or more genes selected from the group consisting of SMAD-7, NCOR2, TIMP-1, and ADAM17. More preferably, the agent decreases or down-regulates the action or expression of one or more genes selected from the group consisting of IL-11, IL-13, EGR1, EGR2, EGR3, CITED2, P300, E2F1, E2F2, E2F3, E2F4, E2F5, MCP3, CXCL5, CCL7, SMAD3, TYMS, and/or GT198, and increases or up-regulates the action or expression of one or more genes selected from the group consisting of SMAD-7, NCOR2, TIMP-1, and ADAM17.

In one embodiment, the agent that modulates expression of a differentially expressed gene in fibrotic tissue is selected from the group consisting of a selective estrogen receptor modulator (such as Roloxifene or other SERM), a selective progesterone receptor modulator (such as Asoprisnil (J867), RU486, or other SPRM), SB-505124, SB-431542, a mast cell inhibitor (such as Tranlist), Cystatin C (CystC), SD-208, LY550410, LY580276, Pitavastatin, 1,5 naphthyridine amiothiazole derivative, 1,5 naphthyridine pyrazole derivative, and ursolic acid (see, for example, Yingling, J. et al., Nat. Rev. Drug Discov., 2004, Dec.; 3(12):1011-22, which is incorporated herein by reference in its entirety). In another embodiment, the agent is one based on a pyrazolopyridine scaffold (Beight, D. W. et al., WO 2004/026871), a pyrazole scaffold (Gellibert, F. et al., J. Med. Chem., 2004, 47:4494-4506), an imidazopyridine scaffold (Lee, W. C. et al., Wo 2004/021989), triazole scaffold (Blumberg, L. C. et al., WO 2004/026307), a pyridopyrimidine scaffold (Chakravarty, S. et al., WO 2000/012497), or an isothiazole scaffold (Munchhof, M. J., WO 2004/147574), each of which is incorporated herein by reference in its entirety. In another embodiment, the agent is a GnRhH agonist or antagonist, such as those disclosed herein.

Preferably, the agent administered to the subject for treatment or prevention of fibrosis is one that inhibits (reduces) TGF-beta signaling (signal transduction). More preferably, the agent administered to the subject is one that inhibits (reduces) TGF-beta II signaling (signal transduction). Preferably, the inhibition is selective, as opposed to “upstream” of TGF-beta II.

In another aspect of the method of the invention, the subject invention includes a method for treating (alleviating symptoms associated with) fibrotic tissue or reducing the likelihood of fibrotic tissue formation, by administering GnRH analog (e.g., GnRH agonist or antagonist) locally to the target site. For example, the GnRH analog can be administered directly to a fibroid to reduce the size of the fibroid.

In another aspect, the present invention includes a method for identifying a modulator of a gene that is differentially-expressed in fibrotic tissue and/or during fibrogenesis, or a polypeptide encoded by the differentially-expressed gene, in a cell population, comprising: contacting the cell population with a test agent under conditions effective for the test agent to modulate a differentially-expressed gene disclosed herein, to modulate the biological activity of a polypeptide encoded by the differentially-expressed gene; and determining whether the test agent modulates the expression of the gene or biological activity of the polypeptide encoded by the gene. In one embodiment, the determining step is carried out by detecting mRNA or the polypeptide of the differentially expressed gene. Preferably, the cell population comprises mammalian cells (such as human cells) of the female reproductive tract (such as endometrial cells). In one embodiment, the differentially expressed gene is selected from the group consisting of IL-11, IL-13, EGR1, EGR2, EGR3, CITED2, P300, E2F1, E2F2, E2F3, E2F4, E2F5, MCP3, CXCL5, CCL7, SMAD3, TYMS, GT198, SMAD-7, NCOR2, TIMP-1, and ADAM17. Preferred modulators are those that decrease the activity of or down-regulate the expression of one or more of IL-11, IL-13, EGR1, EGR2, EGR3, CITED2, P300, E2F1, E2F2, E2F3, E2F4, E2F5, MCP3, CXCL5, CCL7, SMAD3, TYMS, and GT198, or increase the activity of or up-regulate the expression of one or more of SMAD-7, NCOR2, TIMP-1, and ADAM17. More preferably, the modulator decreases the activity of or down-regulates the expression of one or more of IL-11, IL-13, EGR1, EGR2, EGR3, CITED2, P300, E2F1, E2F2, E2F3, E2F4, E2F5, MCP3, CXCL5, CCL7, SMAD3, TYMS, and GT198; and increases the activity of or up-regulates the expression of one or more of SMAD-7, NCOR2, TIMP-1, and ADAM17. In one embodiment, the identified modulator modulates one or more genes (up to and including all the genes) listed in Table 9 herein.

The present invention also includes arrays, such as microfluidic cards, for detecting differential gene expression in samples of fibrotic tissue.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1J show the expression profile of a selected group of genes representing growth factors/cytokines/polypeptide hormones/receptors (FIGS. 1A-1B), intracellular signal transduction pathways (FIGS. 1C-1D), transcription factors (FIGS. 1E-1F), cell cycle (FIGS. 1G-1H) and cell adhesion/ECM/cytoskeletons (FIGS. 1I-1J) in response to time-dependent action of GnRHa in LSMC and MSMC. Values on the x-axis represent an arbitrary unit derived from the mean gene expression value for each factor after supervised analysis, statistical analysis in R programming environment and ANOVA, with gene expression values for the untreated controls (Ctrl) set at 1.

FIGS. 2A-2J show comparative analysis of the expression profile of 10 genes identified as differentially expressed in response to GnRH therapy in leiomyoma and matched myometrium and untreated group by microarray and Realtime PCR. Values on the x-axis represent an arbitrary unit derived from the mean expression value for each gene with values for the untreated controls (Crtl) set at 1. Total RNA isolated from these tissues was used for both microarray analysis and Realtime PCR validating the expression of IL-11, EGR3, CITED2, Nur77, TEIG, TGIF, p27, p57, Gas1 and GPRK5. On the Y-axis untreated myometrium and leiomyoma are designated as Unt-MM and Un-LM, and GnRH-treated as GnRH-Trt MM and GnRH-Trt LM.

FIGS. 3A-3T show comparative analysis of the expression profile of 10 genes identified as differentially expressed and regulated in response to GnRHa time-dependent action in LSMC and MSMC by microarray and Realtime PCR. Values on the x-axis represent an arbitrary unit derived from the mean expression value for each gene, and y-axis represent the time course of GnRHa (0.1 μM) treatment (2, 6 and 12 hours) with untreated control (Crtl) gene expression values set at 1. Total RNA isolated from these cells used for both microarray analysis and Realtime PCR for validating the expression of IL-11, EGR3, TEIG, TGIF, CITED2, Nur77, CDKN1B (p27), CDKN1C (p57), Gas1 and GPRK5.

FIGS. 4A-4E show immunohistochemical localization of IL-11, TGIF, TIEG, Nur77, EGR3, CITED2, p27, p57 and Gas1 in leiomyoma and myometrium. Note the presence of immunoreactive IL-11, TGIF, TIEG, Nur77, EGR3, CITED2, p27, p57 and Gas1 in association with leiomyoma and myometrial smooth muscle cells, and cellular components of connective tissue and vasculature. Both nuclear (EGR3, Nur77, p27, p57) and cytoplasmic (IL-11) staining is observed. Incubation of tissue sections with non-immune mouse (A), rabbit (B) and goat (figure not shown) IgGs instead of primary antibodies during immunostaining served as controls (Ctrl) reduced the staining intensity. Mag: X150 and X300.

FIGS. 5A-5N show the expression profile of a group of genes representing growth factors/cytokines/polypeptide hormones/receptors (FIGS. 5A-5B), intracellular signal transduction pathways (FIGS. 5C-5D), transcription factors (FIGS. 5E-5F), cell cycle (FIGS. 5G-5H) and cell adhesion/ECM/cytoskeletons (FIGS. 5I-5J) in response to time-dependent action of TGF-β in LSMC and MSMC. Values on the x-axis represent an arbitrary unit derived from the mean gene expression value for each factor after supervised analysis, statistical analysis in R programming environment and ANOVA, with gene expression values for the untreated controls (Ctrl) set at 1.

FIGS. 6A-6R show comparative analysis of the expression profile of 12 genes identified as differentially expressed and regulated in response to time-dependent action of TGF-β1 in LSMC and matched MSMC by microarray and Realtime PCR. Values on the x-axis represent an arbitrary unit derived from the mean expression value for each gene and y-axis represent the time course of TGF-β (2.5 ng/ml) treatment (2, 6 and 12 hours) with untreated control (Crtl) gene expression values set at 1. Total RNA isolated from these cells was used for both microarray analysis and Realtime PCR validating the expression of IL-11, EGR3, CITED2, Nur77, TEIG, TGIF, Runx1, Runx2, p27, p57, Gas1 and GPRK5.

FIGS. 7A-7E show a comparative analysis of the expression profile of Runx1 and Runx2 genes in leiomyoma (LM) and matched myometrium (MM) from untreated (un-Trt) and women who received GnRHa therapy (GnRHa-Trt) as well as in leiomyoma and myometrial smooth muscle cells (LSMC and MSMC) in response to GnRHa (0.1 μM) time dependent action (2, 6 and 12 hours) and in response to time-dependent (2, 6 and 12 hours) action of TGF-β1 (2.5 ng/ml) determined by Realtime PCR. In microarray analysis Runx2 expression was not included since its expression value did not reach the study standard. Values on the x-axis represent an arbitrary unit derived from the mean expression value for each gene and y-axis represents the time course of TGF-beta and GnRHa treatments, with untreated control (Crtl) gene expression values set at 1. Total RNA isolated from these cells was used for both microarray analysis and Realtime PCR validation.

FIGS. 8A-8E are bar graphs showing mean ±SEM of relative mRNA expression levels of CCN2, CCN3, CCN4, fibulin-1C and S100A4 in leiomyoma (LM) and matched myometrium (MM) from untreated (Un-Trt) and GnRH treated (GnRH-Trt) groups (N=12) determined by Real-time PCR. Values on the Y-axis represent an arbitrary unit derived from the mean expression value for each gene with values for the untreated MM (Un-TrtMM) set at 1. Total RNA isolated from tissues including tissues used for microarray analysis (Luo X. et al., Endocrinology 146:1074-1095). For CCN2, denotes b, c and d are statistically different from a, and d is different from c. For CCN3 and S100A4 denotes b, c and d are different from a. For CCN4, denotes b and c are different from a. For fibulin-1C, denotes c and d are different from a and b. All with p<0.05.

FIG. 9 shows Western blot analysis of CCN2, CCN3, CCN4 and fibulin-1C in 9 paired myometrium (M) and leiomyoma (L) from proliferative (N=3) and secretory (N=3) phases of the menstrual cycle, and from women who received GnRHa therapy (GnRHa-treated, N=3). Total protein was isolated from these tissues and equal amount of protein was subjected to immunoblotting using antibodies specific to CCN2, CCN3, CCN4 and fibulin-1C.

FIGS. 10A-10L show immunohistochemical localization of CCN2 (FIGS. 10A and 10B), CCN3 (FIGS. 10C and 10D), CCN4 (FIGS. 10E and 10F), fibulin-1C (FIGS. 10G and 10H) and S100A4 (FIGS. 10I and 10J) in leiomyoma and myometrium with immunoreactive proteins in association with leiomyoma and myometrial smooth muscle cells, and cellular components of connective tissue and vasculature. Incubation of tissue sections with non-immune rabbit (FIG. 10K) and goat (FIG. 10L) IgGs, instead of primary antibodies during immunostaining served as controls reduced the staining intensity. Mag: X60.

FIGS. 11A and 11B are bar graphs showing the mean ±SEM of relative mRNA expression of TGF-β1 and TGF-β3 in leiomyoma and matched myometrium. Total RNA was isolated from paired tissues (N=12) and subjected to Realtime PCR. Total protein isolated from these tissues and equal amount of protein was subjected to ELISA before and after activation. Denotes a and b are significantly different from c and d, respectively; and denotes a and c are statistically different from b and d with P<0.05. Arrows point out the significant differences between the expression of TGF-β1 and TGF-β3 mRNA expression and total and active TGF-β1 in leiomyoma and myometrium.

FIGS. 12A-12E are bar graphs whowing relative mRNA expression of CCN2, CCN3, CCN4, fibulin-1C and S100A4 in leiomyoma (LSMC) and myometrial (MSMC) smooth muscle cells following treatment with TGF-β1 (2.5 ng/ml) for 2, 6 and 12 hrs. Total RNA was isolated from treated and untreated control (Ctrl) cells and subjected to Realtime PCR. Results are the mean ±SEM of three experiments performed using independent cell cultures from different tissues. For CCN2, denotes b, b′, c, c′, d and d′; for CCN3 denotes b, b′, c, c′, and d; for CCN4, denotes b, c, c′, d and d′; for fibulin-1C, denotes b and d; and for S100A4 denote c′, d and d′ are statistically different from a and a′ respectively, with P<0.05. Arrows point out the significant differences between the expression of CCNs, fibulin-1C and S100A4 in LSMC and MSMC.

FIGS. 13A-13E are bar graphs showing the relative mRNA expression of CCN2, CCN3, CCN4, fibulin-1C and S100A4 in leiomyoma (LSMC) and myometrial (MSMC) smooth muscle cells following treatment with GnRHa (0.1 μM) for 2, 6 and 12 hrs. Total RNA was isolated from treated and untreated control (Ctrl) cells and subjected to Realtime PCR. Results are the mean ±SEM of three experiments performed using independent cell cultures from different tissues. For CCN2, denotes b, c′, d and d′; for CCN3 denotes b, b′, c, c′, d and d′; for CCN4, denotes b, b′, c, and d′; for fibulin-1C, denotes b, b′, c, c′, d and d′; and for S100A4 denote b, b′, c, c′, d and d′ are statistically different from a and a′, respectively with P<0.05. Arrows point out the significant differences between the expression of CCNs, fibulin-1C and S100A4 in LSMC and MSMC.

FIGS. 14A-14E are bar graphs showing the relative mRNA expression of CCN2, CCN3, CCN4, fibulin-1C and S100A4 in leiomyoma (LSMC) and myometrial (MSMC) smooth muscle cells pretreated with U0126 (U) MEK1/2MAPK inhibitor followed by treatment with GnRHa and TGF-β1. Serum-starved cells were pretreated with U0126 at 20 μM for 2 hrs, washed and then treated with 2.5 ng/ml of TGF-β1, or 0.1 μM of GnRH for 2 hrs. Total RNA was isolated from treated and untreated controls (Ctrl) and subjected to Realtime PCR. Results are the mean ±SEM of three experiments performed using independent cell cultures from different tissues. Denotes * are significantly different from control and **, and denotes *** are significantly different from * and control with P<0.05, respectively. Arrows point out the significant differences between the expression of CCNs, fibulin-1C and S100A4 in LSMC as compared with MSMC.

FIGS. 15A-15E are bar graphs showing relative mRNA expression of CCN2, CCN3, CCN4, fibulin-1C and S100A4 in leiomyoma (LSMC) and myometrial (MSMC) smooth muscle cells transfected with Smad SiRNA (SmadSi) and treatment with TGF-β 1. The cells were transfected with Smad3 SiRNA or scrambled SiRNA for 48 hrs washed and then treated with 2.5 ng/ml of TGF-β1 for 2 hrs. Total RNA was isolated from treated and untreated controls (Ctrl) and subjected to Realtime PCR. Results are the mean ±SEM of three experiments performed using independent cell cultures from different tissues. Denotes * are significantly different from ** and ***, as well as *** are significantly different from ** with P<0.05, respectively. Arrows point out the significant differences between the expression of CCNs, fibulin-1C and S100A4 in LSMC as compared with MSMC.

FIG. 16 is a bar graph showing the relative expression of fibromodulin mRNA in leiomyoma (LM) and matched myometrium (MM) from untreated (Un-Trt) and GnRH treated (GnRH-Trt) groups determined by real-time PCR. Values on the Y-axis represent an arbitrary unit derived from the mean expression value for each gene with values for the untreated MM (Un-TrtMM) set at 1. Total RNA isolated from tissues used for both microarray analysis (Luo, X. et al. Endocrinology, 2005, 146:1074-1096) is included in the results. Denotes * are statistically different from ** and UnTrt-MM (P) with p<0.05. Results are the mean ±SEM of mRNA expression in leiomyoma and matched myometrium form proliferative (N=8) and secretory (N=12) phases of the menstrual and GnRHa-treated group (N=7).

FIG. 17 shows Western blot analysis of fibromodulin in 14 paired myometrium (M) and leiomyoma (L) from proliferative (N=7) and secretory (N=7) phases of the menstrual cycle, and from women who received GnRHa therapy (GnRHa-treated; N=6). Total protein was isolated from these tissues and equal amount of protein was subjected to immunoblotting using antibodies specific to fibromodulin.

FIGS. 18A-18D show immunohistochemical localization of fibromodulin in leiomyoma (A) and myometrium (B) with immunoreactive proteins in association with leiomyoma and myometrial smooth muscle cells, and cellular components of connective tissue and vasculature. Incubation of tissue sections with non-immune and goat IgGs instead of primary antibodies (C and D) during immunostaining served as controls (Ctrl) reduced the staining intensity. Mag: X60.

FIGS. 19A-19D are bar graphs showing relative mRNA expression of fibromodulin in leiomyoma (LSMC) and myometrial (MSMC) smooth muscle cells following treatment with TGF-β1 (2.5 ng/ml) and GnRHa (0.1 mM) for 2, 6 and 12 hrs; or in cells pretreated with 20 μM of U0126 (U) MEK1/2MAPK inhibitor followed by 2 hrs of treatment with TGF-β1 (T) or GnRHa (G). Serum-starved cells were pretreated with U0126 at for 2 hrs, washed and then treated with 2.5 ng/ml of TGF-β1 for 2 hrs. Additionally LSMC and MSMC were transfected with Smad3 SiRNA or scrambled SiRNA for 48 hrs washed and then treated with 2.5 ng/ml of TGF-β1 (T/Si) for 2 hrs Total RNA was isolated from treated and untreated control (Ctrl) cells and subjected to Realtime PCR. Results are the mean ±SEM of three experiments performed using independent cell cultures from different tissues. Denotes *, ** and *** are statistically different from untreated control. In Smad SiRNA-treated cells * is different from ** and *** with P<0.05, respectively. Arrows point out the significant differences between the expression of fibromodulin in LSMC and MSMC.

### DETAILED DISCLOSURE

The study disclosed herein was designed to further define the molecular environments of leiomyoma and matched myometrium during the early-mid luteal phase of the menstrual cycle, which is characterized by elevated production of ovarian steroids, compared with tissues obtained from hormonally suppressed patients on GnRHa therapy. The present inventors further evaluated the direct action of GnRHa on global gene expression and their regulation in leiomyoma and myometrial cells isolated from the untreated tissue cohort. These approaches enabled the identification of expression profiles of genes targeted by GnRHa. The present inventors validated the expression of 10 of these genes in these cohorts, and concluded that local expression and activation of these genes may represent features differentiating leiomyoma and myometrial molecular environments during growth as well as GnRHa-induced regression.

Microarrays have been shown to be of great value in understanding the molecular biology of many diseases, and they have been successfully used to classify various tumors based on their clinical phenotype or genetic background. In this experiment, the present inventors have used gene expression profiling to define the biological relationship between TGF-β and GnRH in tumor growth and regression, and try to unveil the complexity of leiomyoma genesis and development. The present inventors have evaluated the underlying differences between molecular responses directed by TGF-β autocrine/paracrine actions in LSMC and MSMC, and following interference with these actions using TGF-β receptor type II antisense oligomers treatment. Since TGF-β receptors expression is targeted by GnRHa in leiomyoma and myometrium, the present inventors further evaluated the gene expression profiles in response to TGF-β type II receptor antisense treatment and GnRHa-treated LSMC and MSMC to identify the genes whose expression are the specific target of these treatments. Using this approach, several differentially expressed and regulated genes targeted by TGF-β autocrine/paracrine action were evaluated, and the expression of 12 genes in LSMC and MSMC in response to the time-dependent action of TGF-β was validated using Realtime PCR.

Methods involving conventional molecular biology techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Various techniques using polymerase chain reaction (PCR) are described, e.g., in Innis et al., PCR Protocols: A Guide to Methods and Applications, Academic Press: San Diego, 1990. Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

The following publications are specifically incorporated herein by reference in their entirety, including all figures, tables, and sequences, to the extent they are not inconsistent with the explicit teachings of this specification: U.S. patent publication US 2003/0032044 (Chegini et al.), filed Jul. 17, 2002; international publication WO 03/007685 (Chegini et al.), filed Jul. 17, 2002; international publication WO 00/20642 (Chegini et al.), filed Oct. 1, 1999; U.S. patent publication US 2003/0077589 (Hess-Stumpp et al.), filed Sep. 25, 2001; and U.S. patent publication US 2001/0002393 (Palmer et al.), filed Dec. 20, 2000.

### I. Detecting Fibrotic Disorders

The invention provides a method for detecting a fibrotic disorder in the tissue of a subject. This method includes the steps of: (a) providing a biological sample obtained (i.e., derived) from the subject (such as endometrium or peritoneal fluid); (b) analyzing the expression of a differentially expressed gene in the sample; and (c) correlating the expression of the differentially expressed gene with the presence or absence of the fibrotic disorder in the subject.

Examples of reproductive tract disorders include, but are not limited to, leiomyoma, endometriosis, ovarian hyperstimulation syndrome, adhesions, and other tissue fibroses (e.g., fibroids) (Smits G. et al., N. Engl. J. Med., 2003, 349(8):760-766; Elchalal U. et al., Human Reproduction, 1997, 12(6):1129-1137; Stewart E. et al., Human Reproduction Update, 1996, 2(4):295-306; Shozu M. et al., The Journal of Clinical Endocrinology & Metabolism, 86(11):5405-5411; Estaban J. et al., Arch. Pathol. Lab. Med., 1999, 123:960-962; Lee W. et al., The Korean Journal of Pathology, 2003, 37:71-73; and Kurioka H. et al., Human Reproduction, 1998, 13(5):1357-1360).

Differentially expressed genes include those which are differentially expressed in a given fibrotic disorder, including but not limited to, docking protein 1, 62 kD (downstream of tyrosine kinase 1); centromere protein A (17 kD); catenin (cadherin-associated protein), beta 1 (88 kD); nuclear receptor subfamily 1, group I, member 2; v-rel avian reticuloendotheliosis viral oncogene homolog A; LGN Protein; CDC28 protein kinase 1; hypothetical protein; solute carrier family 17 (sodium phosphate), member 1; FOS-like antigen-1; nuclear matrix protein p84; LERK-6 (EPLG6); visinin-like 1; phosphodiesterase 10A; KH-type splicing regulatory protein (FUSE binding protein 2); Polyposis locus (DP1 gene) mRNA; microtubule-associated protein 2; CDC5 (cell division cycle 5, S pombe, homolog)-like; Centromere autoantigen C (CENPC) mRNA; RNA guanylyltransferase and 5′-phosphatase; Nijmegen breakage syndrome I (nibrin); ribonuclease, RNase A family, 4; keratin 10 (epidermolytic hyperkeratosis; keratosis palmaris et plantaris); basic helix-loop-helix domain containing, class B, 2; dual specificity phosphatase 1; annexin A11; putative receptor protein; Human endogenous retrovirus HERV-K(HML6); mitogen-activated protein kinase kinase kinase 12; TXK tyrosine kinase; kynureninase (L-kynurenine hydrolase); ubiquitin specific protease 4 (proto-oncogene); peroxisome biogenesis factor 13; olfactory receptor, family 2, subfamily F, member 1; membrane protein, palmitoylated 3 (MAGUK p55 subfamily member 3); origin recognition complex, subunit 1 (yeast homolog)-like; dTDP-D-glucose 4,6-dehydratase; cytochrome c oxidase subunit VIa polypeptide 2; gamma-tubulin complex protein 2; Monocyte chemotactic protein-3; myelin transcription factor 1; inhibitor of growth family, member 1-like; thyroid hormone receptor, alpha myosin-binding protein C, slow-type; fragile X mental retardation 2; sonic hedgehog (Drosophila) homolog; 6-phosphofructo-2-kinase/fructose-2,6-biphosphatase 2; SFRS protein kinase 2; excision repair cross-complementing rodent repair deficiency; cyclin-dependent kinase 5, regulatory subunit 1 (p35); poly(A)-specific ribonuclease (deadenylation nuclease); solute carrier family 12 (potassium/chloride transporters), member 4; Pseudogene for metallothionein; natriuretic peptide precursor A; intercellular adhesion molecule 2; apoptosis antagonizing transcription factor; similar to rat HREV107; major histocompatibility complex, class II, DP beta 1; MpV17 transgene, murine homolog, glomerulosclerosis; uroporphyrinogen decarboxylase; proteasome (prosome, macropain) 26S subunit, ATPase, 1; fins-related tyrosine kinase 3 ligand; actin, gamma 1; Protein Kinase Pitslre, Alpha, Alt. Splice 1-Feb; nuclear factor of kappa light polypeptide gene enhancer in B-cells inhibitor, alpha; pyruvate kinase, muscle; telomeric repeat binding factor 2; cell division cycle 2, G1 to S and G2 to M; ADP-ribosylation factor 3; NRF1 Protein; H factor (complement)-like 3; serine (or cysteine) proteinase inhibitor, clade B (ovalbumin), member 6; mRNA of muscle specific gene M9; solute carrier family 25 (mitochondrial carrier; phosphate carrier), member 3; ribosomal protein L36a; suppressor of Ty (S. cerevisiae) 4 homolog 1; amino-terminal enhancer of split; ubiquitin A-52 residue ribosomal protein fusion product 1; hydroxyacyl-Coenzyme A dehydrogenase/3-ketoacyl-Coenzyme A thiolase; chaperonin containing TCP1, subunit 2 (beta); tyrosine kinase with immunoglobulin and epidermal growth factor homology; domains; Fc fragment of IgG, receptor, transporter, alpha; NRD1 convertase; ADP-ribosylation factor 5; transcription elongation factor A (S11), 1; like mouse brain protein E46; titin; fibromodulin; Abl-interactor 2 (Abi-2); and other differentially expressed genes disclosed herein. In one embodiment, the differentially expressed gene includes one or more of the genes listed in Table 9. The number of differentially expressed genes analyzed in the sample can be 1, 2, 3, 4, 5, 6, 7, 8, 9, 10, 11, 12, 13, 14, 15, 16, 17, 18, 19, 20, or more.

In another embodiment, the differentially expressed gene is at least one of CDKN1B, CDKN1C, CTGF, fibromodulin, and Abl-interactor 2 (Abi-2).

Suitable subjects for use in the invention can be any human or non-human animal. For example, the subject can be a female animal, such as mammal, like a dog, cat, horse, cow, pig, sheep, goat, chicken, primate, rat, or mouse. Because the experiments presented herein relate to human subjects, a preferred subject for the methods of the invention is a human, such as a human female. Particularly preferred are female subjects suspected of having or at risk for developing a fibrotic disorder within the reproductive tract, e.g., a woman suspected of having or at risk for developing leiomyoma, endometriosis, or peritoneal adhesions based on clinical findings or other diagnostic test results.

The step of providing a biological sample obtained from the subject can be performed by conventional medical techniques. For example, an endometrial tissue sample can be taken from the subject by biopsy. As another example, a sample of peritoneal fluid can be taken from a subject by conventional techniques. Suitable methods are described in more detail in the Examples sections presented below.

The step of analyzing the expression of a differentially expressed gene in the sample can be performed in a variety of different ways. Numerous suitable techniques are known for analyzing gene expression. For example, gene expression can be determined directly by assessing protein expression of cells or fluid of a biological sample (e.g., endometrial tissue or peritoneal fluid). Proteins can be detected using immunological techniques, e.g., using antibodies that specifically bind the protein in assays such as immunofluorescence or immunohistochemical staining and analysis, enzyme-linked immunosorbent assay (ELISA), radioimmunoassay (RIA), immunoblotting (e.g., Western blotting), and like techniques. Expression of differentially expressed genes can also be determined by directly or indirectly measuring the amount of mRNA encoding protein in a cellular sample using known techniques such as Northern blotting and PCR-based methods such as competitive quantitative reverse transcriptase PCR (Q-RT-PCR). Suitable methods for analyzing expression of differentially expressed genes are described below; nonetheless, other suitable methods might also be employed.

The step of correlating the expression of the gene with the presence or absence of the fibrotic disorder in the subject involves comparing the level of gene expression in the test biological sample with levels of gene expression in control samples, e.g., those derived from subjects known to have or not to have the particular disorder. Thus, after quantifying gene expression in a biological sample from a test subject, the test result is compared to levels of gene expression determined from (a) a panel of cells or tissues derived from subjects (preferably matched to the test subject by age, species, strain or ethnicity, and/or other medically relevant criteria) known to have a particular disorder and (b) a panel of cells or tissues derived from subjects (preferably also matched as above) known not to have a particular disorder. If the test result is closer to the levels (e.g., mean or arithmetic average) from the panel of cells or tissues derived from subjects known to have a particular disorder, then the test result correlates with the test subject having the particular disorder. On the other hand, if the test result is closer to the levels (e.g., mean or arithmetic average) from the panel of cells or tissues derived from subjects known not to have a particular disorder, then the test result correlates with the test subject not having the particular disorder. Optionally, the method further comprises selecting and administering a therapy or therapies to the patient to treat for the correlated disorder(s).

### II. Modulating Gene Expression

The present invention also provides a method for modulating the expression of genes that are differentially expressed in fibrotic tissues (such as leiomyoma), compared to normal tissues. Restoration of gene expression to levels associated with normal tissue is expected to ameliorate at least some of the symptoms associated with the fibrotic disorder. This method includes the step of contacting the tissue with an agent that modulates expression of one or more differentially expressed genes in the tissue. Optionally, the method includes the step of diagnosing the subject with the fibrotic disorder prior to contacting the tissue with the agent that modulates expression of one or more differentially expressed genes in the fibrotic tissue.

Differentially expressed genes include those which are differentially expressed in a given fibrotic disorder, including but not limited to, docking protein 1, 62 kD (downstream of tyrosine kinase 1); centromere protein A (17 kD); catenin (cadherin-associated protein), beta 1 (88 kD); nuclear receptor subfamily 1, group I, member 2; v-rel avian reticuloendotheliosis viral oncogene homolog A; LGN Protein; CDC28 protein kinase 1; hypothetical protein; solute carrier family 17 (sodium phosphate), member 1; FOS-like antigen-1; nuclear matrix protein p84; LERK-6 (EPLG6); visinin-like 1; phosphodiesterase 10A; KH-type splicing regulatory protein (FUSE binding protein 2); Polyposis locus (DP1 gene) mRNA; microtubule-associated protein 2; CDC5 (cell division cycle 5, S pombe, homolog)-like; Centromere autoantigen C (CENPC) mRNA; RNA guanylyltransferase and 5′-phosphatase; Nijmegen breakage syndrome 1 (nibrin); ribonuclease, RNase A family, 4; keratin 10 (epidermolytic hyperkeratosis; keratosis palmaris et plantaris); basic helix-loop-helix domain containing, class B, 2; dual specificity phosphatase 1; annexin A11; putative receptor protein; Human endogenous retrovirus HERV-K(HML6); mitogen-activated protein kinase kinase kinase 12; TXK tyrosine kinase; kynureninase (L-kynurenine hydrolase); ubiquitin specific protease 4 (proto-oncogene); peroxisome biogenesis factor 13; olfactory receptor, family 2, subfamily F, member 1; membrane protein, palmitoylated 3 (MAGUK p55 subfamily member 3); origin recognition complex, subunit 1 (yeast homolog)-like; dTDP-D-glucose 4,6-dehydratase; cytochrome c oxidase subunit VIa polypeptide 2; gamma-tubulin complex protein 2; Monocyte chemotactic protein-3; myelin transcription factor 1; inhibitor of growth family, member 1-like; thyroid hormone receptor, alpha myosin-binding protein C, slow-type; fragile X mental retardation 2; sonic hedgehog (Drosophila) homolog; 6-phosphofructo-2-kinase/fructose-2,6-biphosphatase 2; SFRS protein kinase 2; excision repair cross-complementing rodent repair deficiency; cyclin-dependent kinase 5, regulatory subunit 1 (p35); poly(A)-specific ribonuclease (deadenylation nuclease); solute carrier family 12 (potassium/chloride transporters), member 4; Pseudogene for metallothionein; natriuretic peptide precursor A; intercellular adhesion molecule 2; apoptosis antagonizing transcription factor; similar to rat HREV107; major histocompatibility complex, class II, DP beta 1; MpV17 transgene, murine homolog, glomerulosclerosis; uroporphyrinogen decarboxylase; proteasome (prosome, macropain) 26S subunit, ATPase, 1; fms-related tyrosine kinase 3 ligand; actin, gamma 1; Protein Kinase Pitslre, Alpha, Alt. Splice 1-Feb; nuclear factor of kappa light polypeptide gene enhancer in B-cells inhibitor, alpha; pyruvate kinase, muscle; telomeric repeat binding factor 2; cell division cycle 2, G1 to S and G2 to M; ADP-ribosylation factor 3; NRF1 Protein; H factor (complement)-like 3; serine (or cysteine) proteinase inhibitor, clade B (ovalbumin), member 6; mRNA of muscle specific gene M9; solute carrier family 25 (mitochondrial carrier; phosphate carrier), member 3; ribosomal protein L36a; suppressor of Ty (S. cerevisiae) 4 homolog 1; amino-terminal enhancer of split; ubiquitin A-52 residue ribosomal protein fusion product 1; hydroxyacyl-Coenzyme A dehydrogenase/3-ketoacyl-Coenzyme A thiolase; chaperonin containing TCP1, subunit 2 (beta); tyrosine kinase with immunoglobulin and epidermal growth factor homology; domains; Fc fragment of IgG, receptor, transporter, alpha; NRD1 convertase; ADP-ribosylation factor 5; transcription elongation factor A (S11), 1; like mouse brain protein E46; titin; fibromodulin; Abl-interactor 2 (Abi-2); and other differentially expressed genes disclosed herein. In one embodiment, the differentially expressed gene includes one or more of the genes listed in Table 9.

In another embodiment, the differentially expressed gene is at least one of CDKN1B, CDKN1C, CTGF, fibromodulin, and Abl-interactor 2 (Abi-2).

In a preferred embodiment, the agent that modulates expression of a differentially expressed gene in fibrotic tissue (such as leiomyoma) is one that decreases or down-regulates the action or expression of one or more genes selected from the group consisting of IL-11, IL-13, EGR1, EGR2, EGR3, CITED2, P300, E2F1, E2F2, E2F3, E2F4, E2F5, MCP3, CXCL5, CCL7, SMAD3, TYMS, and/or GT198. In a another preferred embodiment, the agent that modulates expression of a differentially expressed gene in fibrotic tissue is one that increases or up-regulates the action or expression of one or more genes selected from the group consisting of SMAD-7, NCOR2, TIMP-1, and ADAM17. More preferably, the agent decreases or down-regulates the action or expression of one or more genes selected from the group consisting of IL-11, IL-13, EGR1, EGR2, EGR3, CITED2, P300, E2F1, E2F2, E2F3, E2F4, E2F5, MCP3, CXCL5, CCL7, SMAD3, TYMS, and/or GT198, and increases or up-regulates the action or expression of one or more genes selected from the group consisting of SMAD-7, NCOR2, TIMP-1, and ADAM17.

In one embodiment, the agent that modulates expression of a differentially expressed gene in fibrotic tissue (such as leiomyoma) is selected from the group consisting of a selective estrogen receptor modulator (such as Roloxifene or other SERM), a selective progesterone receptor modulator (such as Asoprisnil (J867), RU486, or other SPRM), SB-505124, SB-431542, a mast cell inhibitor (such as Tranlist), Cystatin C (CystC), SD-208, LY550410, LY580276, Pitavastatin, 1,5 naphthyridine amiothiazole derivative, 1,5 naphthyridine pyrazole derivative, and ursolic acid (see, for example, Yingling, J. et al., Nat. Rev. Drug Discov., 2004, Dec.; 3(12):1011-22; Chwalisz, K. et al., Semin. Reprod. Med., 2004, 22(2):113-119; Hodl, C. et al., Bioconjug. Che., 2004, 15(2):359-365; Dubey, R. K. et al., J. Clin. Endocrinol. Metab., 2004, 89(2):852-859; DeManno, D. et al., Steroids, 2003, 68(10-13):1019-1032; DaCosta, B. S. et al., Mol. Pharmacol., 65(3):744-752; Sokol, J. P. et al., Mol. Cancer. Res., 2004, 2(3):183-195; Wanatabe, T. et al., Journal of Cell Biology, 2003, 163(6):1303-1311, and Hjelmeland, M. D. et al., Mol. Cancer. Ther., 2004, 3(6):737-745), which are incorporated herein by reference in their entirety). In another embodiment, the agent is one based on a pyrazolopyridine scaffold (Beight, D. W. et al., WO 2004/026871), a pyrazole scaffold (Gellibert, F. et al., J. Med. Chem., 2004, 47:4494-4506), an imidazopyridine scaffold (Lee, W. C. et al., WO 2004/021989), triazole scaffold (Blumberg, L. C. et al., WO 2004/026307), a pyridopyrimidine scaffold (Chakravarty, S. et al., WO 2000/012497), or an isothiazole scaffold (Munchhof, M. J., WO 2004/147574), each of which is incorporated herein by reference in its entirety.

Preferably, the agent administered to the subject for treatment or prevention of fibrosis is one that inhibits (reduces) TGF-beta signaling (signal transduction). More preferably, the agent administered to the subject that inhibits (reduces) TGF-beta II signaling (signal transduction).

In another aspect of the method of the invention, the subject invention includes a method for treating (alleviating symptoms associated with) fibrotic tissue or reducing the likelihood of fibrotic tissue formation, by administering GnRH analog locally to the target site. For example, the GnRH analog can be administered directly to a fibroid to reduce the size of the fibroid.

The tissue for use in this method can be any derived from a human or non-human animal. In some embodiments, the tissue is derived from a female reproductive system, e.g., endometrium, or tissue derived from the uterus, cervix, vagina, fallopian tube, or ovary. Because the experiments presented herein relate to human subjects, a preferred tissue sample for the methods of the invention is one derived from a human. Particularly preferred is tissue derived from a subject suspected of having or at risk for developing a fibrotic disorder (such as a woman suspected of having or at risk for developing leiomyoma, endometriosis, ovarian hyperstimulation syndrome, peritoneal adhesions, or other tissue fibroses) based on clinical findings or other diagnostic test results.

The method of the present invention utilizes one or more agents that modulate expression one or more differentially expressed genes in the tissue. Numerous agents for modulating expression of such genes in a tissue are known. Any of those suitable for the particular system being used may be employed. Typical agents for modulating expression of such genes are proteins, nucleic acids, and small organic or inorganic molecules such as hormones (e.g., natural or synthetic steroids). Preferably, the agent is not a hormone.

An example of a protein that can modulate gene expression is an antibody that specifically binds to the gene product. Such an antibody can be used to interfere with the interaction of the gene product and other molecules that bind the gene product. Products of the differentially expressed genes (or immunogenic fragments or analogs thereof) can be used to raise antibodies useful in the invention. Such gene products (e.g., proteins) can be produced by purification from cells/tissues, recombinant techniques or chemical synthesis as described above. Antibodies for use in the invention include polyclonal antibodies, monoclonal antibodies, single chain antibodies, Fab fragments, F(ab′)₂ fragments, and molecules produced using a Fab expression library. See, for example, Kohler et al., Nature, 1975, 256:495; Kohler et al., Eur. J. Immunol., 1976, 6:511; Kohler et al., Eur. J. Immunol., 1976, 6:292; Hammerling et al., “Monoclonal Antibodies and T Cell Hybridomas,” Elsevier, N.Y., 1981; Ausubel et al., supra; U.S. Pat. Nos. 4,376,110, 4,704,692, and 4,946,778; Kosbor et al., Immunology Today, 1983, 4:72; Cole et al., Proc. Natl. Acad. Sci. USA, 1983, 80:2026; Cole et al., “Monoclonal Antibodies and Cancer Therapy,” Alan R. Liss, Inc., pp. 77-96, 1983; and Huse et al., Science, 1989, 246:1275.

Other proteins that can modulate gene expression include variants of the gene products that can compete with the native gene products for binding ligands such as naturally occurring receptors of these gene products. Such variants can be generated through various techniques known in the art. For example, protein variants can be made by mutagenesis, such as by introducing discrete point mutation(s), or by truncation. Mutation can give rise to a protein variant having substantially the same, or merely a subset of the functional activity of a native protein. Alternatively, antagonistic forms of the protein can be generated which are able to inhibit the function of the naturally occurring form of the protein, such as by competitively binding to another molecule that interacts with the protein. In addition, agonistic (or superagonistic) forms of the protein may be generated that constitutively express one or more functional activities of the protein. Other variants of the gene products that can be generated include those that are resistant to proteolytic cleavage, as for example, due to mutations which alter protease target sequences. Whether a change in the amino acid sequence of a peptide results in a protein variant having one or more functional activities of a native protein can be readily determined by testing the variant for a native protein functional activity (e.g., binding a receptor or inducing a cellular response).

Another agent that can modulate gene expression is a non-peptide mimetic or chemically modified form of the gene product that disrupts binding of the encoded protein to other proteins or molecules with which the native protein interacts. See, e.g., Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. J. Med. Chem., 1986, 29:295; and Ewenson et al. in Peptides: Structure and Function (Proceedings of the 9th American Peptide Symposium) Pierce Chemical Co. Rockland, Ill., 1985), beta-turn dipeptide cores (Nagai et al. Tetrahedron Lett, 1985, 26:647; and Sato et al. J. Chem. Soc. Perkin. Trans., 1986, 1:1231), and beta-aminoalcohols (Gordon et al. Biochem. Biophys. Res. Commun., 1985, 126:419; and Dann et al. Biochem. Biophys. Res. Commun., 1986, 134:71). Proteins may also be chemically modified to create derivatives by forming covalent or aggregate conjugates with other chemical moieties, such as glycosyl groups, lipids, phosphate, acetyl groups and the like. Covalent derivatives of proteins encoded by differentially expressed genes can be prepared by linking the chemical moieties to functional groups on amino acid side chains of the protein or at the N-terminus or at the C-terminus of the polypeptide.

The agent that directly reduces expression of the differentially expressed gene can also be a nucleic acid molecule that reduces expression of the gene. For example, the nucleic acid molecule can be an antisense nucleic acid that hybridizes to mRNA encoding the protein. Antisense nucleic acid molecules for use within the invention are those that specifically hybridize (e.g. bind) under cellular conditions to cellular mRNA and/or genomic DNA encoding a protein in a manner that inhibits expression of the protein, e.g., by inhibiting transcription and/or translation. The binding may be by conventional base pair complementarity, or, for example, in the case of binding to DNA duplexes, through specific interactions in the major groove of the double helix.

In one embodiment, the nucleic acid molecule that directly reduces the expression of the differentially expressed gene is selected from the group consisting of antisense, short interfering RNA (siRNA), and a ribozyme. In a specific embodiment, the nucleic acid molecule is targed to the TGF-beta type II receptor, directly reducing its expression.

Vectors may be used to deliver the nucleic acid molecule to the target site (e.g., the fibrotic tissue) in vitro or in vivo. The vector may be, for example, a viral vector (such as lentivirus) or a non-viral vector (such as a liposome or other cholesterol molecule); see, for example, Soutschek, J. et al., Nature, 2004, 432(7014):173-178, which describes therapeutic silencing of an endogenous gene by administration siRNAs, and which is incorporated herein by reference in its entirety.

Antisense constructs can be delivered as an expression plasmid which, when transcribed in the cell, produces RNA which is complementary to at least a unique portion of the cellular mRNA which encodes the protein. Alternatively, the antisense construct can take the form of an oligonucleotide probe generated ex vivo which, when introduced into a protein expressing cell, causes inhibition of protein expression by hybridizing with an mRNA and/or genomic sequences coding for the protein. Such oligonucleotide probes are preferably modified oligonucleotides that are resistant to endogenous nucleases, e.g. exonucleases and/or endonucleases, and are therefore stable in vivo. Exemplary nucleic acid molecules for use as antisense oligonucleotides are phosphoramidate, phosphothioate and methylphosphonate analogs of DNA (see, e.g., U.S. Pat. Nos. 5,176,996; 5,264,564; and 5,256,775). Additionally, general approaches to constructing oligomers useful in antisense therapy have been reviewed, for example, by Van der Krol et al., Biotechniques, 1988, 6:958-976; and Stein et al., Cancer Res., 1988, 48:2659-2668. With respect to antisense DNA, oligodeoxyribonucleotides derived from the translation initiation site, e.g., between the −10 and +10 regions of a protein encoding nucleotide sequence, are preferred.

Antisense approaches involve the design of oligonucleotides (either DNA or RNA) that are complementary to mRNA encoding the protein to be inhibited. The antisense oligonucleotides will bind to mRNA transcripts and prevent translation. Absolute complementarity, although preferred, is not required. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, the antisense oligonucleotides used in the subject invention are targeted to the TGF-beta type II receptor, such as those disclosed herein.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well (Wagner, R., Nature, 1994, 372:333). Therefore, oligonucleotides complementary to either the 5′ or 3′ untranslated, non-coding regions of a differentially expressed gene could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′, 3′ or coding region of the mRNA, antisense nucleic acids should be at least eighteen nucleotides in length, and are preferably less than about 100 and more preferably less than about 30, 25, 20, or 18 nucleotides in length.

Antisense oligonucleotides of the invention may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxyethyl) uracil, 5-carboxymethylaminomethyl-2-thiouridine, 5-carboxymethylaminomethyluracil, dihydrouricil, beta-D-galactosylqueosine, inosine, N6-isopentenyladenine, 1-methylguanine, 1-methylinosine, 2,2-idimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Antisense oligonucleotides of the invention may also comprise at least one modified sugar moiety selected from the group including but not limited to arabinose, 2-fluoroarabinose, xylulose, and hexose; and may additionally include at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual beta-units, the strands run parallel to each other (Gautier et al., Nucl. Acids Res., 1987, 15:6625-6641). Such oligonucleotide can be a 2′-0-methylribonucleotide (Inoue et al., Nucl. Acids Res., 1987, 15:6131-6148), or a chimeric RNA-DNA analogue (Inoue et al., FEBS Lett., 1987, 215:327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. Nucl. Acids Res., 1988, 16:3209), methylphosphonate oligonucleotides can be prepared by use of controlled pore glass polymer supports (Sarin et al., Proc. Natl. Acad. Sci. U.S.A., 1988, 85:7448-7451).

The antisense molecules should be delivered into cells that express the differentially expressed (e.g., overexpressed) genes in vivo. A number of methods have been developed for delivering antisense DNA or RNA into cells. For instance, antisense molecules can be introduced directly into the tissue site by such standard techniques as electroporation, liposome-mediated transfection, CaCl-mediated transfection, or the use of a gene gun. Alternatively, modified antisense molecules, designed to target the desired cells (e.g., antisense linked to peptides or antibodies that specifically bind receptors or antigens expressed on the target cell surface) can be used.

However, because it is often difficult to achieve intracellular concentrations of the antisense sufficient to suppress translation of endogenous mRNAs, a preferred approach utilizes a recombinant DNA construct in which the antisense oligonucleotide is placed under the control of a strong promoter (e.g., the CMV promoter). The use of such a construct to transform cells will result in the transcription of sufficient amounts of single stranded RNAs that will form complementary base pairs with the endogenous gene transcripts and thereby prevent translation of the mRNA.

Ribozyme molecules designed to catalytically cleave target mRNA transcripts can also be used to prevent translation of mRNA and expression of protein (see, e.g., PCT Publication No. WO 90/11364, published Oct. 4, 1990; Sarver et al., Science, 1990, 247:1222-1225 and U.S. Pat. No. 5,093,246). While ribozymes that cleave mRNA at site-specific recognition sequences can be used to destroy target mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature, 1988, 334:585-591. Preferably the ribozyme is engineered so that the cleavage recognition site is located near the 5′ end of the mRNA; i.e., to increase efficiency and minimize the intracellular accumulation of non-functional mRNA transcripts. Ribozymes within the invention can be delivered to a cell using a vector.

The expression of endogenous genes that are overexpressed in fibrotic disorders can also be reduced by inactivating or “knocking out” the gene or its promoter using targeted homologous recombination. See, e.g., Kempin et al., Nature, 1997, 389:802; Smithies et al., Nature, 1985, 317:230-234; Thomas and Capecchi, Cell, 1987, 51:503-512; and Thompson et al., Cell, 1989, 5:313-321. For example, a mutant, non-functional gene variant (or a completely unrelated DNA sequence) flanked by DNA homologous to the endogenous gene (either the coding regions or regulatory regions of the gene) can be used, with or without a selectable marker and/or a negative selectable marker, to transfect cells that express the gene in vivo.

Alternatively, endogenous gene expression may be reduced by targeting deoxyribonucleotide sequences complementary to the regulatory region of the target gene(s) (i.e., the gene promoter and/or enhancers) to form triple helical structures that prevent transcription of the gene in target cells. (See generally, Helene, C., Anticancer Drug Des., 1991, 6(6):569-84; Helene, C., et al., Ann. N.Y. Acad. Sci., 1992, 660:27-36; and Maher, L. J., Bioassays, 1992, 14(12):807-15).

Antisense nucleic acid, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramide chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters. Alternatively, antisense cDNA constructs that synthesize antisense RNA constitutively or inducibly, depending on the promoter used, can be introduced stably into cell lines.

Another agent that can be used to modulate gene expression in fibrotic tissue is a hormone. Numerous naturally occurring and synthetic hormones are known to cause physiological changes in such tissue and are available commercially. See, e.g., PDR: Physician's Desk Reference, 2002. Those particular hormones which modulate expression of differentially expressed genes in a given sample tissue can be determined empirically by contacting a series of tissue samples with a panel of different hormones and analyzing the tissue samples for changes in phenotype over time. In experiments relating to the invention, it was shown that GnRHa therapy modulated the expression of 297 genes in leiomyoma and myometrium compared to untreated group (P<0.02). In addition, GnRHa, TGF-b and TGF-b receptor type II antisense treatments resulted in differential regulation of 134, 144, and 154 specific genes, respectively (P<0.005 and 0.001). The products of these genes were functionally categorized as key regulators of cell cycle, transcription factors, signal transduction, ECM turnover and apoptosis. Based on (i) expression values, (ii) functional classification and (iii) regulation by GnRH and TGF-b mediated actions, we selected 10 of these genes and validated their expression in leiomyoma and myometrium, and in LSMC and MSMC using RealTime PCR, western blotting and immunohistochemistry. In conclusion, the results provide additional evidence for the difference in gene expression profile between leiomyoma and myometrium, and reveal the profile of previously unrecognized novel genes whose expression are the target of GnRH and TGF-β actions in leiomyoma and myometrium.

The agent that can be used to modulate gene expression in fibrotic tissue may be administered to non-human animals or humans in pharmaceutically acceptable carriers (e.g., physiological saline) that are selected on the basis of mode and route of administration and standard pharmaceutical practice. For example, the pharmaceutical compositions of the invention might include suitable buffering agents such as acetic acid or its salt (1-2% w/v); citric acid or its salt (1-3% w/v); boric acid or its salt (0.5-2.5% w/v); succinic acid; or phosphoric acid or its salt (0.8-2% w/v); and suitable preservatives such as benzalkonium chloride (0.003-0.03% w/v); chlorobutanol (0.3-0.9% w/v); parabens (0.01-0.25% w/v) or thimerosal (0.004-0.02% w/v). Examples of compositions suitable for parenteral administration include sterile aqueous preparations such as water, Ringer's solution, and isotonic sodium chloride solution. In addition, sterile, fixed oils might be used as a solvent or suspending medium. For this purpose, any bland fixed oil may be employed including synthetic mono- or di-glycerides. In addition, fatty acids such as oleic acid find use in the preparation of injectables. Carrier formulations suitable for local, subcutaneous, intramuscular, intraperitoneal or intravenous administrations may be found in Remington's Pharmaceutical Sciences, Mack Publishing Company, Easton, Pa. The pharmaceutical compositions useful in the invention may be delivered in mixtures of more than one pharmaceutical composition.

The compositions of the invention (containing an agent that can be used to modulate gene expression in fibrotic tissue) may be administered to animals or humans by any conventional technique. Such administration might be parenteral (e.g., intravenous, subcutaneous, intramuscular, or intraperitoneal introduction). Preferably, the compositions may also be administered directly to the target site (e.g., a portion of the reproductive tract or peritoneal cavity) by, for example, surgical delivery to an internal or external target site, or by catheter to a site accessible by a blood vessel. Other methods of delivery, e.g., liposomal delivery or diffusion from a device impregnated with the composition, are known in the art. The composition may be administered in a single bolus, multiple injections, or by continuous infusion (e.g., intravenously or by peritoneal dialysis).

The methods of this invention, generally speaking, may be practiced using any mode of administration that is medically acceptable, meaning any mode that produces effective levels of response without causing clinically unacceptable adverse effects. Preferred modes of administration include parenteral, injection, infusion, deposition, implantation, anal or vaginal supposition, oral ingestion, inhalation, and topical administration. Injections can be intravenous, intradermal, subcutaneous, intramuscular, or interperitoneal. For example, the pharmaceutical composition can be injected directly into target site for the prevention of fibrotic disorders, such as leiomyoma, endometriosis, ovarian hyperstimulation syndrome, or adhesion formation. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially fused pellets. Inhalation includes administering the pharmaceutical composition with an aerosol in an inhaler, either alone or attached to a carrier that can be absorbed. For systemic administration, it may be preferred that the pharmaceutical composition is encapsulated in liposomes. The term “parenteral” includes subcutaneous injections, intravenous, intramuscular, intraperitoneal, intrasternal injection or infusion techniques. In certain preferred embodiments of the invention, the administration can be designed so as to result in sequential exposure of the pharmaceutical composition over some period of time, e.g., hours, days, weeks, months or years. This can be accomplished by repeated administrations of the pharmaceutical composition, by one of the methods described above, or alternatively, by a sustained-release delivery system in which the pharmaceutical composition is delivered to the subject for a prolonged period without repeated administrations. By sustained-release delivery system, it is meant that total release of the pharmaceutical composition does not occur immediately upon administration, but rather is delayed for some period of time. Release can occur in bursts or it can occur gradually and continuously. Administration of such a system can be, e.g., by long-lasting oral dosage forms, bolus injections, transdermal patches, and subcutaneous implants.

A therapeutically effective amount is an amount that is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. Toxicity and therapeutic efficacy of the compositions of the invention can be determined by standard pharmaceutical procedures, using cells in culture and/or experimental animals to determine the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents that exhibit large therapeutic indices are preferred. While agents that exhibit toxic side effects may be used, care should be taken to design a delivery system that targets such compounds to the site of the tissues to be treated in order to minimize potential damage to uninvolved tissue and thereby reduce side effects. The data obtained from cell culture assays and animal studies can be used in formulating a range of dosage for use in humans. The dosage of such compounds lies preferably within the range of circulating concentrations that include an ED50 with little or no toxicity. The dosage may vary within this range depending on the dosage form employed and the route of administration utilized.

III. Methods for Identifying Agents that Modulate Fibrosis

The present invention also relates to methods of identifying agents, and the agents themselves, which modulate differentially-expressed genes or polypeptides expressed in endothelial or other fibrosis-forming (e.g., leiomyoma-forming) cells, such as cells of the female reproductive tract. In one embodiment, the fibrosis is uterine fibrosis. These agents can be used to modulate the biological activity of the polypeptide encoded for the gene, or the gene, itself. Agents that regulate the gene or its product are useful in variety of different environments, including as medicinal agents to treat or prevent disorders associated with fibrosis and as research reagents to modify the function of tissues and cells.

The methods for identifying agents, in accordance with the present invention, generally comprise steps in which an agent is placed in contact with the gene, its transcription product, its translation product, or other target, and then a determination is performed to assess whether the agent “modulates” the target. The specific method utilized will depend upon a number of factors, including, e.g., the target (i.e., is it the gene or polypeptide encoded by it), the environment (e.g., in vitro or in vivo), the composition of the agent, etc.

Differentially expressed genes include those which are differentially expressed in a given fibrotic disorder, including but not limited to, docking protein 1, 62 kD (downstream of tyrosine kinase 1); centromere protein A (17 kD); catenin (cadherin-associated protein), beta 1 (88 kD); nuclear receptor subfamily 1, group I, member 2; v-rel avian reticuloendotheliosis viral oncogene homolog A; LGN Protein; CDC28 protein kinase 1; hypothetical protein; solute carrier family 17 (sodium phosphate), member 1; FOS-like antigen-1; nuclear matrix protein p84; LERK-6 (EPLG6); visinin-like 1; phosphodiesterase 10A; KH-type splicing regulatory protein (FUSE binding protein 2); Polyposis locus (DP1 gene) mRNA; microtubule-associated protein 2; CDC5 (cell division cycle 5, S pombe, homolog)-like; Centromere autoantigen C (CENPC) mRNA; RNA guanylyltransferase and 5′-phosphatase; Nijmegen breakage syndrome 1 (nibrin); ribonuclease, RNase A family, 4; keratin 10 (epidermolytic hyperkeratosis; keratosis palmaris et plantaris); basic helix-loop-helix domain containing, class B, 2; dual specificity phosphatase 1; annexin All; putative receptor protein; Human endogenous retrovirus HERV-K(HML6); mitogen-activated protein kinase kinase kinase 12; TXK tyrosine kinase; kynureninase (L-kynurenine hydrolase); ubiquitin specific protease 4 (proto-oncogene); peroxisome biogenesis factor 13; olfactory receptor, family 2, subfamily F, member 1; membrane protein, palmitoylated 3 (MAGUK p55 subfamily member 3); origin recognition complex, subunit 1 (yeast homolog)-like; dTDP-D-glucose 4,6-dehydratase; cytochrome c oxidase subunit VIa polypeptide 2; gamma-tubulin complex protein 2; Monocyte chemotactic protein-3; myelin transcription factor 1; inhibitor of growth family, member 1-like; thyroid hormone receptor, alpha myosin-binding protein C, slow-type; fragile X mental retardation 2; sonic hedgehog (Drosophila) homolog; 6-phosphofructo-2-kinase/fructose-2,6-biphosphatase 2; SFRS protein kinase 2; excision repair cross-complementing rodent repair deficiency; cyclin-dependent kinase 5, regulatory subunit 1 (p35); poly(A)-specific ribonuclease (deadenylation nuclease); solute carrier family 12 (potassium/chloride transporters), member 4; Pseudogene for metallothionein; natriuretic peptide precursor A; intercellular adhesion molecule 2; apoptosis antagonizing transcription factor; similar to rat HREV107; major histocompatibility complex, class II, DP beta 1; MpV17 transgene, murine homolog, glomerulosclerosis; uroporphyrinogen decarboxylase; proteasome (prosome, macropain) 26S subunit, ATPase, 1; fms-related tyrosine kinase 3 ligand; actin, gamma 1; Protein Kinase Pitslre, Alpha, Alt. Splice 1-Feb; nuclear factor of kappa light polypeptide gene enhancer in B-cells inhibitor, alpha; pyruvate kinase, muscle; telomeric repeat binding factor 2; cell division cycle 2, G1 to S and G2 to M; ADP-ribosylation factor 3; NRF1 Protein; H factor (complement)-like 3; serine (or cysteine) proteinase inhibitor, clade B (ovalbumin), member 6; mRNA of muscle specific gene M9; solute carrier family 25 (mitochondrial carrier; phosphate carrier), member 3; ribosomal protein L36a; suppressor of Ty (S. cerevisiae) 4 homolog 1; amino-terminal enhancer of split; ubiquitin A-52 residue ribosomal protein fusion product 1; hydroxyacyl-Coenzyme A dehydrogenase/3-ketoacyl-Coenzyme A thiolase; chaperonin containing TCP1, subunit 2 (beta); tyrosine kinase with immunoglobulin and epidermal growth factor homology; domains; Fc fragment of IgG, receptor, transporter, alpha; NRD1 convertase; ADP-ribosylation factor 5; transcription elongation factor A (SII), 1; like mouse brain protein E46; titin; fibromodulin; Abl-interactor 2 (Abi-2); and other differentially expressed genes disclosed herein. In one embodiment, the differentially expressed gene includes one or more of the genes listed in Table 9. The number of differentially expressed genes analyzed in the sample can be 1, 2, 3, 4, 5, 6, 7, 8, 9, 10, 11, 12, 13, 14, 15, 16, 17, 18, 19, 20, or more.

In another embodiment, the differentially expressed gene is at least one of CDKN1B, CDKN1C, CTGF, fibromodulin, and Abl-interactor 2 (Abi-2).

For modulating the expression of a gene, a method can comprise, in any effective order, one or more of the following steps, e.g., contacting a gene (e.g., in a cell population) with a test agent under conditions effective for the test agent to modulate the expression of the gene, and determining whether the test agent modulates the gene. An agent can modulate expression of a gene at any level, including transcription (e.g., by modulating the promoter), translation, and/or perdurance of the nucleic acid (e.g., degradation, stability, etc.) in the cell.

For modulating the biological activity of polypeptides, a method can comprise, in any effective order, one or more of the following steps, e.g., contacting a polypeptide (e.g., in a cell, lysate, or isolated) with a test agent under conditions effective for the test agent to modulate the biological activity of the polypeptide, and determining whether the test agent modulates the biological activity.

Contacting the gene or polypeptide with the test agent can be accomplished by any suitable method and/or means that places the agent in a position to functionally control expression or biological activity of the gene or its product in the sample. Functional control indicates that the agent can exert its physiological effect through whatever mechanism it works. The choice of the method and/or means can depend upon the nature of the agent and the condition and type of environment in which the gene or its product is presented, e.g., lysate, isolated, or in a cell population (such as, in vivo, in vitro, organ explants, etc.). For instance, if the cell population is an in vitro cell culture, the agent can be contacted with the cells by adding it directly into the culture medium. If the agent cannot dissolve readily in an aqueous medium, it can be incorporated into liposomes, or another lipophilic carrier, and then administered to the cell culture. Contact can also be facilitated by incorporation of agent with carriers and delivery molecules and complexes, by injection, by infusion, etc.

Agents can be directed to, or targeted to, any part of the polypeptide that is effective for modulating it. For example, agents, such as antibodies and small molecules, can be targeted to cell-surface, exposed, extracellular, ligand binding, functional, etc., domains of the polypeptide. Agents can also be directed to intracellular regions and domains, e.g., regions where the polypeptide couples or interacts with intracellular or intramembrane binding partners.

After the agent has been administered in such a way that it can gain access to the gene or gene product (including DNA, mRNA, and polypeptides), it can be determined whether the test agent modulates its expression or biological activity. Modulation can be of any type, quality, or quantity, e.g., increase, facilitate, enhance, up-regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 10-fold, 100-fold, etc. To modulate gene expression means, e.g., that the test agent has an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post-translational processing, etc. To modulate biological activity means, e.g., that a functional activity of the polypeptide is changed in comparison to its normal activity in the absence of the agent. This effect includes, increase, decrease, block, inhibit, enhance, etc.

A test agent can be of any molecular composition, e.g., chemical compounds, biomolecules, such as polypeptides, lipids, nucleic acids (e.g., antisense, siRNA, or ribozyme targeted to a polynucleotide), carbohydrates, antibodies, ribozymes, double-stranded RNA, aptamers, etc. For example, if a polypeptide to be modulated is a cell-surface molecule, a test agent can be an antibody that specifically recognizes it and, e.g., causes the polypeptide to be internalized, leading to its down regulation on the surface of the cell. Such an effect does not have to be permanent, but can require the presence of the antibody to continue the down-regulatory effect. Antibodies can also be used to modulate the biological activity of a polypeptide in a lysate or other cell-free form.

The present invention also relates to methods of identifying modulators of a gene, differentially-expressed in fibrotic tissue or during fibrogenesis, in a cell population capable of forming fibrotic tissue, comprising, one or more of the following steps in any effective order, e.g., contacting the cell population with a test agent under conditions effective for the test agent to modulate a differentially-expressed gene disclosed herein, or a polypeptide thereof. These methods are useful, e.g., for drug discovery in identifying and confirming the pro-fibrotic or anti-fibrotic activity of agents, for identifying molecules in the normal pathway of fibrogenesis, etc.

Any cell population capable of forming (contributing to) fibrotic tissue can be utilized. Cells can include, e.g., endothelial, epithelial, muscle, embryonic and adult stem cells, ectodermal, mesenchymal, endodermal, neoplastic, etc. The phrase “capable of forming fibrotic tissue” does not indicate a particular cell-type, but simply that the cells in the population are able under appropriate conditions to form or contribute to fibrotic tissue structure. In some circumstances, the population may be heterogeneous, comprising more than one cell-type, only some which actually form fibrotic tissue, but others which are necessary to initiate, maintain, etc., the process of fibrogenesis.

The cell population can be contacted with the test agent in any manner and under any conditions suitable for it to exert an effect on the cells, and to modulate the differentially-expressed gene or polypeptide. The means by which the test agent is delivered to the cells may depend upon the type of test agent, e.g., its chemical nature, and the nature of the cell population. Generally, a test agent must have access to the cell population, so it must be delivered in a form (or pro-form) that the population can experience physiologically, i.e., to put in contact with the cells. For instance, if the intent is for the agent to enter the cell, if necessary, it can be associated with any means that facilitate or enhance cell penetrance, e.g., associated with antibodies or other reagents specific for cell-surface antigens, liposomes, lipids, chelating agents, targeting moieties, etc. Cells can also be treated, manipulated, etc., to enhance delivery, e.g., by electroporation, pressure variation, etc.

A purpose of administering or delivering the test agents to cells capable of forming blood vessels is to determine whether they modulate a gene that is differentially expressed in fibrotic tissue, such as those disclosed herein. By the phrase “modulate,” it is meant that the gene or polypeptide affects the polypeptide or gene in some way. Modulation includes effects on transcription, RNA splicing, RNA editing, transcript stability and turnover, translation, polypeptide activity, and, in general, any process involved in the expression and production of the gene and gene product. The modulatory activity can be in any direction, and in any amount, including, up, down, enhance, increase, stimulate, activate, induce, turn on, turn off, decrease, block, inhibit, suppress, prevent, etc.

Any type of test agent can be used, comprising any material, such as chemical compounds, biomolecules, such as polypeptides (including polypeptide fragments and mimics), lipids, nucleic acids (such as short interfering RNA (siRNA), antisense, or ribozymes), carbohydrates, antibodies, small molecules, fusion proteins, etc. Test agents can include, e.g., protamine, heparins, steroids, angiostatins, triazines, endostatins, cytokines, chemokines, FGFs, etc. The agent can be one based on a pyrazolopyridine scaffold (Beight, D. W. et al., WO 2004/026871), a pyrazole scaffold (Gellibert, F. et al., J. Med. Chem., 2004, 47:4494-4506), an imidazopyridine scaffold (Lee, W. C. et al., Wo 2004/021989), triazole scaffold (Blumberg, L. C. et al., WO 2004/026307), a pyridopyrimidine scaffold (Chakravarty, S. et al., WO 2000/012497), or an isothiazole scaffold (Munchhof, M. J., WO 2004/147574), for example.

Whether the test agent modulates a differentially expressed gene or polypeptide encoded by a differentially expressed gene can be determined by any suitable method. These methods include, detecting gene transcription, detecting mRNA, detecting polypeptide and activity thereof. The detection methods include those mentioned herein, e.g., PCR, RT-PCR, Northern blot, ELISA, Western, RIA, etc. In addition to detecting nucleic acid and polypeptide, further downstream targets can be used to assess the effects of modulators, including, the presence or absence of TGF-beta receptor signal transduction (e.g., TGF-beta II receptor signal transduction) as modulated by a test agent.

The method for identifying modulators of differentially expressed genes or polypeptides encoded by differentially expressed genes can include the additional step of evaluating the effects of the test agent on an animal model of fibrosis. The use of an animal model can be used before, during, or after a test agent has been identified as a modulator of a differentially expressed gene or polypeptide encoded by a differentially expressed gene in accordance with the present invention. Animal models that are genetically susceptible to the development of tumors may be used. For example, the Eker rat carries a mutation in the tuberous sclerosis 2 (Tsc-2) tumor suppressor gene and is predisposed to the development of tumors of the digestive tract (renal cell carcinomas) and reproductive tract (uterine leiomyomas) (Everitt J. I. et al., American Journal of Pathology, 1995, 146:1556-1567; Hunter D. S. et al., Cancer Research, 59:3090-3099; Walker C. L. et al., Genes Chromosomes Cancers, 2003, 38(4):349-356; Everitt J. T. et al., Toxicol. Lett., 1995, 82-83:621-625; Yoon H. et al., Am. J. Physiol. Renal. Physiol., 2002, 283:F262-F270; Everitt J. I. et al., American Journal of Pathology, 1995, 146:1556-1567; each of which is incorporated herein by reference in its entirety). Because of their inherited susceptibility to tumor development, Eker rats are an excellent model system for studying the effects of chemical carcinogens on predisposed individuals and for identifying the mechanisms by which chemical carcinogens interact with tumor susceptibility genes. In addition to being useful for studying the effects of carcinogens on tumor susceptibility genes, animal models in which spontaneous tumors occur at a high frequency are also useful in preclinical studies conducted to identify agents that may be used to prevent or treat fibrosis. Thus, test agents may be administered to rats carrying the Eker mutation or other animal model to determine if the test agent is capable of preventing or reducing the growth of fibrotic tissue, such as fibrotic tissue of the uterus.

In another aspect, the invention concerns an array, such as a gene array, including a substrate (such as a solid support) having a plurality of addresses (such as wells), wherein each address disposed thereon has a capture probe that can specifically bind at least one polynucleotide that is differentially expressed in fibrotic disorders, or a complement thereof. In one embodiment, the at least one polynucleotide is selected from the group consisting of docking protein 1, 62 kD (downstream of tyrosine kinase 1); centromere protein A (17 kD); catenin (cadherin-associated protein), beta 1 (88 kD); nuclear receptor subfamily 1, group I, member 2; v-rel avian reticuloendotheliosis viral oncogene homolog A; LGN Protein; CDC28 protein kinase 1; hypothetical protein; solute carrier family 17 (sodium phosphate), member 1; FOS-like antigen-1; nuclear matrix protein p84; LERK-6 (EPLG6); visinin-like 1; phosphodiesterase 10A; KH-type splicing regulatory protein (FUSE binding protein 2); Polyposis locus (DP1 gene) mRNA; microtubule-associated protein 2; CDC5 (cell division cycle 5, S pombe, homolog)-like; Centromere autoantigen C (CENPC) mRNA; RNA guanylyltransferase and 5′-phosphatase; Nijmegen breakage syndrome 1 (nibrin); ribonuclease, RNase A family, 4; keratin 10 (epidermolytic hyperkeratosis; keratosis palmaris et plantaris); basic helix-loop-helix domain containing, class B, 2; dual specificity phosphatase 1; annexin A11; putative receptor protein; Human endogenous retrovirus HERV-K(HML6); mitogen-activated protein kinase kinase kinase 12; TXK tyrosine kinase; kynureninase (L-kynurenine hydrolase); ubiquitin specific protease 4 (proto-oncogene); peroxisome biogenesis factor 13; olfactory receptor, family 2, subfamily F, member 1; membrane protein, palmitoylated 3 (MAGUK p55 subfamily member 3); origin recognition complex, subunit I (yeast homolog)-like; dTDP-D-glucose 4,6-dehydratase; cytochrome c oxidase subunit VIa polypeptide 2; gamma-tubulin complex protein 2; Monocyte chemotactic protein-3; myelin transcription factor 1; inhibitor of growth family, member 1-like; thyroid hormone receptor, alpha myosin-binding protein C, slow-type; fragile X mental retardation 2; sonic hedgehog (Drosophila) homolog; 6-phosphofructo-2-kinase/fructose-2,6-biphosphatase 2; SFRS protein kinase 2; excision repair cross-complementing rodent repair deficiency; cyclin-dependent kinase 5, regulatory subunit 1 (p35); poly(A)-specific ribonuclease (deadenylation nuclease); solute carrier family 12 (potassium/chloride transporters), member 4; Pseudogene for metallothionein; natriuretic peptide precursor A; intercellular adhesion molecule 2; apoptosis antagonizing transcription factor; similar to rat HREV107; major histocompatibility complex, class II, DP beta 1; MpV17 transgene, murine homolog, glomerulosclerosis; uroporphyrinogen decarboxylase; proteasome (prosome, macropain) 26S subunit, ATPase, 1; fins-related tyrosine kinase 3 ligand; actin, gamma 1; Protein Kinase Pitslre, Alpha, Alt. Splice 1-Feb; nuclear factor of kappa light polypeptide gene enhancer in B-cells inhibitor, alpha; pyruvate kinase, muscle; telomeric repeat binding factor 2; cell division cycle 2, G1 to S and G2 to M; ADP-ribosylation factor 3; NRF1 Protein; H factor (complement)-like 3; serine (or cysteine) proteinase inhibitor, clade B (ovalbumin), member 6; mRNA of muscle specific gene M9; solute carrier family 25 (mitochondrial carrier; phosphate carrier), member 3; ribosomal protein L36a; suppressor of Ty (S. cerevisiae) 4 homolog 1; amino-terminal enhancer of split; ubiquitin A-52 residue ribosomal protein fusion product 1; hydroxyacyl-Coenzyme A dehydrogenase/3-ketoacyl-Coenzyme A thiolase; chaperonin containing TCP™, subunit 2 (beta); tyrosine kinase with immunoglobulin and epidermal growth factor homology; domains; Fc fragment of IgG, receptor, transporter, alpha; NRD1 convertase; ADP-ribosylation factor 5; transcription elongation factor A (SII), 1; like mouse brain protein E46; titin; fibromodulin; and Abi-interactor 2 (Abi-2).

In another embodiment of the array, the at least one polynucleotide includes at least one gene selected from the group consisting of CDKN1B, CDKN1C, CTGF, fibromodulin, and Abi-2.

In another embodiment of the array, the at least one polynucleotide includes at least one gene selected from the group consisting of IL-11, IL-13, EGR1, EGR2, EGR3, CITED2, P300, E2F1, E2F2, E2F3, E2F4, E2F5, MCP3, CXCL5, CCL7, SMAD3, TYMS, GT198, SMAD7, NCOR2, TIMP-1, and ADAM17.

In another embodiment of the array, the at least one polynucleotide includes at least one of those genes listed in Table 9.

In another embodiment of the array, the at least one polynucleotide includes at least one gene selected from the group consisting of stanniocalcin 2, interleukin 11, disintegrin and metalloproteinase domain 17, early growth response 3, fibromodulin, collagen type XVIII alpha 1, and interleukin 13.

In another embodiment of the array, the at least one polynucleotide includes a plurality of genes comprising stanniocalcin 2, interleukin 11, disintegrin and metalloproteinase domain 17, early growth response 3, fibromodulin, collagen type XVIII alpha 1, and interleukin 13.

In another embodiment of the array, the array further comprises a capture probe that can specifically bind at least one polynucleotide encoding a house-keeping gene as a control.

In another embodiment of the array, each of the addresses comprises a well, and each of the capture probes comprises a primer for amplifying RNA in a biological sample that is deposited in the well

In one embodiment, the capture probes are polynucleotides that hybridize to the differentially expressed polynucleotides under stringent conditions or mild conditions. In another embodiment of the array, each of the capture probes binds the polynucleotides (e.g., hybridizes with the polynucleotide along the full length of the polynucleotide or along substantially the full length of the polynucleotide) under stringent conditions. As used herein “stringent” conditions for hybridization refers to conditions which achieve the same, or about the same, degree of specificity of hybridization as the conditions employed by the current applicants. Specifically, hybridization of immobilized DNA on Southern blots with 32P-labeled gene-specific probes was performed by standard methods (Maniatis, T., E. F. Fritsch, J. Sambrook [1982] Molecular Cloning: A Laboratory Manual, Cold Spring Harbor Laboratory, Cold Spring Harbor, N.Y.). In general, hybridization and subsequent washes are carried out under stringent conditions that allow for hybridization of target sequences with homology to the capture probes. For double-stranded DNA gene probes, hybridization was carried out overnight at 20-25° C. below the melting temperature (Tm) of the DNA hybrid in 6×SSPE, 5×Denhardt's solution, 0.1% SDS, 0.1 mg/ml denatured DNA. The melting temperature is described by the following formula (Beltz, G. A., K. A. Jacobs, T. H. Eickbush, P. T. Cherbas, and F. C. Kafatos, Methods of Enzymology, 1983, R. Wu, L. Grossman and K. Moldave [eds.] Academic Press, New York 100:266-285).

Tm=81.5° C.+16.6 Log [Na+]+0.41(% G+C)−0.61(% formamide)−600/length of duplex in base pairs.

Washes are typically carried out as follows:

(1) Twice at room temperature for 15 minutes in 1×SSPE, 0.1% SDS (low stringency wash).

(2) Once at Tm-20° C. for 15 minutes in 0.2×SSPE, 0.1% SDS (moderate stringency wash).

For oligonucleotide probes, hybridization was carried out overnight at 10-20° C. below the melting temperature (Tm) of the hybrid in 6×SSPE, 5×Denhardt's solution, 0.1% SDS, 0.1 mg/ml denatured DNA. Tm for oligonucleotide probes can be determined by the “nearest-neighbor” method. See Breslauer et al., “Predicting DNA duplex stability from the base sequence,” Proc. Natl. Acad. Sci. USA, 83 (11): 3746-3750 (June 1986); Rychlik and Rhoads, “A computer program for choosing optimal oligonucleotides for filter hybridization, sequencing and in vitro amplification of DNA,” Nucleic Acids Res., 17 (21): 8543-8551 (Nov. 11, 1989); Santa Lucia et al., “Improved nearest-neighbor parameters for predicting DNA duplex stability,” Biochemistry 35 (11): 3555-3562 (Mar. 19, 1996); Doktycz et al., “Optical melting of 128 octamer DNA duplexes. Effects of base pair location and nearest neighbors on thermal stability,” J. Biol. Chem., 270 (15): 8439-8445 (Apr. 14, 1995). Alternatively, the Tm can be determined by the following formula:

Tm (° C.)=2(number T/A base pairs)+4(number G/C base pairs) (Suggs, S. V., T. Miyake, E. H. Kawashime, M. J. Johnson, K. Itakura, and R. B. Wallace [1981] ICN-UCLA Symp. Dev. Biol. Using Purified Genes, D. D. Brown [ed.], Academic Press, New York, 23:683-693).

Washes are typically carried out as follows:

(1) Twice at room temperature for 15 minutes 1×SSPE, 0.1% SDS (low stringency wash).

(2) Once at the hybridization temperature for 15 minutes in 1×SSPE, 0.1% SDS (moderate stringency wash).

In another embodiment of the array, each polynucleotide bound by the capture probe of each address is unique among the plurality of addresses.

In another embodiment of the array, the substrate has no more than 500 addresses.

In another embodiment of the array, the substrate has 200 to 500 addresses.

The substrate of the array of the invention can be any solid support suitable for disposing the capture probes, such as those materials known in the art used for fabrication of gene arrays and/or microfluidics. “Arraying” refers to the act of organizing or arranging members of a library, or other collection, into a logical or physical array. Thus, an “array” refers to a physical or logical arrangement of, e.g., library members (candidate agent libraries). A physical array can be any “spatial format” or physically gridded format” in which physical manifestations of corresponding library members are arranged in an ordered manner, lending itself to combinatorial screening. For example, samples corresponding to individual or pooled members of a candidate agent library or patient library can be arranged in a series of numbered rows and columns, e.g., on a multiwell plate. Similarly, capture probes can be plated or immobilized (in a lyophilized or other state) or otherwise deposited in microtitered, e.g., 96-well, 384-well, or -1536 well, plates (or trays).

A “solid support” (also referred to herein as a “solid substrate”) has a fixed organizational support matrix that preferably functions as an organization matrix, such as a microtiter tray. Solid support materials include, but are not limited to, glass, polacryloylmorpholide, silica, controlled pore glass (CPG), polystyrene, polystyrene/latex, polyethylene, polyamide, carboxyl modified teflon, nylon and nitrocellulose and metals and alloys such as gold, platinum and palladium. The solid support can be biological, non-biological, organic, inorganic, or a combination of any of these, existing as particles, strands, precipitates, gels, sheets, tubing, spheres, containers, capillaries, pads, slices, films, plates, slides, etc., depending upon the particular application. Other suitable solid substrate materials will be readily apparent to those of skill in the art. The surface of the solid substrate may contain reactive groups, such as carboxyl, amino, hydroxyl, thiol, or the like for the attachement of nucleic acids, proteins, etc. Surfaces on the solid substrate will sometimes, though not always, be composed of the same material as the substrate. Thus, the surface can be composed of any of a wide variety of materials, for example, polymers, plastics, resins, polysaccharides, silica or silica-based materials, carbon, metals, inorganic glasses, membranes, or any of the above-listed substrate materials.

In addition to standard gene arrays, such as the commercially available gene arrays described herein, micro fluidic cards (e.g., 7900 HT Micro Fluidic Card, APPLIED BIOSYSTEMS) may be used to profile gene expression using the comparative C_{T} method of relative quantification. Such cards are also contemplated in the arrays of the present invention. Microfluidic card experiments use a two-step RT-PCR process. In the reverse-transcription (RT) step, cDNA is reverse transcribed from total RNA samples using random primers from the high capacity cDNA archive kit. Additional details about the RT-PCR process are contained in the high capacity cDNA archive kit protocol (PN 4322169). In the PCR step, PCR products are synthesized from cDNA samples using the TAQMAN universal PCR master mix. The PCR step employs the 5′ nuclease assay, which is described in Appendix C of the user's guide for the 7900HT system. Relative gene expression values can be obtained from 7900HT system data using the comparative C_{T} method for relative quantification. In the comparative C_{T} method, quantity is expressed relative to a calibrator sample that is used as the basis for comparative results (see Applied Biosystems 7900HT Micro Fluidic Card Getting Started Guide, APPLIED BIOSYSTEMS, which is incorporated herein by reference in its entirety). Real-time quantitative gene expression results are available as soon as the thermal cycling process is complete.

All wells on the card are connected by a series of channels, and assays are loaded at the factory before shipping. The biological sample is combined with TAQMAN Universal PCR Master Mix and loaded into the card ports. The card may contain any number of wells, such as 96, 192, 384, 500, 1000, etc. Real-time performance can be obtained by using a micro fluidic card in a high throughput 384-well format, 2 microliter reaction volume, and eight loading ports. Briefly, sample (e.g., isolated RNA) is loaded into the micro fluidic card, the card is centrifuged to transfer mixes into the individual wells, and the card is sealed using a sealing device which individually seals each well to avoid diffusion and cross-talk. The sealed card is then ready for real-time PCR. The fill reservoirs are trimmed and the card is loaded on the 7900HT system for real-time PCR. The 384 well format provides configuration flexibility. For example, using one sample per micro fluidic card, 384 genes with single data points, or 96 genes with 4 replicates may be assayed. Using eight samples per micro fluidic card, 48 genes with single data points, 24 genes with 2 replicates, or 12 genes with 4 replicates may be assayed. Isolated RNA from tumor tissues, normal tissues, or cells can be injected into the card. The card can be divided into normal tissue and tumor tissue, for example. Using a 384 well format, 48 genes of four individuals (human or non-human animal subjects) with normal tissue and tumor tissue can be assayed.

The effects of test agents, such as TGF-beta receptor inhibitors (e.g., SB505124/SB431542), TGF-beta signaling inhibitors (halofuginone), and potential environment carcinogens or gene express can be determined using the method of the invention.

For differential expression analysis, it is preferable to include at least one house-keeping gene (as a control gene) whose expression should not change, such as GAPD (GenBank accession number NM_{—}002046), or other house-keeping genes described herein.

Table 9 lists genes that may be used on a micro fluidic card in accordance with the subject invention. For example, one or more genes from each category listed in Table 9 can be assayed for differential expression (e.g., cell adhesion molecule, extracellular matrix, kinase, oxidoreductase, protease, signaling molecule, transcription factor).

Optionally, once a test agent is identified as a modulator, the method of the invention may further include the step of manufacturing the identified modulator. The manufacturing step may involve synthesis of the modulator (e.g., if a small molecule) or genetic engineering, for example. Optionally, the manufacturing step may further comprise combining the manufactured modulator with another active substance and/or a pharmaceutically acceptable carrier or excipient, as a formulated composition.

As used herein, the terms “bind,” “binds,” or “interacts with” mean that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ moles/liter for that second molecule.

By reference to an “antibody that specifically binds” another molecule is meant an antibody that binds the other molecule, and displays no substantial binding to other naturally occurring proteins other than those sharing the same antigenic determinants as other molecule. The term “antibody” includes polyclonal and monoclonal antibodies as well as antibody fragments or portions of immunolglobulin molecules that can specifically bind the same antigen as the intact antibody molecule.

As used herein, a “nucleic acid,” “nucleic acid molecule,” “oligonucleotide,” or “polynucleotide” means a chain of two or more nucleotides such as RNA (ribonucleic acid) and DNA (deoxyribonucleic acid).

The term “subject,” as used herein, means a human or non-human animal, including but not limited to mammals, such as a dog, cat, horse, cow, pig, sheep, goat, chicken, primate, rat, and mouse. In a preferred embodiment, the subject is female, such as a human female.

The term “differentially expressed gene”, as used herein, means a gene that is either over-expressed or underexpressed in fibrotic tissue (such as leiomyoma), compared to normal, non-fibrotic tissue. Accordingly, the method of treatment of the present invention is directed to upregulating the expression of one or more genes that are underexpressed in fibrotic tissue, or increasing the activity of the polypeptide encoded by the gene; and downregulating the expression of one or more genes that are overexpressed in fibrotic tissue, or decreasing the activity of the polypeptide encoded by the gene.

When referring to a differentially expressed gene, the phrase “modulates the expression of” means upregulates or downregulates the amount or functional activity of the gene, or otherwise modifies the activity of the gene product, e.g., the availability of the gene product to interact with a receptor.

The terms, “treat”, “treatment”, and “treating”, as used herein, are intended to include the prevention of a fibrotic disorder and partial or full alleviation of an existing fibrotic disorder within a human or non-human animal subject (e.g., a reduction in the severity of one or more symptoms associated with the fibrotic disorder). For example, treating a fibroid, such as a uterine fibroid, can include a reduction in the size of the fibroid and/or a reduction in the rate of the fibroid's growth.

### Materials and Methods

The following materials and methods describe those utilized in Examples 1-8.

Tissues. Portions of leiomyoma and matched myometrium were collected from premenopausal women (N=6) who were scheduled to undergo hysterectomy for indications related to symptomatic leiomyomas. Three of the patients received GnRHa therapy for three months prior to surgery. The untreated patients did not receive any medications (including hormonal therapy) during the previous 3 months prior to surgery, and based on endometrial histology and the patient's last menstrual period they were from early-mid secretory phase of the menstrual cycle. To maintain a standard, all leiomyomas selected for this study were between 2 to 3 cm in diameter. Following collection, the tissues were divided into several pieces and either immediately snap frozen and stored in liquid nitrogen for further processing, fixed and paraffin embedded for histological evaluation and immunohistochemistry, or used for isolation of leiomyoma and myometrial smooth muscle cells and culturing (Ding, L et al. J Clin Endocrinol Metab, 2004, 89:5549-5557; Xu, J et al. J Clin Endocrinol Metab, 2003, 88:1350-61). The tissues were collected at the University of Florida affiliated Shands Hospital with prior approval obtained from the Institutional Review Board.

Isolation and Culture of Leiomyoma and Myometrial Smooth Muscle Cells. To determine the direct action of GnRHa on global gene expression in leiomyoma and myometrial smooth muscle cells (LSMC and MSMC), the cells were isolated and cultured as previously described (Ding, L et al. J Clin Endocrinol Metab, 2004, 89:5549-5557; Chegini, N et al. Mol Hum Reprod, 2002, 8:1071-8). Only untreated tissues were used for isolation of LSMC and MSMC. Prior to use in these experiments, the primary cell cultures were seeded in 8-well culture slides (Nalge Nunc, Naperville, Ill.) and after 24 hours of culturing they were characterized using immunofluoroscence microscopy and antibodies to α smooth muscle actin, desmin and vimentin (Ding, L et al. J Clin Endocrinol Metab, 2004, 89:5549-5557; Xu, J et al. J Clin Endocrinol Metab, 2003, 88:1350-61). LSMC and MSMC were cultured in 6-well plates at an approximate density of 10⁶ cells/well in DMEM-supplemented media containing 10% FBS. After reaching visual confluence, the cells were washed in serum-free media and incubated for 24 hrs under serum-free, phenol red-free condition (Chegini, N et al. Mol Hum Reprod, 2002, 8:1071-8). The cells were then treated with 0.1 μM of GnRHa (leuprolide acetate, Sigma Chemical, St Louis, Mo.) for a period of 2, 6 and 12 hours (Ding, L et al. J Clin Endocrinol Metab, 2004, 89:5549-5557).

cDNA Microarray and Gene Expression Profiling. Total cellular RNA was isolated from the tissues and cells using Trizol (INVITROGEN, Carlsbad, Calif.). The isolated RNA was treated with DNase I (Roche, Molecular Biochemicals, Indianapolis, Ind.) at 1 unit/10 μg of RNA for 20 min at 25° C., heat-inactivated at 75° C. and subjected to further purification using RNeasy Kit (QIAGEN, Valencia, Calif.). The RNA was then subject to amplification by reverse transcription using SuperScript Choice system (Invitrogen), with final concentrations in 20 μl first-strand reaction of 100 μmol of high performance liquid chromatography-purified T7-(dT)24 primer (Genset Corp, La Jolla, Calif.), 8 μg of RNA, 1× first-strand buffer, 10 mM dithiothreitol, 500M of each dNTP, and 400 units of Superscript II reverse transcriptase (T7 Megascript kit; Ambion, Austin, Tex.). The second-strand cDNA synthesis was performed in a 150 μl reaction consisting of, at the final concentrations, 1× second-strand reaction buffer, 200 μM each dNTP, 10 units of DNA ligase, 40 units of DNA polymerase I, and 2 units of RNase H (INVITROGEN), and double-stranded cDNA was purified by phenol:chloroform extraction using phase lock gels (Eppendorf-5 Prime, Inc. Westbury, N.Y.) and an ethanol precipitation (Chegini, N et al. J Soc Gynecol Investig, 2003, 10:161-71).

Five micrograms of purified cDNA was reverse transcribed using Enzo BioArray high yield RNA transcript labeling kit (AFFYMETRIX, Santa Clara, Calif.) and the product was purified in RNeasy spin columns (QIAGEN) according to manufacture's instructions. Following an overnight ethanol precipitation, cRNA was re-suspended in 15 μl of diethyl pyrocarbonate-treated water (AMBION) and quantified using a Beckman DU530 Life Science UV-visible spectrophotometer. Following quantification of cRNA to reflect any carryover of unlabeled total RNA according to an equation given by Affymetrix (adjusted cRNA yield=cRNA (μg) measured after in vitro transcription (starting total RNA) (fraction of cDNA reaction used in in vitro transcription), 20 g of cRNA was fragmented (0.5 μg/μl) according to Affymetrix instructions using the 5× fragmentation buffer containing 200 mM Tris acetate, pH 8.1, 500 mM potassium acetate and 150 mM magnesium acetate (SIGMA Chemical, St. Louis, Mo.). 20 μg of the adjusted fragmented cRNA was added to a 300 μl of hybridization mixture containing at final concentrations 0.1 mg/ml herring sperm DNA (Promega/Fisher, Madison, Wis.), 0.5 mg/ml acetylated bovine serum albumin (INVITROGEN), and 2×MES hybridization buffer (Sigma). 200 μl of the mixture was hybridized to the human U95A Affymetrix GeneChip arrays, purchased at the same time from the same lot number and used within two weeks of purchase in order to maintain standard. In addition, an aliquot of random samples were first hybridized to an Affymetrix Test 2 Array to determine sample quality according to manufacturer's criteria. After meeting recommended criteria for use of the expression arrays, the hybridization was performed for 16 hrs at 45° C., followed by washing, staining, signal amplification with biotinylated anti-strepavidin antibody, and the final staining step according to manufactures protocol.

Microarray Data Analysis. The Chips were scanned to obtain the raw hybridization values using Affymetrix Genepix 5000A scanner. Difference in the levels of fluorescence spot intensities representing the rate of hybridization between the 25 basepair oligonucleotides and their mismatches were analyzed by multiple decision matrices to determine the presence or absence of gene expression, and to derive an average difference score representing the relative level of gene expression. The fluorescence spot intensities, qualities and local background were assessed automatically by Genepix software with a manual supervision to detect any inaccuracies in automated spot detection. Background and noise corrections were made to account for nonspecific hybridization and minor variations in hybridization conditions. The net hybridization values for each array were normalized using a global normalization method as previously described (Chegini, N et al. J Soc Gynecol Investig, 2003, 10:161-71). To identify the changes in pattern of gene expression, the average and standard deviation (SD) of the globally normalized values were calculated followed by subtraction of the mean value from each observation and division by the SD. The mean transformed expression value of each gene in the transformed data set was set at 0 and the SD at 1 (Chegini, N et al. J Soc Gynecol Investig, 2003, 10:161-71).

The transformed gene expression values were subjected to Affymetrix Analysis Suite V 5.0. Briefly, probe sets that were flagged as absent on all arrays using default settings were removed from the datasets. After application of this filtering, the dataset was reduced from 12,625 probe sets to 8580 probe sets. The gene expression value of the remaining probe sets was then subjected to unsupervised and supervised learning, discrimination analysis, and cross validation (Eisen, M B et al. Proc Natl Acad Sci USA, 1998, 95:14863-14868; Varela, J C et al. Invest Opthalmol V is Sci, 2002, 43:1772-1782; Tusher, V G et al. Proc Natl Acad Sci USA, 2001, 98:5116-5121; Pavlidis, P Methods, 2003, 31:282-289; Peterson, L E Comput Methods Programs Biomed, 2003, 70:107-19; Butte, A Nat Rev Drug Discov, 2002, 1:951-960). After variation filtering, the coefficient of variation was calculated for each probe set across all chips and the probe sets were ranked by the coefficient of variation of the observed single intensities. The expression values of the selected genes were then subjected to R programming analysis that assesses multiple test correction to identify statistically significant gene expression values (Pavlidis, P Methods, 2003, 31:282-289; Peterson, L E Comput Methods Programs Biomed, 2003, 70:107-19; Butte, A Nat Rev Drug Discov, 2002, 1:951-960). The gene expression values having a statistical significance of p≦0.02 (ANOVA, Tukey test) between leiomyoma and myometrium from GnRH-treated and untreated cohorts, and p≦0.005 between GnRHa-treated and untreated cells (control) were selected. The validity of gene sets identified at these p values in predicting treatment class was established using “leave-one-out” cross validation where the data from one array was left out of the training set and probe sets with differential hybridization signal intensities were identified from the remaining arrays (Varela, J C et al. Invest Opthalmol V is Sci, 2002, 43:1772-1782; Butte, A Nat Rev Drug Discov, 2002, 1:951-960). Hierarchical clustering and K-means analysis was performed and viewed with the algorithms in the software packages Cluster and TreeView (Eisen, M B et al. Proc Natl Acad Sci USA, 1998, 95:14863-14868).

Gene Classification and Ontology Assessment. The selected differentially expressed and regulated genes in the above cohorts were subjected to functional annotation and visualization using Database for Annotation, Visualization, and Integrated Discovery (DAVID) software (Dennis G Jr. et al., DAVID: Database for Annotation, Visualization, and Integrated Discovery, Genome Biology, 2003; 4(5):P3; Hosack D. A. et al., Glynn Dennis Jr, Brad T Sherman, HClifford Lane, Richard A Lempicki. Identifying Biological Themes within Lists of Genes with EASE, Genome Biology, 2003, 4(6):P4). The integrated GoCharts assigns genes to specific ontology functional categories based on selected classifications, KeggCharts assigns genes to KEGG metabolic processes and context of biochemical pathway maps, and DomainCharts assigning genes according to PFAM conserved protein domains.

Quantitative RealTime PCR. Realtime PCR was utilized for verification of 10 differentially expressed and regulated genes identified in leiomyoma and myometrium as well as LSMC and MSMC from untreated and GnRHa-treated cohorts. The selection of these genes was based not only on their expression values (up or downregulation), but classification and biological functions important to leiomyoma growth and regression, regulation by ovarian steroids, GnRHa and TGF-β. They are IL-11, CITED2, Nur77, EGR3, TGIF, TIEG, p27, p57, GAS-1 and GPRK5 representing cytokines, transcription factors, cell cycle regulators and signal transduction. Realtime PCR was carried out as previously described using Taqman and ABI-Prism 7700 Sequence System and Sequence Detection System 1.6 software (Ding, L et al. J Clin Endocrinol Metab, 2004, 89:5549-5557). Results were analyzed using the comparative method and following normalization of expression values to the 18S rRNA expression according to the manufacturer's guidelines (Applied Biosystems) as previously described (Ding, L et al. J Clin Endocrinol Metab, 2004, 89:5549-5557).

Western Blot Analysis and Immunohistochemical Localization. For immunoblotting, total protein was isolated from small portions of GnRHa-treated and untreated leiomyoma and myometrium as well as the GnRHa-treated and untreated cells as previously described (Ding, L et al. J Clin Endocrinol Metab, 2004, 89:5549-5557; Chegini, N et al. Mol Cell Endocrinol, 2003, 209:9-16). Following determination of the tissue homogenates and cell lysates protein content an equal amount of sample proteins were subjected to SDS-PAGE and transferred to polyvinyldiene difluoride (PVDF) membrane. The blots were incubated with anti-TIEG antibody, kindly provided by Dr. Thomas Spelsberg, Department of Biochemistry, Mayo Clinic, Rochester, Minn. (Johnsen, S A et al. Oncogene, 2002, 21:5783-90), TGIF, EGR3, p27, p57, Nur77 and Gas1 antibodies purchased from Santa Cruz Biochemical (Santa Cruz, Calif.), IL-11 antibodies purchased from R & D system (Minneapolis, Minn.) for 1 hr at room temperature. The membranes were washed, exposed to corresponding HRP-conjugated IgG for 1 hr and immunostained proteins were visualized using enhanced chemiluminesence reagents (Amersham-Pharmacia Biotech, Piscataway, N.J.) as previously described (Ding, L et al. J Clin Endocrinol Metab, 2004, 89:5549-5557; Chegini, N et al. Mol Cell Endocrinol, 2003, 209:9-16; Xu, J et al. J Clin Endocrinol Metab, 2003, 88:1350-61).

For immunohistochemical localization, tissue sections were prepared from formalin-fixed and paraffin embedded leiomyoma and myometrium. Tissue sections were microwave prior to immunostaining using antibodies to IL-11, TGIF, TIEG, EGR3, Nur77, p27, p57 and Gas1. The antibodies were used at concentrations of 5 μg of IgG/ml for 2-3 hours at room temperature. Following further processing including incubation with biotinylated secondary antibodies and avidin-conjugated HRP (ABC ELITE kit, VECTOR Laboratories, Burlingame, Calif.), the chromogenic reaction was detected with 3,3′-diaminobenzidine tetrahydrochloride solution. In some instances the slides were counter stained with hematoxylin. Omission of primary antibodies or incubation of tissue sections with non-immune mouse IgG instead of primary antibodies at the same concentration during immunostaining served as controls (Ding, L et al. J Clin Endocrinol Metab, 2004, 89:5549-5557; Chegini, N et al. Mol Cell Endocrinol, 2003, 209:9-16; Xu, J et al. J Clin Endocrinol Metab, 2003, 88:1350-61).

Determination of TGF-β1 on global gene expression in LSMC and MSMC. All the materials utilized for this study including isolation of leiomyoma and myometrial cells are identical to those described in detail above. To determine the effect of TGF-β1 on global gene expression in LSMC and MSMC, the cells were cultured in 6-well plates at approximate density of 10⁶ cells/well in DMEM-supplemented media containing 10% FBS. After reaching visual confluence the cells were washed in serum-free media and incubated for 24 hrs under serum-free, phenol red-free condition (Xu, J et al. J Clin Endocrinol Metab, 2003, 88:1350-1361; Ding, L. et al. J Clin Endocrinol Metab, 2004, 89:5549-5557). The cells were then treated with 2.5 ng/ml of TGF-β1 (R & D System, Minneapolis, Mich.) for 2, 6 and 12 hours. To further profile the autocrine/paracrine action of TGF-β1 on gene expression in LSMC and MSMC, the cells were cultured as above and treated with 1 μM of TGF-β type II receptor antisense or sense oligonucleotides for 24 hours as previously described (Xu, J et al. J Clin Endocrinol Metab, 2003, 88:1350-1361; Ding, L. et al. J Clin Endocrinol Metab, 2004, 89:5549-5557). The cells were washed and then treated with TGF-β1 (2.5 ng/ml) for 2 hours. Parallel experiments using untreated cells were used as controls including an additional control for TGF-β type II receptor antisense and sense experiments.

Total cellular RNA was isolated from LSMC- and MSMC-treated and untreated controls and subjected to microarray analysis. To maintain standard and allow for comparative analysis, the GeneChips used in this study were utilized, simultaneously processed and their gene expression values were subjected to global normalization and transformation. Following these unsupervised assessments the coefficient of variation was calculated for each probe set across all the chips used in this study, and the selected gene expression values of this study were independently subjected to supervised learning including R programming analysis and ANOVA with false discovery rate selected at p<0.001 (Moustakas, A. Immunol Lett, 2002, 82:85-91; Verrecchia, F. et al. J Biol Chem, 2001, 276:17058-17062). The genes identified in these cohorts were analyzed for functional annotation and visualized using Database for Annotation, Visualization, and Integrated Discovery (DAVID) software with integrated GoCharts. Following the analysis, we selected 12 of the differentially expressed and regulated genes, including 10 identified and validated in leiomyoma and myometrium from untreated and GnRHa-treated cohorts, as well as LSMC and MSMC treated in vitro with GnRHa, for validation in response to TGF-β-time dependent action using Realtime PCR. They include IL-11, EGR3, CITED2, TIEG, TGIF, Nur77, p27, p57, GAS-1 and GPRK5. In addition, the expression of Runx1 and Runx2, transcription factors that interact with TGF-β receptor signaling pathways (Zavadil, J. et al. Proc Natl Acad Sci USA, 2001, 98:6686-6691), was validated in LSMC and MSMC as well as in leiomyoma and myometrium from GnRHa-treated and untreated cohorts. Detail description of the materials and methods for Realtime PCR as well as data analysis is provided in Chegini, N. et al. J Soc Gynecol Investig, 2003, 10:161-71.

### EXAMPLE 1

### Gene Expression Profiles in Leiomyoma and Normal Myometrium

Global gene expression profiling has been instrumental in identifying the molecular environment of tissues with respect to fingerprints of their physiological and pathological behavior, and in vitro cellular responses to various regulatory molecules. The present inventors used this approach and characterized the gene expression profile of leiomyoma and matched myometrium, and their transcriptional changes in response to hormonal transition induced by GnRHa therapy. The initial assessment of the gene expression values in leiomyoma, myometrium and their isolated smooth muscle cells from untreated as well as GnRHa- and TGF-β-treated cohorts revealed a uniform expression of transcripts for the housekeeping genes glyceraldehyde-3-phosphate dehydrogenase, α-actin and a large number of ribosomal proteins, indicating that the expression profile is consistent with established standards for gene expression analysis. Following global normalization and transformation of the gene expression values, supervised learning, discrimination analysis, cross validation and variation filtering, the gene expression values were subjected to R programming analysis and ANOVA with false discovery rate selected at p≦0.02.

Using the above analysis, the present inventors identified a total of 153 genes, including 19 EST, or 1.23% of the genes, and 122 genes including 21 EST or 0.98% of the genes on the array, as differentially expressed in leiomyoma compared to matched myometrium from untreated and GnRHa-treated tissues, respectively. Hierarchical clustering and Tree-View analysis separated the genes in each cohort into distinctive clusters with sufficient variability allowing division into their respective subgroups. Of the 153 (excluding 19 EST) differentially expressed genes in untreated cohorts, 82 were upregulated and 52 downregulated in leiomyoma compared to myometrium (Table 1). Of the 122 genes (excluding 21 EST) in leiomyoma and myometrium from patients who received GnRHa therapy, 34 transcripts were upregulated and 67 downregulated, in leiomyoma compared to myometrium, respectively (Table 2). Analysis of the variance-normalized mean (K-means) separated the differentially expressed and regulated genes in these cohorts into 4 distinctive clusters, with genes in clusters A and D displaying a tissue-specific response, while genes in cluster B and C showing regulatory response to GnRHa therapy. To further characterize the molecular environment of leiomyoma from myometrium and their response to GnRHa therapy, we compared the gene expression profiles in GnRHa-treated with corresponding untreated tissues. The analysis indicated that the expression of 170 (excluding 26 EST) and 167 (excluding 31 EST) genes are targeted by GnRHa therapy in leiomyoma and myometrium, compared to their respective untreated cohorts (Tables 3 and 4). Of these genes, 96 and 89 transcripts were downregulated in leiomyoma and myometrium, respectively, due to GnRHa therapy, compared to their respective untreated tissues, with 3 transcripts were commonly found among the tissues in these cohorts, with different regulatory pattern of expression (compare Tables 3 and 4).

Table 1 is a categorical list of differentially expressed genes identified in leiomyoma compared to matched myometrium. The genes were identified following unsupervised and supervised analysis of their expression values and subjected to R programming environment and ANOVA with a false-discovery rate of rate of p≦0.02 as described in materials and methods. Of the 153 genes identified as differentially expressed, 82 genes were up (+) and 52 genes were downregulated (−) in leiomyoma compared to myometrium excluding 19 EST.

Table 2 is a categorical list of differentially expressed genes identified in leiomyoma compared to myometrium in response to GnRHa therapy. The genes were identified following unsupervised and supervised analysis of their expression values and statistical analysis in R programming environment and ANOVA with a false-discovery rate selected at p≦0.02. Of the 122 genes identified, the expression of 34 genes was up (+) and 67 gene downregulated (−) in GnRH-treated leiomyoma (LYM) compared to myometrium (MYM) excluding 21 EST).

Table 3 is a categorical list of differentially expressed genes identified in leiomyoma from GnRHa-treated compared to untreated leiomyoma. The genes were identified following unsupervised and supervised analysis of their expression values and statistical analysis in R programming environment and ANOVA with a false-discovery rate selected at p≦0.02. Of the 170 genes identified, the expression of 74 genes was up (+) and 96 genes downregulated (−) in GnRH-treated compared to untreated leiomyoma (LMY) excluding 26 EST.

Table 4 is a categorical list of differentially expressed genes identified in myometrium from GnRHa-treated compared to untreated myometrium. The genes were identified following unsupervised and supervised analysis of their expression values and statistical analysis in R programming environment and ANOVA with a false-discovery rate selected at p≦0.02. Of the 167 genes identified, the expression of 47 genes was up (+) and 89 genes downregulated (−) in GnRH-treated compared to untreated myometrium (MYM) excluding 31 EST.

A few microarray studies have reported the gene expression profile of leiomyoma and myometrium (Tsibris, J C M et al. Fertil Steril, 2002, 78:114-121; Chegini, N et al. J Soc Gynecol Investig, 2003, 10:161-71; Wang, H et al. Fertil Steril, 2003, 80:266-76; Weston, G et al. Mol Hum Reprod, 2003, 9:541-9; Ahn, W S et al. Int J Exp Pathol, 2003, 84:267-79; Quade, B J et al. Genes Chromosomes Cancer, 2004, 40:97-108). The present inventors performed a comparative analysis using the differentially expressed genes identified in the untreated leiomyoma and matched myometrium of this study, with the list of genes reported in four of the other studies, searching for a set of commonly expressed genes. The comparison identified 2 genes in this study in common with at least one of the other studies. However, lowering the false discover rate to p≦0.05 enabled the identification of a larger number of genes (422 including 49 EST), of which 11 transcripts were found in common with other studies (Table 5).

Table 5 is a list of the common genes found in this study of leiomyoma and matched myometrium from early-med secretory phase of the menstrual cycle following unsupervised and supervised analysis of their expression values and statistical analysis in R programming environment and ANOVA with a false-discovery rate selected at p≦0.05 to allow comparison with the results of four other microarray studies utilizing leiomyoma and myometrium from proliferative and secretory phases of the menstrual cycle.

Gene ontology assessment and division of differentially expressed genes into similar functional categories indicated that the products of a large percentage of these genes (40% to 67%), in leiomyoma and myometrium from both GnRHa treated and untreated cohorts, are involved in metabolic processes, catalytic activities, binding, signal transduction, transcriptional and translational activities, cell cycle regulation, cell and tissue structure, etc. (Tables 1-4). In addition, 15% to 23% of the genes were either functionally unclassified, or their roles in biological process are still unknown.

### EXAMPLE 2

### Time-Dependent Action of GnRHa on Gene Expression Profile of Leiomyoma and Myometrial Smooth Muscle Cells (LSMC and MSMC)

Leiomyoma and myometrium and their smooth muscle cells (LSMC and MSMC) express GnRH and GnRH receptors, and GnRH through the activation of specific signal transduction pathways results in transcriptional regulation of several genes downstream from these signals in LSMC and MSMC (Ding, L et al. J Clin Endocrinol Metab, 2004, 89:5549-5557; Chegini, N et al. Mol Cell Endocrinol, 2003, 209:9-16; Xu, J et al. J Clin Endocrinol Metab, 2003, 88:1350-61. To obtain a comprehensive picture of transcriptional changes induced by GnRHa direct action in leiomyoma and myometrium, we isolated LSMC and MSMC from the untreated cohorts. The serum starved LSMC and MSMC were treated with GnRHa (0.1 μM) for 2, 6 and 12 hours and their isolated RNA was subjected to microarray analysis. Based on the same data analysis criteria described above with a false discovery rate of p≦0.005, we identified 281 genes including 36 EST or 2.2% of the genes on the array displaying differential expression and regulation in LSMC and MSMC in response to GnRHa treatment in a time-dependent manner compared to untreated controls. Hierarchical clustering analysis also separated these genes into different clusters in response to time-dependent action of GnRHa in LSMC and MSMC, with expression patterns sufficiently different to cluster into their respective subgroups. Analysis of the variance-normalized mean (K-means) separated the differentially expressed and regulated genes in these cohorts into 4 distinctive clusters, with genes in clusters A and D displaying a cell-specific response, while genes in cluster B and C showing regulatory behaviors to GnRHa time-dependent action. Among the differentially expressed and regulated genes, the transcripts of 48 genes were identified as commonly expressed in LSMC and the original tissues (leiomyoma) from the untreated cohort used (Table 6).

Table 6 is a categorical list of differentially expressed genes in leiomyoma from GnRHa treated and LSMC treated with GnRHa for 2, 6 and 12 hours. The genes were identified following unsupervised and supervised analysis of their expression values and statistical analysis in R programming environment and ANOVA with a false-discovery rate selected at p≦0.005. Of the 130 genes identified, the expression of 34 genes was up-(+) and 96 genes downregulated (−) excluding 26 EST.

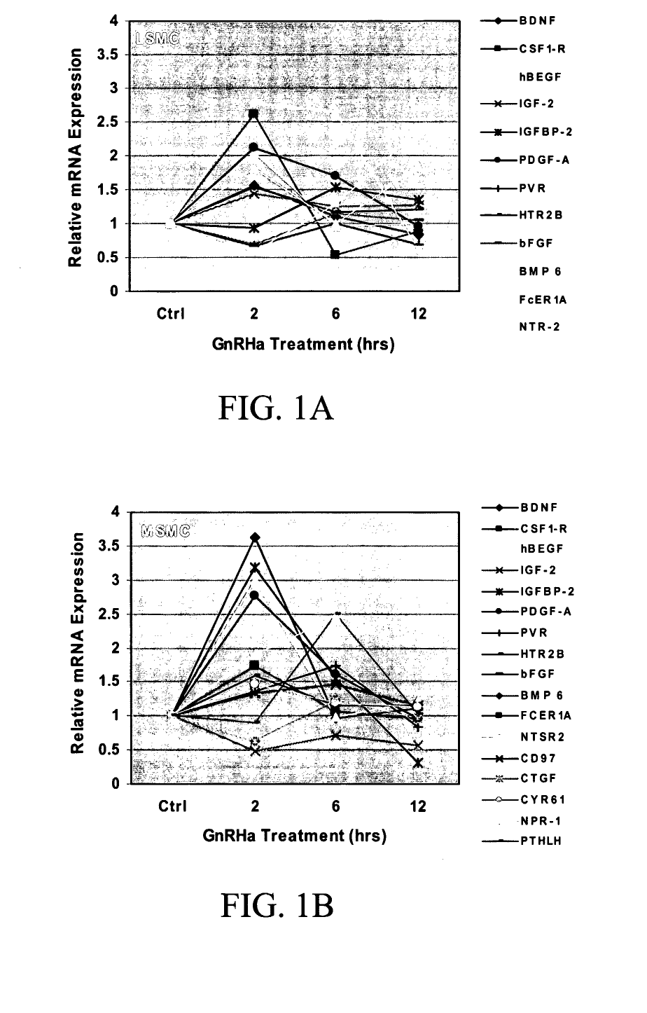

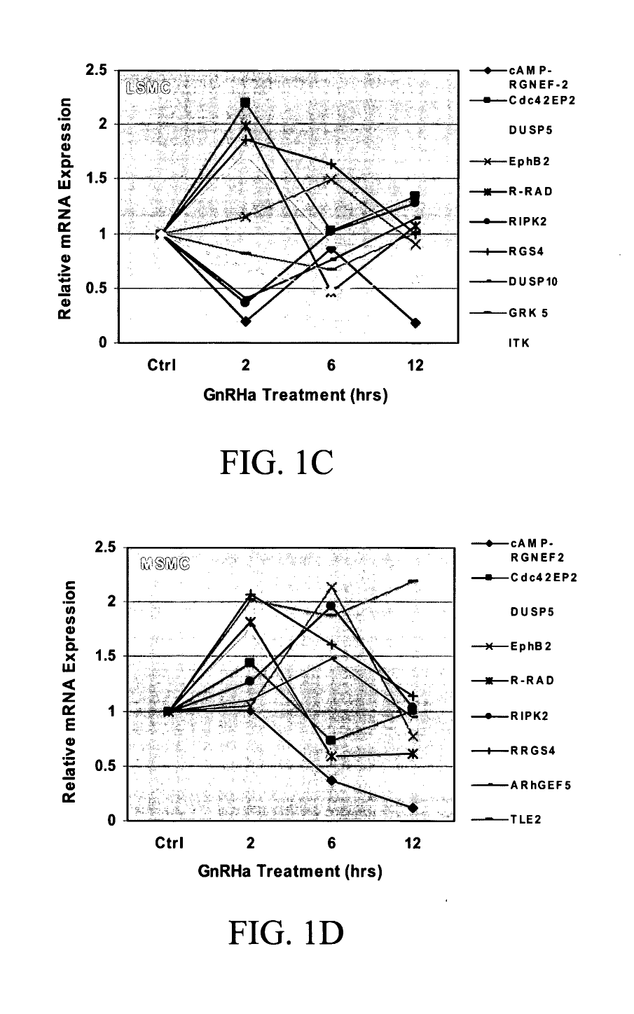

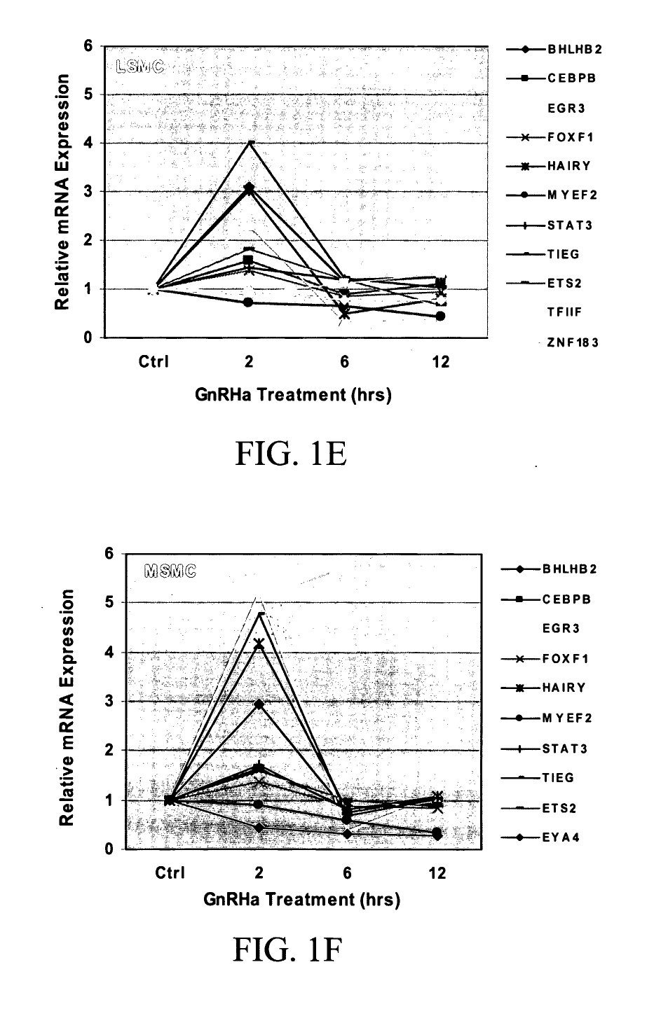

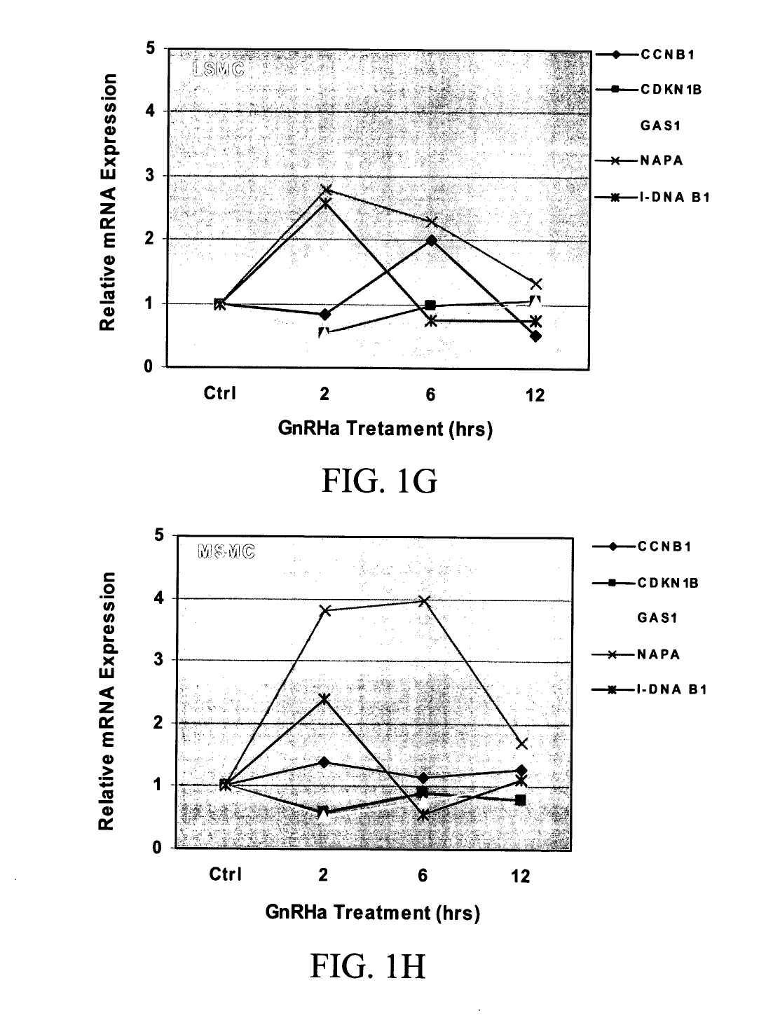

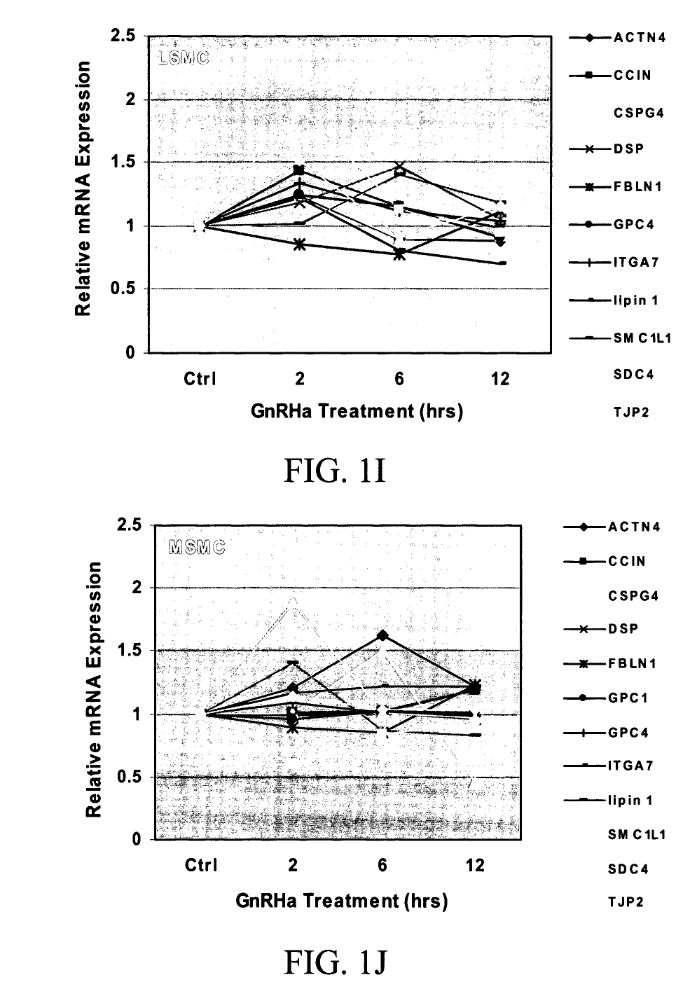

Gene ontology and functional annotation of the differentially expressed and regulated genes into similar functional categories indicated that in LSMC and MSMC, similar to their original tissues, the majority of the gene products are involved in cellular processes, catalytic activities, binding, signal transduction, transcriptional and translational activities, metabolism, cell cycle regulation and cellular structure. The time-dependent action of GnRHa on the expression of a selective group of genes representing growth factors/cytokines/chemokines/receptors, intracellular signal transduction pathways, transcription factors, cell cycle, cell adhesion/receptor/ECM/cytoskeleton in LSMC and MSMC are shown in FIGS. 1A-1J.

### EXAMPLE 3

### Verification of Gene Transcripts in Leiomyoma, Myometrium and LSMC and MSMC

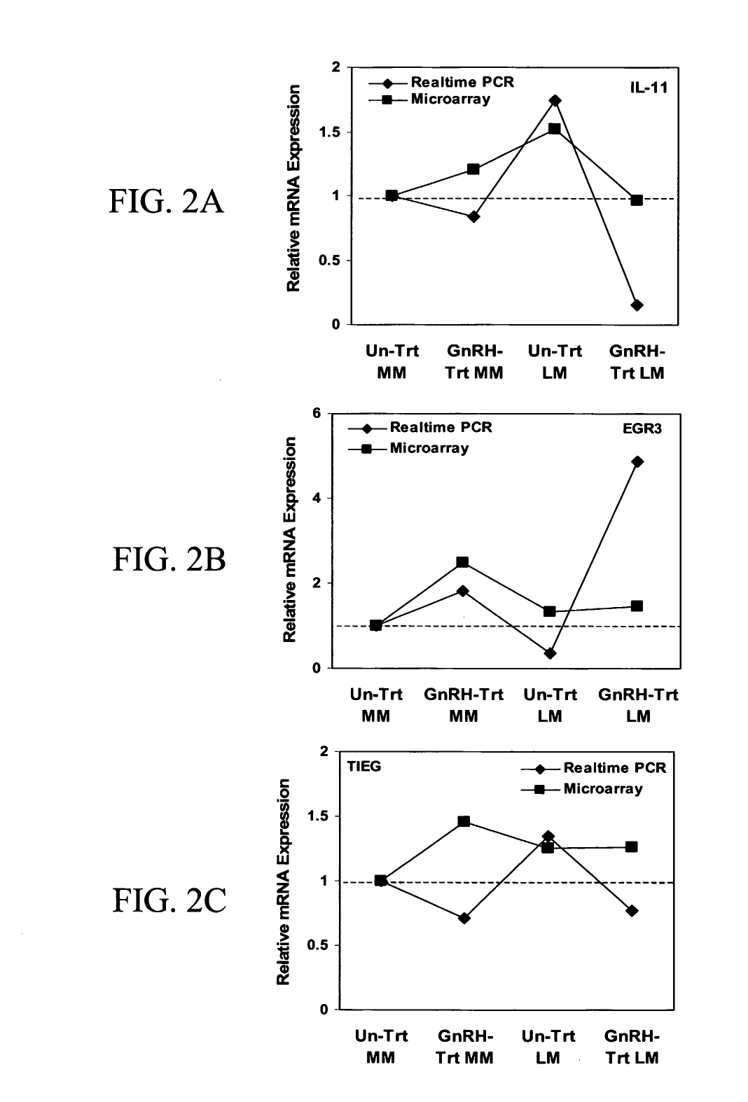

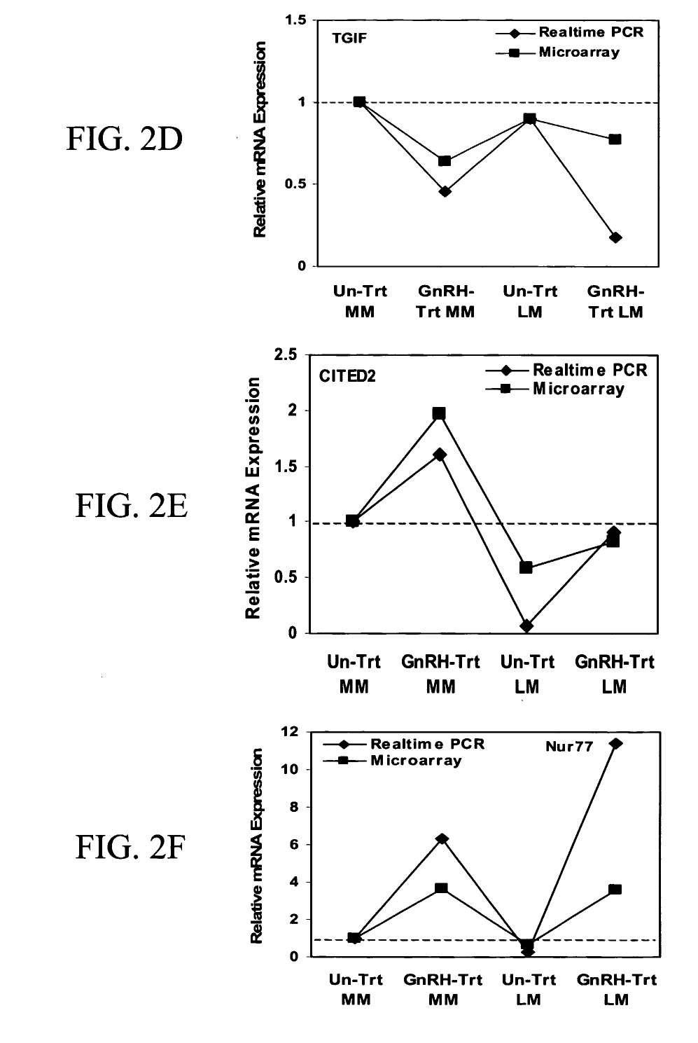

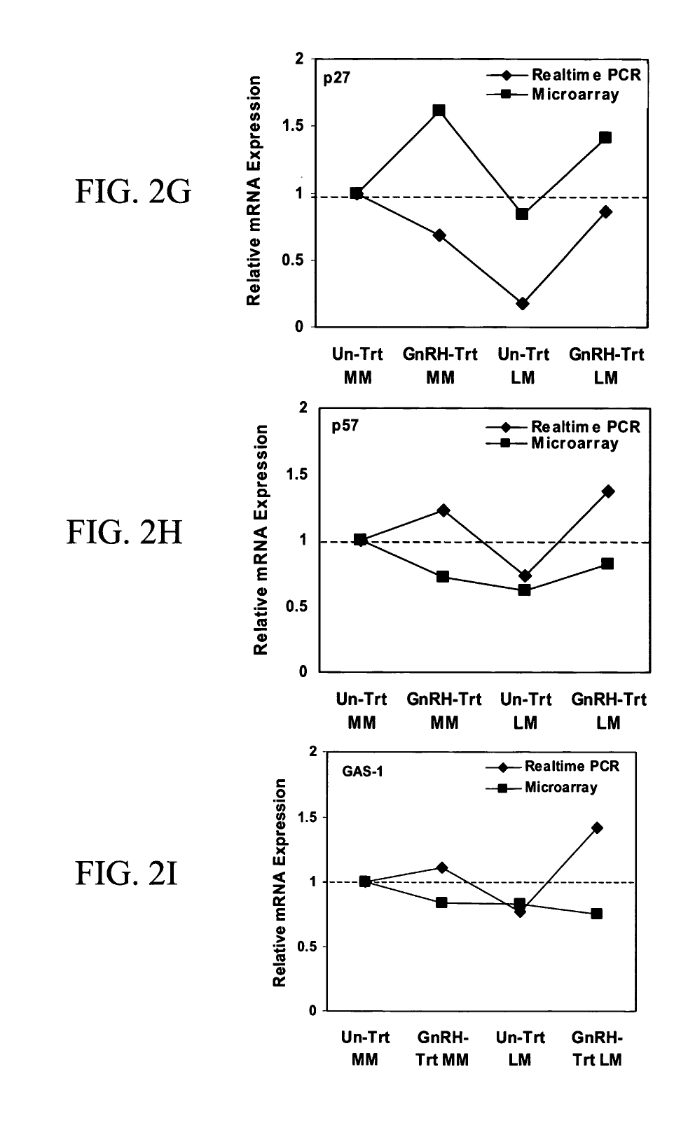

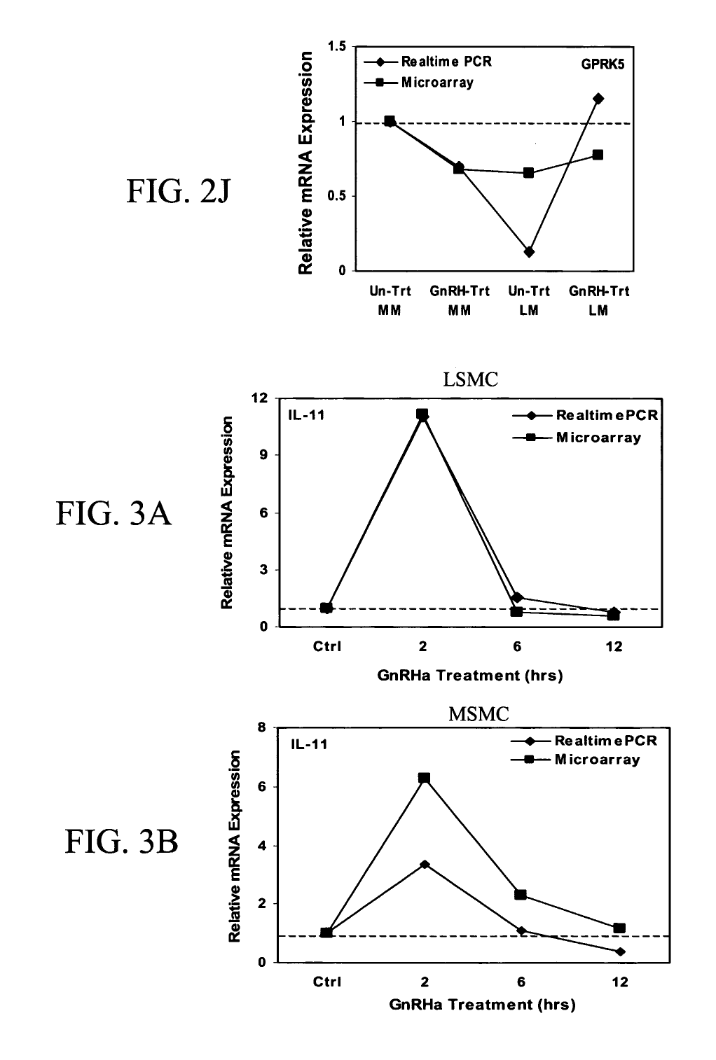

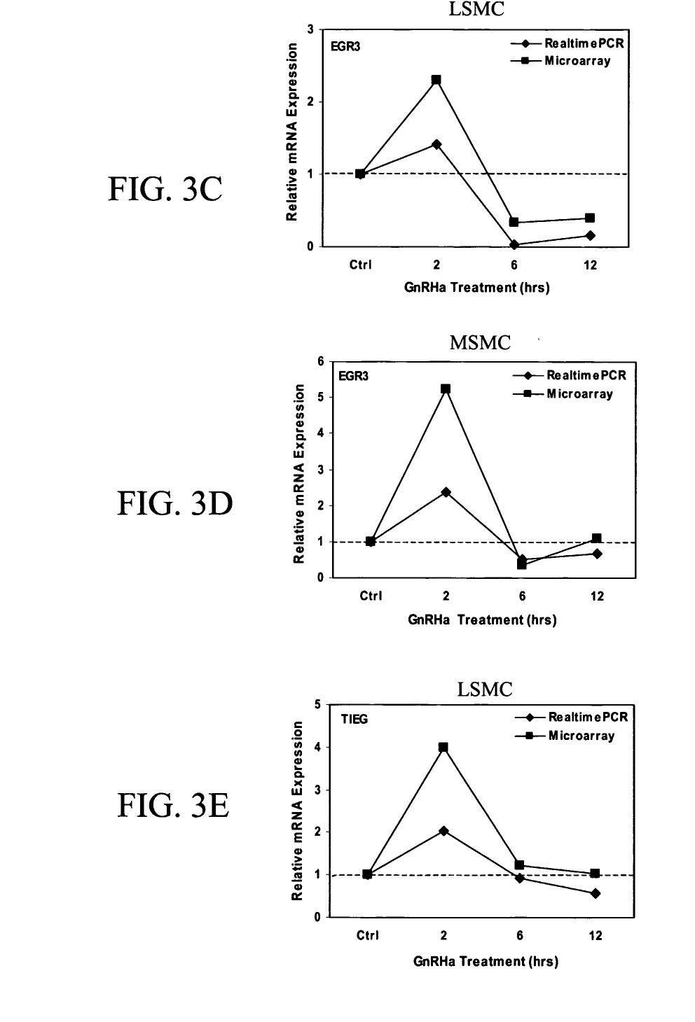

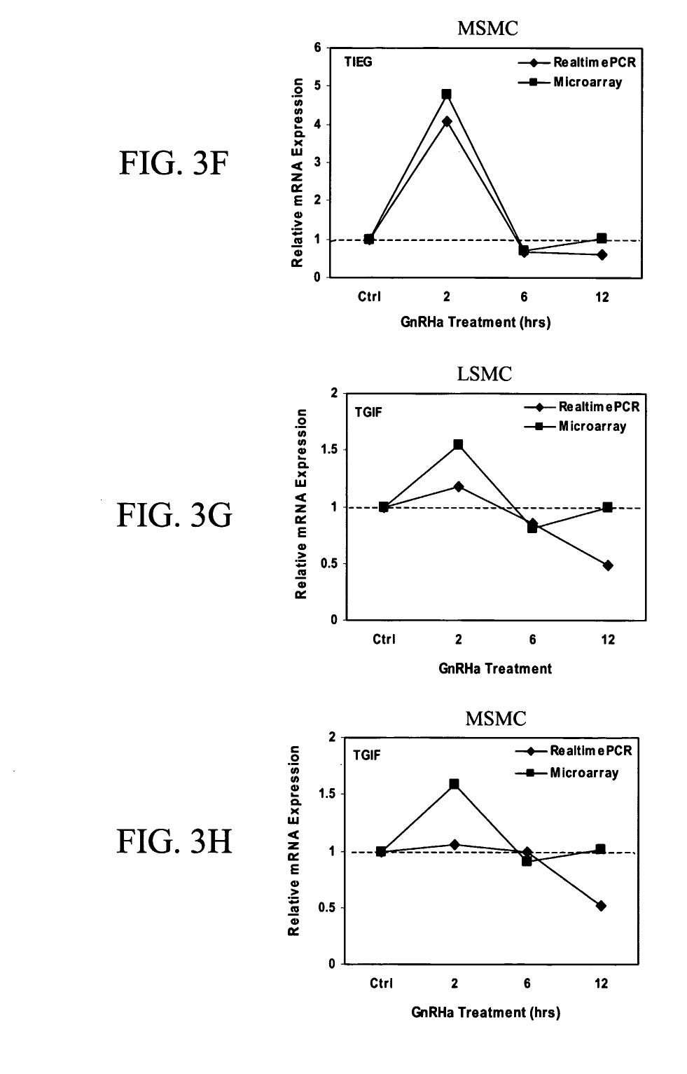

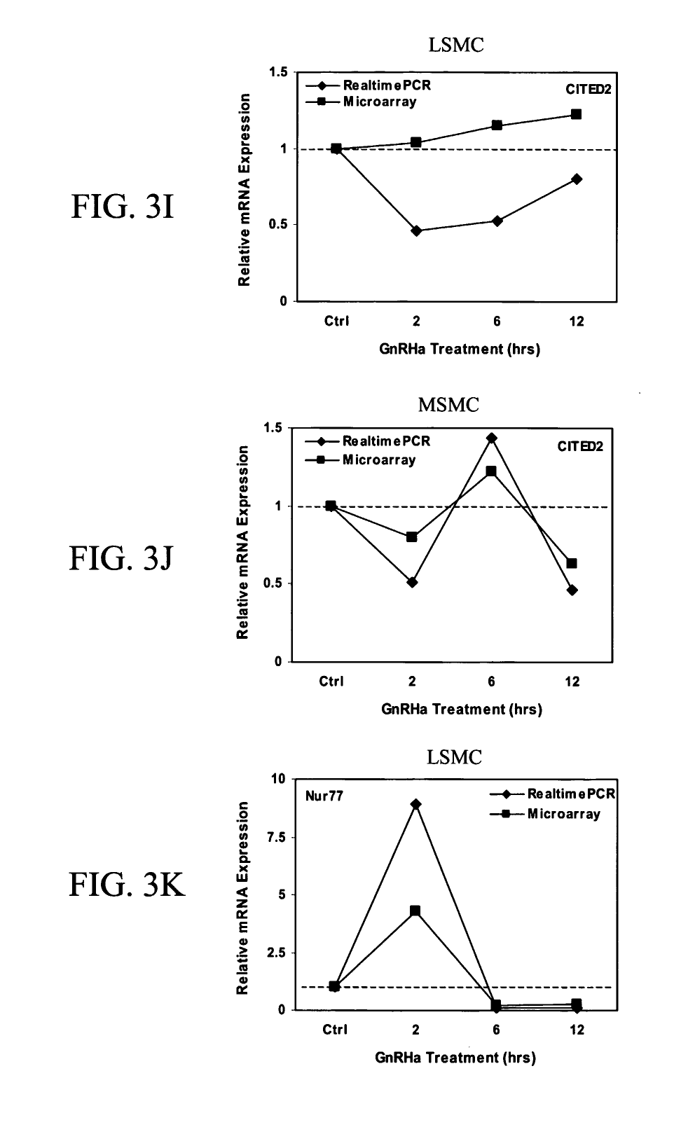

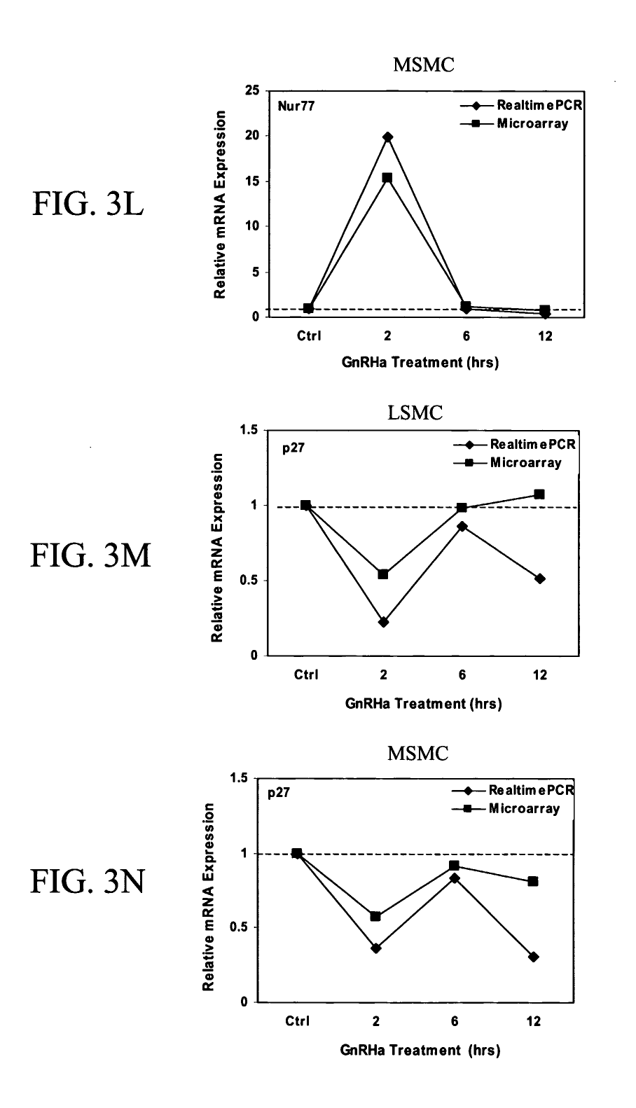

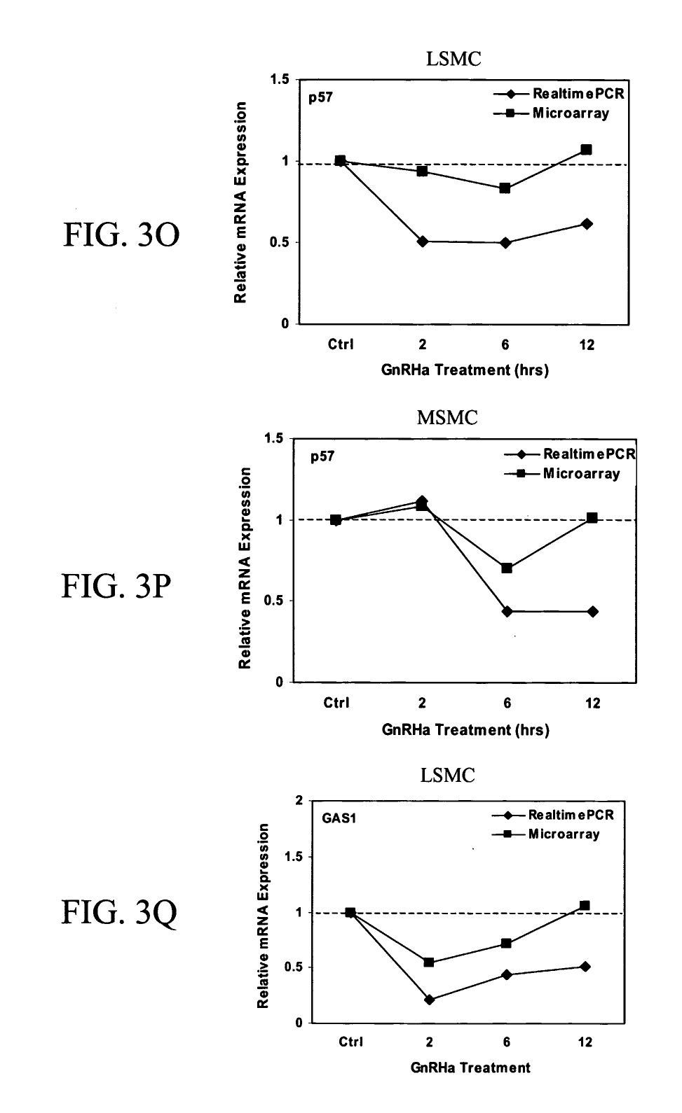

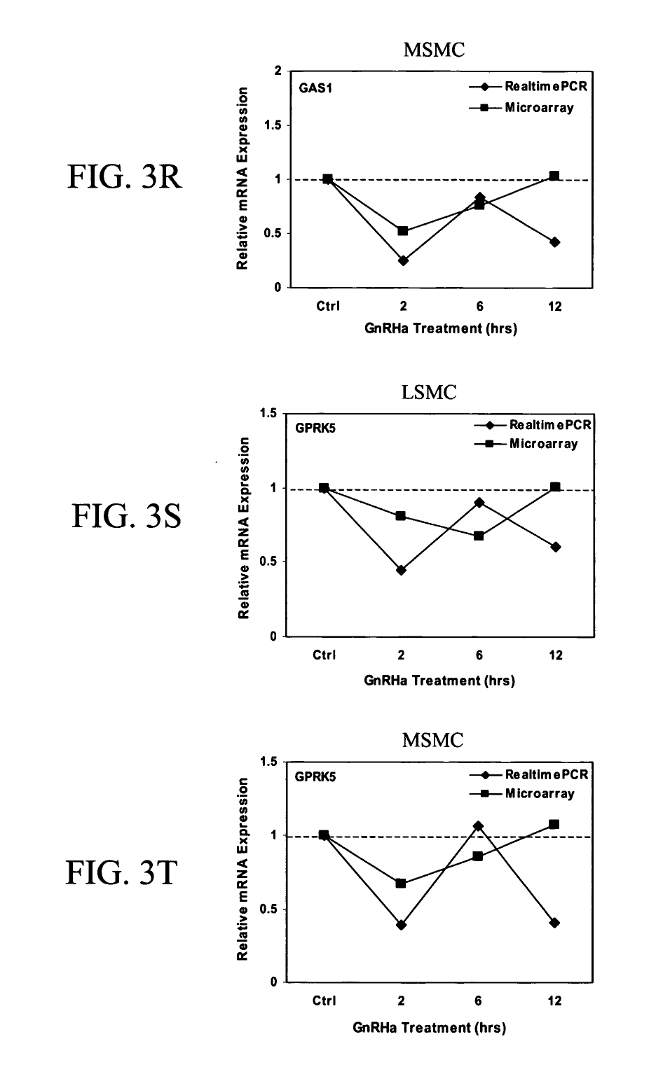

Among the differentially expressed and regulated genes identified in these tissues and cells, we selected 10 genes for verification using Realtime PCR, western blotting and immunohistochemistry. The selection of these genes was based not only on their expression values (up or downregulated), but also on gene classification, biological functions important to leiomyoma growth and regression, and regulation by ovarian steroids, GnRH and TGF-β. The genes selected for validation were IL-11, CITED2, Nur77, EGR3, TGIF, TIEG, CDKN1B (p27), CDKN1C (p57), GAS-1 and GPRK5, representing cytokines, transcription factors, cell cycle regulators, and signal transduction. The pattern of expression of these genes in leiomyoma and myometrium from untreated and GnRHa-treated cohorts (FIGS. 2A-2J), as well as in LSMC and MSMC treated with GnRHa for 2, 6 and 12 hours (FIGS. 3A-3T) as determined by Realtime PCR, closely overlapped with their expression profiles identified by the microarray analysis.

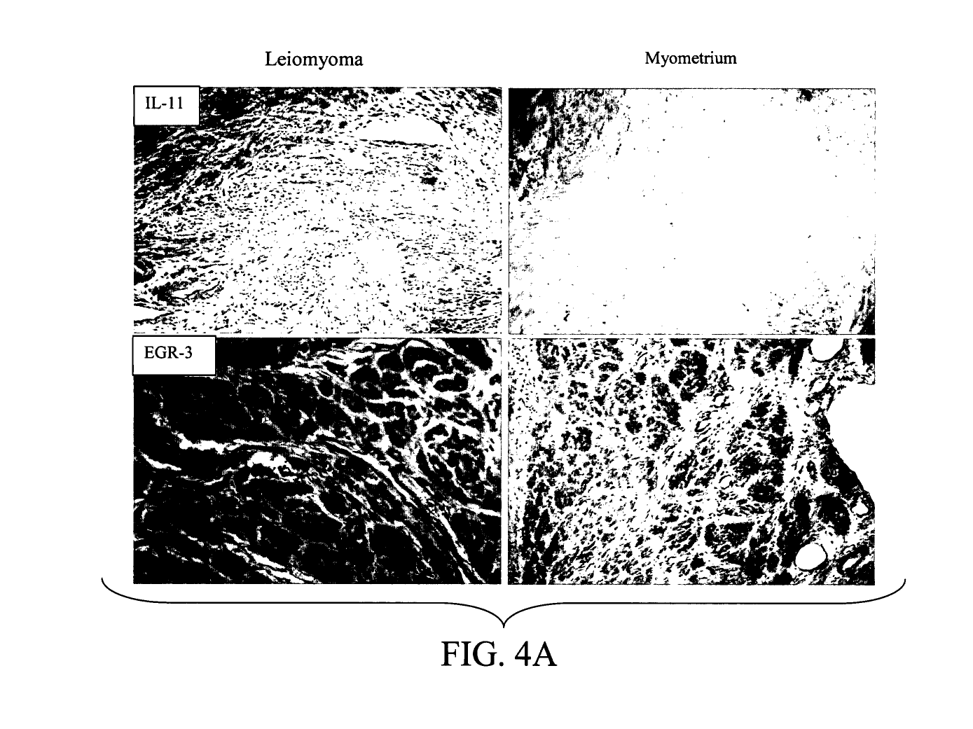

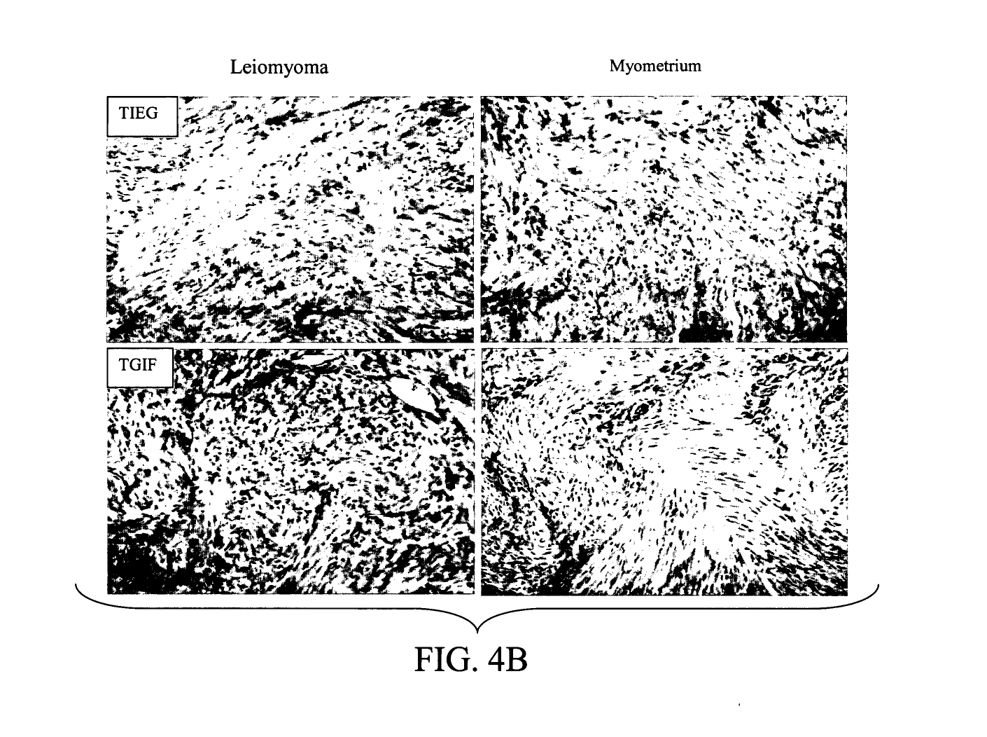

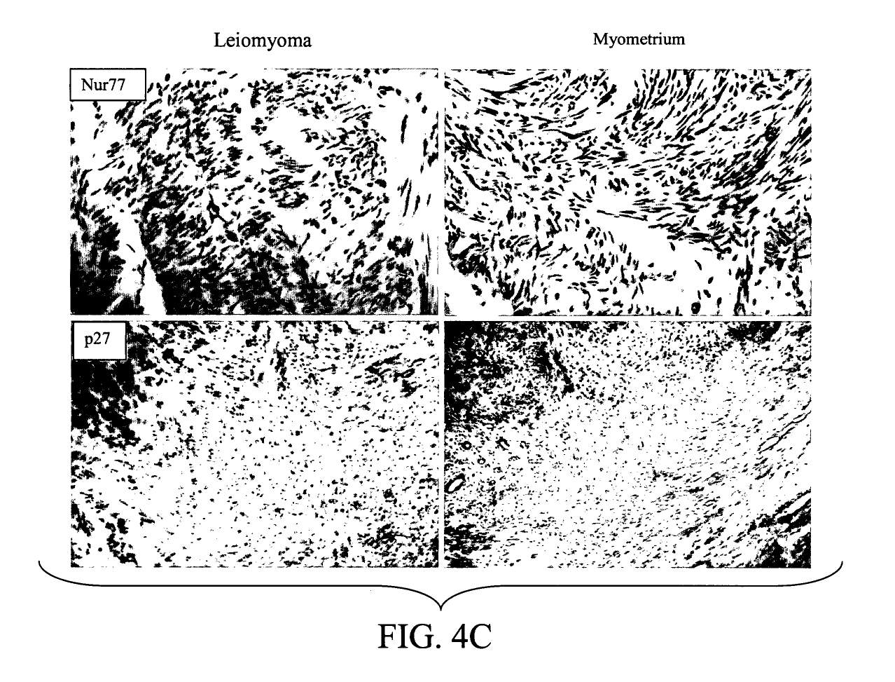

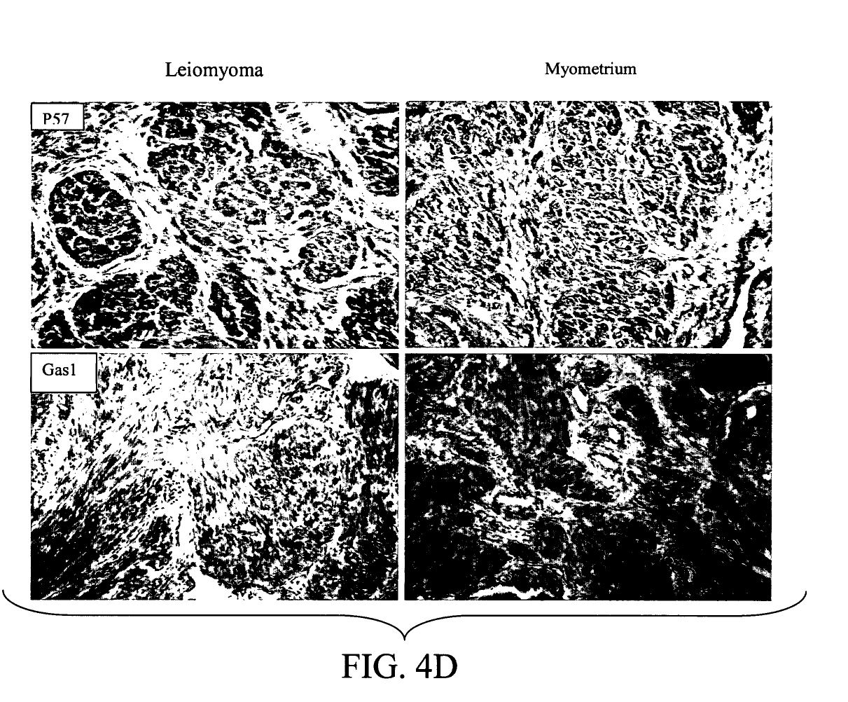

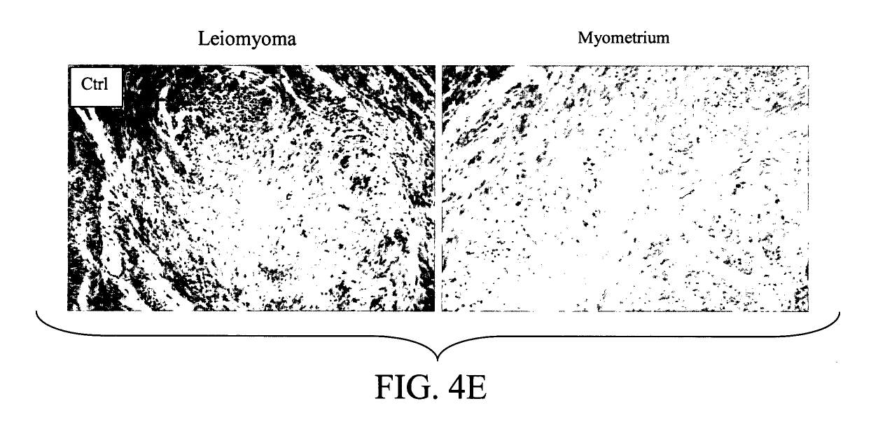

Western blotting also indicated that leiomyoma and myometrium, as well as LSMC and MSMC locally produce IL-11, TGIF, TIEG, Nur77, EGR3, CITED2, p27, p57 and Gas1 proteins. Immunohistochemically, IL-11, TGIF, TIEG, Nur77, EGR3, CITED2, p27, p57 and Gas1 were localized in various cell types in leiomyoma and myometrium, including LSMC and MSMC (FIGS. 4A-4E). The present inventors did not have access to antibody to GPRK5 and have not yet attempted to quantitate the level of IL-11, TGIF, TIEG, Nur77, EGR3, CITED2, p27, p57 and Gas1 production in leiomyoma and myometrium as well as in LSMC and MSMC in response to GnRHa treatment. However, these results provided further support for the microarray and Realtime PCR data, indicating that various cells types contribute to overall expression of these genes in leiomyoma and myometrium. In addition to these genes, the expression of 15 more genes was validated with Realtime PCR including CTGF, Abl-interactor 2 (Abi2), fibromodulin, Runx1 and Runx2 (Levens, E et al. “Differential Expression of fibromodulin and Abl-interactor 2 in leiomyoma and myometrium and regulation by gonadotropin releasing hormone analogue (GnRHa) therapy” Fertil Steril, 2004, (In press)).

Uterine leiomyoma affect 30 to 35% of women during their reproductive years and up to 70 to 80% before menopause (Baird, D D et al. Am. J Obstet Gynecol, 2003, 188: 100-107). The etiology of leiomyoma remains unknown, however they are thought to derive from the transformation of MSMC and/or connective tissue fibroblasts, and display high sensitivity to ovarian steroids for their growth. For this reason, GnRHa therapy is often used for medical management of symptomatic leiomyomas. In addition to GnRHa therapy, clinical and preclinical assessments of selective estrogen and progesterone receptor modulators, either alone, or in combination with GnRHa therapy, have shown efficacy in leiomyoma regression (Steinauer, J et al. Obstet Gynecol, 2004, 103:1331-6; Palomba, S et al. Hum Reprod, 2002, 17:3213-3219; DeManno, D et al. Steroids, 2003, 68:1019-32). Despite their prevalence and the efficacy of these therapies for their medical management, the molecular environment differentiating leiomyoma from adjacent myometrium, and their response to the above therapies is unknown. In the present study, the present inventors characterized gene expression fingerprints of leiomyoma and matched myometrium from the early-mid secretory phase of the menstrual cycle, a period associated with their rapid growth, their response to hormonal transition induced by GnRHa therapy, and to direct action of GnRHa in isolated LSMC and MSMC prepared from the untreated tissues.

Combining global normalization and unsupervised assessment of the gene expression values derived from all the cohorts enabled us to sort potential candidate genes prior to their putative identification in each cohort. Transcripts of many of the genes on the array were found in leiomyoma and myometrium as well as in LSMC and MSMC. However, leiomyoma/LSMC were not distinguished as a single class from myometrium/MSMC based on single gene markers uniformly expressed only in leiomyoma and/or myometrium. This is not unique to leiomyoma/myometrium since many large-scale gene expression profiling studies have shown the existence of a significant degree of shared gene expression between various tumors and their normal tissue counterparts. However, supervised assessment and multiple test correction in R programming environment (Tusher, V G et al. Proc Natl Acad Sci USA, 2001, 98:5116-5121; Pavlidis, P Methods, 2003, 31:282-289; Peterson, L E Comput Method is Programs Biomed, 2003, 70:107-19; Butte, A Nat Rev Drug Discov, 2002, 1:951-960) with reduced false discovery rate, allowed the identification of a specific set of differentially expressed and regulated genes in descending order of significance in each cohort. The analysis separated these genes into several clusters with a sufficient difference allowing their subdivision into their respective subgroup in leiomyoma, myometrium, their isolated cells, as well as due to GnRHa therapy at the tissue and cellular levels. We identified 153 genes (excluding 19 EST) in these cultures as differentially expressed in leiomyoma compared to myometrium, of which 82 genes were upregulated and 52 downregulated in leiomyoma. GnRHa therapy affected the expression of 122 genes (excluding 21 EST), with 34 upregulated and 67 downregulated genes in leiomyoma compared to myometrium. However, their gene profiles in untreated and GnRHa treated leiomyoma/myometrium differed substantially, pointing out a unique molecular environment that is targeted by GnRHa therapy. Analysis of the variance-normalized mean gene expression values divided these genes into 4 clusters with two clusters showing treatment-specific, while other clusters displayed a tissue-specific response to GnRHa therapy. A similar behavior was also observed with gene clusters identified in LSMC and MSMC in response to GnRHa action in vitro. The significance of these findings are related to clinical observations indicating that GnRHa therapy affects both leiomyoma and myometrium, with non-myoma tissue regressing more in response to therapy (Carr, B R et al. J Clin Endocrinol Metab, 1993, 76:1217-1223). The gene expression profiling disclosed herein supports the clinical observations, and further points out that GnRHa therapy targets different genes in leiomyoma and myometrium although they may group in a similar functional category. The recent microarray study using a small-scale array containing probe sets of 1200 known genes (Chegini, N et al. J Soc Gynecol Investig, 2003, 10:161-71) provides support for the current study; however, the present inventors are not aware of any other study using a large-scale gene expression profiling in leiomyoma and myometrium from women who received GnRHa therapy for further comparison.

Since this study was completed, a few other microarray studies have reported the gene expression profiles of leiomyoma and myometrium from the proliferative and secretory phases of the menstrual cycle (Tsibris, J C M et al. Fertil Steril, 2002, 78:114-121; Wang, H et al. Fertil Steril, 2003, 80:266-76; Weston, G et al. Mol Hum Reprod, 2003, 9:541-9; Quade, B J et al. Genes Chromosomes Cancer, 2004, 40:97-108). To broaden the scope of this study, the present inventors compared the genes list identified in untreated leiomyoma and matched myometrium of the present study, with the data sets reported in four of these other studies (Tsibris, J C M et al. Fertil Steril, 2002, 78:114-121; Wang, H et al. Fertil Steril, 2003, 80:266-76; Weston, G et al. Mol Hum Reprod, 2003, 9:541-9; Quade, B J et al. Genes Chromosomes Cancer, 2004, 40:97-108). This comparison resulted in identification of only a few genes in common among these studies. Although intrinsic individual tissue variation may contribute toward differences among these studies, standard of experimental process, utilization of different microarry platforms, utilization of tissues from different phases of the menstrual cycle, differences of leiomyoma size, and most importantly the method of data acquisition and analysis (Tsibris, J C M et al. Fertil Steril, 2002, 78:114-121; Wang, H et al. Fertil Steril, 2003, 80:266-76; Weston, G et al. Mol Hum Reprod, 2003, 9:541-9; Quade, B J et al. Genes Chromosomes Cancer, 2004, 40:97-108) are among other key contributing factors accounting for different study results (Pavlidis, P Methods, 2003, 31:282-289; Peterson, L E Comput Methods Programs Biomed, 2003, 70:107-19; Butte, A Nat Rev Drug Discov, 2002 1:951-960). To maintain a standard, the present inventors used leiomyoma of uniform sizes (2-3 cm in diameters) and matched myometrium, and the untreated cohorts were collected from the early-mid secretory phase of the menstrual cycle, a period associated with leiomyoma maximum growth. However, lowering the false discovery rate of the present study allowed the identification of more transcripts and the appearance of additional common genes with other studies (see Table 5; Refs. Tsibris, J C M et al. Fertil Steril, 2002, 78:114-121; Wang, H et al. Fertil Steril, 2003, 80:266-76; Weston, G et al. Mol Hum Reprod, 2003, 9:541-9; Quade, B J et al. Genes Chromosomes Cancer, 2004, 40:97-108). Considering the presence of a large number of probe sets on these arrays (i.e. 6800-12,500), selection of genes based only on fold change (Tsibris, J C M et al. Fertil Steril, 2002), or higher statistical levels (Wang, H et al. Fertil Steril, 2003, 80:266-76; Weston, G et al. Mol Hum Reprod, 2003, 9:541-9; Ahn, W S et al. Int J Exp Pathol, 2003, 84:267-79; Quade, B J et al. Genes Chromosomes Cancer, 2004, 40:97-108) is no better than what one would expect by chance alone (Pavlidis, P Methods, 2003, 31:282-289; Peterson, L E Comput Methods Programs Biomed, 2003, 70:107-19; Butte, A Nat Rev Drug Discov, 2002 1:951-960). Since the present inventors employed a similar data analysis, a larger number of genes was found in common with our previous microarray study which used only a small-scale array containing about 1200 known genes (Chegini, N et al. et al. J Soc Gynecol Investig, 2003, 10:161-71). The present inventors recognize that exclusion of moderately regulated genes during microarray data analysis does not reflect lack of functional importance, since a number of genes previously identified in leiomyoma and myometrium by conventional methods are not included among the differentially expressed genes in our study and other reports (Chegini, N Implication of growth factor and cytokine networks in leiomyomas. In; Cytokines in human reproduction. J. Hill ed. New York, Wiley & Sons Publisher, 2000, 133-162; Maruo, T et al. Hum Reprod Update, 2004, 10:207-20; Tsibris, J C M et al. Fertil Steril, 2002, 78:114-121; Chegini, N et al. J Soc Gynecol Investig, 2003, 10:161-71; Wang, H et al. Fertil Steril, 2003, 80:266-76; Weston, G et al. Mol Hum Reprod, 2003, 9:541-9; Ahn, W S et al. Int J Exp Pathol, 2003, 84:267-79; Quade, B J et al. Genes Chromosomes Cancer, 2004, 40:97-108). However, the expression of newly identified genes requires verification, and their regulation would allow linking their potential biological functions in leiomyoma growth and regression.

GnRHa therapy and most recently SERM and SPRM have been utilized for medical management of leiomyoma (Takeuchi, H et al. J Obstet Gynaecol Res, 2000, 26:325-331; Steinauer, J et al. Obstet Gynecol, 2004, 103:1331-6; Palomba, S et al. Hum Reprod, 2002, 17:3213-3219; DeManno, D et al. Steroids, 2003, 68:1019-32; Carr, B R et al. J Clin Endocrinol Metab, 1993, 76:1217-1223). Unlike SERM and SPRM that act directly on estrogen/progesterone sensitive tissues such as the uterus (Palomba, S et al. Hum Reprod, 2002, 17:3213-3219; DeManno, D et al. Steroids, 2003, 68:1019-32), GnRHa is traditionally believed to act primarily at the level of the pituitary-gonadal axis to implement its therapeutic benefits (Klausen, C et al. Prog Brain Res, 2002, 141:111-128). However, identification of GnRH and GnRH receptors in several peripheral tissues, including leiomyoma, has led the present inventors to propose an autocrine/paracrine role for GnRH, and an additional site of action for GnRHa therapy (Chegini, N et al. J Clin Endocrinol Metab, 1996, 81:3215-3221; Ding, L et al. J Clin Endocrinol Metab, 2004, 89:5549-5557; Chegini, N et al. Mol Cell Endocrinol, 2003, 209:9-16; Xu, J et al. J Clin Endocrinol Metab, 2003, 88:1350-61). In vitro studies have provided evidence for direct action of GnRHa on several cell types derived from these tissues resulting in alterations of a wide range of cellular activities, including cell growth, apoptosis and gene expression (Ding, L et al. J Clin Endocrinol Metab, 2004, 89:5549-5557; Chegini, N et al. Mol Cell Endocrinol, 2003, 209:9-16; Xu, J et al. J Clin Endocrinol Metab, 2003, 88:1350-61; Chegini, N and Kornberg, L J Soc Gynecol Investig, 2003, 10:21-6; Chegini, N et al. Mol Hum Reprod, 2002, 8:1071-8; Klausen, C et al. Prog Brain Res, 2002, 141:111-128; Mizutani, T et al. J Clin Endocrinol Metab, 1998, 83:1253-1255). Using isolated LSMC and MSMC prepared from the untreated tissues allowed the present inventors to identify novel regulatory functions for GnRHa in leiomyoma and myometrium, and discover a wide range of genes whose expression has not previously been recognized to be the target of GnRHa direct action. Similar to their distinct clustering at tissue levels, the differentially expressed and regulated genes identified in LSMC and MSMC were also divided into clusters according to time-dependent response to GnRHa action. The genes in these clusters were either rapidly induced by GnRHa treatment, or required prolong exposure, while others displayed biphasic patterns of temporal regulation in both treatment- and cell-specific fashions. Despite differences in their profiles, substantial similarity existed in functional grouping of the genes affected by GnRHa therapy in leiomyoma/myometrium, and GnRHa direct action on LSMC/MSMC (in vitro), with the expression of 48 genes commonly identified in tissues and cells. The present inventors propose that the hypoestrogenic condition created by GnRHa therapy and contributions of other cell types to overall gene expression at the tissue level may account for the difference in profiles of gene expression between tissues and cell cultures. Gene ontology and division into similar functional categories indicated that the products of the majority of the genes in these clusters are involved in transcriptional and signal transduction activities, cell cycle regulation, extracellular matrix turnover, cell-cell communication, transport and enzyme regulatory activities.

Among the genes in these functional categories are several growth factors, cytokines and chemokines, and polypeptide hormones, identified as differentially expressed in leiomyoma, myometrium and their isolated smooth muscle cells, and were the target of GnRHa action in vivo and in vitro. Using several conventional methods, previous reports have documented the expression of PDGF, EGF, IGFs, VEGF, FGF, TGF-βs, CTGF, TNF-α, IFN-γ, MCP-1 and IL-8 as well as some of their receptors in leiomyoma and myometrium (Chegini, N “Implication of growth factor and cytokine networks in leiomyomas” In Cytokines in human reproduction. J. Hill ed. New York, Wiley & Sons Publisher, 2000; Maruo, T et al. Hum Reprod Update, 2004, 10:207-20; Ding, L et al. J Clin Endocrinol Metab, 2004, 89:5549-5557; Chegini, N et al. Mol Cell Endocrinol, 2003, 209:9-16; Chegini, N et al. Mol Hum Reprod, 2002, 8:1071-8; Wu, X et al. Acta Obstet Gynecol Scand, 2001, 80:497-504; Senturk, L M et al. Am J Obstet Gynecol, 2001, 184:559-566; Sozen, I et al. Fertil Steril, 1998, 69:1095-1102). However, the expression of some of these and other genes in this category did not meet the selection criteria of this study, a common discrepancy often observed in microarray analysis, particularly in identifying moderately expressed and regulated genes (Varela, J C et al. Invest Opthalmol V is Sci, 2002, 43:1772-1782; Tusher, V G et al. Proc Natl Acad Sci USA, 2001, 98:5116-5121; Pavlidis, P Methods, 2003, 31:282-289; Peterson, L E Comput Methods Programs Biomed, 2003, 70:107-19; Butte, A Nat Rev Drug Discov, 2002, 1:951-960). For example, the expression of TGF-β isoforms, TGF-β receptors and components of their signaling pathway that are well documented in leiomyoma and myometrium, as well as in their isolated smooth muscles cells (Chegini, N et al. Mol Cell Endocrinol, 2003, 209:9-16; Xu, J et al. J Clin Endocrinol Metab, 2003, 88:1350-61; Chegini, N and Kornberg, L J Soc Gynecol Investig, 2003, 10:21-6; Chegini, N et al. Mol Hum Reprod, 2002, 8:1071-8; Dou, Q et al. J Clin Endocrinol Metab, 1996, 81:3222-3230; Arici, A and Sozen, I Fertil Steril, 2000, 73:1006-1011; Lee, B S and Nowak, R A J Clin Endocrinol Metab, 2001, 86:913-920), are not consistently identified in microarray studies (Tsibris, J C M et al. Fertil Steril, 2002, 78:114-121; Chegini, N et al. J Soc Gynecol Investig, 2003, 10:161-71; Wang, H et al. Fertil Steril, 2003, 80:266-76; Weston, G et al. Mol Hum Reprod, 2003, 9:541-9; Ahn, W S et al. Int J Exp Pathol, 2003, 84:267-79; Quade, B J et al. Genes Chromosomes Cancer, 2004, 40:97-108), although in the current and previous (Chegini, N et al. J Soc Gynecol Investig, 2003, 10:161-71) studies we identified most of the members of TGF-β system. Among the cytokines whose expression was identified and validated in the present study is IL-11. IL-11 is recognized to play key regulatory functions in inflammation, angiogenesis and tissue remodeling (Leng, S X and Elias, J A Int J Biochem Cell Biol, 1997, 29:1059-62; Tang, W et al. J Clin Invest, 1996, 98:2845-53; Zhu, Z et al. Am J Respir Crit. Care Med, 2001, 164:S67-70; Zimmerman, M A et al. Am J Physiol Heart Circ Physiol, 2002, 283:H175-80; Bamba, S et al. Am J Physiol Gastrointest Liver Physiol, 2003, 285:G529-38), events that are central to leiomyoma pathophysiology. IL-11 is a member of the IL-6 family and produced by various cell types, including the uterus, and its overexpression is reported to cause sub-epithelial airway fibrosis particularly through interaction with IL-13 and TGF-β (Leng, S X and Elias, J A Int J Biochem Cell Biol, 1997, 29:1059-62; Tang, W et al. J Clin Invest, 1996, 98:2845-53; Zhu, Z et al. Am J Respir Crit. Care Med, 2001, 164:S67-70; Zimmerman, M A et al. Am J Physiol Heart Circ Physiol, 2002, 283:H175-80; Bamba, S et al. Am J Physiol Gastrointest Liver Physiol, 2003, 285:G529-38; Karpovich, N et al. Mol Hum Reprod, 2003, 9:75-80). Evidence has been provided that IL-11, similar to TGF-β and IL-13, is overexpressed in leiomyoma compared to myometrium and GnRHa therapy suppressed their expression in these tissues (Chegini, N et al. Mol Cell Endocrinol, 2003, 209:9-16; Chegini, N et al. Mol Hum Reprod, 2002, 8:1071-8; Dou, Q et al. J Clin Endocrinol Metab, 1996, 81:3222-3230; Ding, L et al. J Soc Gyncol Invest, 2004, 00, 00). At the cellular level, unlike the expression of TGF-β and IL-13, GnRHa increased IL-11 expression in LSMC and MSMC within 2 to 6 hours of treatment, which sharply declined to control levels after 12 hours. Although the nature of differential regulation of IL-11 at the tissue and cellular levels requires detailed investigation, prolonged treatment with GnRHa, the contribution of other cell types and the influence of other autocrine/paracrine regulators, may account for the difference in IL-11 expression between in vivo and in vitro conditions.

Other differentially expressed and regulated genes identified in the present study functionally belong to signal transduction pathways that are recruited and activated by various growth factors/cytokines/chemokines, polypeptide hormones, extracellular matrix and adhesion molecules. However the expression of few of these components and other signal transduction pathways has been documented in leiomyoma and myometrium (Chegini, N “Implication of growth factor and cytokine networks in leiomyomas” In; Cytokines in human reproduction. J. Hill ed. New York, Wiley & Sons Publisher, 2000, 133-162; Ding, L et al. J Clin Endocrinol Metab, 2004, 89:5549-5557; Chegini, N and Kornberg, L J Soc Gynecol Investig, 2003, 10:21-6; Orii, A et al. J Clin Endocrinol Metab, 2002, 87:3754-9), and little is known about their recruitment and activation in LSMC and MSMC. The expression of Smads, MAPK and FAK has been identified in leiomyomas and myometrium and evidence has been provided for their regulation and activation by GnRHa in LSMC and MSMC (Ding, L et al. J Clin Endocrinol Metab, 2004, 89:5549-5557; Xu, J et al. J Clin Endocrinol Metab, 2003, 88:1350-61; Chegini, N and Kornberg, L J Soc Gynecol Investig, 2003, 10:21-6). Here, the present inventors validated the expression of GPRK5 identified as one of the differentially expressed and regulated genes in leiomyoma and myometrium and demonstrated that GnRHa therapy, and in vitro treatment of LSMC and MSMC with GnRHa inhibits GPRK5 expression. G-protein-coupled receptor kinases (GPRKs), consisting of six members GPRK1 to GPRK6, act as key regulators of signaling via GPRKs, and are widely expressed in various tissues and cells (Mak, J C et al. Eur J Pharmacol, 2002, 436:165-72; Simon, V et al. Endocrinology, 2001, 142:1899-905; Simon, V et al. Endocrinology, 2003, 144:3058-66; Krasel, C et al. J Biol Chem, 2001, 276:1911-1915). Previous studies have demonstrated that pregnant and non-pregnant human myometrium as well as cultured myometrial cells express GPRK2, GPRK4γ and GPRK5, however GPRK3 and GPRK4α, β, and δ were not detected in myometrium (Simon, V et al. Endocrinology, 2001, 142:1899-905; Simon, V et al. Endocrinology, 2003, 144:3058-66). GPRK5 has been shown to serve as a substrate for PKC, although PKC-mediated phosphorylation inhibits GPRK5 (Klausen, C et al. Prog Brain Res, 2002, 141:111-128; Krasel, C et al. J Biol Chem, 2001, 276: 1911-1915). In addition, the extreme N terminus of GPRK5 contains a binding site for Ca2+/calmodulin, where upon binding it inhibits GPRK activity, a mechanism suggested to regulate GPRKs activity (Krasel, C et al. J Biol Chem, 2001, 276: 1911-1915). Since GnRH receptors are a member of the G-protein coupled receptor (GPCR) family and recruit and activate the components of several signaling pathways, including PKC and Ca2+/calmodulin, their regulatory interaction with GPRKs may serve in regulating various events downstream from these signals in LSMC and MSMC.

Nuclear translocation of many activated intracellular signaling molecules results in phosphorylation and activation of transcription factors, major elements in these signaling networks that regulate specific gene expression. In previous studies (Chegini, N et al. J Soc Gynecol Investig, 2003, 10:161-71) and the present study, several transcription factors were identified as differentially expressed and regulated in leiomyoma and myometrium and targeted by GnRHa direct action in LSMC and MSMC (see Tables 1-4). Many of these transcription factors are involved in ovarian steroids, polypeptide hormones, inflammatory cytokines, growth factors and ECM receptor mediated-actions, by regulating the promoter of their target genes in various normal and cancer cells. However, little is known regarding the expression and regulation of these and other transcription factors in leiomyoma and myometrium. For this reason, the present inventors placed a greater emphasis on verification of the expression of transcription factors such as Nur77, CITED2, EGR3, TIEG and TGIF in leiomyoma, myometrium and their temporal regulation by GnRHa in LSMC and MSMC.

Nur77 (also known as NR4A1, TR3, NGFI-B, NAK-1) is a member of the orphan nuclear receptor superfamily originally identified as an immediate-early gene in serum-treated fibroblasts (Maira, M et al. Mol and Cell Biol, 2003, 23; 763-776; Drouin, J et al. J Steroid Biochem Mol Biol, 1998, 65:59-63; Fernandez, P et al. Endocrinology, 2000, 141:2392-2400; Gelman, L et al. J Biol Chem, 1999, 274:7681-7688; Sadie, H et al. Endocrinology, 2003, 144:1958-71; Wilson, T E et al. Mol Cell Biol, 1993, 13:861-868; Song, K H et al. Endocrinology, 2001, 142:5116-23; Zhang, P and Mellon, S H Mol Endocrinol, 1997, 11:891-904). It is also identified as NGF-inducible gene, which is constitutively expressed in various tissues and is strongly induced by several stimuli, resulting in regulation of gene expression related to inflammation, angiogenesis, apoptosis and steriodogenesis, including steroid-21 and 17α-hydroxylases and 20α hydroxysteroid dehydrogenase in the hypothalamic-pituitary-adrenal axis (Maira, M et al. Mol and Cell Biol, 2003, 23; 763-776; Drouin, J et al. J. Steroid Biochem Mol Biol, 1998, 65:59-63; Fernandez, P et al. Endocrinology, 2000, 141:2392-2400; Gelman, L et al. J Biol Chem, 1999, 274:7681-7688; Sadie, H et al. Endocrinology, 2003, 144:1958-71; Wilson, T E et al. Mol Cell Biol, 1993, 13:861-868; Song, K H et al. Endocrinology, 2001, 142:5116-23; Zhang, P and Mellon, S H Mol Endocrinol, 1997, 11:891-904). In the anterior pituitary, Nur77 is reported to mediate the stimulatory effect of CRH and the negative-feedback regulation of POMC transcription by glucocorticoids, as well as GnRH-induced GnRH receptor expression (Drouin, J et al. J. Steroid Biochem Mol Biol, 1998, 65:59-63; Sadie, H et al. Endocrinology, 2003, 144:1958-71). LH-induced Nur77 is also reported to regulate cytochorome p450 expression in granulosa and leydig cells (Sadie, H et al. Endocrinology, 2003, 144:1958-71; Wilson, T E et al. Mol Cell Biol, 1993, 13:861-868; Song, K H et al Endocrinology, 2001, 142:5116-23). More importantly, overexpression of Nur77 is implicated as an important regulator of apoptosis in different cells. In response to apoptotic stimuli, Nur77 translocation from the nucleus to mitochondria results in cytochrome C release and apoptosis of LNCaP human prostate cancer cells (Rajpal, A et al. EMBO J, 2003, 22:6526-36; Castro-Obregon, S et al. J Biol Chem, 2004, 279:17543-53; Li, H et al. Science, 2000, 289:1159-1164). The present inventors found a relatively similar expression of Nur77 in myometrium and leiomyoma; however, GnRHa therapy resulted in a significant elevation of Nur77 in both tissues. GnRHa treatment also resulted in a rapid induction of Nur77 in MSMC and LSMC, which subsequently declined to control levels, and in LSMC fell to below the levels detected in untreated cells. Interestingly, GnRH is reported to regulate Nur77 expression in αT3-1 and LβT2 gonadotrope cell lines through PKA pathway and GnRH receptor promoter via a mechanism involving SF-1 with Nur77 acting as a negative regulator of this response (Sadie, H et al. Endocrinology, 2003, 144:1958-71). In a recent study, activation of MAPK pathway involving Raf-1, MEK2 and ERK2 was reported to regulate Nur77 activation resulting in nonapoptotic program cell death (Castro-Obregon, S et al. J Biol Chem, 2004). The present inventors have shown that GnRH signaling through MAPK and transcriptional activation of c-fos and c-jun regulate the expression of several specific genes in LSMC and MSMC. This suggests that GnRH-mediated action through this pathway may regulate nur77 expression thus influencing the outcome of cellular growth arrest and/or apoptosis in leiomyoma.

Recently, a new family of transcriptional co-regulators, the CITED (CBP/p300-interacting transactivator with ED-rich tail) family, was discovered that interact with the first cysteine-histidine-rich region of CBP/p300 (Tien, E S et al. J Biol Chem, 2004, 279:24053-63; Kranc, K R et al Mol Cell Biol, 2003, 23:7658-66). The CITED family contains four members and appears to act as key transcriptional modulators in embryogenesis, inflammation, and stress responses (Tien, E S et al. J Biol Chem, 2004, 279:24053-63) by affecting the transcriptional activity of many transcription factors ranging from AP2, estrogen receptor, and hypoxia-inducible factor 1 (HIF1) and LIM (Yin, Z et al. Proc Natl Acad Sci USA, 2002, 99:10488-10493). The present inventors identified CITED2 among the differentially expressed and regulated genes in leiomyoma, myometrium and their isolated cells, and in response to GnRHa treatment in vivo and in vitro. Unlike GnRHa therapy which increased CITED2 expression in leiomyoma and myometrium, GnRHa had a biphasic effect on CITED2 expression in MSMC, while inhibiting expression in LSMC. Although in vitro culture conditions may directly influence the expression of regulatory molecules that either interact with or regulate CITED2 expression, the exact molecular mechanism resulting in differential expression of CITED2 in vivo and in vitro by GnRHa requires further investigation. Interestingly, the expression of several growth factors, cytokines and HIF1 are the target of ER, PR regulatory action, and CITED2 acting as a repressor of their expression may serve as an important regulator of processes that regulate inflammatory response, angiogenesis and tissue remodeling in leiomyoma. Additionally, CBP/p300 which serve as promiscuous co-activators for an increasing number of transcription factors resulting in proliferation, differentiation and apoptosis in response to diverse biological factors, including ER- and PR-dependent transcriptional activity, is specifically recruited by Nur77 acting as dimers following PKA activation (Maira, M et al. Mol and Cell Biol, 2003, 23; 763-776; Kranc, K et al. Trends Cell Biol, 1997, 7:230-236; Puri, P L et al. EMBO J, 1997, 16:369-383).

In a previous microarray study, it was reported that EGR1, a prototype of a family of zinc-finger transcription factors that includes EGR2, EGR3, EGR4, and NGFI-B (Hjoberg, J et al. Am J Physiol Lung Cell Mol Physiol, 2004, 286:L817-825; Thiel, G and Cibelli, G J Cell Physiol, 2002, 193:287-92), is differentially expressed in leiomyoma and myometrium (Chegini, N et al. J Soc Gynecol Investig, 2003, 10:161-71). Here, the present inventors provide evidence for the expression of EGR3 and differential regulation in response to GnRHa therapy in leiomyoma and myometrium, as well as in LSMC and MSMC in vitro. A recent report demonstrated that EGR1 expression is elevated in leiomyoma compared to corresponding myometrium in women who received GnRHa therapy (Shozu, M et al. Cancer Research, 2004, 64:4677-4684) supporting previous microarray data (Chegini, N et al. J Soc Gynecol Investig, 2003, 10:161-71). EGRs expression is rapidly and transiently induced by a large number of growth factors, cytokines, polypeptide hormones and injurious stimuli and kinetics of their expression is essentially identical to c-fos proto-oncogene (Hjoberg, J et al. Am J Physiol Lung Cell Mol Physiol, 2004, 286:L817-825; Thiel, G and Cibelli, G J Cell Physiol, 2002, 193:287-92; Inoue, A et al. J Mol Endocrinol, 2004, 32:649-61). In addition, induction of EGR1 occurs primarily at the level of transcription and is mediated, in part, through MAPKs, including ERK, JNK, and p38 pathways (Hjoberg, J et al. Am J Physiol Lung Cell Mol Physiol, 2004, 286:L817-825; Thiel, G and Cibelli, G J Cell Physiol, 2002, 193:287-92). It has been demonstrated that GnRHa through the activation of MAPK regulates the expression c-fos and c-jun as well as fibronectin, collagen and PAI-I expression (Ding, L et al. J Clin Endocrinol Metab, 2004, 89:5549-5557). In human fibrosarcoma and glioblastoma cells, EGR directly influences the expression of fibronectin, TGF-β1, and PAI-1 and may regulate the expression of PDGF, tissue factor, and membrane type 1 matrix metalloproteinase (Thiel, G and Cibelli, G J Cell Physiol, 2002, 193:287-92; Liu, C et al. J Biol Chem, 1999, 274:4400-11). Estrogen is also reported to induce EGR3 in various cancer cell lines while is inhibited by progesterone in Schwann cells (Inoue, A et al. J Mol Endocrinol, 2004, 32:649-61; Mercier, G et al. Mol Brain Res, 2001, 97:137-148). Constitutive transgenic expression of EGR3 has recently been shown to increase thymocytes apoptosis, possibly through potent activation of FasL expression (Xi, H and Kersh, G J J Immunol, 2004, 173:340-8). Given the role of ovarian steroids and a large number of growth factors, cytokines and polypeptide hormones in leiomyoma growth, and suppression by GnRHa, their differential influence on EGR1 and EGR3 expression may represent a mechanism resulting in balance between the rate of cell proliferation and apoptosis as well as tissue turnover, affecting leiomyoma growth and regression.

The present study also provides the first evidence of the expression and regulation of TIEG and TGIF, novel three zinc-finger Kruppel-like transcriptional repressors, and key regulators of TGF-β receptor signaling (Johnsen, S A et al. Oncogene, 2002, 21:5783-90; Cook, T and Urrutia, R Am J Physiol Gastrointest Liver Physiol, 2000, 278:G513-21; Ribeiro, A et al. Hepatology, 1999, 30:1490-7; Chen, F et al. Biochem J, 2003, 371:257-63; Melhuish, T A et al. J Biol Chem, 2001, 276:32109-14), by GnRHa in leiomyoma, myometrium, LSMC and MSMC. TIEG regulates TGF-β receptor signaling through a negative feedback mechanism by repressing the inhibitory Smad7 (Johnsen, S A et al. Oncogene, 2002, 21:5783-90). In addition, TGIF through direct binding to DNA or interaction with TGF-β-activated Smads represses TGF-β-responsive gene expression (Chen, F et al. Biochem J, 2003, 371:257-63; Melhuish, T A et al. J Biol Chem, 2001, 276:32109-14). Since GnRHa suppresses TGF-β and TGF-β receptors while enhancing Smad7 expression in leiomyoma and myometrium as well as LSMC and MSMC, differential regulation of TIEG and TGIF may serve as an additional downstream mechanism altering TGF-β autocrine/paracrine actions in leiomyoma. To further understand the regulation of these transcription factors in leiomyoma, the present inventors also provide evidence for their regulation in LSMC and MSMC by TGF-β, further implicating the importance of TGF-β in pathogenesis of leiomyoma (as described in Examples 4-7).

The expression, activation and direct interaction of these and other transcription factors with DNA results in regulation of the expression of various genes whose products influence cell growth, inflammation, angiogenesis, apoptosis and tissue turnover. In previous studies (Chegini, N et al. J Soc Gynecol Investig, 2003, 10:161-71; Ding, L et al. J Clin Endocrinol Metab, 2004, 89:5549-5557) and the present study, several differentially expressed and regulated genes were identified in leiomyoma, myometrium and LSMC and MSMC whose promoters are the target of these transcription factors. Among these genes are members of cell cycle regulatory proteins that play a central role in leiomyoma growth and regression (Chegini, N “Implication of growth factor and cytokine networks in leiomyomas” In; Cytokines in human reproduction. J. Hill ed. New York, Wiley & Sons Publisher, 2000, 133-162; Maruo, T et al. Hum Reprod Update, 2004, 10:207-20; Zhai, Y L et al. Int J Cancer, 1999, 84:244-50), including p27, p57 and Gas1. The present inventors identified p27, p57 and Gas1 as differentially expressed and regulated in leiomyoma and myometrium as well as LSMC and MSMC and in response to GnRHa treatment. Although p27, p57 and Gas1 function as major regulators of cell cycle progression, several studies have also shown Cip/Kip proteins function as transcriptional cofactors by regulating the activity of NFκ-B, STAT3, Myc, Rb, C/EBP, CBP/p300, E2F and AP1 (Coqueret, O Trends Cell Biol, 2003, 13:65-70). A recent report also suggests that p21, p27 and p57 are involved in regulation of apoptosis (Blagosklonny, M V Semin Cancer Biol, 2003, 13:97-105) and their differential regulation in leiomyoma and myometrium is consistent with GnRHa induction of apoptosis related gene in LSMC and MSMC (Chegini, N “Implication of growth factor and cytokine networks in leiomyomas” In; Cytokines in human reproduction. J. Hill ed. New York, Wiley & Sons Publisher, 2000, 133-162; Maruo, T et al. Hum Reprod Update, 2004, 10:207-20; Mizutani, T et al. J Clin Endocrinol Metab, 1998, 83:1253-1255; Zhai, Y L et al. Int J Cancer, 1999, 84:244-50). However, the results disclosed herein are the first to document the expression of Gas1 in leiomyoma and myometrium, and regulation in LSMC and MSMC in response to timed-dependent action of GnRHa. GnRHa has been demonstrated to alter cell cycle progression and programmed cell death in several cell types including leiomyoma smooth muscle cells (Chegini, N “Implication of growth factor and cytokine networks in leiomyomas” In; Cytokines in human reproduction. J. Hill ed. New York, Wiley & Sons Publisher, 2000, 133-162; Mizutani, T et al. J Clin Endocrinol Metab, 1998, 83:1253-1255; Zhai, Y L et al. Int J Cancer, 1999, 84:244-50), and these results provide additional support for the involvement of specific cell cycle and apoptotic related genes in leiomyoma growth and regression. How the expression of these genes is regulated and through what mechanism their products influence LSMC and MSMC cell cycle progression and programmed cell death awaits further investigation.

Leiomyoma growth and GnRHa therapy resulting in leiomyoma regression also involves extracellular matrix turnover. In previous studies (Chegini, N et al. J Soc Gynecol Investig, 2003, 10:161-71), in the present study, and in recent studies by other groups (Tsibris, J C M et al. Fertil Steril, 2002, 78:114-121; Wang, H et al. Fertil Steril, 2003, 80:266-76; Weston, G et al. Mol Hum Reprod, 2003, 9:541-9; Ahn, W S et al. Int J Exp Pathol, 2003, 84:267-79; Quade, B J et al. Genes Chromosomes Cancer, 2004, 40:97-108), several genes in this category were identified displaying differential expression in leiomyoma and myometrium and were targeted by GnRH therapy (Tables 1-4) (Chegini, N “Implication of growth factor and cytokine networks in leiomyomas” In Cytokines in human reproduction. J. Hill ed. New York, Wiley & Sons Publisher, 2000, 133-162; Ding, L et al. J Clin Endocrinol Metab, 2004, 89:5549-5557; Dou, Q et al. Mol Hum Reprod, 1997, 3:1005-1014; Levens, E et al. Fertil Steril, 2004, (In press); Stewart, E A et al. J Clin Endocrinol Metab, 1994, 79:900-6). These include the expression of several collagens, small leucine rich repeat family of proteoglycans, decorin, biglycan, osteomodulin, fibromodulin, versican, and osteoadherin/osteoglycin, fibronectin, desmin and vimentin, several member of proteases such as matrix metalloproteinases (MMPs) and their inhibitors, TIMPs, a disintegrin-like and metalloproteinase proteins (ADAM), etc. It has also been reported that GnRHa regulates the expression of fibronectin, collagen type I, PAI-I, MMPs and TIMPs (Chegini, N “Implication of growth factor and cytokine networks in leiomyomas” In Cytokines in human reproduction. J. Hill ed. New York, Wiley & Sons Publisher, 2000, 133-162; Ding, L et al. J Clin Endocrinol Metab, 2004, 89:5549-5557; Dou, Q et al. Mol Hum Reprod, 1997, 3:1005-1014), as well as decorin, versican, desmin and vimentin (unpublished data) in leiomyoma and myometrium, involving the activation of MAPK in LSMC and MSMC (Ding, L et al. J Clin Endocrinol Metab, 2004, 89:5549-5557). Since ECM turnover is a key regulator of the outcome of tissue fibrosis, and many cytokines, chemokines, growth factors and polypeptide hormones through specific intracellular signal transduction and activation of transcription factors influence the expression of ECM and proteases, further investigation is underway to elucidate their regulatory interactions affecting processes that may influence leiomyoma growth and regression.

In summary, in the present study, the inventors provide a comprehensive assessment of the gene expression profile of leiomyoma and matched myometrium during early-mid luteal phase of the menstrual cycle, a period characterized by elevated production of ovarian steroids and maximal leiomyoma growth, compared with tissues obtained from hormonally suppressed patients on GnRHa therapy and in response to the direct action of GnRHa on LSMC and MSMC. The present inventors identified several common and tissue-specific gene clusters in these cohorts suggesting their co-regulation by the same factors and or mechanism(s) in the same cluster. The present inventors validated the expression of several genes whose products are important in signal transduction, transcription, cell cycle regulation, apoptosis and ECM turnover, events critical to development, growth and regression of leiomyoma. Based on these and previous observations, the present inventors propose that the product of these specific genes, by regulating the local inflammatory and apoptotic processes leading to elaboration of profibrotic cytokines production such as TGF-β is central to the establishment and progression of fibrosis in leiomyoma. Provided in Examples 4-7 is further evidence for the role of TGF-β autocrine/paracrine action in this process.

### EXAMPLE 4

### Gene Expression Profiles of Leiomyoma and Matched Myometrium Cells in Response to TGF-β1

It has been reported that leiomyoma and myometrium express all the components of the TGF-β system, and it has been shown that TGF-β through Smads and MAPK pathways regulates the expression of a specific number of genes in LSMC and MSMC (Chegini, N. et al. J Clin Endocrinol Metab, 1999, 84:4138-43; Chegini, N. et al. Mol Hum Reprod, 2002, 8:1071-1078; Chegini, N. et al. Mol Cell Endocrinol, 2003, 209:9-16; Xu, J. et al. J Clin Endocrinol Metab, 2003, 88:1350-1361; Ding, L. et al. J Clin Endocrinol Metab, 2004, 89:5549-5557; Tang, X. M. et al. Mol Hum Reprod, 1997, 3:233-40). Here, the present inventors performed microarray analysis to further characterize the molecular environment of LSMC and MSMC directed by TGF-β autocrine/paracrine actions. LSMC and MSMC were treated with TGF-β1 (2.5 ng/ml) for 2, 6 and 12 hours, total RNA was isolated and subjected to microarray analysis. Following global normalization and transformation of the gene expression values, supervised learning, discrimination analysis, cross validation and variation filtering, the gene expression values for this study were independently subjected to statistical R programming analysis and ANOVA with false discovery rate selected at p≦0.001. The analysis identified 310 genes or 2.46% of the genes on the array as differentially expressed and regulated in response to time-dependent action of TGF-β in LSMC and MSMC.

Hierarchical clustering analysis separated these differentially expressed genes into distinctive clusters, with sufficient difference in their patterns allowing each cohort to cluster into their respective subgroup. The differentially expressed and regulated genes were separated into five clusters in response to time-dependent action of TGF-β in LSMC and MSMC, with genes in clusters A and B displaying a late response, genes in cluster D displaying early response, and genes in clusters C and E showing biphasic regulatory behaviors. Further analysis of the variance-normalized mean gene expression values divided the genes into 6 clusters, each displaying a different level of response to time-dependent action of TGF-β, with overlapping behavior between LSMC and MSMC with the exception of genes in clusters E and F.

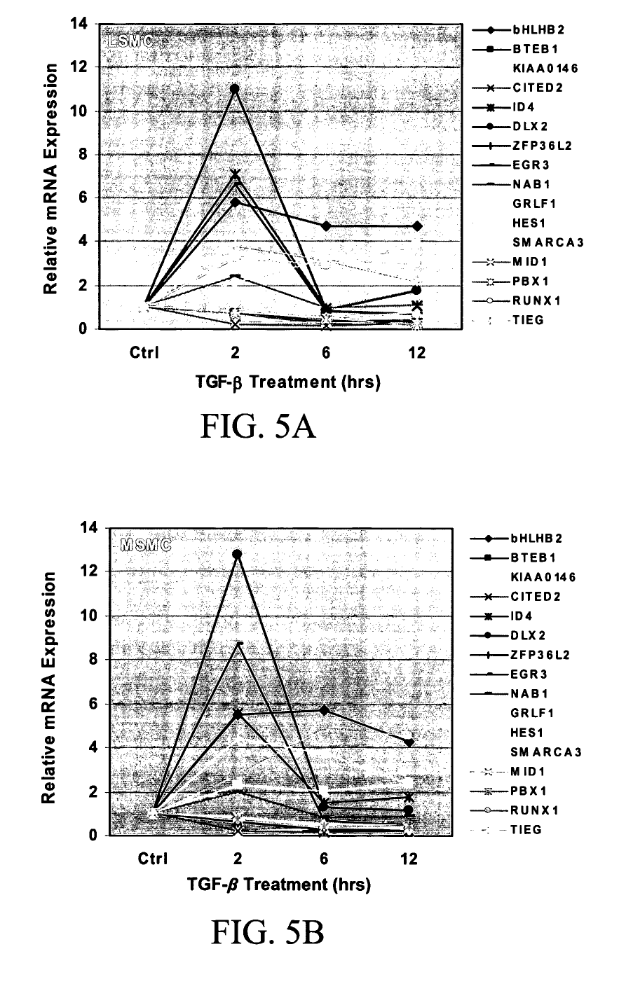

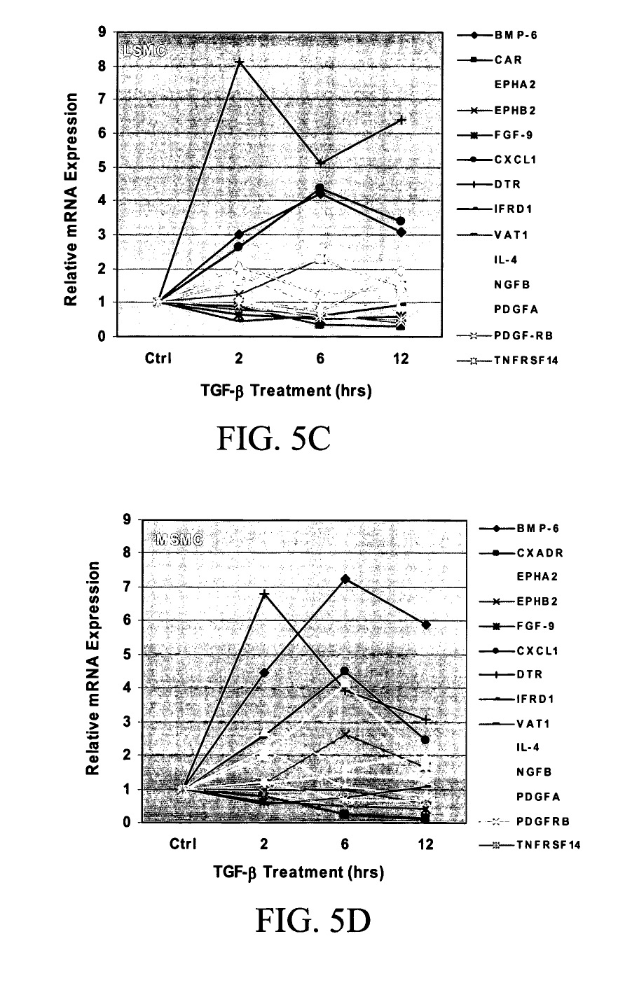

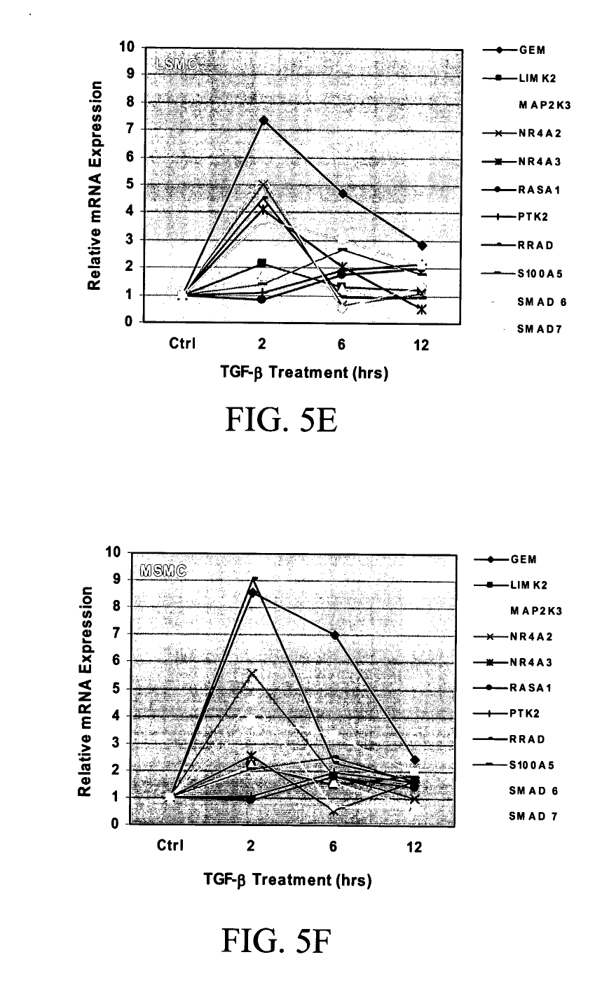

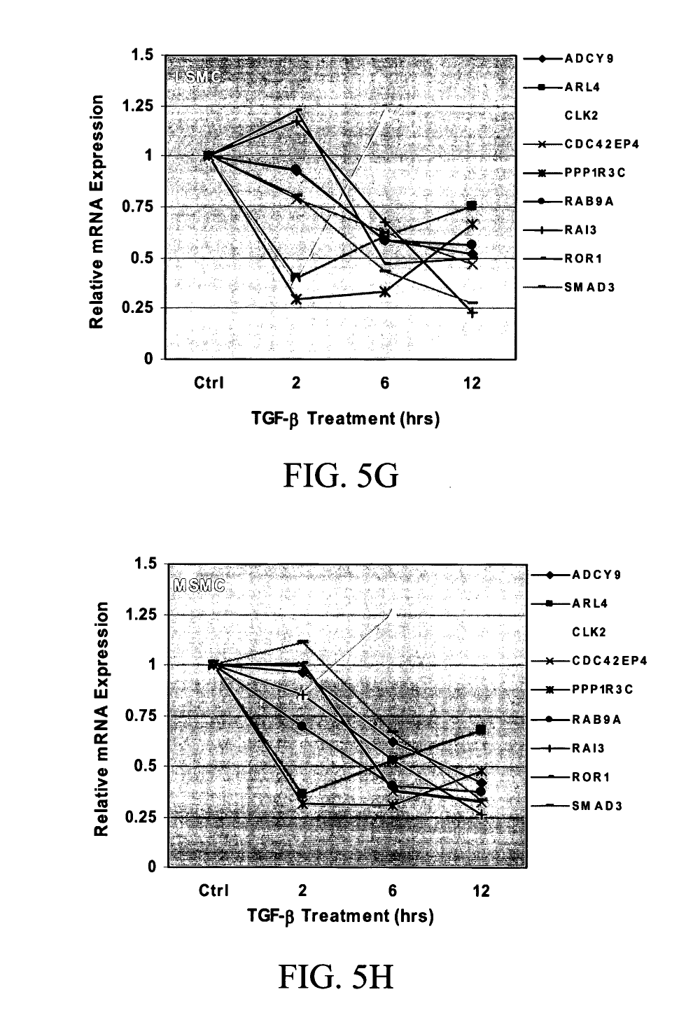

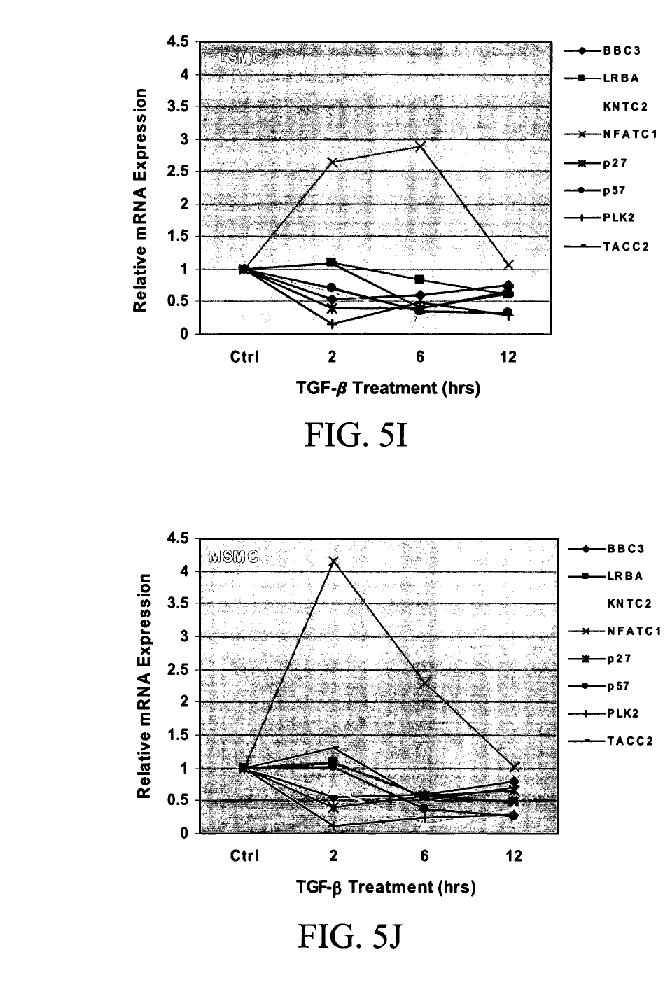

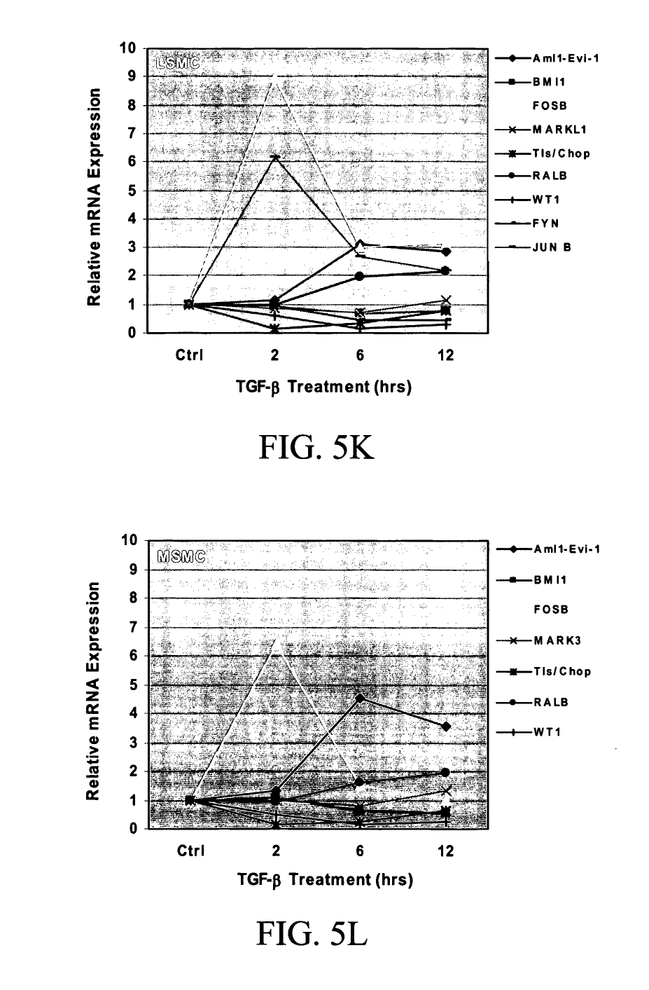

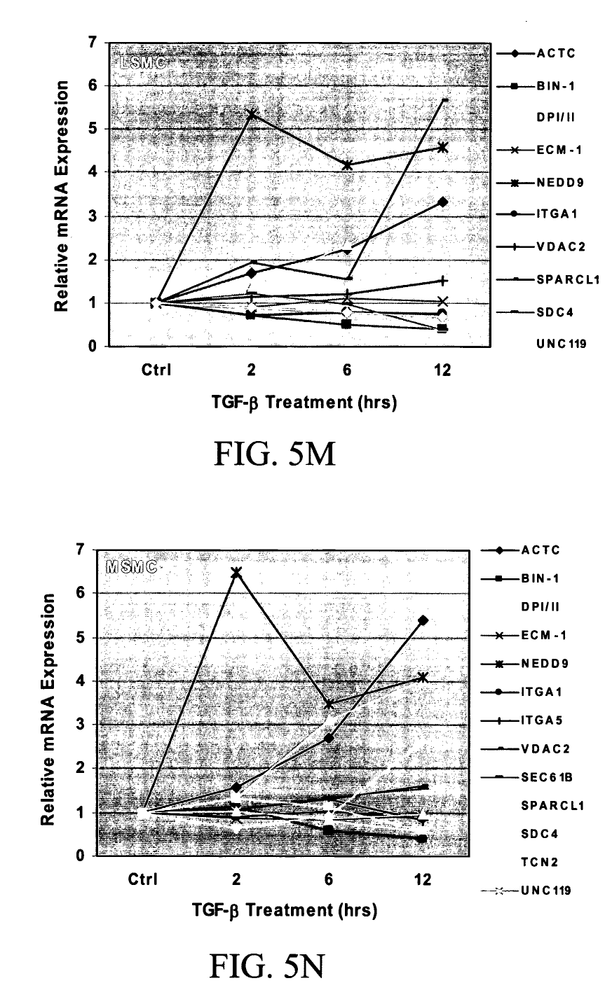

Comparative analysis between gene expression profiles of LSMC and MSMC in response to TGF-β action, with their corresponding leiomyoma and myometrium (tissues) from the untreated group revealed a substantial variability among their profiles (data not shown). However, gene ontology assessment and division into functional categories indicated that the majority of these genes (60 to 70%) are involved in transcriptional regulation and metabolism, cell cycle regulation, extracellular matrix and adhesion molecules, signal transduction and transcription factors. The time-dependent action of TGF-β on expression the profile of a selective group of these genes in the above clusters representing transcription factors, growth factors, cytokines, signal transduction pathways, ECM/adhesion molecules etc. in LSMC and MSMC are presented in FIG. 5A-5N.

### EXAMPLE 5

### Gene Expression Profiles of LSMC and MSMC In Response to TGF-β Following Pretreatment with TGF-β type II Receptor Antisense

To further evaluate the autocrine/paracrine action of TGF-β in leiomyoma and myometrial microenvironments, LSMC and MSMC were pretreated with TGF-β type II receptor (TGF-β type IIR) antisense oligomers to block/reduce TGF-β receptor signaling. Following pretreatments the cells were treated with or without TGF-β for 2 hours and their total RNA was subjected to microarray analysis. Based on the same data analysis described above with false discovery rate of p≦0.001, the present inventors identified 54 differentially expressed and regulated genes in response to TGF-β1 (2.5 ng/ml for 2 hours) in LSMC and MSMC pretreated with TGF-β type IIR antisense. Hierarchical cluster analysis distinctively separated these genes into 3 clusters with each cohort separated into their respective subgroups. The genes in clusters A and C displayed different response to TGF-β type IIR antisense treatment, while genes in cluster B showed overlapping behavior in LSMC and MSMC. However, there was an overlapping pattern between the gene expression profiles in TGF-β type IIR sense- and antisense-treated cells that could be due to the inability of antisense treatment to block all the combined action of autocrine/paracrine and exogenously added TGF-β. Interestingly, antisense treatment altered the expression of many genes known to be the target of TGF-β action, including those validated in this study. Gene ontology assessment and division into similar functional categories indicated that the majority of these genes are involved in transcriptional regulation and metabolism, cell cycle regulation, extracellular matrix and adhesion molecules, and transcription factors.

### EXAMPLE 6

### Comparative Analysis of Gene Expression Profiles in Response to TGF-β Type II Receptor Antisense and GnRHa Treatments in LSMC and MSMC

Since GnRHa alters the expression of TGF-β and TGF-β receptors expression in leiomyoma and myometrium as well as in LSMC and MSMC, the present inventors compared the gene expression profile of TGF-β type IIR antisense-treated with GnRHa-treated LSMC and MSMC, searching for common genes whose expression are affected by these treatments. Based on the same data analysis described above with false discovery rate selected at p≦0.001, the present inventors identified 222 genes differentially expressed and regulated in LSMC and MSMC in response to TGF-β type IIR antisense- and GnRHa-treated cells (Tables 7 and 8). Hierarchical clustering analysis separated these genes into 4 clusters displaying different pattern of regulation allowing their separation into respective subgroup. The genes in cluster A, B and D displayed different response to TGF-β type IIR antisense and GnRHa treatments, with genes in cluster C showing overlapping behavior in LSMC and MSMC.

Table 7 is a categorical list of genes identified as differentially expressed in LSMC pretreated with TGF-β type II receptor (TGF-β type IIR) antisense for 24 hours followed by TGF-β treatment for 2 hrs compared to LSMC treated with GnRHa (0.01 μM) for 2, 6, 12 hours. The genes were identified following supervised analysis of their expression values and statistical analysis in R programming and ANOVA with a false-discovery rate of rate of p≦0.001.

Table 8 is a categorical list of genes identified as differentially expressed in LSMC pretreated with TGF-β type II receptor (TGF-β type IIR) antisense for 24 hrs followed by TGF-b treatment for 2 hrs compared to LSMC treated with GnRHa (0.01 μM) for 2, 6, 12 hours. The genes were identified following supervised analysis of their expression values and statistical analysis in R programming and ANOVA with a false-discovery rate of rate of p≦0.001

### EXAMPLE 7

### Verification of Gene Transcripts in TGF-β-Treated LSMC and MSMC

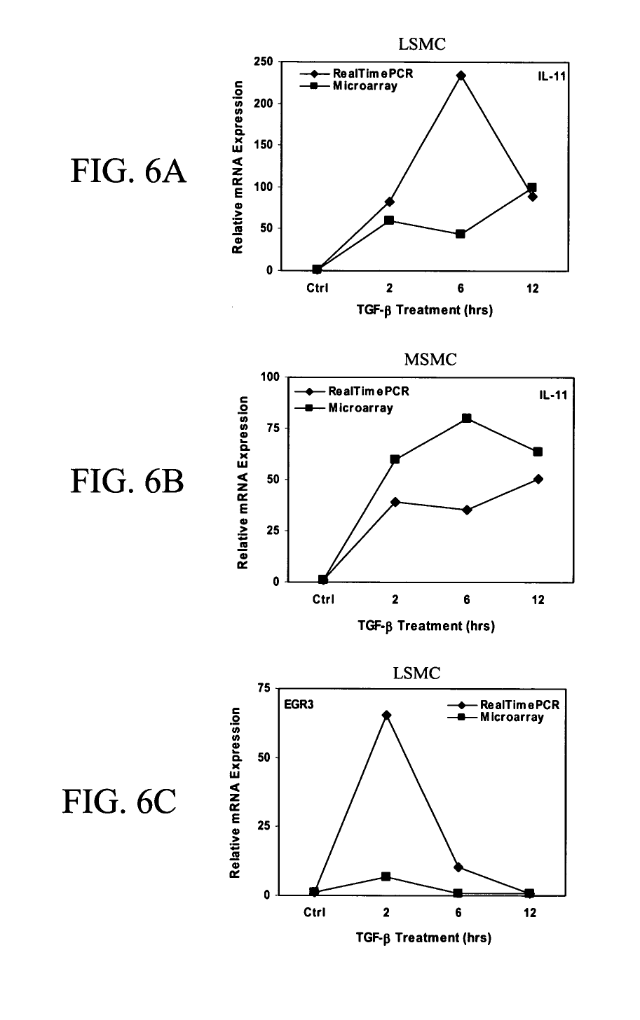

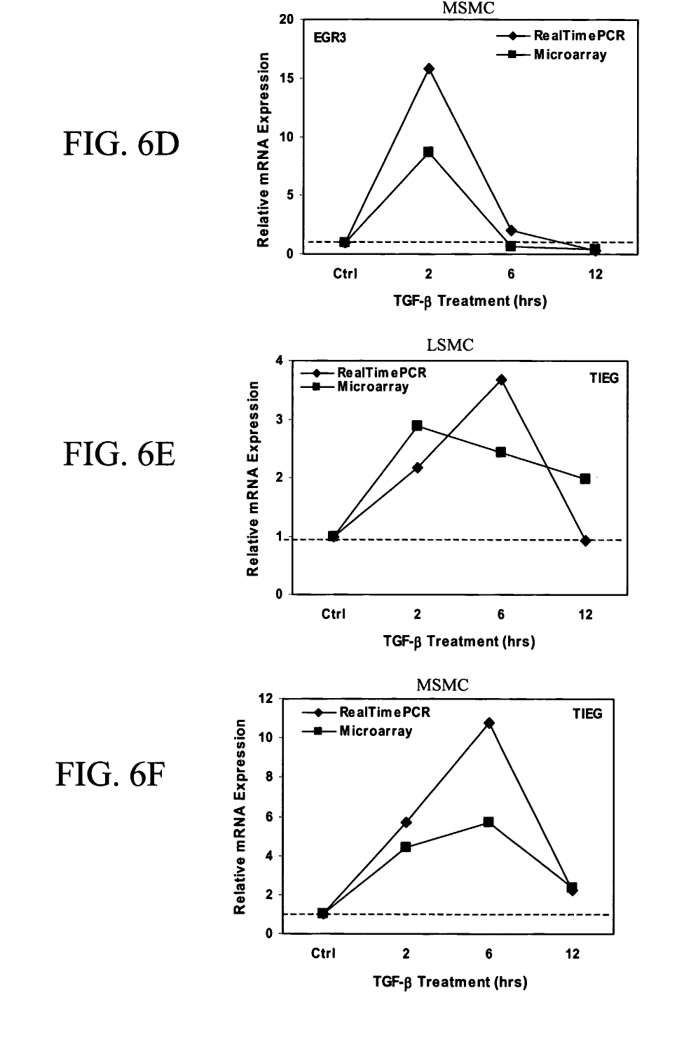

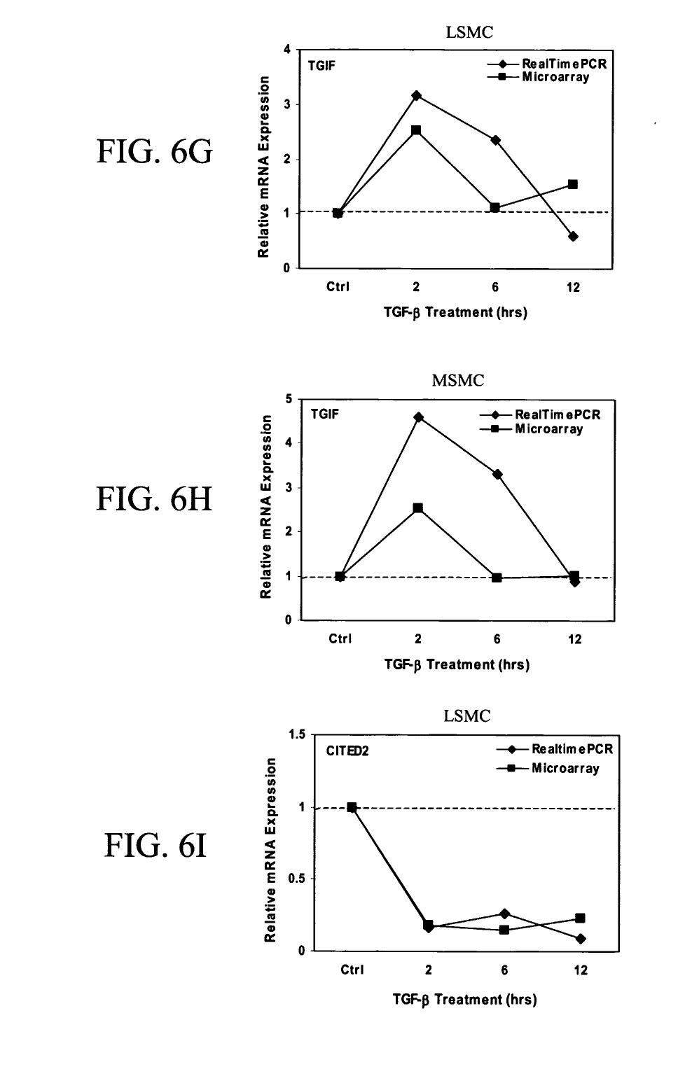

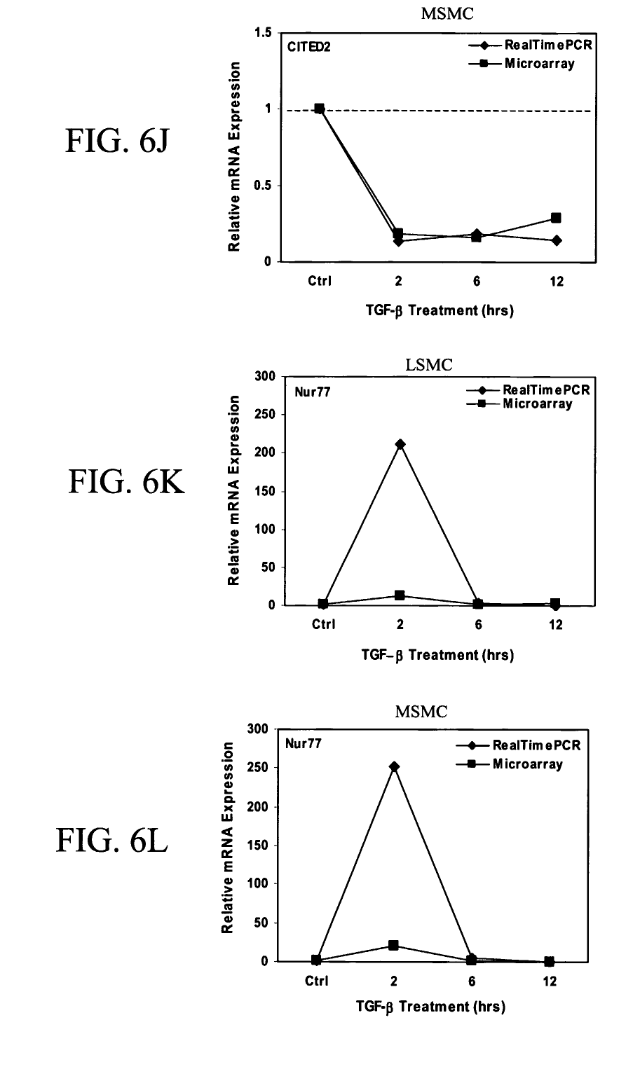

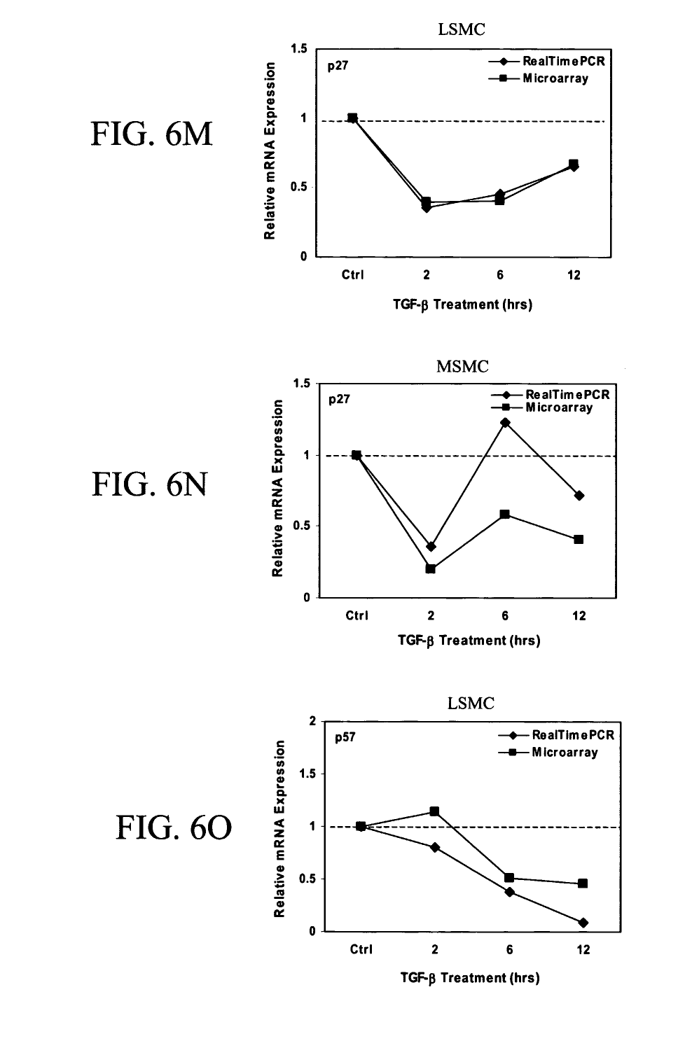

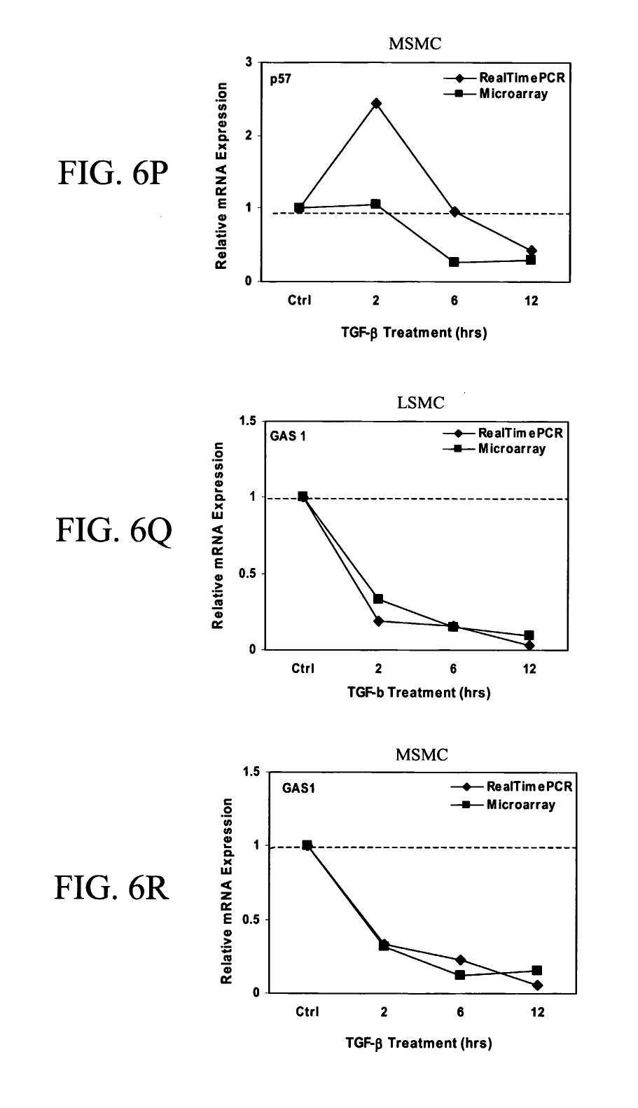

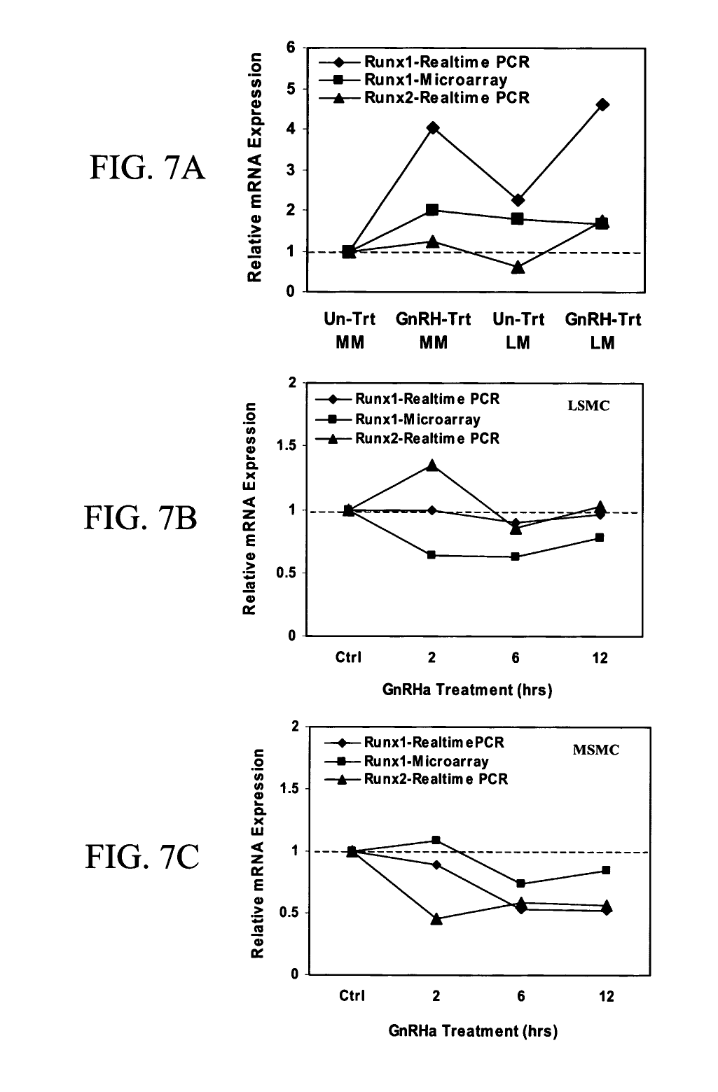

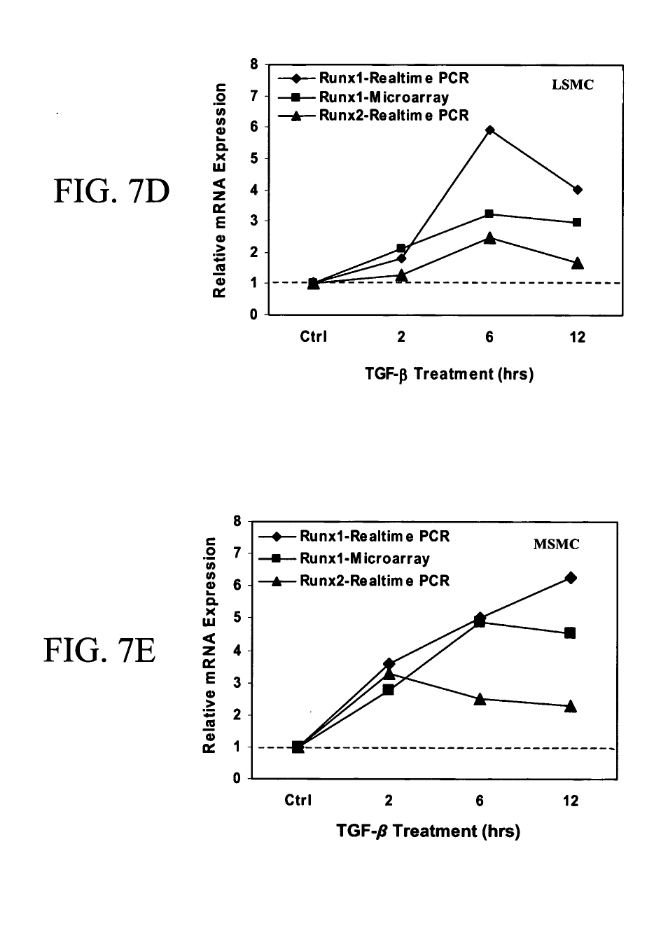

Using Realtime PCR, the present inventors validated the expression of 12 genes in response to time dependent action of TGF-β in LSMC and MSMC (FIGS. 6A-6R). They include IL-11, CITED2, Nur77, EGR3, TIEG, TGIF, p27, p57, GAS-1 and GPRK5, whose expression was also validated in leiomyoma and matched myometrium from untreated and GnRHa-treated cohorts as well as LSMC and MSMC treated in vitro with GnRHa. In addition, the present inventors verified the expression of Runx1 and Runx2. As illustrated TGF-β in a time dependent manner differentially regulate the expression of these genes in LSMC and MSMC with a pattern of expression displaying significant overlap between Realtime PCR and microarray analysis (FIGS. 6A-6R). However, the expression value of GPRK5 and Runx2 genes in microarray analysis of LSMC and MSMC did not meet the standard of analysis and was not included among the list of differentially expressed and regulated genes in response to TGF-β, although Runx2 mRNA is detectable by Realtime PCR (FIGS. 6A-6R). The results indicated that Runx1 and Runx2 expression not only is the target of TGF-β regulatory action, they are also regulated by GnRHa therapy in leiomyoma and myometrium as well as by GnRHa in LSMC and MSMC in vitro, with their time-dependent inhibition in MSMC (FIGS. 6A-6R).

The present inventors verified the expression of IL-11, TIGF, TIEG, p27 and p57 by Western blotting and their cellular distribution using immunohistochemistry in leiomyoma and myometrium. These findings provide further support for the microarray and Realtime PCR data indicating that the products of these genes are expressed in leiomyoma and myometrium. The present inventors are currently investigating time-dependent and dose-dependent regulation of these genes in response to TGF-β.

By extending previous work on the role of TGF-β in leiomyoma, in this study, the present inventors have provided the first example of gene expression fingerprints of LSMC and MSMC in response to autocrine/paracrine action of TGF-β. The present inventors further characterized the molecular environment of these cells following pretreatment with TGF-β type IIR antisense as a tool to interfere with the autocrine/paracrine action of TGF-β isoform s, and comparatively assessed their expression profiles with GnRHa-treated cells, which also inhibits TGF-β receptor expression in these cells (Dou, Q. et al. J Clin Endocrinol Metab, 1996, 81:3222-3230; Chegini, N. et al. Mol Hum Reprod, 2002, 8:1071-1078). Since the aim of this study was to capture the early and late autocrine/paracrine action of TGF-β in these cells, the present inventors selected a treatment strategy based on previous observations reflecting TGF-β time-dependent regulation of c-fos, c-jun, fibronectin, collagen type I, and PAI-1 expression (Ding, L. et al. J Clin Endocrinol Metab, 2004, 89:5549-5557). TGF-β regulates the expression of these genes in LSMC and MSMC through TGF-β receptor activation of Smad and MAPK pathways (Schnaper, H. W. et al. Am J Physiol Renal Physiol, 2003, 284:F243-252; Xu, J. et al. J Clin Endocrinol Metab, 2003, 88:1350-1361; Ding, L. et al. J Clin Endocrinol Metab, 2004, 89:5549-5557), whose promoters are known to contain TGF-β regulatory elements (Miyazono, K. et al. Oncogene, 2004, 23:4232-7; Moustakas, A. et al. Immunol Lett, 2002, 82:85-91). This study design is also consistent with other microarry studies profiling gene expression in response to TGF-β action in human dermal fibroblasts, HaCaT kritonocyte cell line and NMuMG, mouse mammary gland epithelial cell line, in which the cells were treated for 1, 2, 6 and 24 hours, displaying a Smad-mediated regulation of selected number of genes (Verrecchia, F. et al. J Biol Chem, 2001, 276:17058-17062; Zavadil, J. et al. Proc Natl Acad Sci USA, 2001, 98:6686-6691; Xie, L. et al. Breast Cancer Res, 2003, 5:R187-R198 25-27).

Cluster and tree-view analysis revealed a considerable similarity in overall gene expression patterns between LSMC and MSMC in response to TGF-β action; however, there was sufficient difference allowing their separation into respective subgroups. The genes in these clusters displayed different regulatory response to TGF-β action in a cell- and time-specific manner, with genes in clusters A and B displaying a late response, genes in cluster D displaying early responsiveness, and clusters C and E showing a biphasic regulatory behavior. These results suggest that the same factors and/or mechanisms co-regulate the expression of these genes in each cluster, possibly due to the presence of common regulatory elements in their promoters. Whether the expression profile of these genes in LSMC and MSMC respond differently to varying concentration of TGF-β, or other TGF-β isoforms is not established. However, the concentration of TGF-β used in this and other studies examining the effect of TGF-β on the expression of other genes (Xu, J. et al. J Clin Endocrinol Metab, 2003, 88:1350-1361; Ding, L. et al. J Clin Endocrinol Metab, 2004, 89:5549-5557; Tang, X. M. et al. Mol Hum Reprod, 1997, 3:233-40; Arici, A. and Sozen, I. Am J Obstet Gynecol, 2003, 188:76-83; Verrecchia, F. et al. J Biol Chem, 2001, 276:17058-17062; Zavadil, J. et al. Proc Natl Acad Sci USA, 2001, 98:6686-6691; Xie, L. et al. Breast Cancer Res, 2003, 5:R187-R198), is comparable with level of TGF-β produced by these cells, although LSMC produces more TGF-β1 compared to MSMC (Chegini, N. et al. J Clin Endocrinol Metab, 1999, 84:4138-43; Chegini, N. et al. Mol Hum Reprod, 2002, 8:1071-1078). Moreover, based on the profile of TGF-β isoforms's expression in leiomyoma, it has previously been proposed that TGF-β1 and TGF-β3 play an more critical role in leiomyoma (Chegini, N. et al. J Clin Endocrinol Metab, 1999, 84:4138-43), and in vitro studies have indicated a higher growth response to TGF-β1 (personal observations) and TGF-β3 in LSMC compared to MSMC (Lee, B. S. and Nowak, R. A. J Clin Endocrinol Metab, 2001, 86:913-920; Arici, A. and Sozen, I. Fertil Steril, 2000, 73:1006-1011). However, TGF-β isoforms mediate their actions through TGF-β type IIR, and alterations in the TGF-β receptor system may serve as a more accurate indicator of their overall autocrine/paracrine actions in these and other cell types. It has been shown that leiomyoma over-expresses TGF-β type IIR compared to myometrium (Dou, Q. et al. J Clin Endocrinol Metab, 1996, 81:3222-3230; Chegini, N., Luo X, Ding L, Ripley D 2003 The expression of Smads and transforming growth factor beta receptors in leiomyoma and myometrium and the effect of gonadotropin releasing hormone analogue therapy. Mol Cell Endocrinol 209:9-16), and pretreatment of LSMC with TGF-β type IIR antisense oligomers and/or neutralizing antibodies prevented TGF-β receptor-mediated actions (Chegini, N. et al. Mol Hum Reprod, 2002, 8:1071-1078; Xu, J. et al. J Clin Endocrinol Metab, 2003, 88:1350-1361).

These observations as well as identification of specific genes whose expression exhibited sensitivity to pretreatment with TGF-β type IIR antisense, among them genes containing TGF-β regulatory response elements in their promoters, further support TGF-β receptors mediated signaling in regulating the overall expression of these genes in LSMC and MSMC, and possibly in leiomyoma and myometrium. Lack of response of other TGF-β-targeted genes to TGF-β type IIR antisense pretreatment could be due to inability of antisense to block all the autocrine/paracrine, as well as exogenously added TGF-\tilde{β}. However, the expression of these genes may also be regulated as a consequence of TGF-β receptors overexpression and/or their altered intracellular signaling. Interestingly, activin receptor-like kinases (ALK) ALK1 and ALK5, which serve as TGF-β type I receptors and are activated by TGF-β type II receptors, have been shown to regulate the expression of different genes in endothelial cell in response to TGF-β action (Ota, T. et al. J Cell Physiol, 2002, 193:299-318). However, ALK1 functions as a TGF-β type I receptor in endothelial cells, while ALK-5 is expressed in various cells, and through distinct Smad proteins, i.e., Smad1/Smad5 and Smad2/Smad3, respectively, regulate gene expression in response to TGF-β actions (Ota, T. et al. J Cell Physiol, 2002, 193:299-318). The present inventors have identified the expression of all the components of the TGF-β receptor system, including ALK5 and Smad2/3 in leiomyoma and myometrium as well as LSMC and MSMC. However, TGF-β-mediated action through ALK1 could result in the regulation of a different set of genes not involving ALK5. In addition to TGF-β and TGF-β receptors, alteration in Smad expression is also considered to influence the outcome of several disorders targeted by TGF-β including tissue fibrosis (Flanders, K. C. Int J Exp Pathol, 2004, 85:47-64).

Gene ontology dividing the differentially expressed and regulated genes into similar functional categories revealed that the majority of the genes targeted in response to TGF-β treatment of LSMC and MSMC are associated with cellular metabolism, cell growth regulation (cell cycle and apoptosis), cell and tissue structure (ECM, adhesion molecules and microfilements), signal transduction and transcription factors. Despite the differences in their profiles, the present inventors found a substantial degree of similarity in functional annotation among the genes identified at tissue (leiomyoma and myometrium) and cellular (LSMC and MSMC) levels in response to TGF-β1. These differences between gene expression profiles of tissues and LSMC/MSMC in response to TGF-β could be due to the contribution of other cell types to the gene pool, and the influence of other autocrine/paracrine regulators on the overall genes expression at the tissue level. Previous studies from this laboratory and others have reported the expression of a few other genes targeted by TGF-β action in LSMC and MSMC. However, to the present inventors' knowledge, this is the first example of a large-scale gene expression profiling of these cells in response to TGF-β. Using quantitative realtime PCR analysis, the presenti inventors validated the expression of several of these genes in response to time-dependent action of TGF-β in LSMC and MSMC, including the expression of 10 genes validated in leiomyoma/myometrium as well as in LSMC/MSMC in response to GnRHa treatment.

The present inventors demonstrated that LSMC express an elevated level of IL-11 compared to MSMC, and its expression is a major target of TGF-β regulatory action. Although the biological significance of IL-11 expression in leiomyoma and myometrial environments, and consequence of its overexpression in leiomyoma await investigation, IL-11, alone, or through interaction with TGF-β is considered to play a critical role in progression of fibrotic disorders (Leng, S. X. and Elias, J. A. Int J Biochem Cell Biol, 1997, 29:1059-1062; Kuhn, C. et al. Chest, 2000, 117:260 S-262S; Zhu, Z. et al. Am J Respir Crit. Care Med, 2001, 164:S67-70; Chakir, J. et al. J Allergy Clin Immunol, 2003, 111:1293-1298). Other members of the interleukin family, IL-4 and IL-13, and their interactions with TGF-β are also reported to be equally important in this disorder (Wynn, T. A. Nat Rev Immunol, 2004, 4:583-594; Wynn, T. A. Annu Rev Immunol, 2003, 21:425-456). IL-13 expression has recently been identified in leiomyoma, and it has been discovered that exposure of LSMC to IL-13 upregulates the expression of TGF-β and TGF-β type II receptors in LSMC, suggesting a direct, and/or indirect regulatory function for IL-13 in mediating events leading to progression of tissue fibrosis in leiomyoma (Ding, L., Luo, X. Chegini, N. “The expression of IL-13 and IL-15 in leiomyoma and myometrium and their influence on TGF-b and proteases expression in leiomyoma and myometrial smooth muscle cells and SKLM, leiomyosarcoma cell line” J Soc Gyncol Invest, 2004, 00, 00). Other cytokines in this category including IL-4, IL-6, IL-8, IL-15, IL-17, TNF-α and GM-CSF are also expressed in leiomyoma and myometrium (Ding, L., Luo, X. Chegini, N. “The expression of IL-13 and IL-15 in leiomyoma and myometrium and their influence on TGF-b and proteases expression in leiomyoma and myometrial smooth muscle cells and SKLM, leiomyosarcoma cell line” J Soc Gyncol Invest, 2004, 00, 00; Chegini, N. “Implication of growth factor and cytokine networks in leiomyomas” In: Cytokines in human reproduction, J Hill ed. Wiley & Sons New York, 2000, 133-162; Chegini, N. et al. J Soc Gynecol Investig, 2003, 10:161-71). These cytokines are classified as type1/type2 related subsets and predominance toward type II direction is considered to result in inflammatory/immune responses leading to progression of tissue fibrosis (Zhu, Z. et al. Am J Respir Crit. Care Med, 2001, 164:S67-70; Chakir, J. et al. J Allergy Clin Immunol, 2003, 111:1293-1298; Wynn, T. A. Nat Rev Immunol, 2004, 4:583-594; Wynn, T. A. Annu Rev Immunol, 2003, 21:425-456; Lee, C. G. et al. J Exp Med, 2004, 200:377-389). A recent report has further elaborated the participation of IL-11 and TGF-β, and transcription factor EGR1 in tissue fibrosis, through a mechanism involving regulation of the balance between the rate of cellular apoptosis and inflammatory response (Lee, C. G. et al. J Exp Med, 2004, 200:377-389). EGR1 has previously been identified among the differentially expressed genes in leiomyoma and myometrium (Chegini, N. et al. J Soc Gynecol Investig, 2003, 10:161-71) and expression of EGR2 and EGR3 in these tissues and regulation of EGR3 in response to TGF-βaction in LSMC and MSMC is demonstrated herein.

Elevated expression and preferential phosphorylation of EGR1 leads to regulation of target genes whose products are involved in apoptosis as well as angiogenesis and cell survival, including IL-2, TNF-alpha, Flt-1, Fas, Fas ligand, cyclin D1, p15, p21, p53, PDGF-A, angiotensin II-dependent activation of PDGF and TGF-β, VEGF, tissue factor, 5-lipoxygenase, thymidine kinase, superoxide dismutase, intercellular adhesion molecule 1 (ICAM-1), fibronectin, urokinase-type plasminogen activator and matrix metalloproteinase type I (Thiel, G. and Cibelli, G. J Cell Physiol, 2002, 193:287-292; Khachigian, L. M. Cell Cycle, 2004, 3:10-1; Nagamura-Inoue, T. et al. Int Rev Immunol, 2001, 20:83-105; Liu, C. et al. Cancer Gene Ther, 1998, 5:3-28; Liu, C. et al. J Biol Chem, 1999, 274:4400-11; Baoheng, Du. et al. J Biol Chem, 2000, 275:39039-39047). The expression of many of these genes has been documented in myometrium and leiomyoma (Blobe, G. C. et al. N Engl J Med, 2000, 342:1350-1358), and known to be the target of TGF-β regulatory action. EGR1 also acts as a transcriptional repressor of TGF-β type II receptor through direct interaction with SP1 and Ets-like ERT sites in proximal promoter of the gene (Baoheng, Du. et al. J Biol Chem, 2000, 275:39039-39047). Transfection of EGR1 expression vector into a myometrial cell line (KW) expressing low levels of EGR1 is reported to result in a rapid growth inhibition of these cells (Shozu, M. et al. Cancer Res, 2004, 64:4677-4684). To the present inventors' knowledge, this is the first report of the regulatory action of TGF-β on EGR3 expression, not only in LSMC and MSMC, but any other cell types. Based on previous and present observations, the present inventors propose that a local inflammatory response mediated through individual and combined actions of TGF-β, IL-13 and IL-11, as well as regulatory function of TGF-β on EGR expression, results on local expression of set of genes whose products promote apoptotic and non-apoptotic cell death, further enhancing an inflammatory reaction that orchestrate various events leading to progression of fibrosis in leiomyoma.

Additional genes identified as differentially expressed and regulated by TGF-β autocrine/paracrine action in LSMC and MSMC in this functional category include TGIF, TIEG, CITED2, Nur77, Runx1 and Runx2. These transcription factors possess key regulatory functions in the expression of a wide range of genes in response to various stimuli specifically TGF-β. The expression of TGIF, TIEG, CITED2 and Nur77 is highly regulated in LSMC and MSMC, and with the exception of CITED2, TGF-β transiently increased their expression in a time-dependent manner. TGIF is a transcriptional co-repressor that directly associates with Smads and inhibits Smad-mediated transcriptional activation by competing with p300 for Smad association (Chen, F. et al. Biochem J, 2003, 371:257-263; Wotton, D. et al. Cell Growth Differ, 2001, 12:457-63). CITED2, induced by multiple cytokines, growth factors and hypoxia, also interacts with p300 and function as a coactivator for transcription factor AP-2 (Tien, E. S. et al. J Biol Chem, 2004, 279:24053-63). CITED2-mediated action is reported to result in down-regulation of MMP-1 and MMP-13 through interactions with CBP/p300 and other transcription factors such as c-fos, Ets-1, NFκB, and Smads that control MMPs promoter activities (Yokota, H. et al. J Biol Chem, 2003, 278:47275-47280; Shi, Y. and Massague, J. Cell, 2003, 113:685-700). TGF-β targets the expression of these transcription factors and MMPs in many cell types, including LSMC and MSMC (Ding, L. et al. J Clin Endocrinol Metab, 2004, 89:5549-5557; Shi, Y. and Massague, J. Cell, 2003, 113:685-700; Ma, C. and Chegini, N. Mol Hum Reprod, 1999, 5:950-954), thus their differential regulation and interactions with CITED2 and TGIF may serve in regulating the outcome of TGF-β autocrine/paracrine actions in leiomyoma involving cell growth, inflammation, apoptosis and tissue turnover. Unlike TGIF, TIEG is rapidly induced by TGF-β and enhances TGF-β actions through Smad2/3 activation (Johnsen, S. A. et al. Oncogene, 2002, 21:5783-90; Cook, T. and Urrutia, R. Am J Physiol Gastrointest Liver Physiol, 2000, 278:G513-521; Ribeiro, A. et al. Hepatology, 1999, 30:1490-1497). However, TIEG has no effect on gene transcription in the absence of Smad4, or due to overexpression of Smad7, although it is capable of increasing Smad2/3 activity in the absence of Smad7 (Shi, Y. and Massague, J. Cell, 2003, 113:685-700; Johnsen, S. A. et al. Oncogene, 2002, 21:5783-90). It was shown that TGF-β induced a rapid, but transient expression of TIEG in LSMC and MSMC, and the expression of Smad2/3, Smad4 and Smad7 and their differential regulation by TGF-β has been demonstrated in these cells (Xu, J. et al. J Clin Endocrinol Metab, 2003, 88:1350-1361; Ding, L. et al. J Clin Endocrinol Metab, 2004, 89:5549-5557). Based on these observations, the present inventors further propose that TGF-β through a mechanism involving TGIF, TIEG and Smads self regulates its own autocrine/paracrine action in leiomyoma/myometrium. Estrogen has also been shown to increase TIEG expression in breast tumor cell (Johnsen, S. A. et al. Oncogene, 2002, 21:5783-90; Sorbello, V. et al. Int J Biol Markers, 2003, 18:123-9). Since estrogen, a major growth-promoting factor for leiomyoma, induces TGF-β expression in LSMC and MSMC (Chegini, N. et al. J Clin Endocrinol Metab, 1999, 84:4138-43; Chegini, N. et al. Mol Hum Reprod, 2002, 8:1071-1078), E2-induced TGF-β or estrogen directly may regulate TIEG expression in leiomyoma. TIEG is also reported to trigger apoptotic cell programs by a mechanism involving the formation of reactive oxygen species (Ribeiro, A. et al. Hepatology, 1999, 30:1490-1497), often created as a result of local inflammatory response. Whether TGF-β-induced TIEG through the above mechanism results in apoptotic response in leiomyoma is not known; however, formation of reactive oxygen species may enhance local inflammatory response serving as an additional mediator of tissue fibrosis in leiomyoma.

With respect to Nur77, it regulates the expression of a group of genes whose products are involved in cell cycle regulation, differentiation, apoptosis, and malignant transformation (Rajpal, A. et al. EMBO J, 2003, 22:6526-36; Castro-Obregon, S. et al. J Biol Chem, 2004, 279:17543-17553). Evidence has been provided that Nur77 is the target of regulatory action of TGF-β in LSMC and MSMC, with pattern of expression resembling that observed in leiomyoma and myometrium, respectively (Chegini, N. et al. J Soc Gynecol Investig, 2003, 10:161-71). Although the nature and functional significance of Nur77 expression in leiomyoma, and regulation by TGF-β is unknown, malignant transformation in leiomyoma is rare, suggesting Nur77 may function as regulator of cell cycle in leiomyoma and myometrium. In addition to Nur77, the present inventors discovered that the expression of various genes functionally associated with cell cycle regulation and apoptosis are influenced by TGF-β autocrine/paracrine action, and balance of their expression may become a critical factor in leiomyoma growth and regression. Additional transcription factors whose expression was the target of TGF-β action in LSMC and MSMC are Runx1 and Runx2. This family of transcriptional factors consisting of Runx1 to Runx3, are integral components of signaling cascades mediated by TGF-β and bone morphogenetic proteins regulating various biological processes, including cell growth and differentiation, hematopoiesis and angiogenesis (Miyazono, K. et al. Oncogene, 2004, 23:4232-7; Shi, Y. and Massague, J. Cell, 2003, 113:685-700; Levanon, D. and Groner, Y. Oncogene, 2004, 23:4211-4219; McCarthy, T. L. et al. J Biol Chem, 2003, 278:43121-43119; Ito, Y. and Miyazono, K. Curr Opin Genet Dev, 2003, 13:43-47). The present inventors provided the first evidence for regulatory action of GnRHa therapy and GnRHa direct action on Runx1 and Runx2 expression in leiomyoma, myometrium as well as LSMC and MSMC, with GnRHa significantly inhibiting their expression, specifically in MSMC. Although Runx2 is expressed at low levels in leiomyoma and myometrium, Runx1 and Runx2 expression in LSMC and MSMC displayed a rapid response to TGF-β action in vitro, with Runx1 showing a significantly higher response. TGF-β activation of Smad and MAPK cascades regulates the expression of Runx2; however, interaction with Smad3 causes repression of Runx2 and downstream transcription activation of specific genes (Miyazono, K. et al. Oncogene, 2004, 23:4232-7; Shi, Y. and Massague, J. Cell, 2003, 113:685-700; Ito, Y. and Miyazono, K. Curr Opin Genet Dev, 2003, 13:43-47). It has recently been reported that TGF-β and GnRH activate the MAPK pathway (Ding, L. et al. J Clin Endocrinol Metab, 2004, 89:5549-5557), and GnRHa alter TGF-β-activated Smad in LSMC and MSMC (Xu, J. et al. J Clin Endocrinol Metab, 2003, 88:1350-1361), signaling cascade that may regulate Runx1 and Runx2 expression in these cells. Differential regulation of Runx1 and Runx2 by TGF-β and GnRHa imply their potential biological implication, specifically in regulating TGF-β action in leiomyoma microenvironment. This is particularly interesting since estrogen is also reported to enhance Runx2 activity, through a mechanism involving TGF-β type I receptor gene promoter, which contains several Runx binding sequences (McCarthy, T. L. et al. J Biol Chem, 2003, 278:43121-43119). Together, the identification of these and several other key transcription factors in LSMC and LSMC, and their regulation by TGF-β serving as integral components of inflammatory, cell cycle and apoptotic processes, further support the present inventors' hypothesis that a regulatory balance between these events is a key factor in progression of fibrosis mediated by TGF-β in leiomyoma.

Such balance between cell proliferation and apoptosis is critical to tissue homeostasis and central to leiomyoma growth and regression. Since both positive and negative signals determine the outcome of these events, the present inventors searched and identified several genes in this category in previous studies and in the current study as differentially expressed and regulated in leiomyoma and myometrium, as well as in LSMC and MSMC in response to TGF-β. The primary focus here was placed on p27Kip1, p57Kip2 and Gas1 expression, because of their regulation by GnRHa. It was found that TGF-β suppressed the expression of these genes in LSMC, and in a biphasic fashion accompanied by suppression of GAS1 expression in MSMC. TGF-β is known to regulate the expression of several cell cycle regulatory proteins including p27, which bind cyclin-dependent kinase (CDK), and by inhibiting catalytic activity of CDK-cyclin complex, regulate cell cycle progression and apoptosis (Reed, S. I. Nat Rev Mol Cell Biol, 2003, 4:855-64). However, TGF-β regulation of p57 expression is limited (Miyazono, K. et al. Oncogene, 2004, 23:4232-7; Moustakas, A. et al. Immunol Lett, 2002, 82:85-91; Kim, S. J. and Letterio, J. Leukemia, 2003, 17:1731-7) and available data suggests that TGF-β enhances p57 degradation through ubiquitin-proteasome pathway and Smad-mediated signaling (Nishimori, S. et al. J Biol Chem, 2001, 276:10700-10705). TGF-β-induced p57 degradation, CDK2 activation and cell proliferation is blocked by proteasome inhibitors and/or by overexpression of Smad7 (Nishimori, S. et al. J Biol Chem, 2001, 276:10700-10705; Yokoo, T. et al. J Biol Chem, 2003, 278:52919-52923; Brown, K. A. et al. Breast Cancer Res, 2004, 6:R130-R139; Kawaguchi, K. et al. Hum Pathol., 2004, 2004; 35:61-8). TGF-β-induced cell growth is also influenced by c-myc and the expression and activities of G1, G2, CDK and cyclins, and their inhibitors p15INκ4b and p21 (Miyazono, K. et al. Oncogene, 2004, 23:4232-7; Moustakas, A. et al. Immunol Lett, 2002, 82:85-91; Shi, Y. and Massague, J. Cell, 2003, 113:685-700), and they were identified among differentially expressed and regulated genes in LSMC and MSMC by TGF-β (Chegini, N. et al. J Soc Gynecol Investig, 2003, 10:161-71). With respect to Gas1, to the present inventors' knowledge, this observation is the first to demonstrate Gas1 expression in human uterine tissue and its regulation by TGF-β. GAS1 acts as a negative regulator of the cell cycle and has been positively correlated with the inhibition of endothelial cell apoptosis and the integrity of resting endothelium (Spagnuolo, R. et al. Blood, 2004, 103:3005-12). Similar to p15, p21 and p27, myc suppresses the expression of GAS1 by limiting myc-max heterodimers binding to their promoters, (Gartel, A. L. and Shchors, K. Exp Cell Res, 2003, 283:17-21; Lee, T. C. et al. Proc Natl Acad Sci USA, 1997, 94:12886-91). GAS1 is also reported to suppress growth and tumorigenicity of human tumor cells, and overexpression of MDM2, or p53 mutation inhibits Gas1-mediated action (Evdokiou, A. and Cowled, P. A. Exp Cell Res, 1998, 240:359-67). The present inventors have identified max and MDM2 expression in LSMC and MSMC and their regulation by TGF-β, suggesting their potential interactions in leiomyoma cellular environment. It was previously reported that TGF-β isoforms stimulate DNA synthesis, but not cell division in LSMC and MSMC, suggesting that p27, p57 and Gas1, as well as the products of other cell cycle regulators, may influence the effect of TGF-β action on leiomyoma cell growth late in the S to M phases of the cell cycle transition. Collectively, the identification of several genes in this category, whose products regulate cell cycle progression as target of TGF-β autocrine/paracrine action in LSMC and MSMC, further indicate the importance of TGF-β in regulating the balance between cell proliferation, cell cycle arrest and apoptosis whose outcome directs leiomyoma growth and/or regression.

Expression and activation of various components of signal transduction pathways are essential for mediating the cellular actions of growth factors, cytokines, chemokines, polypeptide hormones, and adhesion molecules. The present inventors identified several genes functionally belonging to this category as differentially expressed and regulated in LSMC and MSMC in response to TGF-β action, among them are member of family of Ras/Rho, Smads and MAPK, guanine nucleotide binding protein alpha, GTP-binding protein overexpressed in skeletal muscle, PTK2 protein tyrosine kinase 2, S100 calcium-binding protein A5, adenylate cyclase 9, CDC-like kinase 2, Cdc42 effector protein 4, retinoic acid induced 3, receptor tyrosine kinase-like orphan receptor 1, LIM protein and LIM domin kinase 2, phosphodiesterase 4D (cAMP-specific), protein phosphatase alpha, serine/threonine kinase 17a (apoptosis-inducing), focal adhesion kinase 2, STATs, etc. Although, Smad and MAPK pathways are known to be recruited and activated by TGF-β receptors, including in LSMC and MSMC, the components of other pathways are not the target of TGF-β. However, many growth factors, cytokines, chemokines, polypeptide hormones and adhesion molecules, expressed by LSMC and MSMC, either alone or through crosstalk with TGF-β receptor signaling may activate various components of the other pathways (Blobe, G. C. et al. N Engl J Med, 2000, 342:1350-1358; Chegini, N. “Implication of growth factor and cytokine networks in leiomyomas” In: Cytokines in human reproduction, J Hill ed. Wiley & Sons New York, 2000, 133-162; Chegini, N. et al. J Soc Gynecol Investig, 2003, 10:161-71), although only the expression and activation of a few of these molecules has been demonstrated in leiomyoma and myometrium, and in LSMC and MSMC. Since GPRK5 expression was detected in leiomyoma and myometrium and was the target of GnRHa action in LSMC and MSMC, the present inventors further investigated and found GPRK5 expression is regulated by TGF-β. The biological implication of GPRK5 and regulation by TGF-β in LSMC and MSMC is unclear; however, GPKs serve as negative regulators of GPCR mediated biological responses through the generation of second messengers, such as cAMP and calcium/calmodulin, and down-regulation of their activity (desensitization) (Luo, J. and Benovic, J. L. J Biol Chem, 2003, 278:50908-14; Miyagawa, Y. et al. Biochem Biophys Res Commun, 2003, 300:669-73; Cornelius, K. et al. J. Biol. Chem., 2001, 276:1911-1915). Activation of calcium/calmodulin is reported to alter Smad function, with inhibition of calmodulin resulting in an increase in activin-dependent induction of target genes, whereas its overexpression decreased activin- and TGF-βaction (Miyazono, K. et al. Oncogene, 2004, 23:4232-7; Moustakas, A. et al. Immunol Lett, 2002, 82:85-91; Shi, Y. and Massague, J. Cell, 2003, 113:685-700). The result suggests that GPRK may act as downstream regulator of TGF-β receptor singling possibly through modulation of PKC, MAPK and/or calmodulin and hence influencing TGF-β autocrine/paracrine action in leiomyoma.

Tissue remodeling is also a critical step in progression of fibrotic disorders and modulation of ECM, adhesion molecules and protease expression, and phenotypic changes toward a myofibroblastic phenotype are essential components of this process (Blobe, G. C. et al. N Engl J Med, 2000, 342:1350-1358; Gabbiani, G. J Pathol, 2003, 200:500-3; Phan, S. H. Chest, 2002, 122:286 S-289S; Shephard, P. et al. Thromb Haemost, 2004, 92:262-74; Gauldie, J. et al. Curr Top Pathol, 1999, 93:35-45). In this study and the previous study, the presenti inventors identified the expression of several genes in this category in leiomyoma and myometrium, as well as LSMC and MSMC including fibronectin, collagens, decorin, versican, desmin, vimentin, fibromodulin, several member of intergrin family, desmoplakin, extracellular matrix protein 1, enhancer of filamentation 1, porin, SPARC-like 1, syndecan 4, endothelial cell-specific molecule 1, as well as MMPs, TIMPs and ADAMs (Chegini, N. et al. J Soc Gynecol Investig, 2003, 10:161-71). The expression of fibronectin, vimentin, collagen type 1, fibromodulin, MMP1, MMP2 and MMP9, TIMPs in leiomyoma and myometrium has been demonstrated and showed that TGF-β, through the activation of MAPK, regulates the expression of some of these genes (Ding, L. et al. J Clin Endocrinol Metab, 2004, 89:5549-5557; Yokota, H. et al. J Biol Chem, 2003, 278:47275-47280; Dou, Q. et al. Mol Hum Reprod, 1997, 3:1005-14). Of particular interest are the elevated expression of decorin, vimentin and fibromodulin in leiomyoma since they are considered to regulate the outcome of tissue fibrosis and their ability to bind TGF-β, thus controlling TGF-β autocrine/paracrine action (Blobe, G. C. et al. N Engl J Med, 2000, 342:1350-1358; Chegini, N. “Implication of growth factor and cytokine networks in leiomyomas” In: Cytokines in human reproduction, J Hill ed. Wiley & Sons New York, 2000, 133-162; Levens E, Luo X, Ding L, Williams R S, Chegini N “Differential Expression of fibromodulin and Abl-interactor 2 in leiomyoma and myometrium and regulation by gonadotropin releasing hormone analogue (GnRHa) therapy” Fertil Steril, 2004, (In press); Chakravarti, S. Glycoconj J, 2002, 19:287-93). Since leiomyoma is believed to derive from transformation of myometrial connective tissue fibroblast and/or smooth muscle cells, the expression of vimentin in leiomyoma/LSMC imply that these cells have adopted a myofibroblastic characteristic. While granulation tissue myofibroblasts are derived from local fibroblasts, other cell types including smooth muscle cells have the potential to acquire a myofibroblastic phenotype (Lee, C. G. et al. J Exp Med, 2004, 200:377-389; Gabbiani, G. J Pathol, 2003, 200:500-3; Phan, S. H. Chest, 2002, 122:286 S-289S; Shephard, P. et al. Thromb Haemost, 2004, 92:262-74). These cells express various cytokines including GM-CSF, IL-11 and TGF-β of which GM-CSF is considered to participate in fibroblasts transformation into myofibroblasts and enhancing their TGF-β expression (Gabbiani, G. J Pathol, 2003, 200:500-3; Phan, S. H. Chest, 2002, 122:286 S-289S; Shephard, P. et al. Thromb Haemost, 2004, 92:262-74). It has been shown that GM-CSF is a key regulator of TGF-β in LSMC, and their interaction and as well as the involvement of other cytokines such as IL-11 and IL-13 regulate various events leading to leiomyoma formation and progression of fibrosis (Ding, L. et al. J Clin Endocrinol Metab, 2004, 89:5549-5557; Ding L, Luo X Chegini N “The expression of IL-13 and IL-15 in leiomyoma and myometrium and their influence on TGF-b and proteases expression in leiomyoma and myometrial smooth muscle cells and SKLM, leiomyosarcoma cell line” J Soc Gyncol Invest, 2004, 00, 00). IL-11 either alone or through the induction of TGF-β is reported to alter myofibroblasts ECM turnover resulting in the progression of tissue fibrosis (Lee, C. G. et al. J Exp Med, 2004, 200:377-389; Bamba, S. et al. Am J Physiol Gastrointest Liver Physiol, 2003, 285:G529-38). Despite the importance of tissue turnover in the pathophysiology of leiomyoma, little data are currently available of the extent of ECM expression and the difference that may exist compared to myometrium, that contribute to the fibrotic characteristic of leiomyoma.

In conclusion, as a continuation of work with TGF-β, the present inventors have provided the first large-scale example of gene expression profile of LSMC and MSMC identifying specific cluster of genes whose expression is targeted by autocrine/paracrine action of TGF-β. The present inventors validated the expression of a selective number of these genes functionally recognized to regulate inflammatory response, angiogenesis, cell cycle, apoptotic and non-apoptotic cell death, and ECM matrix turnover, events that are central to leiomyoma pathobiology. Based on the present work and previous work with TGF-β, the present inventors propose that the individual and combined action of TGF-β with other profibrotic cytokines such as IL-11, orchestrate local inflammatory responses mediated through and influenced by the expression of genes whose products regulate cell cycle progression, angiogenesis, apoptosis and tissue turnover, providing an environment leading to the progression of fibrosis.

### EXAMPLE 8

### Differential Expression of Fibromodulin and Abl-Interactor 2 in Leiomyoma and Myometrium and Regulation by Gonadotropin Releasing Hormone Analogue (GnRHa) Therapy

To validate the expression of fibromodulin and Abl-interactor 2 (Abi-2) that were identified as being differentially expressed in leiomyomata and myometrium and were regulated by GnRHa therapy. Fibromodulin is considered to have an anti-fibrotic role in wound repair and may be a biologically relevant modulator of TGF-beta activity during scar formation. Abl-interactor 2 encodes a non-receptor tyrosine kinase, c-Abl, that has been implicated in a variety of cellular processes including cell growth, reorganization of cytoskeleton, cell death and stress responses. Accordingly, a prospective study determining the tissue gene expression profile of myometrium and elimyoma using Real-time polymerase chain reaction (PCR) was carried out. Portions of leiomyoma and matched unaffected myometrium were collected from premenopausal women (N=27) who were scheduled to undergo hysterectomy for indications related to symptomatic leiomyoma. Seven of the patients received GnRHa therapy for three months prior to surgery. The untreated patients did not receive any medications (including hormonal therapy) during the 3 months prior to surgery.

Based on endometrial histology and the patient's last menstrual period, the tissue samples were identified as being from the proliferative (N=8) or the secretory (N=12) phase of the menstrual cycle. Total RNA was isolated and subjected to Real-time PCR. The results were analyzed using unpaired Student-test and Tuckey test (ANOVA) with a probability level of P<0.05 considered significant. These results for the first time document expression of fibromodulin and Abi-2 in leiomyoma and myometrium and provide evidence that the expression of these genes is influenced by ovarian steroids and possibly by a direct action of GnRHa on myometrial and leiomyoma cells.

### Materials and Methods

The following materials and methods describe those utilized in Examples 9-13. All the materials for Realtime PCR, immunoblotting and immunohistochemistry were purchased from APPLIED BIOSYSTEM (Foster City, Calif.), BIORAD (Hercules, Calif.), and VECTOR Laboratories (Burlingame, Calif.), respectively. Leuprolide acetate (GnRHa) was purchased from SIGMA Chemical (St Louis, Mo.), human recombinant TGF-β1, polyclonal antibody to CCN4 (WISP-1) were purchased from R&D System (Minneapolis, Minn.). Polyclonal antibodies to CTGF (CCN2), NOV (CCN-3), fubulin-1C and SA100A4 were purchased from SANTA CRUZ Biotechnology (Santa Cruz, Calif.). U0126, MEK1/2 synthetic inhibitor was purchased from CALBIOCHEM (San Diego, Calif.).

Portions of leiomyoma and matched myometrium were collected from premenopausal women (N=27) who were scheduled to undergo hysterectomy for symptomatic uterine leiomyomas at the University of Florida affiliated Shands Hospital. Of these patients seven received GnRHa therapy for a period of three months prior to surgery. The untreated patients did not receive any medications during the 3 months prior to surgery and, based on endometrial histology and patient last menstrual cycle, they were from proliferative (N=8) and secretory (N=12) phases of the menstrual cycle. To maintain a standard, leiomyomas used in this study were 2 to 3 cm in diameter. Prior approval was obtained from the University of Florida Institutional Review Board for the experimental protocol of this study.

Isolation and Culture of Leiomyoma and Myometrial Smooth Muscle Cells. Leiomyoma and myometrial smooth muscle cells (LSMC and MSMC) were isolated and cultured as previously described (Chegini, N. et al. Mol Hum Reprod, 2002, 8:1071-1078). Prior to use in these experiments, the primary cell cultures were characterized using immunofluorescence microscopy and antibodies to [symbol] smooth muscle actin, desmin and vimentin (Chegini, N. et al. Mol Hum Reprod, 2002, 8:1071-1078). LSMC and MSMC were cultured in 6-well plates at an approximate density of 10⁶ cells/well in DMEM-supplemented media containing 10% FBS. After reaching visual confluence, the cells were washed in serum-free media and incubated for 24 hrs under serum-free, phenol red-free condition (Chegini, N. et al. Mol Hum Reprod, 2002, 8:1071-1078; Ding, L. et al. J Clin Endocrinol Metab, 2004, 89:5549-5557). These cells were used for the following experiments.

The Expression of CCNs. Fibulin-1C and S100A4 and Regulation by TGF-beta and GnRHa. To determine whether TGF-beta and GnRHa influence the expression of CCNs, fibulin-1C and S100A4, LSMC and MSMC cultured as above were treated with TGF-β1 (2.5 ng/ml) or GnRHa (0.1 μM) for 2, 6 and 12 hrs (Ding, L. et al. J Clin Endocrinol Metab, 2004, 89:5549-5557). Since TGF-beta and GnRHa action in LSMC and MSMC is mediated in part through activation of MAPK pathway (Ding, L. et al. J Clin Endocrinol Metab, 2004, 89:5549-5557), the present inventors further determined whether inhibition of MAPK activation alters TGF-beta and GnRHa effects on CCNs, fibulin-1C and S100A4 expression. LSMC and MSMC were cultured as above and following pretreatment with U0126 (20 μg/ml), a synthetic inhibitor of ERK1/2, for 2 hrs (Ding, L. et al. J Clin Endocrinol Metab, 2004, 89:5549-5557), the cells were treated with TGF-beta1 (2.5 ng/ml) or GnRHa (0.1 M) for 2 hrs.

Activation of Smad also serves a major signaling pathway for TGF-β mediated action in LSMC and MSMC (Shi, Y. and Massague, J. Cell, 2003, 113:685-700; Xu, J. et al. J Clin Endocrinol Metab, 2003, 88:1350-1361). To determine whether TGF-β action in regulating the expression of CCNs, fibulin-1C and S100A4 is mediated through Smad pathway, LSMC and MSMC were cultured as above and transfected with Smad3 SiRNA designed using Dharmacon Inc (Lafayette, Colo.) tool with the target sequence of 5′-UCCGCAUGAGCUUCGUCAAAdTdT-3′ as previously described (Kim, B. C. et al. J Biol. Chem., 2004, 279:28458-28465). LSMC and MSMC at 80% confluence were transfected with SiRNA using transfectamine 2000 reagent according to the manufacturer's instructions (Inveritogen, Carlsbad, Calif.), with 200 μmol of SiRNA and 10 μl of transfection reagent for 48 hrs. The cells were then treated with TGF-β1(2.5 ng/ml) for 2 hrs. Untreated or cells treated with scrambled Smad3 SiRNA were used a negative control. Total RNA was isolated from the treated and untreated controls cells and subjected to Realtime PCR.

Realtime PCR. Total RNA was isolated using Trizol Reagent (invitrogen) and the level of TGF-β1, TGF-β3, CCNs, fibulin-1C and S100A4 mRNA expression was determined by Realtime PCR as previously described using Taqman and ABI-Prism 7700 (Applied Biosystems) and Sequence Detection System 1.91 software (Ding, L. et al. J Clin Endocrinol Metab, 2004, 89:5549-5557). Results were analyzed using comparative method following normalization of expression values to the 18S rRNA expression as previously described (Ding, L. et al. J Clin Endocrinol Metab, 2004, 89:5549-5557).

Western Blot Analysis and Immunohistochemical Localization. For Western blotting, total protein was isolated from small portions of GnRHa-treated and untreated leiomyoma and myometrium as previously described (Xu, J. et al. J Clin Endocrinol Metab, 2003, 88:1350-1361; Ding, L. et al. J Clin Endocrinol Metab, 2004, 89:5549-5557). The homogenates' protein contents were determined, and an equal amount was subjected to SDS-PAGE and transferred to polyvinyldiene difluoride membrane. The blots were incubated with anti-CCN2, CCN3, CCN4, fibulin-1C, and S100A4 antibodies for 1 hr at room temperature. The membranes were exposed to corresponding HRP-conjugated IgG and immunostained proteins were visualized using enhanced chemiluminesence reagents (AMERSHAM-PHARMACIA Biotech, Piscataway, N.J.) as previously described (Xu, J. et al. J Clin Endocrinol Metab, 2003, 88:1350-1361; Ding, L. et al. J Clin Endocrinol Metab, 2004, 89:5549-5557).

For immunohistochemical localization, tissue sections were prepared from formalin-fixed and paraffin-embedded leiomyoma and myometrium and subjected to standard processing. The sections were then immunostained using antibodies to CCN2, CCN3, CCN4, fibulin-1C, and S100A4 at 5 μg of IgG/ml for 2-3 hrs at room temperature. Following further processing including incubation with biotinylated secondary antibodies and avidin-conjugated HRP (ABC ELITE kit, VECTOR Laboratories, Burlingame, Calif.), the chromogenic reaction was detected with 3,3′-diaminobenzidine tetrahydrochloride solution. Omission of primary antibodies, or incubation of tissue sections with non-immune mouse-rabbit and -goat IgGs instead of primary antibodies at the same concentration during immunostaining served as controls (Xu, J. et al. J Clin Endocrinol Metab, 2003, 88:1350-1361).

All the experiments were performed at least three times in duplicate using independent cell cultures. Where appropriate the results are expressed as mean ±SEM and statistically analyzed using unpaired Student t-test and ANOVA. A probability level of P<0.05 was considered significant.

### EXAMPLE 9

### Expression of CCNs, Fibulin-1C and S100A4 in Leiomyoma and Myometrium and the effect of GnRHa Therapy

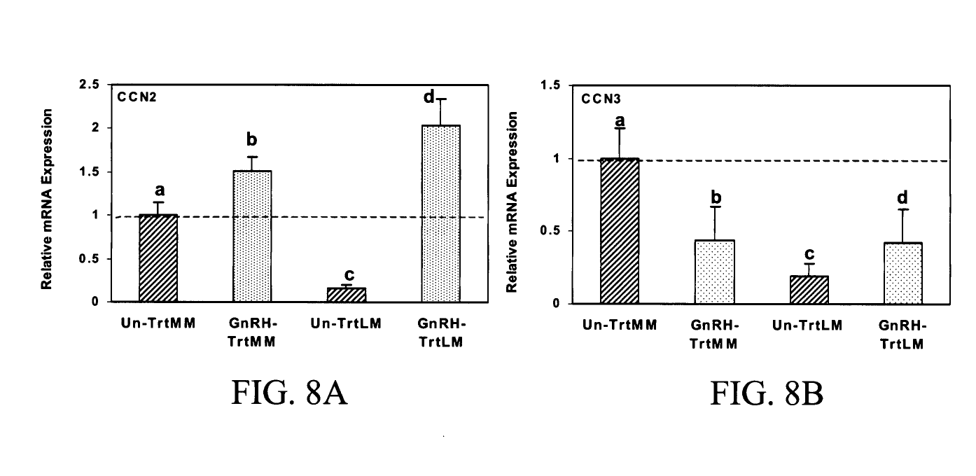

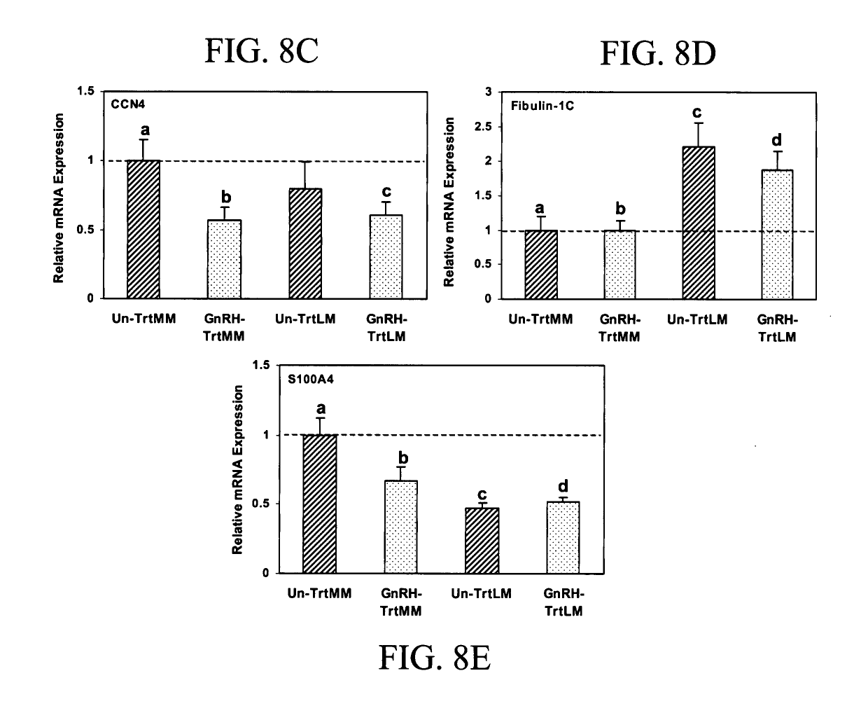

Using Realtime PCR the present inventors validated the expression of CCN2 (CTGF), CCN3 (NOV), CCN4 (WISP-1), fibulin-1C and S100A4 mRNA in leiomyoma and myometrium, demonstrating a significantly lower expression of CCN2, CCN3 and S100A4, with higher expression of fibulin 1C in leiomyoma as compared to myometrium (FIGS. 8A-8E; p<0.05). The level of CCN4 mRNA displayed a trend toward lower expression as compared to myometrium, but these levels did not reach statistical significance. GnRHa therapy resulted in significant reduction in CCN3, CCN4, and S100A4 expression in myometrium. Conversely, GnRHa therapy did significantly affect the expression of the above genes in leiomyoma with the exception of CCN2 (p<0.05; FIGS. 8A-8E).

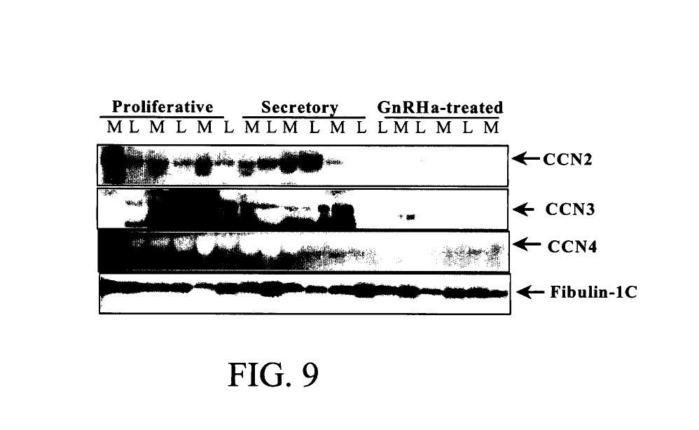

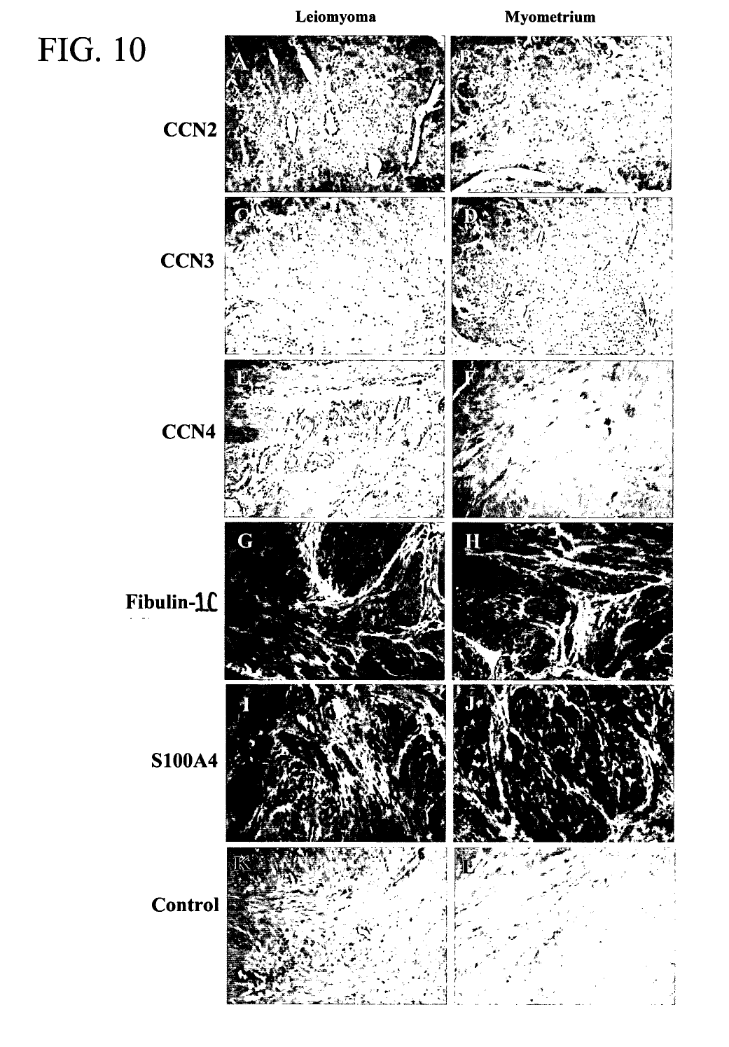

As illustrated in FIG. 9, leiomyoma and matched myometrium from proliferative and secretory phase of the menstrual cycle express variable levels of CCN2, CCN3, CCN4 and fibulin-1C proteins; however, quantitating their levels was not attempted in this study. The SA100A4 antibody was not useful for Western analysis and several attempts failed to detect any immunoreactive proteins in either tissue or cell extracts. Immunohistochemically, CCN2, CCN3, CCN4, fibulin-1C and S100A4 were localized in leiomyoma and myometrial smooth muscle cells, connective tissue fibroblasts and vasculature (FIGS. 10A-10L). The present inventors observed mostly cytoplasmic localization with a considerable heterogeneity in immunostaining intensity among various cell types. Incubation with normal rabbit (FIG. 10K) or goat (FIG. 10L) sera resulted in a considerable reduction in immunostaining intensity associated with these cells.

### EXAMPLE 10

### Correlation of CCNs with TGF-β Expression

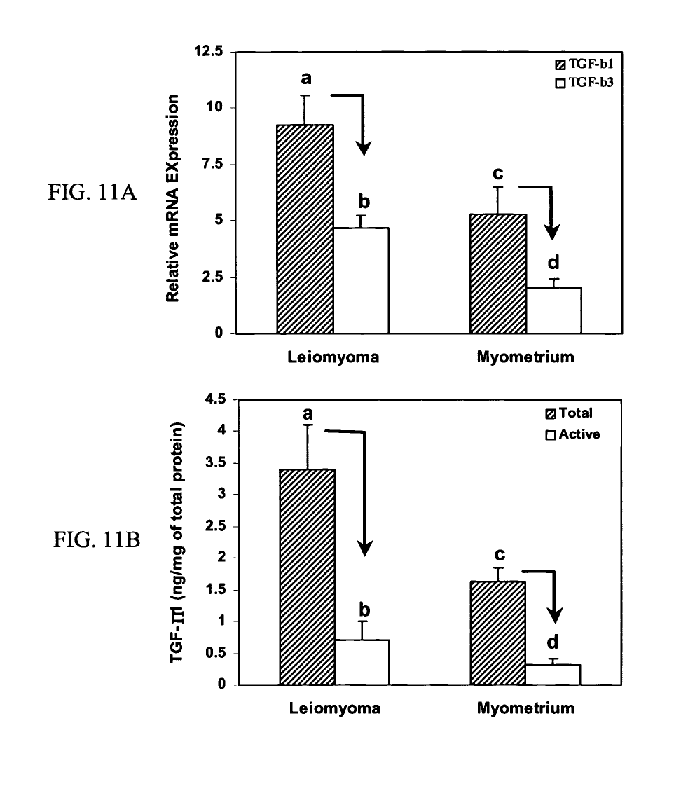

The present inventors have previously reported that leiomyoma and LSMC express elevated levels of TGF-β isoforms (TGF-β1, β2 and β3) as compared to myometrium and MSMC (Chegini, N. et al. J Clin Endocrinol Metab, 1999, 84:4138-4143; Chegini, N. et al. Mol Hum Reprod, 2002, 8:1071-1078; Chegini, N. et al. Mol Cell Endocrinol, 2003, 209:9-16; Xu, J. et al. J Clin Endocrinol Metab, 2003, 88:1350-1361; Ding, L. et al. J Clin Endocrinol Metab, 2004, 89:5549-5557; Tang, X. M. et al. Mol Hum Reprod, 1997, 3:233-240; Arici, A. and Sozen, I. Am J Obstet Gynecol, 2003, 188:76-83; Lee, B. S. and Nowak, R. A. J Clin Endocrinol Metab, 2001, 86:913-920; Arici, A. and Sozen, I. Fertil Steril, 2000, 73:1006-1011). Here, the present inventors confirmed these results showing that leiomyoma expressed a higher level of TGF-β1 compared to TGF-β3, with elevated levels as compared to myometrium (p<0.05; FIGS. 11A and 11B). In addition, leiomyoma express significantly higher levels of total and active TGF-β1 as compared to myometrium (p<0.05, FIGS. 11A and 11B). Since TGF-β action on tissue fibrosis is considered to be indirect and mediated through the induction of CCN2, the present inventors compared the expression of CCN2 with that of TGF-β1 and TGF-β3 in leiomyoma and myometrium. As shown in FIGS. 11A-11B and 8A-8E, not only the expression CCN2, but also the expression of CCN3 and CCN4 were inversely correlating with the expression of TGF-β1 and TGF-β3 in leiomyoma and myometrium.

### EXAMPLE 11

### The Expression of CCNs, Fibulin1C and S100A4 in LSMC and MSMC and regulation by TGF-β

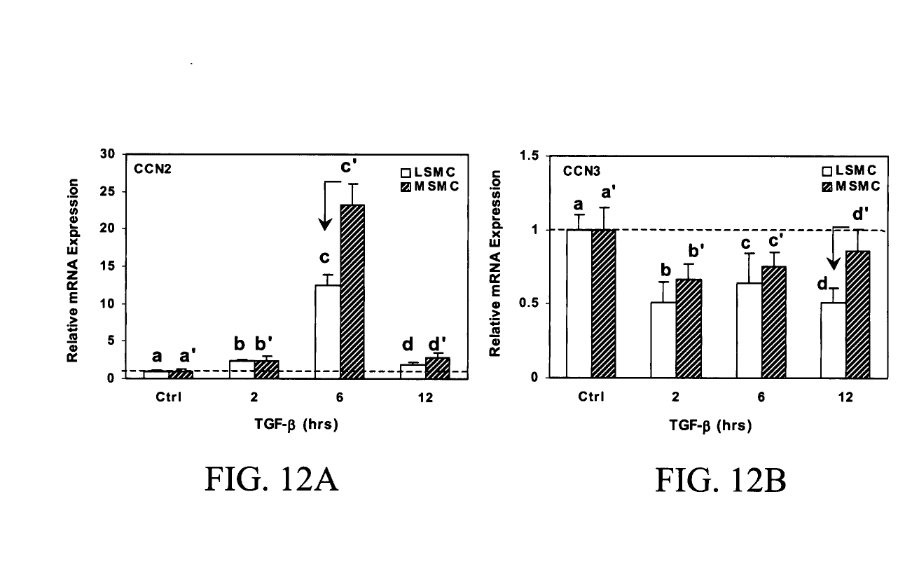

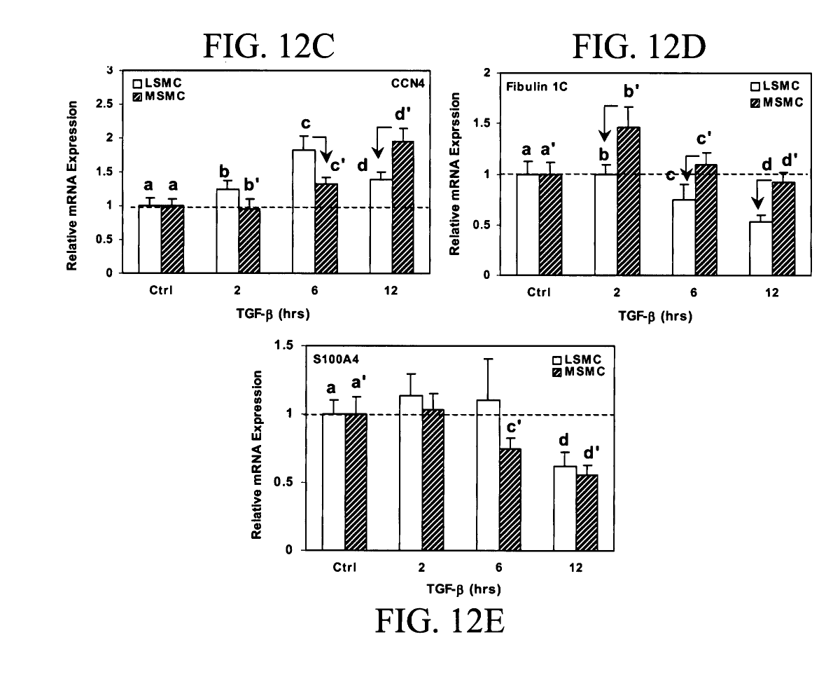

To evaluate whether TGF-β regulates the expression of CCN2 in leiomyoma and myometrium, the present inventors isolated LSMC and MSMC from these tissues and showed that these cells express CCNs, fibulin1-C and S100A4 and regulated by TGF-β1 (FIGS. 12A-12E). As shown in FIGS. 12A-12E, TGF-β in a cell- and time-dependent manner significantly increased the expression of CCN2 by 10 to 25 fold, and CCN4 by two fold, while inhibiting the expression of CCN3 (P<0.05). However, TGF-β1 had a limited effect on the expression of fibulin-1C and S100A4, moderately inhibiting their expression in LSMC and MSMC, while increasing fibulin-1C expression in MSMC (p<0.05; FIGS. 12A-12E).

### EXAMPLE 12

### The Effect of GnRHa on the Expression of CCNs, Fibulin1C and S100A4 in LSMC and MSMC

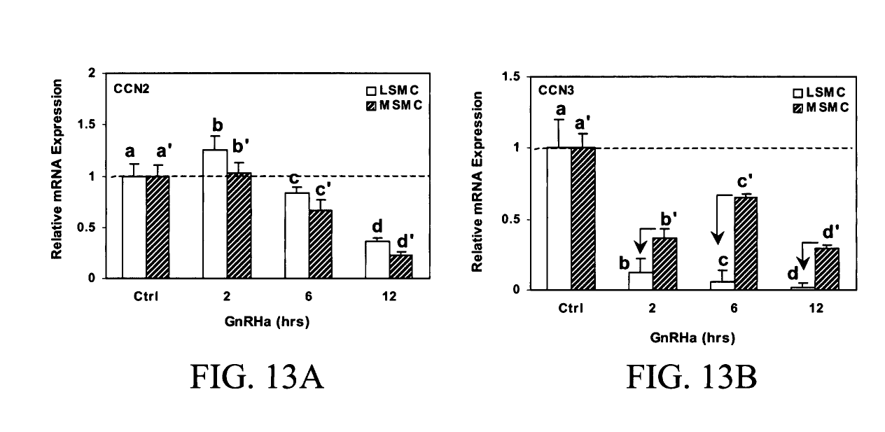

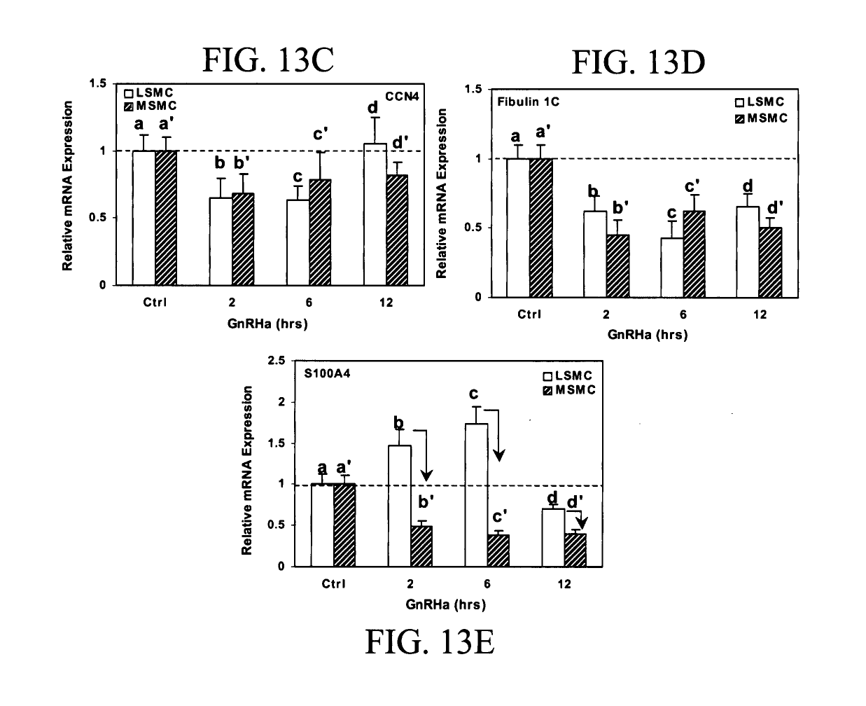

Conventional and microarray studies, including the inventors' own, have identified the expression profile of several genes targeted by GnRHa in leiomyoma and myometrial smooth muscle cells (Luo, X. et al. Endocrinology, 2005, 146:1074-1095; Luo, X. et al. Endocrinology, 2005, 146:1096-1118). The present inventors further assessed the direct action of GnRHa on CCNs, fibulin-1C and S100A4 expression following treatment of serum-starved LSMC and MSMC with GnRHa. As illustrated in FIGS. 13A-13E, GnRHa (0.1 μM) treatment for 2, 6 and 12 hrs in a time- and cell-dependent manner inhibited the expression of CCN2, CCN3, CCN4, fibluin-1C and S100A4 in LSMC and MSMC, with an increased expression of S100A4 in LSMC after 2 and 6 hrs of treatment as compared to MSMC (p<0.05).

### EXAMPLE 13

### Inhibition of MAPK and Smad3 Pathways on TGF-β and GnRHa-Mediated Action

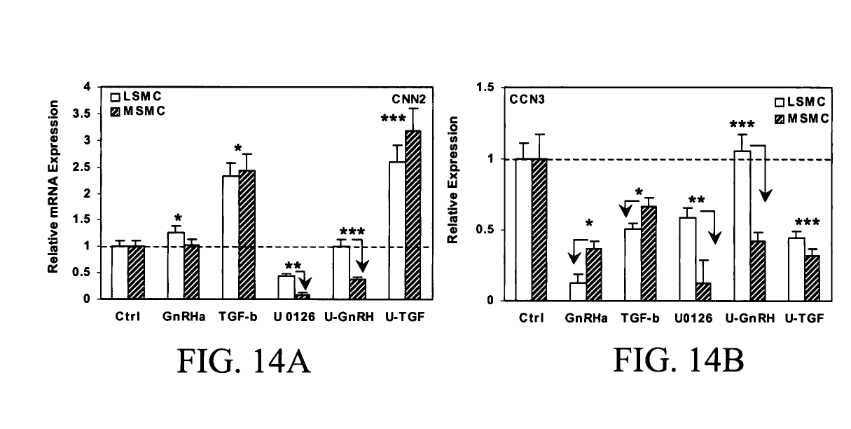

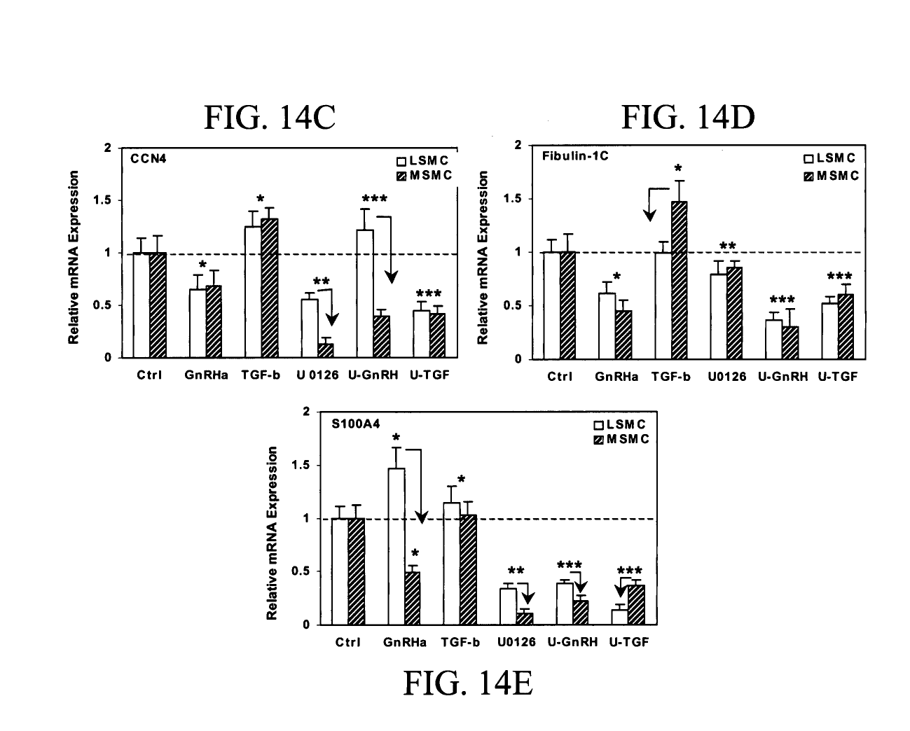

TGF-β and GnRH recruit and activate Smad and MAPK signaling pathways, respectively targeting the expression of many genes including fibronectin, collagen, MMPs, TIMPs, plasminogen activator inhibitor (PAI-1), c-fos and c jun in LSMC and MSMC (Xu, J. et al. J Clin Endocrinol Metab, 2003, 88:1350-1361; Ding, L. et al. J Clin Endocrinol Metab, 2004, 89:5549-5557; Arici, A. and Sozen, I. Fertil Steril, 2000, 73:1006-1011; Dou, Q. et al. Mol Hum Reprod, 1997, 3:1005-1014; Ma, C. and Chegini, N. Mol Hum Reprod, 1999, 5:950-954; Luo, X. et al. Endocrinology, 2005, 146:1074-1095; Luo, X. et al. Endocrinology, 2005, 146:1096-1118). To determine whether TGF-β and GnRHa regulate the expression of CCNs, fibulin-1C and S100A4 in LSMC and MSMC through these pathways, the cells were pretreated with MEK1/2 inhibitor (U0126). As shown in FIGS. 14A-14E pretreatment with U0126 altered the basal expression of CCN2, CCN3, CCN4, fibulin-1C and S100A4 in LSMC and MSMC, with a limited effect on TGF-β-mediated action on CCN2, but inhibited CCN3 expression in MSMC, and CCN4, fibulin-1C and S100A4 expression in both LSMC and MSMC (p<0.05). Pretreatment with U0126 also altered GnRHa-mediated action on CCN2, CCN3, CCN4, fibulin-1C and S100A4 expression in LSMC and MSMC in cell specific manner (FIGS. 14A-14E).

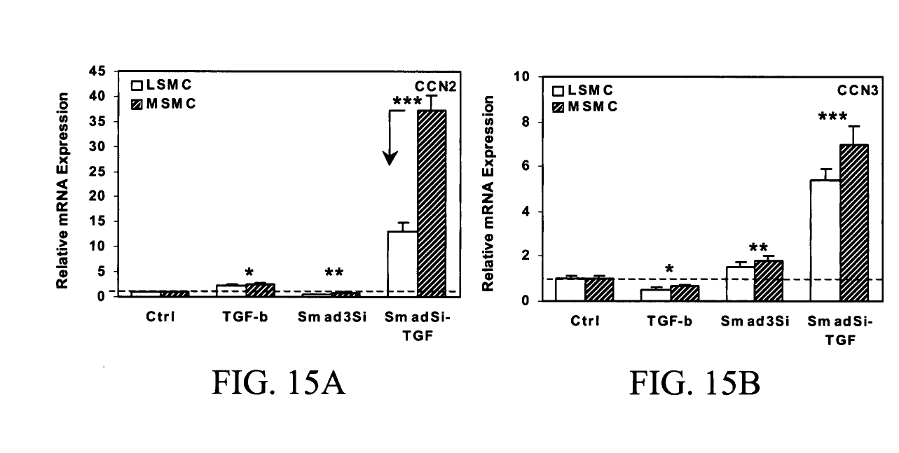

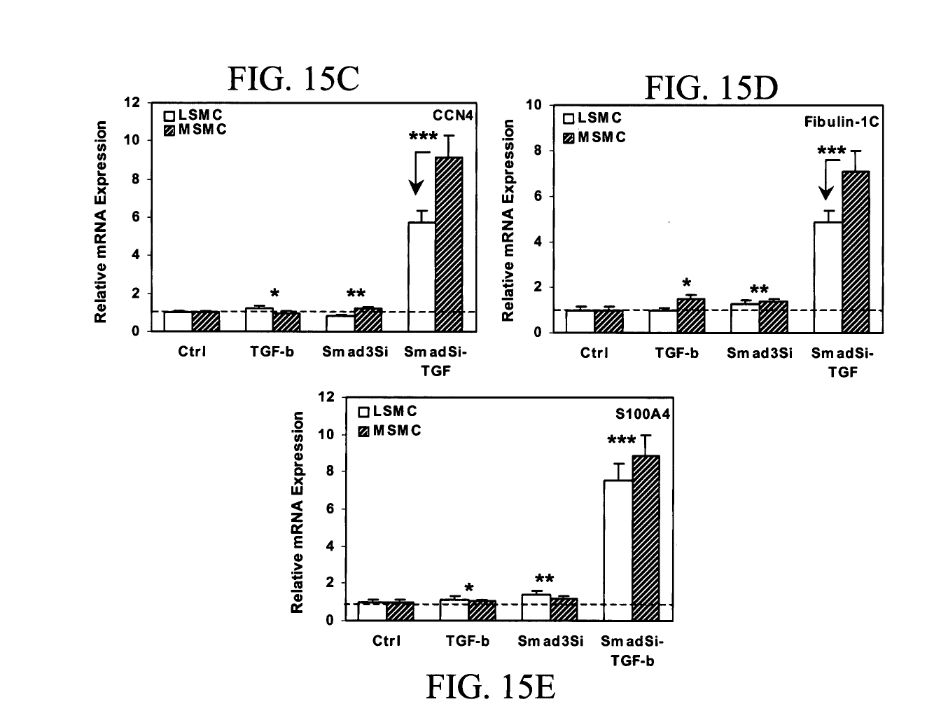

Transfection of LSMC and MSMC with Smad3 SiRNA, but not scrambled SiRNA significantly reduced the expression of Smad3 mRNA in LSMC and MSMC. Transfection with Smad3 SiRNA had a limited effect on the expression of CCN2, CCN4, fibulin-1C or S100A4 expression, although it increased the expression of CCN3 in both MSMC and LSMC (FIGS. 15A-15E). Treatment of Smad3 SiRNA-transfected cells with TGF-β1 for 2 hrs resulted in a significant enhancement of TGF-β1-mediated action on CCNs, fibulin-1C and S100A4 in both LSMC and MSMC (FIGS. 15A-15E).

In the present study, the present inventors demonstrated that leiomyoma and myometrium expresses several components of CCN family, as well as fibulin-1C and S100A4. The present inventors showed that leiomyoma expresses significantly lower levels of CCN2, CCN3 and S100A4, while expressing more fibulin-1C as compared to myometrium, with several cell types including LSMC and MSMC as their major source of local expression. The present inventors also provided the first evidence that GnRHa therapy alters the expression of CCN2 without affecting CCN3, CCN4 or fibulin-1C expression. The present inventors extended these observations and further demonstrated the expression of these genes in LSMC and MSMC and their regulation by TGF-β1 and GnRH through Smad and MAPK signaling pathway, respectively.

With respect to leiomyoma and myometrial expression of CCNs, fibulin-1C and S100A4 a limited correlation between levels of their expression and the phases of the menstrual cycle was found. Other studies have also reported a lack of menstrual cycle-dependent and lower expression of CCN1 (Cyr61), CCN2 and CCN5 in leiomyoma as compared to myometrium, except with higher expression of CCN5 in tissues from proliferative phase of the menstrual cycle and lowest expression detected during menstrual period (Sampath, D. et al. J Clin Endocrinol Metab, 2001, 86:1707-1715; Weston, G. et al. Mol Hum Reprod, 2003, 9:541-549; Mason, H. R. et al. Mol Hum Reprod, 2004, 10:181-187). Estrogen has been reported to regulate the expression of CCN5 in rat uterus (Mason, H. R. et al. Mol Hum Reprod, 2004, 10:181-187) and in human breast cancer cell lines (Sampath, D. et al. Endocrine, 2002, 18:147-159), as well as the expression of CCN1 in myometrial, but not in leiomyoma's explant cultures, whereas progesterone receptor agonist, RS020, alone or in combination with E2 had no effect (Sampath, D. et al. J Clin Endocrinol Metab, 2001, 86:1707-1715; Sampath, D. et al. Endocrine, 2002, 18:147-159; Sampath, D. et al. Endocrinology, 2001, 142:2540-2548). Considering that leiomyoma overexpresses estrogen and progesterone receptors as compared to myometrium, the expression profile of CCNs in these tissues suggests either a lack of, or an equal regulatory function for ovarian steroids. Since GnRHa therapy creates a hypoestrogenic condition, alteration in the expression of these genes in GnRHa-treated group imply a regulatory function for ovarian steroids. However, GnRHa therapy only affected the expression of CCN2, suggesting factors other than ovarian steroids may influence the expression of other members of CCN family in leiomyoma and myometrium. In this context, bFGF has been shown to increase the expression of CCN1 in myometrial, but not leiomyoma explants (Sampath, D. et al. J Clin Endocrinol Metab, 2001, 86:1707-1715). Unlike bFGF action on CCN1 expression, the present inventors found that TGF-β1 is equally effective in regulating the expression of CCN2, CCN3 and CCN4 in LSMC and MSMC, by increasing the expression of CCN2 and CCN4, while inhibiting CCN3.

TGF-β is a key profibrotic cytokine whose action on tissue fibrosis is considered to be indirect and mediated through the induction of CCN2 (Schnaper, H. W. et al. Am J Physiol Renal Physiol, 2003, 284:F243-F252; Ihn, H. Curr Opin Rheumatol, 2002, 14:681-685; Leask, A. and Abraham, D. J. Biochem Cell Biol, 2003, 81:355-363). Leiomyomas have several characteristic features typical of fibrotic disorder, including overexpression of TGF-β, TGF-β receptors and Smads as compared to normal myometrium (Dou, Q. et al. J Clin Endocrinol Metab, 1996, 81:3222-3230; Chegini, N. et al. J Clin Endocrinol Metab, 1999, 84:4138-4143; Chegini, N. et al. Mol Hum Reprod, 2002, 8:1071-1078; Chegini, N. et al. Mol Cell Endocrinol, 2003, 209:9-16; Xu, J. et al. J Clin Endocrinol Metab, 2003, 88:1350-1361; Ding, L. et al. J Clin Endocrinol Metab, 2004, 89:5549-5557; Tang, X. M. et al. Mol Hum Reprod, 1997, 3:233-240; Arici, A. and Sozen, I. Am J Obstet Gynecol, 2003, 188:76-83; Lee, B. S. and Nowak, R. A. J Clin Endocrinol Metab, 2001, 86:913-920; Arici, A. and Sozen, I. Fertil Steril, 2000, 73:1006-1011). Based on their expression profiles the present inventors have previously proposed that TGF-β1 and TGF-β3 play a more critical role in leiomyoma as compared to TGF-β2 (Dou, Q. et al. J Clin Endocrinol Metab, 1996, 81:3222-3230; Chegini, N. et al. J Clin Endocrinol Metab, 1999, 84:4138-4143; Chegini, N. et al. Mol Hum Reprod, 2002, 8:1071-1078; Chegini, N. et al. Mol Cell Endocrinol, 2003, 209:9-16; Xu, J. et al. J Clin Endocrinol Metab, 2003, 88:1350-1361; Ding, L. et al. J Clin Endocrinol Metab, 2004, 89:5549-5557). The present inventors provided further evidence in support of the inventors' previous observations and showed that leiomyoma express significantly higher levels of TGF-β1 and TGF-β3 as compared to matched myometrium, and with significantly higher TGF-β1 expression compared to TGF-β3. However, the expression profile of TGF-β1 and TGF-β3 in leiomyoma was inversely correlated, not only with CCN2 (CTGF), but also with CCN3 and CCN4 expression. Since most evidence supporting the involvement of CCN2 as a downstream signal in mediating TGF-β-induced tissue fibrosis comes from in vitro studies (Ihn, H. Curr Opin Rheumatol, 2002, 14:681-685; Leask, A. and Abraham, D. J. Biochem Cell Biol, 2003, 81:355-363), the present inventors isolated LSMC and MSMC from these tissues and showed, as expected, that TGF-β1 significantly increased the expression of CCN2 in these cells. The present inventors also found that TGF-β1 positively regulates the expression of CCN4, while suppressing CCN3 expression in these cells. To the present inventors' knowledge, this is the first study to demonstrate a differential regulatory function for TGF-β1 on CCN2, CCN3 and CCN4 expression in LSMC and MSMC, although TGF-β is known for regulating the expression of CCN2 in several cell types, with a few documented examples of regulation of CCN3 (Schnaper, H. W. et al. Am J Physiol Renal Physiol, 2003, 284:F243-F252; Ihn, H. Curr Opin Rheumatol, 2002, 14:681-685; Leask, A. and Abraham, D. J. Biochem Cell Biol, 2003, 81:355-363; Perbal, B. Lancet, 2004, 363:62-64; Brigstock, D. R. J Endocrinol, 2003, 178:169-175; Perbal, B. Mol Pathol, 2001, 54:57-79). To the present inventors' knowledge, this study is also the first to provide evidence for divergence between the expression of TGF-β isoforms and CCNs expression and regulation at tissue and cellular levels originating from these tissues. In hypertrophic scars gene expression profiling also indicated a lower expression of CCN2 accompanied by elevated expression of TGF-β1 as compared to normal skin (Tsou, R. et al. J Burn Care Rehabil, 2000, 21:541-550). The results of these studies indicate that a direct correlation between TGF-β and CCN2 expression may not serve as a common feature of all fibrotic disorders as previously proposed (Ihn, H. Curr Opin Rheumatol, 2002, 14:681-685; Leask, A. and Abraham, D. J. Biochem Cell Biol, 2003, 81:355-363).

TGF-β regulates its own expression in LSMC and MSMC and acting through downstream signaling from Smad and MAPK pathways regulates the expression of many other genes in different functional categories including cell cycle, transcription factors, cell and tissue structure, signal transduction and apoptosis (Dou, Q. et al. J Clin Endocrinol Metab, 1996, 81:3222-3230; Chegini, N. et al. J Clin Endocrinol Metab, 1999, 84:4138-4143; Xu, J. et al. J Clin Endocrinol Metab, 2003, 88:1350-1361; Ding, L. et al. J Clin Endocrinol Metab, 2004, 89:5549-5557; Arici, A. and Sozen, I. Fertil Steril, 2000, 73:1006-1011; Dou, Q. et al. Mol Hum Reprod, 1997, 3:1005-1014; Ma, C. and Chegini, N. Mol Hum Reprod, 1999, 5:950-954; Luo, X. et al. Endocrinology, 2005, 146:1074-1095; Luo, X. et al. Endocrinology, 2005, 146:1096-1118). Here, the present inventors demonstrated that pretreatment of LSMC and MSMC with U0126, a synthetic inhibitor of MEK1/2 inhibits the basal expression of CCNs expression and reverses TGF-β1 action. However, treatment of Smad3 SiRNA-transfected LSMC and MSMC with TGF-β1 resulted in a significant increase in CCNs expression. Although the results provide further evidence that components of both MAPK and Smad pathways are involved in mediating TGF-β action on the expression of CCNs (Ihn, H. Curr Opin Rheumatol, 2002, 14:681-685; Leask, A. and Abraham, D. J. Biochem Cell Biol, 2003, 81:355-363; Perbal, B. Lancet, 2004, 363:62-64; Brigstock, D. R. J Endocrinol, 2003, 178:169-175; Perbal, B. Mol Pathol, 2001, 54:57-79), including in LSMC and MSMC, a sharp increase in the expression of these genes in Smad3 SiRNA-transfected cells following TGF-β treatment was unexpected. The present inventors propose that crosstalk with components of other signaling pathways activated by TGF-β receptors may have opposing effect on TGF-β-induced CCNs, fibulin-1C and S100A4 expression in LSMC and MSMC. A recent study has reported that inhibition of ERK and c-jun NH(2)-terminal kinase (JNK), but not of p38 MAPK and PI3K, blocked TGF-β1-induced CCN2 expression and Smad2/3 phosphorylation in airway smooth muscle cells (Xie, S. et al. Am J Physiol Lung Cell Mol Physiol, 2005, 288:L68-L76). However, the inhibitory action of TGF-β on CCN4 expression in NCI H295R, adrenocortical cell line has been reported to be mediated through c-Jun in a Smad-independent manner (Lafont, J. et al. J Biol Chem, 2002, 277:41220-41229). The present inventors have recently reported that TGF-β through MEK1/2 regulates the expression of c-Jun in LSMC and MSMC (Ding, L. et al. J Clin Endocrinol Metab, 2004, 89:5549-5557), further supporting the involvement of multiple signaling pathways in TGF-β regulation of CCNs expression in LSMC and MSMC. Further consideration for TGF-β enhancement of CCNs expression in Smad3 SiRNA-transfected LSMC and MSMC may relate to elevated expression of Smad3 in leiomyoma (Xu, J. et al. J Clin Endocrinol Metab, 2003, 88:1350-1361), which similar to the expression of TGF-β1/β3, it is inversely correlate with CCNs expression. Such a condition may explain why the inhibition of Smad3 expression resulted in an increase in CCNs expression in LSMC and MSMC. Interestingly, plasminogen activator inhibitor (PAI-1) mRNA expression, a well known gene targeted by TGF-β was significantly inhibited following treatment of Smad3 SiRNA-transfected LSMC and MSMC with TGF-β (unpublished observation). In addition to TGF-β, other cytokines such as IL-4 and IL-13 that are expressed in leiomyoma (Ding, L. et al J Soc Gyncol Invest, 2004, 11:319A) also reported to attenuate TGF-β1-induced CCN2 expression by inhibiting TGF-β-stimulated ERK1/2 and Smad2/3 activation, while TNF-α and IL-1β reduced TGF-β-induced CCN2 without affecting TGF-α-induced Smad2/3 (Xie, S. et al. Am J Physiol Lung Cell Mol Physiol, 2005, 288:L68-L76). A functional Smad binding site and TGF-β responsive enhancer (TGFβRE) in CCN2 promoter has been found to be necessary for basal promoter activity in normal fibroblasts, whereas Smad element is not required for high CCN2 promoter activity in scleroderma fibroblasts (Leask, A. and Abraham, D. J. Biochem Cell Biol, 2003, 81:355-363).

These results with Smad3 SiRNA transfected LSMC and MSMC contrast with reports indicating the involvement of Smad pathway activation in TGF-β-induced CCN2 expression in other cell types (Ihn, H. Curr Opin Rheumatol, 2002, 14:681-685; Leask, A. and Abraham, D. J. Biochem Cell Biol, 2003, 81:355-363; Perbal, B. Lancet, 2004, 363:62-64; Brigstock, D. R. J Endocrinol, 2003, 178:169-175; Perbal, B. Mol Pathol, 2001, 54:57-79; Chen, Y. et al. Kidney International, 2002, 62:1149-1159). Although transfection with Smad3 SiRNA resulted in a significant inhibition of Smad3 mRNA expression in LSMC and MSMC, Smad3 inhibition coincided with significant increase, not only in CCN2 expression, but also CCN3, CCN4, fibulin-1C and S100A4 expression following TGF-β treatment. The mechanism underlying TGF-β induction of these genes is not clear from this study; however, TGF-β-induced CCN2 expression in dermal fibroblasts has been reported to involve a functional Smad binding site in the CTGF promoter since deletion or mutation at this site abolished the ability of TGF-β to induce CTGF promoter activity (Leask, A. and Abraham, D. J. Biochem Cell Biol, 2003, 81:355-363; Chen, Y. et al. Kidney International, 2002, 62:1149-1159; Holmes, A. et al. J Biol Chem, 2001, 276:10594-10601). Mutation of Smad element also reduced constitutive CTGF promoter activity, suggesting that the promoter is necessary for both basal and TGF-β-induced CTGF transcription (Leask, A. and Abraham, D. J. Biochem Cell Biol, 2003, 81:355-363; Chen, Y. et al. Kidney International, 2002, 62:1149-1159). However, in normal and scleroderma dermal fibroblasts mutation of Smad element is reported to affect TGF-α-induced, but not basal CTGF promoter activity (Chen, Y. et al. Kidney International, 2002, 62:1149-1159; Holmes, A. et al. J Biol Chem, 2001, 276:10594-10601). Smads alone is considered not activate transcription rather acting through recruitment of transcription factors to the promoter of their target genes and synergistic interactions with other signaling cascades they activate gene expression. Among the signaling pathway that interacts with Smads is MAPK (Shi, Y. and Massague, J. Cell, 2003, 113:685-700). The present inventors found that MEK1/2 inhibitor, U0126, in a cell specific manner reduced basal and TGF-β-induced CCN4, fibluin-1C and S100A4, but not TGF-β-induced CCN2 expression in LSMC and MSMC. Previous reports in other cells types indicated that preincubation with U0126, as well as tyrosine kinase, serine/threonine and protein kinase C inhibitors reduced the basal and TGF-β-induced CTGF promoter activity (Leask, A. and Abraham, D. J. Biochem Cell Biol, 2003, 81:355-363; Chen, Y. et al. Kidney International, 2002, 62:1149-1159; Holmes, A. et al. J Biol Chem, 2001, 276:10594-10601). Interestingly, MEK1 inhibitor (PD98059) did not affect TGF-β-induced CTGF, suggesting that the TGF-β induction of CTGF in mesangial cells requires MEK2, but not MEK1 (Chen, Y. et al. Kidney International, 2002, 62:1149-1159).

The present inventors also identified the expression of fibulin-1C and S100A4 in leiomyoma and myometrium, and in LSMC and MSMC and found that GnRHa therapy at tissue level and in vitro in a time- and cell-dependent manner altered their expression in LSMC and MSMC. TGF-β1 had a limited effect on the expression of fibulin-1C and S100A4 in these cells; it inhibited fibulin-1C and S100A4 in LSMC, while increasing fibulin-1C expression in MSMC. To the present inventors' knowledge, this is the first study to provide evidence for the expression of fibulin-1C and S100A4 at tissue level and their regulation in cell derived from these tissues in vitro. While this study was completed, a report showed that leiomyoma and myometrium expresses several members of S100 family including S100A4 using standard RT-PCR, and further demonstrated that S100A11 act as a suppressor of LSMC proliferation (Kanamori, T. et al. Mol Hum Reprod, 2004, 10:735-742). Although the biological significance of S100A4 in leiomyoma and myometrium is not clear from the present inventors' study, S100A4 expression has been associated with elevated levels of wild-type p53, and their physical interactions stimulate cells entry into the S phase of the cell cycle (Kanamori, T. et al. Mol Hum Reprod, 2004, 10:735-742; Grigorian, M. et al. J Biol Chem, 2001, 276:22699-22708). Furthermore, transfection of S100A4-negative cells with S100A4 constructs resulted in clonal death that was prevented by co-transfection with the anti-apoptotic gene bcl-2, which control calcium entry in different subcellular compartments (Chen, H. et al. Biochem Biophys Res Commun, 2001, 286:1212-1217; Brooke, J. S. et al. BMC Cell Biol, 2002, 3:2). Similar to CCN3 pro-angiogenic activities (Perbal, B. Lancet, 2004, 363:62-64; Brigstock, D. R. J Endocrinol, 2003, 178:169-175; Perbal, B. Mol Pathol, 2001, 54:57-79), S100A4 also promotes angiogenesis by acting directly as an angiogenic factor (Barraclough, R. Biochim Biophys Acta, 1998, 1448:190-199; Chen, H. et al. Biochem Biophys Res Commun, 2001, 286:1212-1217). Thus, the inhibitory action of GnRHa on CCN3 and S100A4 expression in leiomyoma may represent a mechanism by which GnRHa therapy regresses leiomyoma growth.

The interaction between fibulin-1C and CCN3 has been considered as an important step in CCN signaling involving ECM, cytoskeleton proteins and calcium (Perbal, B. et al. Proc Natl Acad Sci USA, 1999, 96:869-874; Argraves, W. S. et al. EMBO Rep, 2003, 4:1127-1131; Timpl, R. et al. Nat Rev Mol Cell Biol, 2003, 4:479-489; Tran, H. et al. J Biol Chem, 1995, 270:19458-19464). Similar to CCN3, fibulin-1C also contains a calcium-binding type II EGF-like domain enabling fibulin-1C to interact with extracellular domain of heparin-binding EGF (HB-EGF) (Perbal, B. et al. Proc Natl Acad Sci USA, 1999, 96:869-874; Argraves, W. S. et al. EMBO Rep, 2003, 4:1127-1131; Timpl, R. et al. Nat Rev Mol Cell Biol, 2003, 4:479-489; Tran, H. et al. J Biol Chem, 1995, 270:19458-19464; Tran, H. et al. J Biol Chem, 1997, 272:22600-22606). This EGF-like domain is also present in fibronectin and their interaction is considered to result in modification of calcium levels in surrounding cellular environment (Chegini, N. “Implication of growth factor and cytokine networks in leiomyomas” In: Cytokines in human reproduction, J Hill ed. Wiley & Sons, New York, pp. 133-162, 2000). Yeast two-hybrid screens have indicated that latent TGF-β binding protein (LTBP-3) also interacts with proHB-EGF through the EGF-like domains, and interaction among HB-EGF, LTBP-3 and fibulin-1C to serve as a novel function for HB-EGF action between cell and ECM (Grigorian, M. et al. J Biol Chem, 2001, 276:22699-22708). Since EGF, HB-EGF, TGF-BP and their receptors as well as fibronectin are expressed in leiomyoma and myometrium (Sherbet, G. V. and Lakshmi, M. S. Anticancer Res, 1998, 18:2415-2421), it is likely that their interactions may also influence communication between cellular and ECM compartment in leiomyoma. CCN3 has also been reported to interact with Notchl, a member of a family of highly conserved transmembrane receptors, involved in differentiation, proliferation and apoptosis, fundamental biological processes during embryonic development (Perbal, B. Lancet, 2004, 363:62-64; Brigstock, D. R. J Endocrinol, 2003, 178:169-175; Perbal, B. Mol Pathol, 2001, 54:57-79; Lin, C. G. et al. J Biol Chem, 2003, 278:24200-24208; Yu, C. et al. J Pathol, 2003, 201:609-615; Sakamoto, K. et al. J Biol Chem, 2002, 277:29399-29405; Soon, L. L. et al. J Biol Chem, 2003, 278:11465-11470; Margalit, O. et al. Br J Cancer, 2003, 89:314-319; Xie, D. et al. Cancer Res, 2001, 61:8917-8923; Saxena, N. et al. Mol Cell Biochem, 2001, 228:99-104). CCN3 is expressed in many different types of tumors and shows positive or negative effects on tumorigenesis and metastasis, however S100A4 is not tumorigenic rather it is elevated during metastasis suggesting a role in tumor progression (Brooke, J. S. et al. BMC Cell Biol, 2002, 3:2; Davies, M. et al. DNA Cell Biol, 1995, 14:825-832). Immunohistochemically, CCN2, CCN3 and CCN4 as well as fibulin 1C and S100A4 were detected in association with ECM and cytoplasmic compartments of various cell types in leiomyoma and myometrium with significant overlap in their distribution. CCN3 is detected in ECM, culture conditioned media, cytoplasm and nucleus, while S100A4 is essentially a cytoplasmic protein, although it is also secreted (Perbal, B. Lancet, 2004, 363:62-64; Brigstock, D. R. J Endocrinol, 2003, 178:169-175; Perbal, B. Mol Pathol, 2001, 54:57-79; Duarte, W. R. et al. Biochem Biophys Res Commun, 1999, 255:416-420). The results suggest that CCNs, fibulin-1C and S100A4 could interact intra- and extra-cellularly, influencing various cellar events during physiological and pathological conditions. For instance CCN3 through interaction with S100A4 might alter cytoskeletal organization, facilitate cell motility and cell proliferation, since CCN3 decreases adhesive capacity while increasing motility of Ewing's transfected cells (Margalit, O. et al. Br J Cancer, 2003, 89:314-319), and S100A4 affecting cytoskeleton assembly (Heizmann, C. W. and Cox, J. A. Biometals, 1998, 11:383-397; Barraclough, R. Biochim Biophys Acta, 1998, 1448:190-199). Inhibition of S100A4 has also been reported to decrease matrix metalloproteinases expression a mechanism that may account for S100A4 reduction in cellular migration (Merzak, A. et al. Neuropathol Appl Neurobiol, 1994, 20:614-619; Bjornland, K. et al. Cancer Res, 1999, 59:4702-4708).

In conclusion, the present inventors have provided further evidence that leiomyoma expresses elevated levels of TGF-β1 and TGF-β3 compared to myometrium whose expression inversely correlates with CCN2 as well as CCN3 and CCN4 expression in leiomyoma. The expression of CCNs as well as fibulin-1C and S100A4 is targeted by GnRHa therapy, and under in vitro condition TGF-β acting through MAPK/ERK and Smad pathways differential regulates their expression in LSMC and MSMC. Taken together, to the present inventors' knowledge, this study is the first to provide evidence for divergence of TGF-β and CCNs expression and regulation at cell and tissue levels from the same origin implying that CCN2 may not represent a common feature of fibrotic disorder associated with TGF-β overexpression.

### Materials and Methods

The following materials and methods describe those utilized in Examples 14-16. The materials for Realtime PCR, Western blotting and immunohistochemistry were purchased from Applied Biosystem (Foster City, Calif.), BioRad (Hercules, Calif.), and Vector Laboratories (Burlingame, Calif.), respectively as previously described (Ding, L. et al. J Clin Endocrinol Metab., 2004, 89:5549-5557; Xu, J. et al. J Clin Endocrinol Metab., 2003, 88:1350-1361). Polyclonal antibody generated in goat against recombinant FMOD was purchased from Santa Cruz Biotechnology (Santa Cruz, Calif.).

Portions of leiomyoma and matched myometrium were collected from premenopausal women (N=27) who were scheduled to undergo hysterectomy for symptomatic uterine leiomyomas at the University of Florida affiliated Shands Hospital. Of these patients, seven received GnRHa therapy for a period of three months prior to surgery. The untreated patients did not receive any medications during the previous 3 months prior to surgery and based on endometrial histology and the patient's last menstrual period they were identified as being from proliferative (N=8) or secretory (N=12) phases of the menstrual cycle. To maintain a standard, leiomyomas used in this study were 2 to 3 cm in diameter. Prior approval was obtained from the University of Florida Institutional Review Board for the experimental protocol of this study. Following collection, total RNA and protein was isolated from these tissues and subjected to Realtime PCR, Western blotting or processed for immunohistochemistry and cell culturing as previously described (Ding, L. et al. J Clin Endocrinol Metab., 2004, 89:5549-5557; Xu, J. et al. J Clin Endocrinol Metab., 2003, 88:1350-1361).

Realtime PCR. Briefly, total RNA was isolated from leiomyoma and matched myometrium using Trizol Reagent (INVITROGEN, Carlsbad, Calif.) and complimentary DNA was generated from 2 μg of total RNA using Taqman reverse transcription reagent. The newly synthesized cDNA was used for PCR performed in 96-well optical reaction plates with cDNA equivalent to 100 ng RNA in a volume of 50 μl reaction containing 1× Taqman Universal Master Mix, optimized concentrations of FAM-labeled probe and specific forward and reverse primer for FMOD selected from Assay on Demand (APPLIED BIOSYSTEMS). Controls included RNA subjected to RT-PCR without reverse transcriptase and PCR with water replacing cDNA. The results were analyzed using a comparative method and the values were normalized to the 18S rRNA expression and converted into fold change based on a doubling of PCR product in each PCR cycle, according to the manufacturer's guidelines as previously described (Ding, L. et al. J Clin Endocrinol Metab., 2004, 89:5549-5557; Luo, X. et al. Endocrinology, 2005, 146:1074-1096).

Western Blot Analysis and Immunohistochemistry. For Western blotting small pieces of tissues were lysed in a lysis buffer, centrifuged and the supernatants were collected and their total protein content was determined using a conventional method (Pierce, Rockford, Ill.) as previously described (Xu, J. et al. J Clin Endocrinol Metab., 2003, 88:1350-1361; Ding, L. et al. J Clin Endocrinol Metab., 2004, 89:5549-5557). Equal amounts of sample proteins were subjected to PAGE, transferred to polyvinylidiene difluoride (PVDF) membranes, and following further processing, the blots were incubated with FMOD antibody for 1 hr at room temperature. The blots were washed with washing buffer and exposed to corresponding HRP-conjugated IgG, and immunostained proteins were visualized using enhanced chemiluminescence reagents (Amersham-Pharmacia Biotech, Piscataway, N.J.).

For immunohistochemistry, tissue sections were prepared from formalin-fixed and paraffin embedded leiomyoma and myometrium and following standard processing immunostained using antibodies to FMOD at 5 μg of IgG/ml for 2-3 hrs at room temperature. Following further standard processing, chromogenic reaction was detected with 3,3′-diaminobenzidine tetrahydrochloride solution (Xu, J. et al. J Clin Endocrinol Metab., 2003, 88:1350-1361). Omission of primary antibody, or incubation of tissue sections with non-immune goat IgG instead of primary antibody at the same concentration served as controls.

The Expression and Regulation of Fibromodulin in LSMC and MSMC by TGF-beta and GnRHa. Leiomyoma and myometrial smooth muscle cells (LSMC and MSMC) were isolated, characterized and cultured as previously described (Chegini, N. et al. Mol Hum Reprod., 2002, 8:1071-1078). LSMC and MSMC were cultured in 6-well plates at an approximate density of 10⁶ cells/well in DMEM-supplemented media containing 10% FBS. After reaching visual confluence, the cells were washed in serum-free media and incubated for 24 hrs under serum-free, phenol red-free conditions (Chegini, N. et al. Mol Hum Reprod., 2002, 8:1071-1078).

To determine whether TGF-β and GnRHa influence the expression of FMOD, LSMC and MSMC cultured as above were treated with TGF-β1 (2.5 ng/ml) or GnRHa (0.1 μM) for 2, 6 and 12 hrs (Xu, J. et al. J Clin Endocrinol Metab., 2003, 88:1350-1361; Ding, L. et al. J Clin Endocrinol Metab., 2004, 89:5549-5557). Since TGF-β mediates its action in part through activation of the MAPK pathway (Ding, L. et al. J Clin Endocrinol Metab., 2004, 89:5549-5557), the present inventors determined whether inhibition of the MAPK pathway alter TGF-β mediated action in regulating the expression of FMOD. LSMC and MSMC were cultured as above and following pretreatment with U0126 (20 μM), a synthetic inhibitor of ERK1/2, for 2 hrs, the cells were treated with TGF-β1 or GnRHa for 2 hrs (Ding, L. et al. J Clin Endocrinol Metab., 2004, 89:5549-5557). Activation of Smad also serves as a major signaling pathway for TGF-β mediated action including in LSMC and MSMC (Xu, J. et al. J Clin Endocrinol Metab., 2003, 88:1350-1361). To determine whether TGF-β mediated action through the Smad pathway regulates the expression of FMOD, LSMC and MSMC were cultured as above and transfected with Smad3 SiRNA as previously described (Luo, X. et al. Endocrinology, 2005, 146:1097-1118). LSMC and MSMC at 80% confluence were transfected with 200 pmol of SiRNA using transfectamine 2000 reagent (10 μl) according to the manufacturer's instructions (INVITROGEN, Carlsbad, Calif.) for 48 hrs. The cells were then treated with TGF-β1 (2.5 ng/ml) for 2 hrs. Untreated or cells treated with scrambled Smad3 SiRNA were used as a negative control. Total RNA was isolated from the treated and untreated controls cells and subjected to Realtime PCR.

Where appropriate, the results are expressed as mean ±SEM and statistically analyzed using unpaired Student t-test and variance (ANOVA) using Tukey test. A probability level of P<0.05 was considered significant.

### EXAMPLE 14

### Expression of FMOD in Leiomyoma and Myometrium

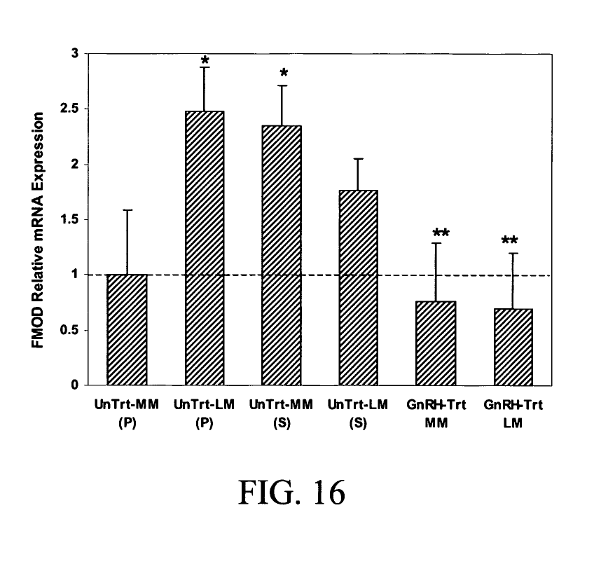

Using Realtime PCR, the present inventors demonstrated that leiomyoma and matched myometrium used for microarray analysis express FMOD mRNA with a considerable overlap between microarray analysis and Realtime PCR data. The present inventors evaluated the relative expression of FMOD and the influence of the menstrual cycle using total RNA isolated from leiomyoma and matched myometrium from proliferative (N=8) and secretory (N=12) phases of the menstrual cycle with Realtime PCR. The results indicated that FMOD is expressed at a significantly higher level in leiomyoma as compared to matched myometrium from the proliferative phase of the menstrual cycle (p<0.05; FIG. 16). There was a trend toward a lower expression of FMOD in leiomyoma compared to myometrium from the secretory phase, however these values did not reach statistical significance (FIG. 16). The relative level of FMOD expression was significantly elevated in myometrium from the secretory phase compared to proliferative phase (p<0.05) with a trend toward lower expression in leiomyoma (FIG. 16). The expression of FMOD was significantly reduced in both leiomyoma and myometrium in women who received GnRHa therapy (N=7), reaching the levels observed in myometrium from the proliferative phase (P=0.05; FIG. 16).

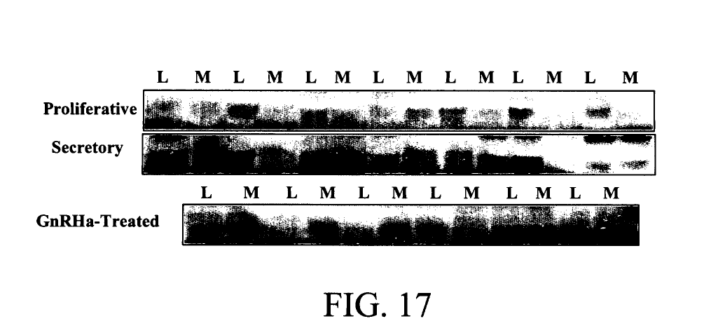

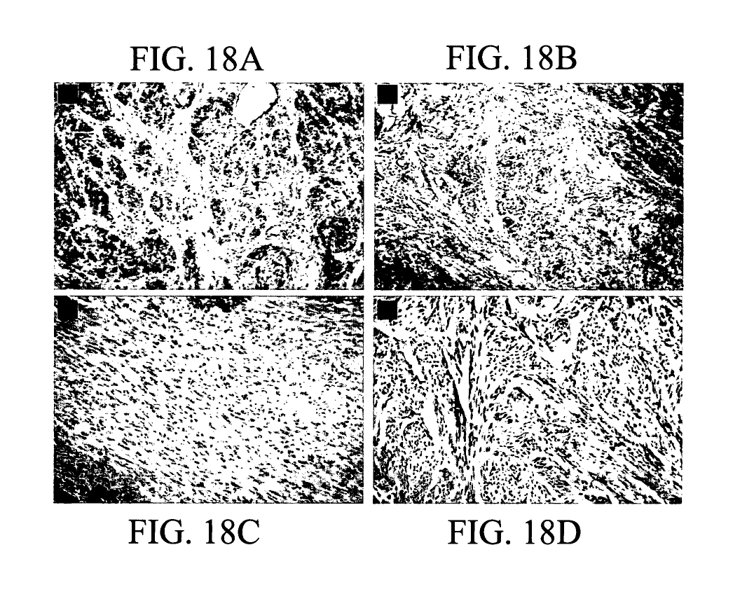

To further assess the expression of FMOD, total protein was isolated from these tissues and subjected to Western blot analysis. As shown in FIG. 17, leiomyoma (L) and matched myometrium (M) from proliferative and secretory phases of the menstrual cycle contain immunoreactive FMOD and with higher intensity in L compared with M in tissue from the proliferative phase, with an increase in intensity in tissues from the secretory phase. There was a reduction in FMOD immunoreactive intensity in L and M from the GnRHa treated group compared to tissues from the secretory phase (FIG. 17). Immunoreactive FMOD was also localized in leiomyoma and myometrial tissue sections with staining associated with myometrial and leiomyoma smooth muscle cells, as well as connective tissue fibroblasts and vasculature (FIGS. 18A-18D). Incubation of tissue sections with non-immune goat IgGs instead of primary antibody at the same concentration served as control and showed a substantial reduction in staining intensity associated with these cells.

### EXAMPLE 15

### Expression of FMOD in LSMC and MSMC and Regulation by TGF-β

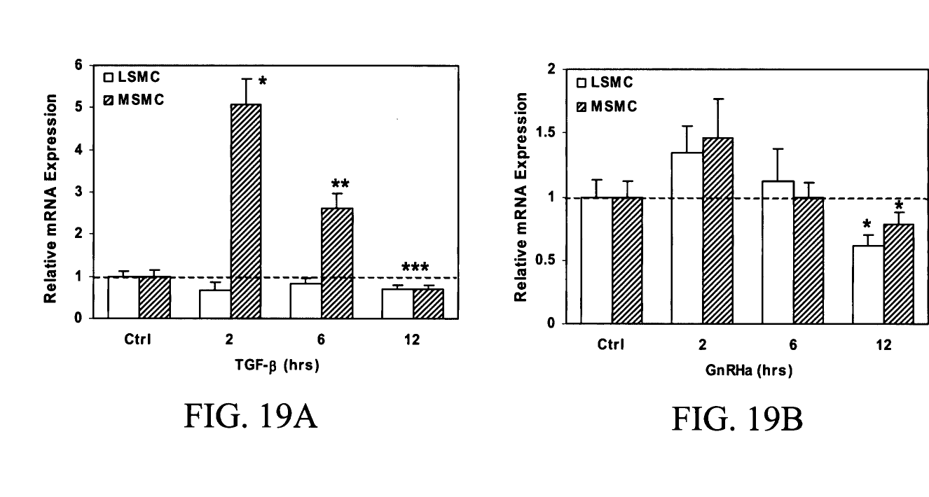

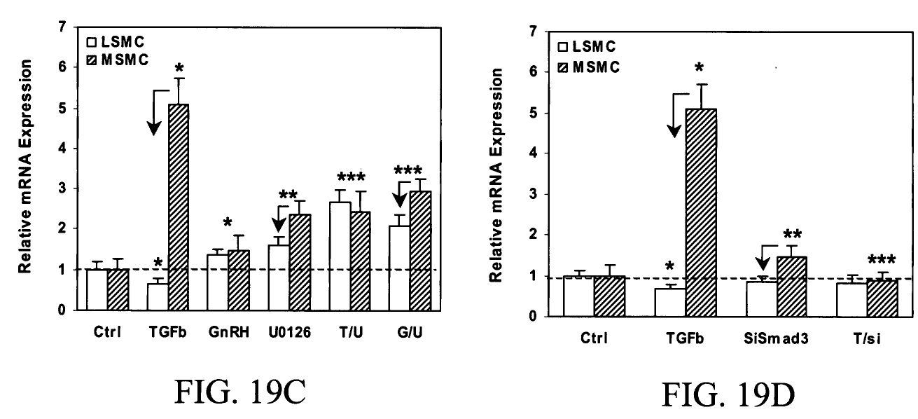

The present inventors have recently characterized the expression profile of LSMC and MSMC in response to TGF-β and GnRHa using gene microarray which indicated that the expression of several components of ECM including FMOD are the target of their regulatory action (Luo, X. et al. Endocrinology, 2005, 146:1097-1118). To further evaluate the influence of TGF-β on FMOD expression in leiomyoma and myometrium, the present inventors isolated LSMC and MSMC and following treatment with TGF-β1 (2.5 ng/ml) determined the expression of FMOD in these cells. As shown in FIGS. 19A-19D, treatment with TGF-β1 in a cell- and time-dependent manner significantly increased the expression of FMOD in MSMC with a gradual reduction in expression reaching control levels after 12 hrs (P<0.05). TGF-β had either no effect, or inhibited FMOD expression in LSMC after 12 hrs of treatment (FIGS. 19A-19D; P<0.05). Treatment of LSMC and MSMC with GnRHa (0.1 μM) for 2 and 6 hrs had no significant effect on FMOD expression; however, it inhibited FMOD after 12 hrs of treatment (FIGS. 19A-19D; P<0.05).

### EXAMPLE 16

### Inhibition of MAPK and Smad3 Pathways on TGF-β- and GnRHa-Mediated Actions

TGF-β recruits and activates several intracellular signaling pathways, specifically Smad and MAPK pathways. TGF-β through the activation of these pathways regulates the expression of many genes including fibronectin and collagen in LSMC and MSMC (Xu, J. et al. J Clin Endocrinol Metab., 2003, 88:1350-1361; Ding, L. et al. J Clin Endocrinol Metab., 2004, 89:5549-5557; Luo, X. et al. Endocrinology, 2005, 146:1097-1118). To determine whether TGF-β regulates the expression of FMOD through these pathways, LSMC and MSMC were pretreated with U0126 followed by treatment with TGF-β1 (2.5 ng/ml) for 2 hrs. As shown in FIGS. 19A-19D, pretreatment with U0126 increased the basal expression of FMOD in LSMC and MSMC and TGF-β-mediated action in LSMC, while inhibiting TGF-β-mediated action in MSMC (p<0.05). Pretreatment with U0126 also increased the expression of FMOD in MSMC and LSMC treated with GnRHa as compared to untreated control and U0126-treated cells, respectively (FIGS. 19A-19D; P<0.05).

Transfection of LSMC and MSMC with Smad3 SiRNA, but not scrambled SiRNA significantly inhibited the expression of Smad3 in both cell types, and resulted in a trend toward increased basal expression of FMOD in MSMC and LSMC (FIGS. 19A-19D). However Smad3 SiRNA transfection significantly reduced TGF-β-induced FMOD in MSMC reaching control levels, without affecting LSMC (FIGS. 19A-19D; P<0.05).

Using microarray gene expression profiling, the present inventors have identified fibromodulin (FMOD) among the differentially expressed genes in leiomyoma and myometrium and in LSMC and MSMC treated with TGF-β1 (Luo, X. et al. Endocrinology, 2005, 146:1074-1096; Luo, X. et al. Endocrinology, 2005, 146:1097-1118). In the present study, the present inventors validated the expression of FMOD using Realtime PCR showing a considerable overlap with microarray observations. The present inventors extended this work and demonstrated the menstrual cycle-dependent expression of FMOD in leiomyoma and myometrium. These results indicated that the expression of FMOD is significantly higher in leiomyoma compared to myometrium from the proliferative, but not the secretory phase of the menstrual cycle, suggesting a regulatory function for ovarian steroids on FMOD expression. The influence of the menstrual cycle on the expression of FMOD appears to be tissue specific, because of an increase in myometrial expression of FMOD from the secretory phase compared to the proliferative phase, with lower levels in leiomyoma. Since GnRHa therapy creates a hypoestrogenic condition, these results, as well as a significant reduction in the expression of FMOD in both leiomyoma and myometrium in women who received GnRHa therapy, further support the involvement of ovarian steroids in regulating FMOD expression in these tissues. The present inventors also demonstrated the expression of FMOD in LSMC and MSMC, and showed differential regulation by TGF-β1 and GnRHa through Smad and MAPK signaling pathways, respectively.

The biological significance of FMOD expression in leiomyoma and myometrium await detailed investigation, however, FMOD was found in association with several cell types in leiomyoma and myometrium and was differentially regulated by TGF-β in MSMC and to a certain extend in LSMC. Fibromodulin is a collagen-binding protein widely expressed in many connective tissues and appears to play an important role in ECM remodeling, specifically in tissues that undergo extensive tissue turnover such as cervix during ripening, fetal wound healing, atherosclerosis and bleomycin-induced lung fibrosis (Westergren-Thorsson, G. et al. Biochim Biophys Acta., 1998, 1406:203-213; Strom, A. et al. Histol Histopathol., 2004, 19:337-347; Soo, C. et al. Am J Pathol., 2000, 157:423-433; Venkatesan, N. et al. Am J Respir Crit. Care Med, 2000, 161:2066-2073). Fibromodulin is a member of the proteoglycan family including biglycan, decorin, lumican and chondroadherin small molecules with important roles in binding to other matrix molecules either to aid fibrillogenesis or act as bridging molecules between various tissue elements (Blochberger, T. C. et al. J Biol Chem, 1992, 267: 347-352; Noonan, D. M. and Hassell, J. R. Kidney Int, 1993, 43:53-60; Yanagishita, M. Acta Pathol Jpn, 1993, 43:283-293). It has been reported that for each collagen molecule there is at least one FMOD binding site, however these sites are limited in number and are highly specific (Hedbom, E. and Heinegard, D. J Biol Chem, 1993, 268:27307-27312). Evidence suggests that FMOD regulates the formation of the collagen fibrils network through its interaction with collagen types I, II and XII (Font, B. et al. Matrix Biol, 1996, 15:341-348), whose expressions have been documented in leiomyoma and myometrium (Stewart, E. A. et al. J Clin Endocrinol Metab., 1994, 79:900-906; Stewart, E. A. et al. J Soc Gynecol Investig., 1998, 5:44-47; Ding, L. et al. J Clin Endocrinol Metab., 2004, 89:5549-5557; Leppert, P. C. et al. Fertil Steril., 2004, 82(Suppl 3):1182-1187). Fibromodulin, like decorin, binds to type I and type II collagens and through interaction with TGF-β regulates the local biological activity and retention of TGF-β within the ECM (Fukushima, D. et al. J Biol Chem, 1993, 268:22710-22715; Hildebrand, A. et al. Biochem J, 1994, 302:527-534). Since leiomyoma and myometrium express biglycan and decorin (Luo, X. et al. Endocrinology, 2005, 146:1074-1096; Luo, X. et al. Endocrinology, 2005, 146:1097-1118; personal observations), alteration in the expression of FMOD could influence the organization of collagen and local availability of TGF-β, thus influencing the outcome of fibrosis in leiomyoma.

Leiomyomas have several characteristic features typical of fibrotic disorders, including overexpression of TGF-β, TGF-β receptors and Smads as compared to normal myometrium (Dou, Q. et al. Mol Hum Reprod., 1997, 3:1005-1014; Chegini, N. et al. Mol Hum Reprod., 2002, 8:1071-1078; Chegini, N. et al. J Soc Gynecol Investig., 2003, 10:161-171; Chegini, N. et al. Mol Cell Endocrinol., 2003, 209:9-16; Chegini, N. and Kornberg, L. J Soc Gynecol Investig., 2003, 10:21-26; Xu, J. et al. J Clin Endocrinol Metab., 2003, 88:1350-1361). Since leiomyoma also express a higher level of FMOD compared to myometrium the present inventors expected a positive regulatory function for TGF-β on the expression of FMOD in LSMC as compared to MSMC. However, under culture conditions of the present inventors' study, TGF-β resulted in a significant increase (5-10 fold) in FMOD expression in MSMC which declined to control levels, compared to a slight reduction in the expression in LSMC in a time-dependent manner. How TGF-β causes differential regulation of FMOD expression in MSMC and LSMC is unclear from this study and requires detailed investigation; however, it is clear that TGF-β mediated signaling though MAPK/ERK and Smad in MSMC are involved in differential regulation of TGF-β action in these cells. In other tissues such as the cervix during ripening, the expression of collagen type I and III, versican, biglycan, decorin and FMOD as well as TGF-β1 are reported to induce no significant change in small proteoglycans expression despite an almost 50% decrease in their concentration (Westergren-Thorsson, G. et al. Biochim Biophys Acta., 1998, 1406:203-213). However, in a rat model that transits from scarless fetal-type repair to adult-type repair, the expression of FMOD is reported to decrease as compared to TGF-β and TGF-β receptors, and when compared to adult wound healing (Soo, C. et al. Am J Pathol., 2000, 157:423-433). These results in a rat model of wound healing and scar tissue formation is comparable to the present inventors' observations in leiomyoma, suggesting that FMOD may act as a biologically relevant modulator of TGF-β activity during tissue fibrosis. TGF-β1 is reported to modulate the synthesis and accumulation of decorin, biglycan, and FMOD in cartilage explants cultured under conditions in which aggrecan synthesis remains relatively constant, with FMOD content most rapidly augmented in response to TGF-β1 (Burton-Wurster, N. et al. Osteoarthritis Cartilage, 2003, 11:167-176). In addition to TGF-β regulation of FMOD in dermal skin fibroblasts, CTGF has also been reported to increase the expression of FMOD, as well as the expression of type I and III collagens and basic fibroblast growth factor, without influencing the expression of HSP47, decorin, biglycan, and versican (Wang, J. F. et al. Wound Repair Regen., 2003, 11:220-229). In the gene expression profiling studies described herein, the present inventors found a significantly lower expression of CTGF in leiomyoma as compared to matched myometrium, however it was increased in TGF-β-treated LSMC and MSMC (Luo, X. et al. Endocrinology, 2005, 146:1097-1118). These results suggest that these cytokines could influence FMOD expression at the tissue level differently when compared to their action in vitro. Furthermore, the present inventors have reported that TGF-β self-regulates its own expression and the expression of CTGF and TGF-β through the activation of MAPK pathway regulates the expression of type I collagen and fibronectin in LSMC and MSMC (Ding et. al., 2004). In mouse uterus, analysis of decorin, biglycan, lumican and FMOD expression from day 1 to day 7 of pregnancy indicated that decorin was present together with lesser amounts of lumican in the stroma before the onset of decidualization, whereas biglycan and FMOD were almost absent (San Martin, S. et al. Reproduction, 2003, 125:585-595). Fibromodulin was weakly expressed in the non-decidualized stroma, but only after implantation (San Martin, S. et al. Reproduction, 2003, 125:585-595).

Fibromodulin expression has been found only in mitotic, but not in mitomycin C-induced postmitotic skin fibroblasts, or in endothelial cells and keratinocytes, and is considered to serve as a specific marker for mitotic activity which could indicate cell ageing (Petri, J. B. et al. Mol Cell Biol Res Commun., 1999, 1:59-65). Interestingly, matrix metallporteinases (MMPs) such as MMP-2, -8 and -9, and specifically MMP-13 are reported to effectively cleave FMOD in fresh articular cartilage, and the cleaved product was found to be identical to that observed in cleaved FMOD from cartilage explant cultures treated with IL-1 (Heathfield, T. F. et al. J Biol. Chem., 2004, 279:6286-6295). Since leiomyoma and myometrium express several MMPs including 2, 8, 9 and 13, and proinflammatory cytokines such as IL-1, they may target FMOD degradation in a manner similar to that demonstrated in other tissues (Dou, Q. et al. Mol Hum Reprod., 1997, 3:1005-1014, Lee, B. S. et al. J Clin Endocrinol Metab., 1998, 83:219-223, Tang, X. M. et al. Mol Hum Reprod., 1997, 3:233-240; Palmer, S. S. et al. J Soc Gynecol Investig., 1998, 5:203-209). Fibromodulin deficiency is reported to lead to a significant reduction in tendon stiffness in FMOD (−/−) mice, with irregular collagen fibrils and increased frequency of small diameter fibrils, suggesting that FMOD is required early in collagen fibrillogenesis (Chakravarti, S. Glycoconj J, 2002, 19:287-293). Thus, altered expression of FMOD would be expected to impact the organization of collagen in various fibrotic disorders such as leiomyoma.

In summary, these results document the first example of expression of FMOD in leiomyoma and myometrium and provide evidence for direct regulatory action of GnRHa and TGF-β on its expression in LSMC and MSMC. Since FMOD acts as key regulator of connective tissue remodeling its differential expression in leiomyoma and myometrium may influence leiomyoma fibrotic characteristics.

All patents, patent applications, provisional applications, and publications referred to or cited herein, whether supra or infra, are incorporated by reference in their entirety, including all figures, tables, and sequences, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

TABLE 1

| | | Change in |
|---|---|---|
| | | Expression |
| | | LYM vs |
| | | MYM |
| Gene Accession # | Gene Symbol | (P ≦ 0.02) |
| Transcription factors | | |
| AB020634 | NFAT5 | + |
| M97388 | DR1 | + |
| U26914 | RREB1 | + |
| AF040253 | SUPT5H | − |
| AB002386 | EZH1 | − |
| L38933 | HUMGT198A | − |
| AB022785 | ASH2L | − |
| AB014558 | CRY2 | − |
| Cell cycle regulators | | |
| X60188 | MAPK3 | − |
| U66469 | CGRRF1 | − |
| Cell adhesion receptors/proteins | | |
| AF106861 | ATRN | + |
| Z29083 | TPBG | + |
| AB002382 | CTNND1 | − |
| Extracellular transport/carrier | | |
| proteins | | |
| U09210 | SLC18A3 | + |
| Oncogenes and tumor suppressors | | |
| X57110 | CBL | + |
| M16038 | LYN | + |
| X60287 | MAX | + |
| U96078 | HYAL1 | − |
| Stress response proteins | | |
| W28616 | HSPCB | + |
| X83573 | ARSE | − |
| D87953 | NDRG1 | − |
| Membrane channels and | | |
| transporters | | |
| AF027153 | SLC5A3 | + |
| M55531 | SLC2A5 | + |
| X57303 | SLC7A1 | + |
| X91906 | CLCN5 | − |
| Extracellular matrix proteins | | |
| U05291 | FMOD | − |
| AB011792 | ECM2 | − |
| Trafficking/targeting proteins | | |
| D89618 | KPNA3 | + |
| AC004472 | VCP | + |
| AA890010 | SEC22L1 | + |
| L43964 | PSEN2 | + |
| X97074 | AP2S1 | + |
| AA192359 | TNPO3 | + |
| U32315 | STX3A | − |
| Metabolism | | |
| D50840 | UGCG | + |
| M21186 | CYBA | + |
| AC005329 | NDUFS7 | + |
| U44111 | HNMT | + |
| M84443 | GALK2 | + |
| X14608 | PCCA | + |
| AF014402 | PPAP2A | + |
| AF035555 | HADH2 | + |
| U84371 | AK2 | + |
| AA526497 | UQCRH | + |
| AI557064 | NDUFV2 | + |
| D55654 | MDH1 | + |
| AL049954 | AHCYL1 | − |
| AA420624 | MAOA | − |
| M93107 | BDH | − |
| Post-translational modification | | |
| U84404 | UBE3A | − |
| Translation | | |
| L36055 | EIF4EBP1 | + |
| Apoptosis associated proteins | | |
| Z70519 | TNFRSF6 | + |
| AJ006288 | BCL10 | + |
| U04806 | FLT3LG | − |
| RNA processing, turnover, and | | |
| transport | | |
| U40763 | PPIG | + |
| AB007510 | PRPF8 | − |
| X85237 | SF3A1 | − |
| U76421 | ADARB1 | − |
| Cell receptors (by ligand) | | |
| J03171 | IFNAR1 | + |
| M33210 | NDRG1 | − |
| AJ225028 | GABBR1 | − |
| D15050 | TCF8 | − |
| AF030339 | PLXNC1 | − |
| Intracellular | | |
| transducers/modulators | | |
| AB007881 | SMG1 | + |
| AB004904 | SOCS3 | + |
| D89094 | PDE5A | + |
| Z50053 | GUCY1A2 | + |
| X95632 | ABI2 | + |
| Y13493 | DYRK2 | + |
| D88532 | PIK3R3 | + |
| Y18206 | PPP1R3D | + |
| M96995 | GRB2 | + |
| AF015254 | AURKB | + |
| U02680 | PTK9 | + |
| AF052135 | STAMBP | + |
| U46461 | DVL1 | + |
| AB003698 | CDC7 | + |
| AI961669 | ARFGEF2 | + |
| X70218 | PPP4C | + |
| X99325 | STK25 | + |
| L36151 | PIK4CA | − |
| AL049970 | PRKRIR | − |
| AI671547 | RAB9A | − |
| AF103905 | RAPGEF3 | − |
| X95735 | ZYX | − |
| M33552 | LSP1 | − |
| X62048 | WEE1 | − |
| S76965 | PKIA | − |
| U25771 | ARF4L | − |
| AF035299 | DOK1 | + |
| Protein turnover | | |
| X87212 | CTSC | + |
| AL080090 | ANAPC10 | + |
| AJ132583 | NPEPPS | − |
| AF099149 | ARIH2 | − |
| Cell receptors (by activities) | | |
| AF084645 | NR1I2 | + |
| AB020639 | ESRRG | + |
| Cytoskeleton/motility | | |
| proteins | | |
| AB008515 | NOL7 | + |
| AI056696 | CETN3 | − |
| Functionally unclassified | | |
| AF035444 | PHLDA2 | + |
| U79299 | OLFM1 | + |
| U22963 | MR1 | + |
| U15552 | HSU15552 | + |
| AB015332 | AKAP8L | + |
| AF068195 | UBADC1 | + |
| AB011542 | EGFL5 | + |
| Z78368 | C1orf8 | − |
| AF053356 | LRCH4 | − |
| AF009426 | C18orf1 | − |
| not classified | | |
| AB011096 | SARM1 | + |
| AJ236885 | ZNF148 | + |
| N42007 | NUP50 | + |
| Z48570 | DDX24 | + |
| M19650 | CNP | + |
| AB002348 | KIAA0350 | + |
| AB014564 | KIAA0664 | − |
| M29551 | PPP3CB | − |
| AB020699 | KIAA0892 | − |
| AB002370 | KIAA0372 | − |
| AB023181 | DLGAP4 | − |
| AB011106 | ATRNL1 | − |
| D88152 | SLC33A1 | − |
| AF082657 | ERAL1 | + |
| AB023163 | HIP14 | − |
| AF040964 | C4orf15 | + |
| U33838 | RELA | + |
| M22919 | MYL6 | − |
| U93869 | POLR3F | + |
| X59417 | PSMA6 | + |
| AJ224326 | RPE | + |
| U60644 | PLD3 | + |
| AB018257 | ZNF294 | − |

TABLE 2

| | | Change in |
|---|---|---|
| | | Expression |
| | | LYM vs |
| | | MYM |
| Gene Accesssion# | Gene Symbol | (P ≦ 0.02) |
| Cell surface/Matrix Protein | | |
| D26579 | ADAM8 | + |
| Transcription Factor | | |
| U15655 | ERF | + |
| L39059 | TAF1C | + |
| M96577 | E2F1 | + |
| AF025654 | RNGTT | − |
| U15642 | E2F5 | − |
| AB015132 | KLF7 | − |
| U63810 | CIAO1 | − |
| U52960 | SURB7 | − |
| U65093 | CITED2 | − |
| AJ001183 | SOX10 | − |
| Cell cycle | | |
| U03106 | CDK1A | + |
| L23959 | TFDP1 | − |
| M80629 | CDC2L5 | − |
| X77794 | CCNG1 | − |
| Cell adhesion | | |
| receptors/proteins | | |
| AF007194 | Mucin 3 | + |
| X15606 | ICAM2 | − |
| D14705 | CTNNA1 | − |
| S66213 | ITGA6 | − |
| Oncogenes and tumor | | |
| suppressors | | |
| U96078 | HYAL1 | − |
| Stress response proteins | | |
| AI972631 | ARS2 | − |
| Membrane channels and | | |
| transporters | | |
| X89066 | TRPC1 | − |
| AB021981 | SLC35A3 | − |
| D50312 | KCNJ8 | − |
| Extracellular matrix | | |
| proteins | | |
| U37283 | MFAP5 | − |
| Trafficking/targeting | | |
| proteins | | |
| AF002163 | AP3D1 | + |
| X96783 | SYT5 | − |
| Metabolism | | |
| AJ004832 | NTE | + |
| AF062529 | NUDT3 | + |
| D38537 | PPOX | + |
| AI345944 | NDUFB1 | − |
| AI766078 | COQ7 | − |
| D14710 | ATP5A1 | − |
| Post-translational | | |
| modification | | |
| U31525 | GYG | − |
| Apoptosis associated | | |
| proteins | | |
| Y09392 | TNFRSF25 | + |
| AF015451 | CFLAR | − |
| M16441 | LTA | − |
| RNA processing, turnover, | | |
| and transport | | |
| L35013 | SF3B4 | + |
| AJ007509 | HNRPUL1 | + |
| AF016369 | PRPF4 | − |
| M96954 | TIA1 | − |
| Chromatin proteins | | |
| AF045184 | SKIIP | − |
| Cell Surface receptors | | |
| X06614 | RARA | + |
| AF109134 | OGFR | + |
| D16827 | SSTR5 | − |
| X61615 | LIFR | − |
| M64347 | FGFR3 | − |
| M15169 | ADRB2 | − |
| U23850 | ITPR1 | − |
| Growth Factor/Cyt/Chemo/Polypept- | | |
| Horm | | |
| U79716 | RELN | + |
| M63582 | TRH | + |
| M13982 | IL4 | + |
| X52599 | NGFB | + |
| Intracellular iransducers/modulators | | |
| U39064 | MAPKK6 | + |
| X82260 | RANGAP1 | + |
| Z15108 | PRKCZ | + |
| R54564 | MINK | + |
| U09284 | LIMS1 | + |
| U12779 | MAPKAPK2 | + |
| U18420 | RAB5C | + |
| AL050268 | RAB1A | − |
| AB005047 | SH3BP5 | − |
| X52213 | LTK | − |
| | GNEF1 | − |
| D85758 | ERH | − |
| AF014398 | IMPA2 | − |
| AJ011736 | GRAP2 | − |
| U59913 | SMAD5 | − |
| X17576 | NCK1 | − |
| U48730 | STAT5B | − |
| U17743 | MAP2K4 | − |
| U43885 | GAB1 | − |
| Protein turnover | | |
| D49742 | HABP2 | + |
| U80034 | MIPEP | − |
| Cytoskeleton/motility proteins | | |
| W27148 | MAP1B | − |
| DNA synthesis, recombination, repair | | |
| X91992 | ALKBH | − |
| Y15572 | RAD51L3 | − |
| AF007871 | DYT1 | − |
| AF058696 | NBS1 | − |
| Functionally unclassified | | |
| AI924594 | TSPAN-2 | − |
| Z68747 | mitochondrial | − |
| | ribosomal | |
| | protein S31 | |
| AB018285 | zinc finger protein | − |
| Not classified | | |
| D42085 | NUP93 | − |
| D87437 | C1orf16 | + |
| X77548 | NCOA4 | − |
| D79990 | RASSF2 | − |
| U05861 | AKR1C1 | − |
| L49054 | MLF1 | − |
| AB007884 | ARHGEF9 | − |
| AF044896 | C1orf38 | − |
| AJ223352 | HIST1H2BK | − |
| AA043348 | HSPA4 | − |
| Z85986 | C6orf69 | − |
| W26677 | FLJ35827 | + |
| AB011133 | MAST3 | + |
| AB018274 | LARP | + |
| U92896 | EFNA2 | + |
| AF064801 | RNF139 | + |
| U47924 | GRCA | − |
| AB007896 | KIAA0436 | − |
| AJ002428 | VDAC1 | − |

TABLE 3

| | | Change in |
|---|---|---|
| | | Expression |
| Gene Accesssion # | Gene Symbol | (p ≦ 0.02) |
| Cell surface antigens | | |
| X84746 | ABO | + |
| AF004876 | YIF1 | + |
| Transcription/activators/repressors | | |
| X98253 | ZNF183 | − |
| D38251 | POLR2E | − |
| U22431 | HIF1A | − |
| AB002332 | CLOCK | + |
| U33838 | RELA | − |
| U15306 | NFX1 | − |
| AF040253 | SUPT5H | + |
| L19067 | RELA | + |
| M74099 | CUTL1 | + |
| U48436 | FMR2 | + |
| AA478904 | KLF7 | + |
| M69043 | NFKBIA | − |
| Cell cycle-regulating kinases | | |
| U17743 | MAP2K4 | − |
| D88357 | CDC2 | − |
| L04658 | CDK5 | − |
| X66357 | CDK3 | + |
| M74091 | CCNC | − |
| L23959 | TFDP1 | − |
| Cell adhesion receptors/proteins | | |
| X69819 | ICAM3 | − |
| Z29083 | TPBG | − |
| AF007194 | Mucin 3, Intestinal | + |
| Oncogenes and tumor suppressors | | |
| J03069 | MYCL2 | + |
| X72631 | NR1D1 | + |
| U09577 | HYAL2 | − |
| AI743606 | RAB8A | − |
| U04313 | SERPINB5 | + |
| AF013168 | TSC1 | + |
| Trafficking/targeting proteins | | |
| X99459 | AP3S2 | − |
| AW044624 | RER1 | − |
| U60644 | PLD3 | − |
| AA890010 | SEC22L1 | − |
| AC004472 | VCP | − |
| AF034546 | SNX3 | − |
| Z12830 | SSR1 | − |
| AF044671 | GABARAP | − |
| Metabolism | | |
| AC005329 | NDUFS7 | − |
| M22976 | CYB5 | + |
| AF047181 | NDUFB5 | − |
| D16294 | ACAA2 | − |
| AI345944 | NDUFB1 | − |
| D14710 | ATP5A1 | − |
| X06994 | CYC1 | − |
| AI540957 | QP-C | − |
| AI557064 | NDUFV2 | − |
| U19822 | ACACA | + |
| AF047469 | ASNA1 | − |
| Protein modification enzymes | | |
| D29643 | DDOST | − |
| AD000092 | CALR | − |
| AF035280 | EIF2B2 | − |
| L36055 | EIF4EBP1 | − |
| L34600 | MTIF2 | − |
| D28483 | RBMS2 | − |
| RNA processing/turnover/ | | |
| transport | | |
| U51334 | TAF15 | + |
| D59253 | NP25 | − |
| Z48501 | PABPC1 | − |
| L36529 | THOC1 | + |
| AF083190 | DNAJC8 | + |
| D28423 | SFRS3 | − |
| Growth factors/cytokines/ | | |
| chemokines | | |
| J00219 | IFNG | + |
| U32324 | IL11RA | + |
| Z70519 | TNFRSF6 | − |
| X04571 | EGF | + |
| X72308 | CCL7 | + |
| X78686 | CXCL5 | + |
| J04513 | FGF2 | − |
| S74221 | IK | − |
| U43368 | VEGFB | + |
| AL021155 | NPPA | + |
| Intracellular | | |
| transducers/modulators | | |
| X75958 | NTRK2 | + |
| S76475 | NTRK3 | + |
| U43885 | GAB1 | − |
| X84709 | FADD | − |
| M96995 | GRB2 | − |
| U46461 | DVL1 | − |
| AF051323 | SCAP2 | − |
| X66363 | PCTK1 | − |
| AB018330 | CAMKK2 | + |
| L13616 | PTK2 | − |
| U02680 | PTK9 | − |
| X72964 | CETN2 | − |
| Y17711 | CBARA1 | − |
| U51004 | HINT1 | − |
| U94747 | HAN11 | − |
| U78733 | SMAD2 | − |
| GTP/GDP/G-protein/GTPase | | |
| modulators | | |
| D13988 | GDI2 | − |
| U18420 | RAB5C | + |
| U34806 | GPR15 | + |
| U18550 | GPR3 | − |
| Amino- and | | |
| carboxypeptidases | | |
| L13977 | PRCP | − |
| Metalloproteinases | | |
| U80034 | MIPEP | − |
| Proteosomal proteins | | |
| D26600 | PSMB4 | − |
| AB009398 | PSMD13 | − |
| X59417 | PSMA6 | − |
| D26598 | PSMB3 | − |
| D38048 | PSMB7 | − |
| Cytoskeleton/motility proteins | | |
| AB007862 | PCNT2 | + |
| U48734 | ACTN4 | + |
| U01828 | MAP2 | + |
| U39226 | MYO7A | + |
| AI540958 | DNCL1 | + |
| AF020267 | MYO9B | + |
| U43959 | ADD2 | + |
| AL096717 | EML2 | + |
| AI961040 | TUBGCP2 | + |
| Extracellular matrix and | | |
| carrier proteins | | |
| M12625 | LCAT | + |
| AF093118 | FBLN5 | + |
| M20776 | COL6A1 | − |
| U80034 | MIPEP | − |
| AB006190 | AQP7 | + |
| AB021981 | SLC35A3 | − |
| U90313 | GSTO1 | − |
| X67301 | IGHM | − |
| M92303 | CACNB1 | + |
| X91906 | CLCN5 | + |
| AB023173 | ATP11B | + |
| M20471 | CLTA | − |
| U27467 | BCL2A1 | + |
| U30872 | CENPF | − |
| AI857458 | UCN | − |
| D87432 | SLC7A6 | + |
| N80906 | CST6 | + |
| D38535 | ITIH4 | + |
| M31767 | MGMT | + |
| AB007884 | ARHGEF9 | − |
| AC004472 | KIAA1539 | − |
| Functionally unclassified | | |
| W28869 | TEGT | − |
| Z68747 | MRPS31 | − |
| L07758 | PWP1 | − |
| AJ007014 | NCBP2 | − |
| U72508 | B7 | − |
| AA524058 | C6orf74 | − |
| D86062 | C21orf33 | − |
| D87343 | DSCR3 | + |
| AF042384 | BC-2 | − |
| AF068195 | UBADC1 | − |
| AL021937 | RFPL3S | + |
| U80744 | TNRC5 | − |
| AF035444 | PHLDA2 | − |
| not classified | | |
| AL031177 | APG4A | + |
| AB007884 | ARHGEF9 | − |
| AC004472 | KIAA1539 | + |
| AF040964 | C4orf15 | − |
| D87742 | FLJ39207 | + |
| AB006628 | FCHO1 | + |
| AB014592 | KIAA0692 | + |
| AB023214 | ZBTB1 | + |
| AB028964 | FOXJ3 | + |
| U54999 | GPSM2 | + |
| L49054 | MLF1 | − |
| AA926959 | CKS1B | + |
| NM_00635 | Ras-Like | − |
| | Protein Tc4 | |
| AB002292 | ARHGEF10 | + |
| M24899 | THRA | + |
| U92896 | EFNA2 | + |
| AJ222967 | CTNS | + |
| AL031983 | OR2H3 | + |
| U05681 | BCL3 | + |
| AF014398 | IMPA2 | − |
| X67325 | IFI27 | − |
| U90907 | PIK3R3 | − |
| AF030107 | RGS13 | + |
| AL049634 | PTPNS1L2 | + |
| AF091071 | RER1 | + |
| AC005525 | IGSF4C | + |
| U49278 | UBE2V1 | − |
| U39318 | UBE2D3 | + |
| AF075599 | UBE2M | − |
| AJ002428 | VDAC1 | − |
| U84388 | CRADD | − |
| X63657 | FVT1 | + |

TABLE 4

| | | Change in |
|---|---|---|
| | | Expression |
| Gene Accesssion # | Gene Symbol | (p ≦ 0.02) |
| Cell surface/Matrix protein | | |
| AF106861 | ATRN | − |
| AJ001683 | KLRC4 | − |
| D26579 | ADAM8 | − |
| M33308 | VCL | + |
| U12255 | FCGRT | + |
| Transcription Factors | | |
| AJ001183 | SOX10 | − |
| AB004066 | BHLHB2 | − |
| AF012108 | NCOA3 | − |
| AF025654 | RNGTT | − |
| AF035262 | SMARCE1 | − |
| D42123 | CRIP2 | − |
| D80003 | NCOA6 | − |
| L19067 | RELA | + |
| L19871 | ATF3 | + |
| L38933 | HUMGT198A | + |
| L39059 | TAF1C | + |
| L49380 | SF1 | + |
| M81601 | TCEA1 | + |
| U37251 | ZNF177 | + |
| U63810 | CIAO1 | + |
| U68727 | PKNOX1 | + |
| X99720 | PRCC | + |
| Metabolism | | |
| AF104421 | UROD | − |
| AL049954 | AHCYL1 | − |
| D16294 | ACAA2 | − |
| D16481 | HADHB | − |
| D28137 | BST2 | − |
| D38537 | PPOX | − |
| D55639 | KYNU | − |
| U25849 | ACP1 | + |
| U91316 | BACH | + |
| X58965 | NME2 | + |
| X76228 | ATP6V1E1 | + |
| RNA processing transport | | |
| AA205857 | SNRPD3 | − |
| AB007510 | PRPF8 | − |
| AB017019 | HNRPDL | − |
| AL008726 | ZSWIM3 | − |
| U40763 | PPIG | + |
| Growth factor/chemokine | | |
| and receptors | | |
| X78686 | CXCL5 | − |
| X81882 | CUL5 | − |
| D13168 | EDNRB | − |
| D14582 | EPIM | − |
| D26070 | ITPR1 | − |
| J03278 | PDGFRB | − |
| J03634 | INHBA | − |
| M91211 | AGER | + |
| S67368 | GABRB2 | + |
| U23850 | ITPR1 | + |
| U78110 | NRTN | + |
| X06614 | RARA | + |
| X60592 | TNFRSF5 | + |
| X64116 | PVR | + |
| Non-receptor protein kinases | | |
| AI341656 | LIM | − |
| L13738 | ACK1 | + |
| L27071 | TXK | + |
| X54637 | TYK2 | + |
| Non-receptor phosphatases | | |
| AI739548 | | − |
| J03805 | PPP2CB | − |
| L36151 | PIK4CA | + |
| M29893 | RALA | + |
| M64929 | PPP2R2A | + |
| X68277 | DUSP1 | + |
| Nuclear receptors | | |
| AB020639 | ESRRG | − |
| AF084645 | NR1I2 | − |
| AF109134 | OGFR | − |
| X75918 | NR4A2 | + |
| Translation/post-trans | | |
| modification | | |
| D84273 | NARS | − |
| M34539 | FKBP1A | + |
| Death receptor | | |
| proteins/adaptors | | |
| AF006041 | DAXX | − |
| U04806 | FLT3LG | + |
| U50062 | RIPK1 | + |
| X98176 | CASP8 | + |
| Chaperones/heat shock | | |
| proteins | | |
| W28616 | HSPCB | − |
| L26336 | HSPA2 | + |
| X04106 | CAPNS1 | + |
| Cell signaling/EC | | |
| communication | | |
| AI658639 | ENSA | − |
| L19605 | ANXA11 | + |
| M32886 | SRI | + |
| U37283 | MFAP5 | + |
| U79716 | RELN | + |
| Adaptor/receptor-associated | | |
| proteins | | |
| AF015767 | BRE | − |
| U09284 | LIMS1 | + |
| GTP/GDP and G-protein | | |
| GTPase activity modulators | | |
| AB002349 | RALGPS1 | − |
| AI961929 | ARHGAP1 | − |
| M85169 | PSCD1 | + |
| U57629 | RPGR | + |
| Trafficking/targeting proteins | | |
| AF002163 | AP3D1 | − |
| D63476 | ARHGEF7 | − |
| U00957 | AKAP10 | + |
| X07315 | NUTF2 | + |
| DNA replications | | |
| J05249 | RPA2 | + |
| L20046 | ERCC5 | + |
| L26336 | HSPA2 | + |
| L26339 | RCD-8 | + |
| L78833 | VAT1 | + |
| M62302 | GDF1 | + |
| M84820 | RXRB | + |
| Other functional protein | | |
| M20681 | SLC2A3 | − |
| AA631972 | NK4 | − |
| AB026891 | SLC7A11 | − |
| AF047472 | BUB3 | − |
| AI972631 | ARS2 | − |
| AL008726 | | − |
| AL050254 | FBXO7 | − |
| D44466 | PSMD1 | − |
| D87953 | NDRG1 | − |
| L43964 | PSEN2 | + |
| M76558 | CACNA1D | + |
| M83664 | HLA-DPB1 | + |
| M95178 | ACTN1 | + |
| U40705 | TERF1 | + |
| U59913 | SMAD5 | + |
| U72263 | EXT2 | + |
| X01703 | TUBA3 | + |
| X14487 | KRT10 | + |
| X51602 | FLT1 | + |
| X58199 | ADD2 | + |
| X76538 | MPV17 | + |
| X78338 | ABCC1 | + |
| Z24727 | TPM1 | + |
| Functionally unclassified | | |
| AA923149 | WSB2 | − |
| AB002322 | SRRM2 | − |
| AB007879 | CP110 | − |
| AB007890 | LKAP | − |
| AB007915 | KIAA0446 | − |
| AB007931 | RBAF600 | − |
| AB011133 | MAST3 | − |
| AB011151 | BDG29 | − |
| AB014515 | N4BP1 | − |
| AB014564 | KIAA0664 | − |
| AB014599 | BICD2 | − |
| AB018344 | DDX46 | − |
| AB023186 | PEPP3 | − |
| AB028995 | PPM1E | − |
| AB028998 | TENC1 | − |
| AB029012 | EST1B | − |
| AF051941 | NME6 | − |
| AF058696 | NBS1 | |
| AL031228 | VPS52 | − |
| AL031282 | FLJ13052 | − |
| AL046940 | FLJ46603 | − |
| D29677 | HELZ | − |
| D50645 | SDF2 | − |
| D50920 | THRAP4 | − |
| D79990 | RASSF2 | − |
| D87119 | TRIB2 | − |
| Not classified | | |
| S59184 | RYK | + |
| U01062 | ITPR3 | + |
| U12597 | TRAF2 | + |
| U41737 | | + |
| U85611 | CIB1 | + |
| U89358 | L3MBTL | + |
| U93869 | POLR3F | + |
| W25974 | MTX1 | + |
| W27949 | HEBP2 | + |
| X16281 | ZNF44 | + |
| X52851 | PPIA | + |
| X65784 | SPG7 | + |
| X92814 | HRASLS3 | + |
| XM29054 | | + |
| Y09305 | DYRK4 | + |
| | GEF | + |
| NM_003242 | Protein Kinase | + |
| | Pitslre, Alpha, | |
| | Proto-Oncogene | |
| | N-Cym, | |
| | Single-Stranded | |
| | DNA-Binding | |
| | Protein Mssp- | |

TABLE 5

| Gene Symbol | Gene Name | Ref#9 | Ref#11 | Ref#12 | Ref#14 |
|---|---|---|---|---|---|
| BCL10 | B-cell CLL/lymphoma 10 | − | + | − | − |
| CDH2 | Cadherin 2A | + | − | − | − |
| F13A1 | Coagulation factor XIII | − | − | + | − |
| CRH | Corticotropin Releasing Hormone | − | + | − | − |
| ECM2 | Extracellular Matrix Protein 2 | + | − | − | − |
| HOXD4 | Homeo box D4 | − | − | − | + |
| ENO1 | c-myc binding protein | − | − | − | + |
| PIPPIN | Ortholog of rat Pippin | − | − | − | + |
| PPIB | Peptidylprolyl isomerase B | − | − | − | + |
| RY1 | Putative ucleic acid binding protein | − | − | − | + |
| TYMS | Thymidylate synthetase | + | + | − | + |

Ref#9: Tsibris, J. et al. Fertil Steril, 2002, 78: 114-121
Ref#11: Wang, H. et al. Fertil Steril, 2003, 80: 266-276
Ref#12: Weston, G. et al. Mol Hum Reprod, 2003, 9: 541-549
Ref#14: Quade, B. J. et al. Cancer, 2004, 40: 97-108

TABLE 6

| Gene | |
|---|---|
| Accesssion # | Gene Symbol |
| | Transcription |
| | activators/repressors |
| AJ000041 | HOXC11 |
| NM_001130 | AES |
| NM_006164 | NFE2L2 |
| | Cell cycle-regulating kinases |
| M84489 | MAPK1 |
| | Oncogene/tumor suppressors |
| NM_002315 | LMO1 |
| M24898 | NR1D1 |
| NM_002350 | LYN |
| | Membrane channels and |
| | transporters |
| NM_006358 | SLC25A17 |
| | Trafficking |
| NM_005829 | AP3S2 |
| | Metabolism |
| NM_001355 | DDT |
| NM_000819 | GART |
| NM_004317 | ASNA1 |
| | Translation/post-translational |
| NM_006156 | NEDD8 |
| NM_003758 | EIF3S1 |
| | Death receptor-associated |
| | proteins |
| AF015956 | DAXX |
| | RNA processing/turnovert |
| NM_002568 | PABPC1 |
| | Neuropeptides/growth factors |
| NM_003353 | UCN |
| NM_002006 | FGF2 |
| | Extracellular communication |
| NM_001405 | EFNA2 |
| NM_004279 | EEEF1E1 |
| | Intracellular |
| | transducers/effectors |
| NM_005079 | TPD52 |
| NM_006012 | CLPP |
| | Intracellular kinases (non- |
| | receptor) |
| AF-068864 | PAK3 |
| L13616 | PTK2 |
| NM_003177 | SYK |
| NM_002822 | PTK9 |
| NM_012290 | TLK1 |
| | GPs/GTPase activity |
| | modulators |
| M28212 | RAB6A |
| AF030107 | RGS13 |
| | Kinase activators/inhibitors |
| X82240 | TCL1A |
| NM_003629 | PIK3R3 |
| | Cytoskeleton/motility |
| | proteins |
| X58199 | ADD2 |
| | Functionally unclassified |
| NM_004487 | GOLGB1 |
| NM_004337 | C8orf1 |
| NM_006992 | B7 |
| | Not classified |
| NM_021964 | ZNF148 |
| NM_021999 | ITM2B |
| NM_014629 | ARHGEF10 |
| NM_030913 | SEMA6C |
| NM_012263 | TTLL1 |
| NM_020150 | SARA1 |
| | PPIA |
| | RPE |
| | MAFK |
| | LRIG2 |
| | DKFZP586F242 |
| | KIAA0290 |
| | Homeotic Protein Hox5.4 |

TABLE 7

| | | GnRHa |
|---|---|---|
| | | 2h vs |
| | | TGF- |
| | | βRII |
| | | antisense |
| Gene Accession# | Gene Symbol | p ≦ 0.001 |
| BC003576 | ACTN1 | − |
| Adenylyl Cyclase-AP2 | | + |
| M12271 | ADH1A | + |
| AB014605 | AIP1 | + |
| BC000171 | AMD1 | − |
| AK092006 | ANXA2 | + |
| BC001429 | ANXA5 | − |
| AK098588 | APEX1 | + |
| AF038954 | ATP6V1G1 | − |
| AB020680 | BAG5 | − |
| AF019413 | BF | + |
| AB004066 | BHLHB2 | − |
| BC009050 | BTG1 | + |
| AB030905 | CBX3 | + |
| BC008816 | CCBP2 | + |
| BC032518 | CCNG2 | + |
| AU130185 | CDH6 | + |
| AJ011497 | CLDN7 | − |
| AJ006267 | CLPX | + |
| BC005159 | COL6A1 | − |
| AK098615 | CRY1 | − |
| AL833597 | CSF2RA | − |
| AF013611 | CTSW | − |
| AK025446 | DKFZP564M182 | − |
| AJ005821 | DMXL1 | − |
| AF088046 | DNAJA2 | − |
| BC039596 | DNM2 | − |
| AF139463 | EGR2 | − |
| N66802 | EGR3 | − |
| AF001434 | EHD1 | − |
| AF208852 | EIF4A2 | + |
| BC000738 | EMD | − |
| AF103905 | EPAC | + |
| AF052181 | EPIM | + |
| BC003384 | FKBP2 | + |
| AF085357 | FLOT1 | + |
| AY358917 | FSTL3 | − |
| L13698 | GAS1 | + |
| AF169253 | GATA2 | − |
| AF144713 | GDI2 | + |
| AC000051 | GGT1 | + |
| NM_000855 | GUCY1A2 | + |
| X83412 | HAB1 | + |
| AF103884 | HB-1 | + |
| AF264785 | HES1 | − |
| BC022283 | HFL3 | + |
| | IGF I | + |
| D86989 | IGL2 | + |
| AF038953 | ITM2A | − |
| NM_005354 | JUND | − |
| AB014765 | JWA | + |
| AB002308 | KIAA0310 | − |
| AB014548 | KIAA0648 | + |
| AK129875 | LAPTM4A | + |
| AB017498 | LRP5 | + |
| AF027964 | MADH2 | + |
| AK026690 | MADH3 | + |
| AB025247 | MAFF | − |
| AB025186 | MAPRE3 | − |
| AB017335 | MAZ | − |
| AF061261 | MBNL2 | + |
| BC012396 | MGC40157 | + |
| AF125532 | MKNK2 | + |
| BC001122 | MSH2 | + |
| AF508978 | MTA1 | − |
| AF057354 | MTMR1 | + |
| NM_005593 | MYF5 | − |
| AB011179 | NCDN | − |
| AF047181 | NDUFB5 | + |
| AB014887 | ORM1 | + |
| BC009610 | PC4 | + |
| AK023529 | PCBP2 | − |
| AB029821 | PEMT | − |
| AF254253 | PHKG1 | + |
| AF220656 | PHLDA1 | − |
| AF025439 | PKM2 | − |
| A18757 | PLAUR | − |
| AB006746 | PLSCR1 | − |
| A24059 | PNLIP | + |
| AB005754 | POLS | − |
| AF042385 | PPIE | + |
| BC047502 | PPP1R3D | + |
| AK091875 | PPP2CB | − |
| AI800682 | PTPN21 | − |
| BC028038 | PTPRD | + |
| BC001390 | QP-C | + |
| BC003608 | RBPMS | − |
| AF019413 | RDBP | + |
| AF086557 | RPL10A | + |
| AB007147 | RPS2 | + |
| BC011645 | RRAD | − |
| D10570 | RUNX1 | − |
| AB028976 | SAMD4 | − |
| AF070614 | SCHIP1 | − |
| BC005927 | SERPINE1 | − |
| AJ000051 | SF1 | − |
| AK097315 | SF3B4 | − |
| BC004534 | SFPQ | − |
| AL110214 | SFRS6 | − |
| AB020410 | SHH | + |
| AB001328 | SLC15A1 | + |
| AF519179 | SMOX | − |
| AK096917 | SREBF2 | − |
| AF261072 | TCBAP0758 | + |
| BC003151 | TCFL1 | + |
| BC000125 | TGFB1 | − |
| AF050110 | TIEG | − |
| AF087143 | TOP2B | + |
| AC002481 | TUSC4 | + |
| AC002400 | UBPH | + |
| AF060538 | VAMP1 | + |
| AF134726 | VARS2 | − |
| BC000165 | VDAC2 | − |
| AF007132 | ABHD5 | − |
| AL831821 | ACADSB | + |
| AJ306929 | AFURS1 | − |
| AB031083 | AKR1C1 | + |
| AC002366 | AMELX | + |
| AB084454 | ANGPT1 | + |
| AF168956 | APLP2 | − |
| AF047432 | ARF6 | + |
| AK000379 | ASNS | − |
| AF022224 | BAG1 | + |
| BC019307 | BCL2L1 | + |
| AC006378 | BET1 | + |
| AB004066 | BHLHB2 | − |
| AF002697 | BNIP3 | − |
| AL021917 | BTN3A3 | + |
| AB059429 | BUCS1 | + |
| AJ420534 | C6orf145 | − |
| AF111344 | CASP10 | + |
| AK022697 | CBARA1 | − |
| BC009356 | CDC42EP1 | − |
| AF002713 | CENPB | − |
| AK128741 | CHD4 | + |
| AF136185 | COL17A1 | + |
| AB014764 | COPS7A | − |
| AF452623 | CRELD1 | − |
| AK098615 | CRY1 | − |
| AL833597 | CSF2RA | − |
| AB014595 | CUL4B | + |
| AB015051 | DAXX | − |
| AJ313463 | DF | + |
| BC015800 | DXYS155E | + |
| BC014410 | EFEMP1 | − |
| AF139463 | EGR2 | − |
| BC028412 | ELL2 | + |
| AK092872 | ERCC2 | + |
| AK000818 | FLJ20811 | + |
| AK074486 | FLJ90005 | − |
| AK130009 | FRZB | + |
| AJ251501 | GAD2 | + |
| AC004976 | GARS | − |
| AK094782 | GLUD1 | − |
| AF070597 | GNB1 | − |
| AK023082 | GORASP2 | + |
| AF077204 | GTPBP1 | + |
| BC035837 | HAS1 | + |
| AK097824 | HSPA2 | + |
| BC009696 | IFITM2 | + |
| AC005369 | IK | + |
| L25851 | ITGAE | + |
| AF003521 | JAG2 | − |
| BC002646 | JUN | − |
| AF081484 | K-ALPHA-1 | − |
| AF056022 | KATNA1 | + |
| AK025504 | KIAA0251 | − |
| AB002301 | KIAA0303 | − |
| AB014528 | KIAA0628 | + |
| AB014548 | KIAA0648 | + |
| AB040969 | KIAA1536 | − |
| AB040972 | KIAA1539 | − |
| AF061809 | KRT16 | + |
| BC009971 | KRTHA3B | + |
| AB014581 | L3MBTL | + |
| AF000177 | LSM1 | + |
| AB025186 | MAPRE3 | − |
| AB018266 | MATR3 | − |
| AC005943 | MBD3 | − |
| AY032603 | MCM3 | − |
| AF508978 | MTA1 | − |
| AK130664 | MTHFD2 | − |
| AB023192 | NISCH | + |
| AC004663 | NOTCH3 | − |
| AB005060 | NRG2 | + |
| AK025458 | NUCB1 | − |
| | NCOR 2 | − |
| AF109134 | OGFR | + |
| AJ238420 | PDGFA | − |
| AB005754 | POLS | − |
| AB051763 | POR | − |
| AA846273 | PRCC | + |
| AF044206 | PTGS2 | − |
| AY449732 | PTHR1 | + |
| BC002438 | RAB4A | + |
| AF080561 | RBM14 | − |
| BC003608 | RBPMS | − |
| AL031228 | RING1 | + |
| AB078417 | RIS1 | + |
| AK096243 | RPN2 | + |
| D10570 | RUNX1 | − |
| BC002829 | S100A2 | + |
| AB011096 | SARM1 | + |
| BC020740 | SGCD | − |
| AC004000 | SLC25A5 | − |
| AY142112 | SLC4A3 | + |
| AF053134 | SNCB | + |
| AB061546 | SRP14 | + |
| AK125542 | SRPX | + |
| AB015718 | STK10 | + |
| BC012085 | STK38 | + |
| AF064804 | SUPT3H | + |
| BC000125 | TGFB1 | − |
| AI290070 | THBS1 | + |
| AY117678 | TPT1 | + |
| AF062174 | TRIAD3 | − |
| BC014243 | TYK2 | − |
| AB003730 | UBC | + |
| AB014610 | USP52 | + |
| BC030810 | ZNF230 | − |
| AJ245587 | ZNF248 | + |
| BI547129 | ZW10 | − |
| AC006020 | AASS | + |
| AF245699 | AGTR1 | + |
| AC002366 | AMELX | + |
| D12775 | AMPD3 | + |
| AB084454 | ANGPT1 | + |
| AF019225 | APOL1 | + |
| BC014450 | B7 | + |
| AB004066 | BHLHB2 | − |
| AB062484 | CALD1 | + |
| AB023172 | CARD8 | + |
| BC002609 | CBX1 | − |
| AF213700 | CDKN1B | + |
| AF018081 | COL18A1 | + |
| BC000326 | COPB2 | + |
| AF062536 | CUL1 | − |
| NM_005491 | CXorf6 | − |
| AC004634 | DTR | − |
| AA053720 | EDIL3 | + |
| AF174496 | EEF1A1 | + |
| AF139463 | EGR2 | − |
| N66802 | EGR3 | − |
| AF000670 | ELF4 | − |
| AF083633 | EXTL1 | − |
| BC001786 | FKBP4 | − |
| AY358917 | FSTL3 | − |
| AB014560 | G3BP2 | − |
| AK022142 | GAB1 | + |
| AF169253 | GATA2 | − |
| AL031659 | GHRH | + |
| BC026329 | GJA1 | + |
| AF052693 | GJB5 | + |
| AF493902 | GNA13 | + |
| K03460 | H2-ALPHA | − |
| AF264785 | HES1 | − |
| AB017018 | HNRPDL | + |
| AF056979 | IFNGR1 | − |
| AC005369 | IK | + |
| AJ271736 | IL9R | + |
| AF007140 | ILF3 | + |
| AY351902 | IQGAP2 | + |
| AB007893 | KIAA0433 | + |
| AB014528 | KIAA0628 | + |
| AB028956 | KIAA1033 | − |
| AB014581 | L3MBTL | + |
| BC016618 | LCP2 | + |
| AF211969 | LENG4 | − |
| AF004230 | LILRB1 | + |
| BC017263 | LMAN2 | + |
| AF055581 | LNK | − |
| AK095843 | LOC169834 | + |
| AB025247 | MAFF | − |
| AC005943 | MBD3 | − |
| BC012396 | MGC40157 | + |
| AF508978 | MTA1 | − |
| AK130664 | MTHFD2 | − |
| NM_005593 | MYF5 | − |
| AB020673 | MYH11 | + |
| BC005318 | MYL1 | + |
| AB014887 | ORM1 | + |
| AK125499 | P5 | + |
| AJ238420 | PDGFA | − |
| AK055119 | PDK2 | − |
| AB051763 | POR | − |
| AF042385 | PPIE | + |
| AF345987 | PRKCG | + |
| M95929 | PRRX1 | − |
| AF119836 | RAB6A | + |
| AF019413 | RDBP | + |
| AF055026 | RPIP8 | + |
| BC020740 | SGCD | + |
| AF519179 | SMOX | − |
| AF391283 | SSA1 | − |
| BC012088 | TAF10 | − |
| BC000125 | TGFB1 | − |
| AF050110 | TIEG | − |
| AY065346 | TNFAIP1 | − |
| AF019413 | TNXB | − |
| AK025459 | TRA1 | + |
| AJ440721 | TXNDC5 | + |
| AB062290 | TYMS | + |
| BC000379 | UBB | + |
| AB003730 | UBC | + |
| AF002224, | UBE3A | − |
| AF001787 | UCP3 | + |
| AF135372 | VAMP2 | − |
| AB029013 | WHSC1 | − |
| AB023214 | ZBTB1 | − |
| AF060865 | ZNF205 | + |
| AF055077 | ZNF42 | + |

TABLE 8

| | | GnRHa |
|---|---|---|
| | | 2h vs |
| | | TGF- |
| | | βRII |
| | | antisense |
| Gene Accesion# | Gene Symbol | p ≦ 0.001 |
| AK000002 | ABCC10 | + |
| AF129756 | AIF1 | + |
| AA114994 | ARGBP2 | + |
| BC014450 | B7 | − |
| AB005298 | BAI2 | − |
| AF090947 | BBS4 | + |
| AB038670 | BDNF | + |
| AC006378 | BET1 | + |
| AB018271 | BPAG1 | + |
| AC000391 | BRD3 | + |
| AF016270 | BRD8 | + |
| AJ420534 | C6orf145 | − |
| AB029331 | C6orf18 | + |
| AF072164 | C9orf33 | + |
| AC002543 | CAPZA2 | − |
| BC015799 | CASP7 | + |
| BC036787 | CTF1 | − |
| AF280107 | CYP3A5 | + |
| BC000485 | DDC | − |
| AB018284 | EIF5B | + |
| AF253417 | EPHX1 | − |
| AI879202 | ETHE1 | − |
| BC001325 | FUBP3 | − |
| AB058690 | GPS2 | + |
| AY136740 | GPSM2 | + |
| NM_000855 | GUCY1A2 | + |
| X83412 | HAB1 | + |
| | HERV-K(HML6) | − |
| AF299094 | HSF1 | − |
| AY136751 | HTR2B | + |
| BC015335 | ICT1 | + |
| AF011889 | IDS | + |
| BC002793 | IFNAR2 | − |
| AF117108 | IMP-3 | + |
| AF003837 | JAG1 | + |
| AF072467 | JRK | + |
| AF361886 | KEAP1 | − |
| AB014564 | KIAA0664 | − |
| BC034041 | LMO2 | + |
| AK074703 | LOC89944 | + |
| AF000177 | LSM1 | + |
| AK025599 | MAN1A1 | + |
| AK124738 | MAP4K5 | + |
| AK025602 | MGC2747 | + |
| AB037859 | MKL1 | − |
| AF102544 | MOCS3 | − |
| BC006491 | MPZ | + |
| AB037663 | MYLK | + |
| AF113003 | NCOR2 | − |
| AF044958 | NDUFB8 | + |
| BC002421 | NEF3 | + |
| AB010710 | OLR1 | − |
| AY189737 | OVGP1 | + |
| AB014608 | PARC | + |
| AL133335 | PFDN4 | + |
| AJ419231 | PHC2 | − |
| AF006501 | POLR2F | + |
| AK095191 | POU6F1 | − |
| AF045569 | PRKCH | + |
| NM_006256 | PRKCL2 | + |
| AF007157 | PRNPIP | − |
| | N-Cym | + |
| AK074531 | PRR3 | − |
| AF332577 | PSMA6 | + |
| AF000231 | RAB11A | − |
| AF125393 | RAB27A | + |
| BC002585 | RAB7L1 | + |
| D38076 | RANBP1 | − |
| AB112074 | RBBP6 | + |
| BC007102 | RQCD1 | − |
| AF072825 | RREB1 | + |
| AC004381 | SAH | + |
| AF015224 | SCGB2A2 | + |
| AF029081 | SFN | + |
| AK127319 | SLC16A3 | − |
| BC041164 | SMPD1 | − |
| AB046845 | SMURF1 | − |
| AB030036 | ST14 | + |
| AF070532 | SUPT6H | − |
| AJ549245 | TAF1 | + |
| BC029891 | TFEC | + |
| BC000866 | TIMP1 | + |
| AF139460 | ZNF288 | + |
| BC015961 | ADM | + |
| AF129756 | AIF1 | + |
| AY341427 | AP2B1 | + |
| BC004537 | ATP6V0C | − |
| BC008861 | ATP6V0D1 | − |
| AB009598 | B3GAT3 | + |
| AB029331 | C6orf18 | + |
| AF078803 | CAMK2B | + |
| BC015799 | CASP7 | + |
| AB025105 | CDH1 | + |
| AB001090 | CDH13 | + |
| AB037187 | CHST7 | + |
| AK122769 | CKMT2 | + |
| AB032372 | CKTSF1B1 | + |
| AF000959 | CLDN5 | + |
| AF053318 | CNOT8 | + |
| BC022069 | CRABP1 | + |
| BC003015 | DGCR14 | + |
| BC038231 | DUSP8 | + |
| BC020746 | DXS1283E | + |
| J03066 | EN2 | + |
| BC002706 | ERBB3 | − |
| BC002706 | ERBB3 | − |
| AI879202 | ETHE1 | − |
| AF241235 | FXYD2 | + |
| AF124491 | GIT2 | + |
| | Glial Growth Factor 2 | + |
| AL133324 | GSS | + |
| AB032481 | HOXD13 | + |
| AF299094 | HSF1 | − |
| AF441399 | HSGP25L2G | + |
| AF275719 | HSPCB | + |
| AB030304 | HUMGT198A | + |
| BC014972 | IL2RG | + |
| AB012853 | ING1L | + |
| AF361886 | KEAP1 | − |
| BC005407 | KIAA0169 | + |
| BC014932 | KIAA0280 | − |
| AB007887 | KIAA0427 | − |
| AB028953 | KIAA1030 | + |
| BC014781 | LCAT | + |
| AB016485 | LDB1 | − |
| AF072814 | M96 | + |
| AF010193 | MADH7 | − |
| AL137667 | MAPK8 | + |
| AY032603 | MCM3 | − |
| AL137295 | MLLT10 | + |
| AB051340 | MRPL23 | + |
| AB046613 | MYL6 | + |
| NM_004998 | MYO1E | + |
| AF113003 | NCOR2 | − |
| AF013160 | NDUFS2 | + |
| AF020351 | NDUFS4 | + |
| BC013789 | NHLH1 | + |
| | Nuclear Factor 1A | + |
| BC011539 | ORC1L | + |
| AB014887 | ORM1 | + |
| BC006268 | PEX7 | + |
| AK093558 | PFDN1 | + |
| AL133335 | PFDN4 | + |
| BC009899 | PIK3R4 | + |
| BC037246 | PNMT | + |
| AF055028 | POLR2B | + |
| BC031043 | PRH1 | + |
| AB026491 | PRKCABP | + |
| AK074531 | PRR3 | − |
| AF332577 | PSMA6 | + |
| AK023775 | PTPRF | − |
| AF263016 | PTPRR | − |
| BC001390 | QP-C | + |
| BC015460 | QPCT | + |
| AF000231 | RAB11A | − |
| AK055170 | RAE1 | + |
| AF127761 | RBM8A | + |
| AF155595 | RCOR | − |
| BX537448 | SEC14L1 | − |
| AF153609 | SGK | − |
| AF078544 | SLC25A14 | + |
| BC009409 | TACSTD2 | + |
| AF142482 | TEAD3 | − |
| BC000866 | TIMP1 | + |
| AF017146 | TOP3B | + |
| BC016804 | TRAM2 | − |
| BC014243 | TYK2 | − |
| AB028980 | USP24 | + |
| AB017103 | YWHAE | − |
| BC000292 | ACTG1 | + |
| AF023476 | ADAM12 | + |
| AF001042 | ADARB1 | − |
| AB018327 | ADNP | + |
| AF245699 | AGTR1 | + |
| AF129756 | AIF1 | + |
| D45915 | ALK | + |
| AK057883 | AP2M1 | + |
| AK023088 | ARL6IP | − |
| AF001307 | ARNT | + |
| AB018271 | BPAG1 | + |
| AK096489 | BZW1 | + |
| AB029331 | C6orf18 | + |
| AF037335 | CA12 | + |
| AF070589 | CACNA1C | − |
| BC005334 | CETN2 | + |
| AY497547 | CMKLR1 | + |
| NM_001886 | CRYBA4 | + |
| AF361370 | DIA1 | + |
| AF498961 | DRD1 | + |
| AK057845 | EFNA1 | + |
| AI879202 | ETHE1 | − |
| AC002389 | GAPDS | + |
| AF015257 | GPR30 | + |
| AF103803 | H41 | − |
| X83412 | HAB1 | + |
| BC005240 | HAX1 | + |
| AK058013 | HPGD | + |
| BC000290 | IGHMBP2 | + |
| BC015752 | IRF4 | + |
| AK074047 | ITGAX | + |
| AF135158 | JIK | + |
| AF233882 | JUP | − |
| AB020638 | KIAA0831 | + |
| AF115510 | LRRFIP1 | − |
| AF010193 | MADH7 | − |
| AL137667 | MAPK8 | + |
| AK025602 | MGC2747 | + |
| AF125532 | MKNK2 | + |
| BC006491 | MPZ | + |
| AB051340 | MRPL23 | + |
| AF113003 | NCOR2 | − |
| AF013160 | NDUFS2 | + |
| | E6-Ap, | − |
| | Papillomavirus | + |
| BC011539 | ORC1L | + |
| BC000398 | PAFAH1B2 | + |
| AL117618 | PDHB | + |
| AB002107 | PER1 | − |
| BC062602 | PNN | − |
| AK095191 | POU6F1 | − |
| BC013154 | PPP2R5E | − |
| AK055139 | PTK2 | − |
| AF218026 | PTOV1 | − |
| AF008591 | RAC3 | − |
| AL701206 | RARG | + |
| AF127761 | RBM8A | − |
| AF155595 | RCOR | + |
| AB007148 | RPS3A | − |
| BC007102 | RQCD1 | − |
| BC005927 | SERPINE1 | + |
| AB007897 | SETBP1 | + |
| BC009362 | SETDB1 | + |
| AF029081 | SFN | + |
| AF368279 | SGTA | − |
| AK000416 | SLC16A5 | + |
| AF078544 | SLC25A14 | + |
| AK127096 | SLC30A3 | + |
| AY142112 | SLC4A3 | + |
| BC009409 | TACSTD2 | + |
| AB006630 | TCF20 | − |
| AF142482 | TEAD3 | + |
| BC000866 | TIMP1 | + |
| BC029516 | TNP1 | + |
| AF038009 | TPST1 | − |
| AY245544 | TRB2 | + |
| AF104927 | TTLL1 | + |
| BX537824 | TXNIP | + |
| AB002155 | UPK1B | + |
| AF122922 | WIF1 | |

TABLE 9

| Category | Group | Gene Symbol | Gene Name |
|---|---|---|---|
| | All Group | FBLN5 | fibulin 5 |
| | All Group | ECM2 | extracellular matrix |
| | | | protein 2, female organ |
| | | | and adipocyte specific |
| Cell adhesion | Other cell adhesion | SDC4 | syndecan 4 |
| molecule | molecule | | |
| Cell adhesion | Kinase modulator | ICAM2 | intercellular adhesion |
| molecule | | | molecule 2 |
| Extracellular matrix | Extracellular matrix | THBS1 | thrombospondin 1 |
| | glycoprotein | | |
| Extracellular matrix | Extracellular matrix | COL7A1 | collagen, type VII, |
| | structural protein | | alpha 1 |
| Extracellular matrix | Other extracellular | FMOD | fibromodulin |
| | matrix | | |
| Extracellular matrix | Extracellular matrix | COL18A1 | collagen, type XVIII, |
| | structural protein | | alpha 1 |
| Kinase | Protein kinase | WEE1 | WEE1 homolog (S. pombe) |
| Molecular function | Miscellaneous function | TNFRSF5 | tumor necrosis factor |
| unclassified | | | receptor superfamily 5 |
| Transcription factor | Miscellaneous function | NCOA6 | nuclear receptor |
| | | | coactivator 6 |
| Molecular function | Miscellaneous function | GAS1 | growth arrest-specific 1 |
| unclassified | | | |
| Molecular function | Molecular function | ESM1 | endothelial cell- |
| unknown | unknown | | specific molecule 1 |
| Oxidoreductase | Oxygenase | HMOX1 | heme oxygenase |
| | | | (decycling) 1 |
| Protease | Cysteine-type protease | CASP8 | caspase 8, apoptosis- |
| | | | related cysteine |
| | | | protease |
| Protease | Cam family adhesion | ADAM17 | a disintegrin and |
| | molecule | | metalloproteinase |
| | | | domain 17 (tumor |
| | | | necrosis factor, alpha, |
| | | | converting enzyme) |
| Receptor | G-protein coupled | GPR30 | G protein-coupled |
| | receptor | | receptor 30 |
| Receptor | Cytokine receptor | TNFRSF6 | tumor necrosis factor |
| | | | receptor superfamily 6 |
| Select regulatory | Kinase modulator | CCND2 | cyclin D2 |
| molecule | | | |
| Select regulatory | Protease inhibitor | CST7 | cystatin F |
| molecule | | | (leukocystatin) |
| Select regulatory | Protease inhibitor | CST6 | cystatin E/M |
| molecule | | | |
| Select regulatory | Kinase modulator | CCNE1 | cyclin E1 |
| molecule | | | |
| Signaling molecule | Protein/peptide hormone | EDN1 | endothelin 1 |
| Signaling molecule | Protein/peptide hormone | STC2 | stanniocalcin 2 |
| Signaling molecule | Cytokine | IL11 | interleukin 11 |
| Signaling molecule | Chemokine | CCL3 | chemokine (C-C motif) |
| | | | ligand 3 |
| Signaling molecule | Cytokine | IL15 | interleukin 15 |
| Signaling molecule | Other signaling molecule | CTNNB1 | catenin (cadherin- |
| | | | associated protein), b1 |
| Signaling molecule | Other signaling molecule | HUMGT198A | GT198, complete ORF |
| Signaling molecule | Cytokine | CXCL10 | chemokine (C-X-C |
| | | | motif) ligand 10 |
| Signaling molecule | Growth factor | CXCL12 | chemokine (C-X-C |
| | | | motif) ligand 12 |
| | | | (stromal cell-derived |
| | | | factor 1) |
| Signaling molecule | Cytokine | IL17 | interleukin 17 |
| | | | (cytotoxic T- |
| | | | lymphocyte-associated |
| | | | serine esterase 8) |
| Signaling molecule | Chemokine | CXCL5 | chemokine (C-X-C |
| | | | motif) ligand 5 |
| Signaling molecule | Cytokine | IL13 | interleukin 13 |
| Synthase and | Synthase | TYMS | thymidylate synthetase |
| synthetase | | | |
| Transcription factor | Zinc finger transcription | TIEG | TGFB inducible early |
| | factor | | growth response |
| Transcription factor | Homeobox transcription | TGIF | TGFB-induced factor |
| | factor | | (TALE family |
| | | | homeobox) |
| Transcription factor | Other transcription factor | RUNX3 | runt-related |
| | | | transcription factor 3 |
| Transcription factor | Zinc finger transcription | LHX1 | LIM homeobox 1 |
| | factor | | |
| Transcription factor | Other transcription factor | E2F1 | E2F transcription factor 1 |
| Transcription factor | Zinc finger transcription | EGR3 | early growth response 3 |
| | factor | | |
| Transcription factor | Transcription cofactor | CITED2 | Cbp/p300-interacting |
| | | | transactivator, with |
| | | | Glu/Asp-rich carboxy- |
| | | | terminal domain, 2 |
| Transcription factor | Transcription cofactor | EP300 | E1A binding protein |
| | | | p300 |
| Transcription factor | Nuclear hormone | NR4A1 | nuclear receptor |
| | receptor | | subfamily 4, group A, |
| | | | member 1 |
| Transcription factor | Other transcription factor | RUNX1 | runt-related |
| | | | transcription factor 1 |
| | | | (acute myeloid |
| | | | leukemia 1; aml1 |
| | | | oncogene) |
| Transferase | Methyltransferase | MGMT | O-6-methylguanine- |
| | | | DNA methyltransferase |

1: A method for identifying a modulator of at least one gene that is differentially-expressed in fibrotic tissue or during fibrogenesis, or a polypeptide encoded by the differentially-expressed gene, in a cell population, comprising: (a) contacting the cell population with a test agent under conditions effective for the test agent to modulate a differentially-expressed gene, to modulate the biological activity of the polypeptide encoded by the differentially-expressed gene; and (b) determining whether the test agent modulates the expression of the gene or biological activity of the polypeptide encoded by the gene.

2: The method of claim 1, wherein said determining step comprises detecting mRNA or the polypeptide encoded by the differentially-expressed gene.

3. (canceled)

4: The method of claim 1, wherein the cell population comprises endometrial cells of the female reproductive tract.

5: The method of claim 1, wherein the cell population comprises human cells.

6: The method of claim 1, wherein the at least one differentially expressed gene includes at least one gene selected from the group consisting of docking protein 1, 62 kD (downstream of tyrosine kinase 1); centromere protein A (17 kD); catenin (cadherin-associated protein), beta 1 (88 kD); nuclear receptor subfamily 1, group I, member 2; v-rel avian reticuloendotheliosis viral oncogene homolog A; LGN Protein; CDC28 protein kinase 1; hypothetical protein; solute carrier family 17 (sodium phosphate), member 1; FOS-like antigen-1; nuclear matrix protein p84; LERK-6 (EPLG6); visinin-like 1; phosphodiesterase 10A; KH-type splicing regulatory protein (FUSE binding protein 2); Polyposis locus (DP1 gene) mRNA; microtubule-associated protein 2; CDC5 (cell division cycle 5, S pombe, homolog)-like; Centromere autoantigen C (CENPC) mRNA; RNA guanylyltransferase and 5′-phosphatase; Nijmegen breakage syndrome 1 (nibrin); ribonuclease, RNase A family, 4; keratin 10 (epidermolytic hyperkeratosis; keratosis palmaris et plantaris); basic helix-loop-helix domain containing, class B, 2; dual specificity phosphatase 1; annexin A11; putative receptor protein; Human endogenous retrovirus HERV-K(HML6); mitogen-activated protein kinase kinase kinase 12; TXK tyrosine kinase; kynureninase (L-kynurenine hydrolase); ubiquitin specific protease 4 (proto-oncogene); peroxisome biogenesis factor 13; olfactory receptor, family 2, subfamily F, member 1; membrane protein, palmitoylated 3 (MAGUK p55 subfamily member 3); origin recognition complex, subunit 1 (yeast homolog)-like; dTDP-D-glucose 4,6-dehydratase; cytochrome c oxidase subunit VIa polypeptide 2; gamma-tubulin complex protein 2; Monocyte chemotactic protein-3; myelin transcription factor 1; inhibitor of growth family, member 1-like; thyroid hormone receptor, alpha myosin-binding protein C, slow-type; fragile X mental retardation 2; sonic hedgehog (Drosophila) homolog; 6-phosphofructo-2-kinase/fructose-2,6-biphosphatase 2; SFRS protein kinase 2; excision repair cross-complementing rodent repair deficiency; cyclin-dependent kinase 5, regulatory subunit 1 (p35); poly(A)-specific ribonuclease (deadenylation nuclease); solute carrier family 12 (potassium/chloride transporters), member 4; Pseudogene for metallothionein; natriuretic peptide precursor A; intercellular adhesion molecule 2; apoptosis antagonizing transcription factor; similar to rat HREV 107; major histocompatibility complex, class II, DP beta 1; MpV17 transgene, murine homolog, glomerulosclerosis; uroporphyrinogen decarboxylase; proteasome (prosome, macropain) 26S subunit, ATPase, 1; fms-related tyrosine kinase 3 ligand; actin, gamma 1; Protein Kinase Pitslre, Alpha, Alt. Splice 1-Feb; nuclear factor of kappa light polypeptide gene enhancer in B-cells inhibitor, alpha; pyruvate kinase, muscle; telomeric repeat binding factor 2; cell division cycle 2, G1 to S and G2 to M; ADP-ribosylation factor 3; NRF1 Protein; H factor (complement)-like 3; serine (or cysteine) proteinase inhibitor, clade B (ovalbumin), member 6; mRNA of muscle specific gene M9; solute carrier family 25 (mitochondrial carrier; phosphate carrier), member 3; ribosomal protein L36a; suppressor of Ty (S. cerevisiae) 4 homolog 1; amino-terminal enhancer of split; ubiquitin A-52 residue ribosomal protein fusion product 1; hydroxyacyl-Coenzyme A dehydrogenase/3-ketoacyl-Coenzyme A thiolase; chaperonin containing TCP1, subunit 2 (beta); tyrosine kinase with immunoglobulin and epidermal growth factor homology; domains; Fc fragment of IgG, receptor, transporter, alpha; NRD1 convertase; ADP-ribosylation factor 5; transcription elongation factor A (SII), 1; like mouse brain protein E46; titin; fibromodulin; and Abi-interactor 2 (Abi-2).

7: The method of claim 1, wherein the at least one differentially expressed gene includes at least one gene selected from the group consisting of CDKN1B, CDKN1C, CTGF, fibromodulin, and Abi-2.

8: The method of claim 1, wherein the at least one differentially expressed gene includes at least one of IL-11, IL-13, EGR1, EGR2, EGR3, CITED2, P300, E2F1, E2F2, E2F3, E2F4, E2F5, MCP3, CXCL5, CCL7, SMAD3, TYMS, GT198, SMAD7, NCOR2, TIMP-1, and ADAM17.

9: The method of claim 1, wherein the at least one differentially-expressed gene includes at least one of those genes listed in Table 9.

10: The method of claim 1, wherein the at least one differentially-expressed gene includes at least one gene selected from the group consisting of stanniocalcin 2, interleukin 11, disintegrin and metalloproteinase domain 17, early growth response 3, fibromodulin, collagen type XVIII alpha 1, and interleukin 13.

11: The method of claim 1, wherein the at least one differentially expressed gene includes a plurality of genes comprising stanniocalcin 2, interleukin 11, disintegrin and metalloproteinase domain 17, early growth response 3, fibromodulin, collagen type XVIII alpha 1, and interleukin 13.

12: A method for detecting a fibrotic disorder in a subject by: (a) providing a biological sample obtained from the subject; (b) analyzing the expression of at least one gene that is differentially expressed in the fibrotic disorder of interest as compared to normal tissue; and (c) correlating the expression of the at least one differentially expressed gene with the presence or absence of the fibrotic disorder in the subject.

13: The method of claim 12, wherein the fibrotic disorder is a fibrotic disorder of the female reproductive tract.

14: The method of claim 12, wherein the fibrotic disorder is a uterine fibrosis.

15: The method of claim 12, wherein the fibrotic disorder is a fibrotic disorder of the female reproductive tract selected from the group consisting of leiomyoma, endometriosis, ovarian hyperstimulation syndrome, adhesion, and endometrial cancer.

16: The method of claim 12, wherein the sample comprises smooth muscle cells, endometrium, or peritoneal fluid.

17. (canceled)

18: The method of claim 12, wherein the normal tissue comprises myometrium.

19: The method of claim 12, wherein the at least one differentially expressed gene includes at least one gene selected from the group consisting of docking protein 1, 62 kD (downstream of tyrosine kinase 1); centromere protein A (17 kD); catenin (cadherin-associated protein), beta 1 (88 kD); nuclear receptor subfamily 1, group I, member 2; v-rel avian reticuloendotheliosis viral oncogene homolog A; LGN Protein; CDC28 protein kinase 1; hypothetical protein; solute carrier family 17 (sodium phosphate), member 1; FOS-like antigen-1; nuclear matrix protein p84; LERK-6 (EPLG6); visinin-like 1; phosphodiesterase 10A; KH-type splicing regulatory protein (FUSE binding protein 2); Polyposis locus (DP1 gene) mRNA; microtubule-associated protein 2; CDC5 (cell division cycle 5, S pombe, homolog)-like; Centromere autoantigen C (CENPC) mRNA; RNA guanylyltransferase and 5′-phosphatase; Nijmegen breakage syndrome 1 (nibrin); ribonuclease, RNase A family, 4; keratin 10 (epidermolytic hyperkeratosis; keratosis palmaris et plantaris); basic helix-loop-helix domain containing, class B, 2; dual specificity phosphatase 1; annexin A11; putative receptor protein; Human endogenous retrovirus HERV-K(HML6); mitogen-activated protein kinase kinase kinase 12; TXK tyrosine kinase; kynureninase (L-kynurenine hydrolase); ubiquitin specific protease 4 (proto-oncogene); peroxisome biogenesis factor 13; olfactory receptor, family 2, subfamily F, member 1; membrane protein, palmitoylated 3 (MAGUK p55 subfamily member 3); origin recognition complex, subunit 1 (yeast homolog)-like; dTDP-D-glucose 4,6-dehydratase; cytochrome c oxidase subunit VIa polypeptide 2; gamma-tubulin complex protein 2; Monocyte chemotactic protein-3; myelin transcription factor 1; inhibitor of growth family, member 1-like; thyroid hormone receptor, alpha myosin-binding protein C, slow-type; fragile X mental retardation 2; sonic hedgehog (Drosophila) homolog; 6-phosphofructo-2-kinase/fructose-2,6-biphosphatase 2; SFRS protein kinase 2; excision repair cross-complementing rodent repair deficiency; cyclin-dependent kinase 5, regulatory subunit 1 (p35); poly(A)-specific ribonuclease (deadenylation nuclease); solute carrier family 12 (potassium/chloride transporters), member 4; Pseudogene for metallothionein; natriuretic peptide precursor A; intercellular adhesion molecule 2; apoptosis antagonizing transcription factor; similar to rat HREV 107; major histocompatibility complex, class II, DP beta 1; MpV17 transgene, murine homolog, glomerulosclerosis; uroporphyrinogen decarboxylase; proteasome (prosome, macropain) 26S subunit, ATPase, 1; fms-related tyrosine kinase 3 ligand; actin, gamma 1; Protein Kinase Pitslre, Alpha, Alt. Splice 1-Feb; nuclear factor of kappa light polypeptide gene enhancer in B-cells inhibitor, alpha; pyruvate kinase, muscle; telomeric repeat binding factor 2; cell division cycle 2, G1 to S and G2 to M; ADP-ribosylation factor 3; NRF1 Protein; H factor (complement)-like 3; serine (or cysteine) proteinase inhibitor, clade B (ovalbumin), member 6; mRNA of muscle specific gene M9; solute carrier family 25 (mitochondrial carrier; phosphate carrier), member 3; ribosomal protein L36a; suppressor of Ty (S. cerevisiae) 4 homolog 1; amino-terminal enhancer of split; ubiquitin A-52 residue ribosomal protein fusion product 1; hydroxyacyl-Coenzyme A dehydrogenase/3-ketoacyl-Coenzyme A thiolase; chaperonin containing TCP1, subunit 2 (beta); tyrosine kinase with immunoglobulin and epidermal growth factor homology; domains; Fc fragment of IgG, receptor, transporter, alpha; NRD1 convertase; ADP-ribosylation factor 5; transcription elongation factor A (SII), 1; like mouse brain protein E46; titin; fibromodulin; and Abi-interactor 2 (Abi-2).

20: The method of claim 12, wherein the at least one differentially expressed gene includes at least one gene selected from the group consisting of CDKN1B, CDKN1C, CTGF, fibromodulin, and Abi-2.

21: The method of claim 12, wherein the at least one differentially expressed gene includes at least one of IL-11, IL-13, EGR1, EGR2, EGR3, CITED2, P300, E2F1, E2F2, E2F3, E2F4, E2F5, MCP3, CXCL5, CCL7, SMAD3, TYMS, GT198, SMAD7, NCOR2, TIMP-1, and ADAM17, wherein elevated expression of IL-11, IL-13, EGR1, EGR2, EGR3, CITED2, P300, E2F1, E2F2, E2F3, E2F4, E2F5, MCP3, CXCL5, CCL7, SMAD3, TYMS, and/or GT198 is indicative of a fibrotic disorder; and wherein reduced expression of SMAD7, NCOR2, TIMP-1, and/or ADAM17 is indicative of a fibrotic disorder.

22: The method of claim 12, wherein the at least one differentially expressed gene includes at least one of those genes listed in Table 9.

23: The method of claim 12, wherein the at least one differentially expressed gene includes at least one gene selected from the group consisting of stanniocalcin 2, interleukin 11, disintegrin and metalloproteinase domain 17, early growth response 3, fibromodulin, collagen type XVIII alpha 1, and interleukin 13.

24: The method of claim 12, wherein the at least one differentially expressed gene includes a plurality of genes comprising stanniocalcin 2, interleukin 11, disintegrin and metalloproteinase domain 17, early growth response 3, fibromodulin, collagen type XVIII alpha 1, and interleukin 13.

25: The method of claim 12, wherein the subject is human.

26: The method of claim 12, further comprising diagnosing the subject based on said correlating.

27: A method for modulating gene expression in fibrotic tissue, comprising contacting the fibrotic tissue in vitro or in vivo with an agent that modulates expression of at least one differentially expressed gene in the tissue.

28: The method of claim 27, wherein the agent is a TGF-beta signaling inhibitor.

29: The method of claim 27, wherein the agent is a TGF-beta II receptor inhibitor.

30. (canceled)

31: The method of claim 27, wherein the at least one differentially expressed gene includes at least one gene selected from the group consisting of docking protein 1, 62 kD (downstream of tyrosine kinase 1); centromere protein A (17 kD); catenin (cadherin-associated protein), beta 1 (88 kD); nuclear receptor subfamily 1, group I, member 2; v-rel avian reticuloendotheliosis viral oncogene homolog A; LGN Protein; CDC28 protein kinase 1; hypothetical protein; solute carrier family 17 (sodium phosphate), member 1; FOS-like antigen-1; nuclear matrix protein p84; LERK-6 (EPLG6); visinin-like 1; phosphodiesterase 10A; KH-type splicing regulatory protein (FUSE binding protein 2); Polyposis locus (DP1 gene) mRNA; microtubule-associated protein 2; CDC5 (cell division cycle 5, S pombe, homolog)-like; Centromere autoantigen C (CENPC) mRNA; RNA guanylyltransferase and 5′-phosphatase; Nijmegen breakage syndrome 1 (nibrin); ribonuclease, RNase A family, 4; keratin 10 (epidermolytic hyperkeratosis; keratosis palmaris et plantaris); basic helix-loop-helix domain containing, class B, 2; dual specificity phosphatase 1; annexin A11; putative receptor protein; Human endogenous retrovirus HERV-K(HML6); mitogen-activated protein kinase kinase kinase 12; TXK tyrosine kinase; kynureninase (L-kynurenine hydrolase); ubiquitin specific protease 4 (proto-oncogene); peroxisome biogenesis factor 13; olfactory receptor, family 2, subfamily F, member 1; membrane protein, palmitoylated 3 (MAGUK p55 subfamily member 3); origin recognition complex, subunit 1 (yeast homolog)-like; dTDP-D-glucose 4,6-dehydratase; cytochrome c oxidase subunit VIa polypeptide 2; gamma-tubulin complex protein 2; Monocyte chemotactic protein-3; myelin transcription factor 1; inhibitor of growth family, member 1-like; thyroid hormone receptor, alpha myosin-binding protein C, slow-type; fragile X mental retardation 2; sonic hedgehog (Drosophila) homolog; 6-phosphofructo-2-kinase/fructose-2,6-biphosphatase 2; SFRS protein kinase 2; excision repair cross-complementing rodent repair deficiency; cyclin-dependent kinase 5, regulatory subunit 1 (p35); poly(A)-specific ribonuclease (deadenylation nuclease); solute carrier family 12 (potassium/chloride transporters), member 4; Pseudogene for metallothionein; natriuretic peptide precursor A; intercellular adhesion molecule 2; apoptosis antagonizing transcription factor; similar to rat HREV107; major histocompatibility complex, class II, DP beta 1; MpV17 transgene, murine homolog, glomerulosclerosis; uroporphyrinogen decarboxylase; proteasome (prosome, macropain) 26S subunit, ATPase, 1; fms-related tyrosine kinase 3 ligand; actin, gamma 1; Protein Kinase Pitslre, Alpha, Alt. Splice 1-Feb; nuclear factor of kappa light polypeptide gene enhancer in B-cells inhibitor, alpha; pyruvate kinase, muscle; telomeric repeat binding factor 2; cell division cycle 2, G1 to S and G2 to M; ADP-ribosylation factor 3; NRF1 Protein; H factor (complement)-like 3; serine (or cysteine) proteinase inhibitor, clade B (ovalbumin), member 6; mRNA of muscle specific gene M9; solute carrier family 25 (mitochondrial carrier; phosphate carrier), member 3; ribosomal protein L36a; suppressor of Ty (S. cerevisiae) 4 homolog 1; amino-terminal enhancer of split; ubiquitin A-52 residue ribosomal protein fusion product 1; hydroxyacyl-Coenzyme A dehydrogenase/3-ketoacyl-Coenzyme A thiolase; chaperonin containing TCP1, subunit 2 (beta); tyrosine kinase with immunoglobulin and epidermal growth factor homology; domains; Fc fragment of IgG, receptor, transporter, alpha; NRD1 convertase; ADP-ribosylation factor 5; transcription elongation factor A (SII), 1; like mouse brain protein E46; titin; fibromodulin; and Abi-interactor 2 (Abi-2).

32: The method of claim 27, wherein the at least one differentially expressed gene includes at least one gene selected from the group consisting of CDKN1B, CDKN1C, CTGF, fibromodulin, and Abi-2.

33: The method of claim 27, wherein the at least one differentially expressed gene includes at least one of IL-11, IL-13, EGR1, EGR2, EGR3, CITED2, P300, E2F1, E2F2, E2F3, E2F4, E2F5, MCP3, CXCL5, CCL7, SMAD3, TYMS, GT198, SMAD7, NCOR2, TIMP-1, and ADAM17.

34: The method of claim 27, wherein the at least one differentially expressed gene includes at least one of those genes listed in Table 9.

35: The method of claim 27, wherein the at least one differentially expressed gene includes at least one gene selected from the group consisting of stanniocalcin 2, interleukin 11, disintegrin and metalloproteinase domain 17, early growth response 3, fibromodulin, collagen type XVIII alpha 1, and interleukin 13.

36: The method of claim 27, wherein the at least one differentially expressed gene includes a plurality of genes comprising stanniocalcin 2, interleukin 11, disintegrin and metalloproteinase domain 17, early growth response 3, fibromodulin, collagen type XVIII alpha 1, and interleukin 13.

37: The method of claim 27, wherein the agent is a selective estrogen receptor modulator (SERM), selective progesterone receptor modulator (SPRM), or mast cell inhibitor.

38-39. (canceled)

40: The method of claim 27, wherein the agent has a pyrazolopyridine scaffold, a pyrazole scaffold, an imadazpyridine scaffold, a triazole scaffold, a pyridopyrimidine scaffold, or an isothiazole scaffold.

41: The method of claim 27, wherein the agent is a GnRh agonist or antagonist.

42: The method of claim 27, wherein the agent is at least one selected from the group consisting of roloxifene; asoprisnil (J867); RU486; SB-505124; SB-431542; tranlist; cystatin C (CystC); SD-208; LY550410; LY580276; pitavastatin; 1,-5 naphthyridine amiothiazole derivative; 1,5 naphthyridine pyrazole derivative; and ursolic acid.

43: An array comprising a substrate having a plurality of addresses, wherein each address disposed thereon has a capture probe that can specifically bind at least one polynucleotide that is differentially expressed in fibrotic disorders, or a complement thereof.

44: The array of claim 43, wherein the at least one polynucleotide is selected from the group consisting of docking protein 1, 62 kD (downstream of tyrosine kinase 1); centromere protein A (17 kD); catenin (cadherin-associated protein), beta 1 (88 kD); nuclear receptor subfamily 1, group I, member 2; v-rel avian reticuloendotheliosis viral oncogene homolog A; LGN Protein; CDC28 protein kinase 1; hypothetical protein; solute carrier family 17 (sodium phosphate), member 1; FOS-like antigen-1; nuclear matrix protein p84; LERK-6 (EPLG6); visinin-like 1; phosphodiesterase 10A; KH-type splicing regulatory protein (FUSE binding protein 2); Polyposis locus (DP1 gene) mRNA; microtubule-associated protein 2; CDC5 (cell division cycle 5, S pombe, homolog)-like; Centromere autoantigen C (CENPC) mRNA; RNA guanylyltransferase and 5′-phosphatase; Nijmegen breakage syndrome 1 (nibrin); ribonuclease, RNase A family, 4; keratin 10 (epidermolytic hyperkeratosis; keratosis palmaris et plantaris); basic helix-loop-helix domain containing, class B, 2; dual specificity phosphatase 1; annexin A11; putative receptor protein; Human endogenous retrovirus HERV-K(HML6); mitogen-activated protein kinase kinase kinase 12; TXK tyrosine kinase; kynureninase (L-kynurenine hydrolase); ubiquitin specific protease 4 (proto-oncogene); peroxisome biogenesis factor 13; olfactory receptor, family 2, subfamily F, member 1; membrane protein, palmitoylated 3 (MAGUK p55 subfamily member 3); origin recognition complex, subunit 1 (yeast homolog)-like; dTDP-D-glucose 4,6-dehydratase; cytochrome c oxidase subunit VIa polypeptide 2; gamma-tubulin complex protein 2; Monocyte chemotactic protein-3; myelin transcription factor 1; inhibitor of growth family, member 1-like; thyroid hormone receptor, alpha myosin-binding protein C, slow-type; fragile X mental retardation 2; sonic hedgehog (Drosophila) homolog; 6-phosphofructo-2-kinase/fructose-2,6-biphosphatase 2; SFRS protein kinase 2; excision repair cross-complementing rodent repair deficiency; cyclin-dependent kinase 5, regulatory subunit 1 (p35); poly(A)-specific ribonuclease (deadenylation nuclease); solute carrier family 12 (potassium/chloride transporters), member 4; Pseudogene for metallothionein; natriuretic peptide precursor A; intercellular adhesion molecule 2; apoptosis antagonizing transcription factor; similar to rat HREV107; major histocompatibility complex, class II, DP beta 1; MpV17 transgene, murine homolog, glomerulosclerosis; uroporphyrinogen decarboxylase; proteasome (prosome, macropain) 26S subunit, ATPase, 1; fins-related tyrosine kinase 3 ligand; actin, gamma 1; Protein Kinase Pitslre, Alpha, Alt. Splice 1-Feb; nuclear factor of kappa light polypeptide gene enhancer in B-cells inhibitor, alpha; pyruvate kinase, muscle; telomeric repeat binding factor 2; cell division cycle 2, G1 to S and G2 to M; ADP-ribosylation factor 3; NRF1 Protein; H factor (complement)-like 3; serine (or cysteine) proteinase inhibitor, clade B (ovalbumin), member 6; mRNA of muscle specific gene M9; solute carrier family 25 (mitochondrial carrier; phosphate carrier), member 3; ribosomal protein L36a; suppressor of Ty (S. cerevisiae) 4 homolog 1; amino-terminal enhancer of split; ubiquitin A-52 residue ribosomal protein fusion product 1; hydroxyacyl-Coenzyme A dehydrogenase/3-ketoacyl-Coenzyme A thiolase; chaperonin containing TCP1, subunit 2 (beta); tyrosine kinase with immunoglobulin and epidermal growth factor homology; domains; Fc fragment of IgG, receptor, transporter, alpha; NRD1 convertase; ADP-ribosylation factor 5; transcription elongation factor A (SII), 1; like mouse brain protein E46; titin; fibromodulin; and Abi-interactor 2 (Abi-2).

45: The array of claim 43, wherein the at least one polynucleotide includes at least one gene selected from the group consisting of CDKN1B, CDKN1C, CTGF, fibromodulin, and Abi-2.

46: The array of claim 43, wherein the at least one polynucleotide includes at least one gene selected from the group consisting of IL-11, IL-13, EGR1, EGR2, EGR3, CITED2, P300, E2F1, E2F2, E2F3, E2F4, E2F5, MCP3, CXCL5, CCL7, SMAD3, TYMS, GT198, SMAD7, NCOR2, TIMP-1, and ADAM17.

47: The array of claim 43, wherein the at least one polynucleotide includes at least one of those genes listed in Table 9.

48: The array of claim 43, wherein the at least one polynucleotide includes at least one gene selected from the group consisting of stanniocalcin 2, interleukin 11, disintegrin and metalloproteinase domain 17, early growth response 3, fibromodulin, collagen type XVIII alpha 1, and interleukin 13.

49: The array of claim 43, wherein the at least one polynucleotide includes a plurality of genes comprising stanniocalcin 2, interleukin 11, disintegrin and metalloproteinase domain 17, early growth response 3, fibromodulin, collagen type XVIII alpha 1, and interleukin 13.

50-55. (canceled)
